(19) United States
(12) Patent Application Publication
(10) Pub. No.: US 2024/0018198 A1
(43) Pub. Date: Jan. 18, 2024

(54) MU-DIGUETOXIN-DC1A VARIANT POLYPEPTIDES FOR PEST CONTROL

(71) Applicant: Vestaron Corporation, Durham, NC (US)

(72) Inventors: Kyle SCHNEIDER, Kalamazoo, MI (US); Alexandra HAASE, Martin, MI (US); Breck DAVIS, Kalamazoo, MI (US)

(73) Assignee: Vestaron Corporation, Durham, NC (US)

(21) Appl. No.: 18/028,712

(22) PCT Filed: Sep. 27, 2021
(86) PCT No.: PCT/US2021/052259

§ 371 Date: Mar. 27, 2023

Related U.S. Application Data
(60) Provisional application No. 63/084,339, filed on Sep. 28, 2020.

(51) Int. Cl. C07K 14/435; A01N 63/50; C12N 15/82; C07K 14/39; A61P 33/14

(52) U.S. Cl. CPC C07K 14/43518; A01N 63/50; C12N 15/8286; C07K 14/39; A61P 33/14; A01P 7/04

(57) ABSTRACT

New insecticidal peptides, polypeptides, proteins, and nucleotides; their expression in culture and plants; methods of producing the peptides, polypeptides, proteins, and nucleotides; new processes; new production techniques; new formulations; and new organisms, are disclosed. The present disclosure is also related to a novel type of peptide named Dc1a-Variant Polypeptides (DVPs) that are a non-naturally occurring, modified-form of the peptide, Mu-diguetoxin-Dc1a, isolated from the American Desert Spider (Diguetia canities). Here we describe: genes encoding DVPs; various formulations and combinations of both genes and peptides; and methods for using the same that are useful for the control of insects. Further, the present invention relates to novel, recombinant cysteine rich proteins (CRPs) with a cystine knot (CK) architecture, created by removing one or more disulfide bonds from a polypeptide having four or more disulfide bonds.

### CROSS REFERENCE TO RELATED APPLICATIONS

This application claims the benefit of, and priority to, U.S. Provisional Application Ser. No. 63/084,339, filed on Sep. 28, 2020. The entire contents of the aforementioned application are incorporated herein.

### SEQUENCE LISTING

This application incorporates by reference in its entirety the Sequence Listing entitled “225312-497884_ST25.txt” (126 kilobytes), which was created on Sep. 27, 2021, at 6:32 PM, that is 126 KB, and filed electronically herewith.

### TECHNICAL FIELD

The present disclosure provides insecticidal proteins, nucleotides, peptides, their expression in plants, methods of producing the peptides, new formulations, and methods for the control of insects are described.

### BACKGROUND

Deleterious insects represent a worldwide threat to human health and food security. Insects pose a threat to human health because they are a vector for disease. One of the most notorious insect-vectors of disease is the mosquito. Mosquitoes in the genus Anopheles are the principal vectors of Zika virus, Chikungunya virus, and malaria—a disease caused by protozoa in the genus Trypanosoma. Another mosquito, Aedes aegypti, is the main vector of the viruses that cause Yellow fever and Dengue. And, Aedes spp. mosquitos are also the vectors for the viruses responsible for various types of encephalitis. Wuchereria bancrofti and Brugia malayi, parasitic roundworms that cause filariasis, are usually spread by mosquitoes in the genera Culex, Mansonia, and Anopheles.

Similar to the mosquito, other members of the Diptera order have likewise plagued humankind since time immemorial. In addition to producing painful bites, Horseflies and deerflies transmit the bacterial pathogens of tularemia (Pasteurella tularensis) and anthrax (Bacillus anthracis), as well as a parasitic roundworm (Loa loa) that causes loiasis in tropical Africa.

Blowflies (Chrysomya megacephala) and houseflies (Musca domestica) will in one moment take off from carrion and dung, and in the next moment alight in our homes and on our food-spreading dysentery, typhoid fever, cholera, poliomyelitis, yaws, leprosy, and tuberculosis in their wake.

Eye gnats in the genus Hippelates can carry the spirochaete pathogen that causes yaws (Treponema pertenue), and may also spread conjunctivitis (pinkeye). Tsetse flies in the genus Glossina transmit the protozoan pathogens that cause African sleeping sickness (Trypanosoma gambiense and T. rhodesiense). Sand flies in the genus Phlebotomus are vectors of a bacterium (Bartonella bacilliformis) that causes Carrion's disease (Oroyo fever) in South America. In parts of Asia and North Africa, they spread a viral agent that causes sand fly fever (Pappataci fever) as well as protozoan pathogens (Leishmania spp.) that cause Leishmaniasis.

Human food security is also threatened by insects. Insect pests indiscriminately target food crops earmarked for commercial purposes and personal use alike; indeed, the damage caused by insect pests can run the gamut from mere inconvenience to financial ruin in the former, to extremes such as malnutrition or starvation in the latter. Insect pests also cause stress and disease in domesticated animals. And, insect pests once limited by geographical and climate boundaries have expanded their range due to global travel and climate change.

### SUMMARY

The present disclosure describes a diguetoxin variant polypeptide (DVP) having insecticidal activity against one or more insect species. Here, the DVP comprises an amino acid sequence that is at least 80%, 85%, 90%, or at least 95% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T; or a pharmaceutically acceptable salt thereof.

In addition, the present disclosure describes a composition consisting of a DVP, a DVP-insecticidal protein, or combinations thereof, and an excipient.

The present disclosure describes a polynucleotide operable to encode a DVP, where the DVP comprises an amino acid sequence that is at least 80%, 85%, 90%, or at least 95% identical to the amino acid sequence according to Formula (I): A-X₁-D-G-D-V-E-G-P-A-G-C—K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F—F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T, or a complementary nucleotide sequence thereof.

In addition, the present disclosure describes a method of producing a DVP, the method comprising: preparing a vector comprising a first expression cassette comprising a polynucleotide operable to encode a DVP, and/or a complementary nucleotide sequence thereof, said DVP comprising an amino acid sequence that is at least 80%, 85%, 90%, or at least 95% identical to the amino acid sequence according to Formula (I): A-X₁-D-G-D-V-E-G-P-A-G-C-K—K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁—X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T; introducing the vector into a yeast cell; and growing the yeast cell in a growth medium under conditions operable to enable expression of the DVP and secretion into the growth medium.

The present disclosure describes a method of combating, controlling, or inhibiting a pest comprising, applying a pesticidally effective amount of the composition consisting of a DVP, a DVP-insecticidal protein, or combinations thereof, and an excipient, to the locus of the pest, or to a plant or animal susceptible to an attack by the pest.

In addition, the present disclosure describes a vector comprising a polynucleotide operable to encode a DVP having an amino sequence that is at least 80%, 85%, 90%, or at least 95% identical to an amino acid sequence as set forth in any one of SEQ ID NOs: 6-43, 45-51, 53, 128, 130, 136, 139-140, 144, 146-147, 187-191,202-215, or 217-219.

The present disclosure also describes a yeast strain comprising: a first expression cassette comprising a polynucleotide operable to encode a DVP, said DVP comprising an amino acid sequence that is at least 80%, 85%, 90%, or at least 95% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁—X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T.

In addition, the present disclosure provides a recombinant CRP comprising, consisting essentially of, or consisting of, a cystine knot (CK) architecture according to Formula (II):

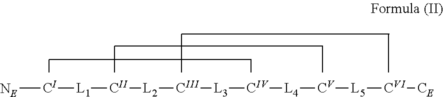

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; wherein said recombinant CRP is created by modifying a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, wherein the modifiable CRP is modified by removing one or more non-CK disulfide bonds from a modifiable CRP having one or more non-CK disulfide bonds; wherein removing the one or more disulfide bonds from the modifiable CRP having one or more non-CK disulfide bonds, results in the recombinant CRP having the CK architecture according to Formula (II); and wherein the recombinant CRP having the CK architecture according to Formula (II) has an increased level of expression relative to a level of expression of a modifiable CRP that does not have the CK architecture according to Formula (II).

In addition, the present disclosure describes a method of making a recombinant cysteine-rich protein (CRP) comprising a cystine knot (CK) architecture according to Formula (II):

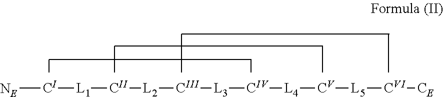

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; said method comprising: (a) providing a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, and (b) modifying the modifiable CRP by removing one or more non-CK disulfide bonds from a modifiable CRP having one or more non-CK disulfide bonds; wherein removing the one or more disulfide bonds from the modifiable CRP having one or more non-CK disulfide bonds, results in the recombinant CRP having the CK architecture according to Formula (II); and wherein the recombinant CRP having the CK architecture according to Formula (II) has an increased level of expression relative to a level of expression of a modifiable CRP that does not have the CK architecture according to Formula (II).

The present disclosure also describes a method of increasing the yield of a recombinant cysteine-rich protein (CRP), said method comprising: (a) creating a recombinant CRP having a cystine knot (CK) architecture according to Formula (II):

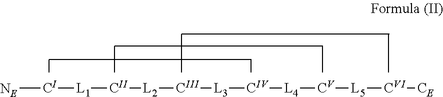

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; wherein said recombinant CRP is created according to the following process: (b) providing a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, (c) modifying the modifiable CRP by removing one or more non-CK disulfide bonds from the modifiable CRP having one or more non-CK disulfide bonds; wherein removing the one or more disulfide bonds from the modifiable CRP having one or more non-CK disulfide bonds results in the recombinant CRP having the CK architecture according to Formula (II); and wherein the recombinant CRP having the CK architecture according to Formula (II) has an increased level of expression relative to a level of expression of a modifiable CRP that does not have the CK architecture according to Formula (II).

In addition, the present disclosure describes a diguetoxin variant polypeptide (DVP) having insecticidal activity against one or more insect species, said DVP comprising an amino acid sequence that is at least 80%, 85%, 90%, or at least 95% identical to the amino acid sequence set forth in any one of SEQ ID NOs: 6-43, 45-51, 53, 128, 130, 136, 139-140, 144, 146-147, 187-191, 202-215, or 217-219, or a pharmaceutically acceptable salt thereof.

In addition, the present disclosure describes a diguetoxin variant polypeptide (DVP) having insecticidal activity against one or more insect species, said DVP consisting of an amino acid sequence that is at least 80%, 85%, 90%, or at least 95% identical to the amino acid sequence set forth in any one of SEQ ID NOs: 6-43, 45-51, 53, 128, 130, 136, 139-140, 144, 146-147, 187-191, 202-215, or 217-219, or a pharmaceutically acceptable salt thereof.

In addition, the present disclosure describes a diguetoxin variant polypeptide (DVP) having insecticidal activity against one or more insect species, said DVP consisting of an amino acid set forth in any one of SEQ ID NOs: 6-43, 45-51, 53, 128, 130, 136, 139-140, 144, 146-147, 187-191, 202-215, or 217-219, or a pharmaceutically acceptable salt thereof.

In addition, the present disclosure describes a diguetoxin variant polypeptide (DVP) having insecticidal activity against one or more insect species, said DVP comprising an amino acid sequence that is at least 80%, 85%, 90%, or at least 95% identical to the amino acid sequence set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a pharmaceutically acceptable salt thereof.

In addition, the present disclosure describes a diguetoxin variant polypeptide (DVP) having insecticidal activity against one or more insect species, said DVP consisting of an amino acid sequence that is at least 80%, 85%, 90%, or at least 95% identical to the amino acid sequence set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a pharmaceutically acceptable salt thereof.

In addition, the present disclosure describes a diguetoxin variant polypeptide (DVP) having insecticidal activity against one or more insect species, said DVP consisting of an amino acid set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a pharmaceutically acceptable salt thereof.

In addition, the present disclosure describes a diguetoxin variant polypeptide (DVP) having insecticidal activity against one or more insect species, said DVP comprising an amino acid sequence that is at least 80%, 85%, 90%, or at least 95% identical to the amino acid sequence set forth in any one of SEQ ID NOs: 47, 53, 136, 139-140, 144, 146-147, 187-191, 210-215, or 217-219, or a pharmaceutically acceptable salt thereof.

In addition, the present disclosure describes a diguetoxin variant polypeptide (DVP) having insecticidal activity against one or more insect species, said DVP consisting of an amino acid sequence that is at least 80%, 85%, 90%, or at least 95% identical to the amino acid sequence set forth in any one of SEQ ID NOs: 47, 53, 136, 139-140, 144, 146-147, 187-191, 210-215, or 217-219, or a pharmaceutically acceptable salt thereof.

In addition, the present disclosure describes a diguetoxin variant polypeptide (DVP) having insecticidal activity against one or more insect species, said DVP consisting of an amino acid set forth in any one of SEQ ID NOs: 47, 53, 136, 139-140, 144, 146-147, 187-191, 210-215, or 217-219, or a pharmaceutically acceptable salt thereof.

In addition, the present disclosure describes a diguetoxin variant polypeptide (DVP) having insecticidal activity against one or more insect species, said DVP comprising an amino acid sequence that is at least 80%, 85%, 90%, or at least 95% identical to the amino acid sequence set forth in any one of SEQ ID NOs: 213, or 217-219, or a pharmaceutically acceptable salt thereof.

In addition, the present disclosure describes a diguetoxin variant polypeptide (DVP) having insecticidal activity against one or more insect species, said DVP consisting of an amino acid sequence that is at least 80%, 85%, 90%, or at least 95% identical to the amino acid sequence set forth in any one of SEQ ID NOs: 213, or 217-219, or a pharmaceutically acceptable salt thereof.

In addition, the present disclosure describes a diguetoxin variant polypeptide (DVP) having insecticidal activity against one or more insect species, said DVP consisting of an amino acid set forth in any one of SEQ ID NOs: 213, or 217-219, or a pharmaceutically acceptable salt thereof.

In addition, the present disclosure describes a diguetoxin variant polypeptide (DVP) having insecticidal activity against one or more insect species, said DVP comprising an amino acid set as forth in SEQ ID NOs: 213, or a pharmaceutically acceptable salt thereof.

In addition, the present disclosure describes a diguetoxin variant polypeptide (DVP) having insecticidal activity against one or more insect species, said DVP consisting of an amino acid set as forth in SEQ ID NOs: 213, or a pharmaceutically acceptable salt thereof.

In addition, the present disclosure describes a diguetoxin variant polypeptide (DVP) having insecticidal activity against one or more insect species, said DVP comprising an amino acid set as forth in SEQ ID NOs: 217, or a pharmaceutically acceptable salt thereof.

In addition, the present disclosure describes a diguetoxin variant polypeptide (DVP) having insecticidal activity against one or more insect species, said DVP consisting of an amino acid set as forth in SEQ ID NOs: 217, or a pharmaceutically acceptable salt thereof.

In addition, the present disclosure describes a diguetoxin variant polypeptide (DVP) having insecticidal activity against one or more insect species, said DVP comprising an amino acid set as forth in SEQ ID NOs: 218, or a pharmaceutically acceptable salt thereof.

In addition, the present disclosure describes a diguetoxin variant polypeptide (DVP) having insecticidal activity against one or more insect species, said DVP consisting of an amino acid set as forth in SEQ ID NOs: 218, or a pharmaceutically acceptable salt thereof.

In addition, the present disclosure describes a diguetoxin variant polypeptide (DVP) having insecticidal activity against one or more insect species, said DVP comprising an amino acid set as forth in SEQ ID NOs: 219, or a pharmaceutically acceptable salt thereof.

In addition, the present disclosure describes a diguetoxin variant polypeptide (DVP) having insecticidal activity against one or more insect species, said DVP consisting of an amino acid set as forth in SEQ ID NOs: 219, or a pharmaceutically acceptable salt thereof.

In addition, the present disclosure describes a fusion protein comprising one or more DVPs operably linked to an alpha mating factor (alpha-MF) peptide; wherein said one or more DVPs have an amino acid sequence that is at least 80%, 85%, 90%, or at least 95% identical to the amino acid sequence according to Formula (I): A-X₁-D-G-D-V-E-G-P-A-G-C-K—K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the DVP comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T, or a pharmaceutically acceptable salt thereof.

### BRIEF DESCRIPTION OF THE DRAWINGS

FIG. 1 shows the high-performance liquid chromatography (HPLC) standard curve for wild-type (WT) Dc1a.

FIG. 2 shows an HPLC chromatogram for pure WT Dc1a.

FIG. 3 depicts a graph showing the relative yield of DVPs C41T/C51A and C41T/C51A/W31F/Y32S/P36A. The DVP C41T/C51A/W31F/Y32S/P36A had a 69% increase in expression compared to C41T/C51A.

FIG. 4 depicts a chromatogram of C41T/C51A. Peaks indicating the background, folded, and misfolded variants are shown in brackets.

FIG. 5 depicts a chromatogram of C41T/C51A/D38A/L42V. Peaks indicating the background, and folded variants are indicated by labels.

FIG. 6 depicts a graph showing a summary of the relative expression of DVPs, showing increased expression without loss of activity. Here, WT-Dc1a, and the following DVPs were analyzed: (1) C41T/C51A; (2) C41T/C51A/D38A; (3) C41T/C51A/D38A/L42V; and (4) C41S/C51S/D38A/L42V.

FIG. 7 shows the results of a fly knockdown experiment evaluating the effect of WT-Dc1a and the following DVPs: (1) C41T/C51A; (2) C41T/C51A/D38A; and (3) C41S/C51S/D38A/L42V. Dose-response curves were generated by assessing flies for percent knockdown (i.e., the inability to walk) at 24 hours (% Knockdown at 24 hr).

FIG. 8 depicts a graph showing percent knockdown for wild-type (triangle), and the DVPs: (1) C41T/C51A/D38A (SEQ ID NO:29) (diamond) and C41S/C51S/D38A/L42V (SEQ ID NO:53) (square), at 24 hours.

FIG. 9 depicts a schematic of a DVP-insecticidal protein. Here, the components are defined as follows: “ERSP” refers to the endoplasmic reticulum signal peptide; “UBI” refers to a ubiquitin monomer; “DVP” refers to a Mu-diguetoxin variant polypeptide; “L” refers to intervening linker peptide; and “HIS” refers to Histidine tag.

FIG. 10 depicts a His-Tag western blot of plant expressed WT Dc1a and DVP-insecticidal proteins. Each lane represents crude plant extracts run under denaturing protein gel conditions and visualized with standard western blot techniques. The short name for the samples tested in the western blot are listed above the image along with a rating system for expression. The symbol (−) indicates that there is no protein detected on the blot and if protein is detected, the symbol (+) to (+++) indicate the amount detected. The lane indicated “LADDER” shows the molecular weight marker. Lanes “PLANT NEG” show the negative control (i.e., GFP expressing tobacco protein extract). Lanes labeled with “M #” indicate the short name for the DVP-insecticidal protein evaluated. Lane “WT” shows an insecticidal protein having the WT Mu-diguetoxin-Dc1a protein.

FIG. 11 shows a graph demonstrating the yield of high yield DVPs compared to a background DVP. Here, point mutations were made on a background DVP having the following mutations: D38A, C41S, and C51S. Mutations to the background DVP included: L42I; K2L; Y32S; K2L+Y32S; D38T; D38S; and D38M. Yield was assessed via rpHPLC and normalized to the background DVP. DVPs with the additional mutations L42I; K2L; Y32S; K2L+Y32S; D38T; and D38S; all possessed improved yield relative to the C41S/C51S/D38A DVP background (SEQ ID NO: 47) control.

FIG. 12 shows a graph showing the result of K2L, Y32S, and L42I mutations. Here, the yield of the DVPs: (1) K2L/Y32S/L42I (SEQ ID NO: 217); and (2) K2L/Y32S/D38A/L42I/C41S/C51S (SEQ ID NO: 218); were compared to the yield of WT Dc1a (SEQ ID NO: 2). Combining the mutations K2L, Y32S, and L42I resulted in dramatic increases in the level of expression.

FIG. 13 depicts a schematic showing Formula (II), which describes a recombinant cysteine rich protein (CRP) having a cystine knot (CK) architecture. Here, C^{I} to C^{VI} are cysteine residues; cysteine residues C^{I} and C^{IV} are connected by a first disulfide bond; C^{II} and C^{V} are connected by a second disulfide bond; and C^{III} and C^{VI} are connected by a third disulfide bond; (disulfide bonds are indicated by lines connecting cysteine residues). The first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif. N_{E}, L₁, L₂, L₃, L₄, L₅, and C_{E} are peptide subunits each comprising an amino acid sequence having a length of 1 to 13 amino acid residues. In some embodiments, wherein N_{E}, L₃, C_{E}, or any combination thereof, are optionally absent.

FIG. 14 shows the relative yield of WT ApsIII and ApsIII cysteine deletion (dCys) as determined by HPLC. (n=8). The dashed line shows the median; dotted lines show the boundaries of the interquartile ranges.

### DETAILED DESCRIPTION

### Definitions

The term “5′-end” and “3′-end” refers to the directionality, i.e., the end-to-end orientation of a nucleotide polymer (e.g., DNA). The 5′-end of a polynucleotide is the end of the polynucleotide that has the fifth carbon.

“5′- and 3′-homology arms” or “5′ and 3′ arms” or “left and right arms” refers to the polynucleotide sequences in a vector and/or targeting vector that homologously recombine with the target genome sequence and/or endogenous gene of interest in the host organism in order to achieve successful genetic modification of the host organism's chromosomal locus.

“ACTX” or “ACTX peptide” or “atracotoxin” refers to a family of insecticidal ICK peptides that have been isolated from spiders belonging to the Atracinae family. One such spider is known as the Australian Blue Mountains Funnel-web Spider, which has the scientific name Hadronyche versuta. Examples of ACTX peptides from Atracinae family species are the Omega-ACTX, Kappa-ACTX, and U-ACTX peptides.

“ADN1 promoter” refers to the DNA segment comprised of the promoter sequence derived from the Schizosaccharomyces pombe adhesion defective protein 1 gene.

“Affect” refers to how a something influences another thing, e.g., how a peptide, polypeptide, protein, drug, or chemical influences an insect, e.g., a pest.

“Alignment” refers to a method of comparing two or more sequences (e.g., nucleotide, polynucleotide, amino acid, peptide, polypeptide, or protein sequences) for the purpose of determining their relationship to each other. Alignments are typically performed by computer programs that apply various algorithms, however, it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW (see Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV (see Larkin M. A., et al., CLUSTALW2, ClustalW and ClustalX version 2, Bioinformatics 23(21): 2947-2948, 2007); Mafft; Kalign; ProbCons; and T-Coffee (see Notredame et al., T-Coffee: A novel method for multiple sequence alignments, Journal of Molecular Biology 302: 205-217, 2000). Exemplary programs that implement one or more of the foregoing algorithms include, but are not limited to, MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif 92121). In some embodiments, an alignment will introduce “phase shifts” and/or “gaps” into one or both of the sequences being compared in order to maximize the similarity between the two sequences, and scoring refers to the process of quantitatively expressing the relatedness of the aligned sequences.

“Alpha mating factor (alpha-MF) peptide” or “alpha-MF signal” or “alpha-MF” or “alpha mating factor secretion signal” or “αMF secretion signal” (all used interchangeably) refers to a signal peptide that allows for secreted expression in a recombinant expression system, when the alpha-MF peptide is operably linked to a recombinant peptide of interest (e.g., a DVP). The Alpha-MF peptide directs nascent recombinant polypeptides to the secretory pathway of the recombinant expression system (e.g., a yeast recombinant expression system).

“Agent” refers to one or more chemical substances, molecules, nucleotides, polynucleotides, peptides, polypeptides, proteins, poisons, insecticides, pesticides, organic compounds, inorganic compounds, prokaryote organisms, or eukaryote organisms, and agents produced therefrom.

“Agriculturally-acceptable carrier” covers all adjuvants, inert components, dispersants, surfactants, tackifiers, binders, etc. that are ordinarily used in pesticide formulation technology; these are well known to those skilled in pesticide formulation.

“Agroinfection” means a plant transformation method where DNA is introduced into a plant cell by using Agrobacteria A. tumefaciens or A. rhizogenes.

“BAAS” means barley alpha-amylase signal peptide, and is an example of an ERSP. One example of a BAAS is a BAAS having the amino acid sequence of SEQ ID NO:60 (NCBI Accession No. AAA32925.1).

“Biomass” refers to any measured plant product.

“Binary vector” or “binary expression vector” means an expression vector which can replicate itself in both E. coli strains and Agrobacterium strains. Also, the vector contains a region of DNA (often referred to as t-DNA) bracketed by left and right border sequences that is recognized by virulence genes to be copied and delivered into a plant cell by Agrobacterium.

“bp” or “base pair” refers to a molecule comprising two chemical bases bonded to one another forming a. For example, a DNA molecule consists of two winding strands, wherein each strand has a backbone made of an alternating deoxyribose and phosphate groups. Attached to each deoxyribose is one of four bases, i.e., adenine (A), cytosine (C), guanine (G), or thymine (T), wherein adenine forms a base pair with thymine, and cytosine forms a base pair with guanine.

“C-terminal” refers to the free carboxyl group (i.e., —COOH) that is positioned on the terminal end of a polypeptide.

“C_{E}” refers to a peptide subunit having an N-terminus that is operably linked to the sixth cysteine residue that participates in the disulfide bond formation the cystine knot motif (i.e., C^{VI}), in the CK architecture according to Formula (II).

As used herein, the letter “C” with a superscript roman numeral, i.e., “C^{I}”, “C^{II}”, “C^{III}”, “C^{IV}”, “C^{V}”, and “C^{VI}”, refers to the cysteine residues that take part in disulfide bond formation, wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, and wherein the first disulfide bond, second disulfide bond, and third disulfide bond are the only disulfide bonds that form the cystine knot motif. Accordingly, a modifiable CRP can have one or more cysteine residues that are operable to form one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif Thus, the superscript roman numerals I, II, III, IV, V, and VI indicate a given cysteine residue that is the first, second, third, fourth, fifth, and sixth cysteine residue to take part in disulfide bond formation, respectively, and wherein those disulfide bonds are the aforementioned first disulfide bond, second disulfide bond, and third disulfide bond form a cystine knot motif. The cysteine residues labeled as “C^{III}”, “C^{II}”, “C^{III}”, “C^{IV}”, “C^{V}”, and “C^{VI}”, and/or the superscript roman numerals I, II, III, IV, V, and VI are not meant to indicate, nor should they be construed as the first, second, third, fourth, fifth, and sixth cysteine residues in an amino acid sequence, as other cysteine residues may be present in a modifiable CRP, regardless of whether those other cysteine residues form a non-CK disulfide bond. For example, a modifiable CRP may have one or more cysteine residues present in its amino acid sequence (reading from the N-terminus to the C-terminus) that occur in the amino acid sequence before the C^{I} residue. Likewise, one or more cysteine residues may be present in the peptide subunits, that may or may not form a non-CK disulfide bond.

“cDNA” or “copy DNA” or “complementary DNA” refers to a molecule that is complementary to a molecule of RNA. In some embodiments, cDNA may be either single-stranded or double-stranded. In some embodiments, cDNA can be a double-stranded DNA synthesized from a single stranded RNA template in a reaction catalyzed by a reverse transcriptase. In yet other embodiments, “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein. In some embodiments, “cDNA” refers to a DNA that is complementary to and derived from an mRNA template.

“CEW” refers to Corn earworm.

“CK architecture” or “cystine knot architecture” refers to the shared structural similarity between peptides, polypeptides, or proteins having an CK motif, e.g., comprising three disulfide bonds, and wherein cysteines C^{I} and C^{IV}; C^{II} and C^{V}; and C^{III} and C^{VI} are connected by a disulfide bond. In some embodiments, “shared structural similarity” refers to the presence of shared structural features, e.g., the presence and/or identity of particular amino acids at particular positions. In yet other embodiments the term “shared structural similarity” refers to presence and/or identity of structural elements (for example: loops, sheets, helices, H-bond donors, H-bond acceptors, glycosylation patterns, salt bridges, and disulfide bonds). In some embodiments, the term “shared structural similarity” refers to three dimensional arrangement and/or orientation of atoms or moieties relative to one another (for example: distance and/or angles between or among them between an agent of interest and a reference agent). In some embodiments, the CK architecture comprises the following scaffold, framework, architecture, and/or backbone: N_{E}—C^{I}-L₁-C^{II}-L₂-C^{III}-L3-C^{IV}-L4-C^{V}-L₅-C^{VI}-C_{E}; wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; and wherein N_{E}, L₃, C_{E}, or any combination thereof, are optionally absent.

“Cleavable Linker” see Linker.

“Cloning” refers to the process and/or methods concerning the insertion of a DNA segment (e.g., usually a gene of interest, for example dvp) from one source and recombining it with a DNA segment from another source (e.g., usually a vector, for example, a plasmid) and directing the recombined DNA, or “recombinant DNA” to replicate, usually by transforming the recombined DNA into a bacteria or yeast host.

“Coding sequence” or “CDS” refers to a polynucleotide or nucleic acid sequence that can be transcribed (e.g., in the case of DNA) or translated (e.g., in the case of mRNA) into a peptide, polypeptide, or protein, when placed under the control of appropriate regulatory sequences and in the presence of the necessary transcriptional and/or translational molecular factors. The boundaries of the coding sequence are determined by a translation start codon at the 5′ (amino) terminus and a translation stop codon at the 3′ (carboxy) terminus. A transcription termination sequence will usually be located 3′ to the coding sequence. In some embodiments, a coding sequence may be flanked on the 5′ and/or 3′ ends by untranslated regions. In some embodiments, a coding sequence can be used to produce a peptide, a polypeptide, or a protein product. In some embodiments, the coding sequence may or may not be fused to another coding sequence or localization signal, such as a nuclear localization signal. In some embodiments, the coding sequence may be cloned into a vector or expression construct, may be integrated into a genome, or may be present as a DNA fragment.

“Codon optimization” refers to the production of a gene in which one or more endogenous, native, and/or wild-type codons are replaced with codons that ultimately still code for the same amino acid, but that are of preference in the corresponding host.

“Complementary” refers to the topological compatibility or matching together of interacting surfaces of two polynucleotides as understood by those of skill in the art. Thus, two sequences are “complementary” to one another if they are capable of hybridizing to one another to form a stable anti-parallel, double-stranded nucleic acid structure. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence 5′-TATAC-3′ is complementary to a polynucleotide whose sequence is 5′-GTATA-3′.

“Conditioned medium” means the cell culture medium which has been used by cells and is enriched with cell derived materials but does not contain cells.

“Copy number” refers to the number of identical copies of a vector, an expression cassette, an amplification unit, a gene or indeed any defined nucleotide sequence, that are present in a host cell at any time. For example, in some embodiments, a gene or another defined chromosomal nucleotide sequence may be present in one, two, or more copies on the chromosome. An autonomously replicating vector may be present in one, or several hundred copies per host cell.

“Culture” or “cell culture” refers to the maintenance of cells in an artificial, in vitro environment.

“Culturing” refers to the propagation of organisms on or in various kinds of media. For example, the term “culturing” can mean growing a population of cells under suitable conditions in a liquid or solid medium. In some embodiments, culturing refers to fermentative recombinant production of a heterologous polypeptide of interest and/or other desired end products (typically in a vessel or reactor).

“Cystine” refers to an oxidized cysteine-dimer. Cystines are sulfur-containing amino acids obtained via the oxidation of two cysteine molecules, and are linked with a disulfide bond.

“Cystine knot motif” or “CK motif” refers to protein structural motif comprising 3 disulfide bonds. The term “cystine-knot motif” as used herein refers to a structural motif containing 3 disulfide bonds: a first disulfide bond, a second disulfide bond, and a third disulfide bond wherein the sections of peptide that occur between two of the disulfide bonds form a loop, through which a third disulfide bond passes, forming a rotaxane substructure. The first disulfide bond occurs between cysteine residues C^{I} and C^{IV}; the second disulfide bond occurs between cysteine residues C^{II} and C^{V}; and the third disulfide bond occurs between cysteine residues C^{III} and C^{VI}; wherein the first disulfide bond, second disulfide bond, and third disulfide bond have a disulfide bond topology that forms the cystine knot motif, and wherein the first disulfide bond, the second disulfide bond, and the third disulfide bond are the only disulfide bonds that form the cystine knot motif. In some embodiments, the disulfide bond topology forms one of the following cystine knot motifs: an inhibitor cystine knot (ICK) motif, a growth factor cystine knot (GFCK) motif, or a cyclic cystine knot (CCK) motif.

“Dc1a” or “Mu-diguetoxin-Dc1a” refers to a polypeptide isolated from the American Desert Spider (Diguetia canities), also known as “the desert bush spider.” One example of a wild-type Mu-diguetoxin-Dc1a is a polypeptide having the amino acid sequence of SEQ ID NO:1 (NCBI Accession No. P49126.1).

“Defined medium” means a medium that is composed of known chemical components but does not contain crude proteinaceous extracts or by-products such as yeast extract or peptone.

“Degeneracy” or “codon degeneracy” refers to the phenomenon that one amino acid can be encoded by different nucleotide codons. Thus, the nucleic acid sequence of a nucleic acid molecule that encodes a protein or polypeptide can vary due to degeneracies. As a result of the degeneracy of the genetic code, many nucleic acid sequences can encode a given polypeptide with a particular activity; such functionally equivalent variants are contemplated herein.

“Disulfide bond” or “disulfide bridges” refers to a covalent bond between two cysteine amino acids derived by the coupling of two thiol groups on their side chains. In some embodiments, a disulfide bond occurs via the oxidative folding of two different thiol groups (—SH) present in a polypeptide, e.g., a CRIP. In some embodiments, a polypeptide can comprise at least six different thiol groups (i.e., six cysteine residues each containing a thiol group); thus, in some embodiments, a polypeptide can form three, or more intramolecular disulfide bonds.

“Disulfide bond topology” or “disulfide bond linkage pattern” or “disulfide bond connectivity” refers to the linking pattern of disulfide bonds and cysteine residues. In some embodiments, a CRIP with the CK architecture of Formula (II) comprises six conserved cysteine residues (numbered I-VI) that form three disulfide bonds with the following disulfide bond connectivities: C^{I} and C^{IV}; C^{II} and C^{V}; and C^{III} and C^{VI}. In some embodiments, the disulfide bonding connectivity is topologically constant, meaning the disulfide bonds can only be changed by unlinking one or more disulfides such as using redox conditions.

“Double expression cassette” refers to two DVP expression cassette s contained on the same vector.

“Double transgene peptide expression vector” or “double transgene expression vector” means a yeast expression vector that contains two copies of the DVP expression cassette.

“DNA” refers to deoxyribonucleic acid, comprising a polymer of one or more deoxyribonucleotides or nucleotides (i.e., adenine [A], guanine [G], thymine [T], or cytosine [C]), which can be arranged in single-stranded or double-stranded form. For example, one or more nucleotides creates a polynucleotide.

“dNTPs” refers to the nucleoside triphosphates that compose DNA and RNA.

“dvp” or “Mu-diguetoxin-Dc1a variant polynucleotide” or “Dc1a variant polynucleotide” or “variant Mu-diguetoxin-Dc1a polynucleotide” refers to a polynucleotide sequence operable to encodes a DVP. The term “Mu-diguetoxin-Dc1a variant polynucleotide” when used to describe the Mu-diguetoxin-Dc1a variant polynucleotide sequence contained in a DVP ORF, its inclusion in a vector, and/or when describing the polynucleotides encoding an insecticidal protein, is described as “dvp” and/or “Dvp”.

“DVP” or “Mu-diguetoxin-Dc1a Variant Polypeptides” refer to peptide, polypeptide, or protein mutants or variants that differ in some way from the wild-type mature Mu-diguetoxin-Dc1a (SEQ ID NO:2); for example, in some embodiments, this variance can be an amino acid substitution, amino acid deletion/insertion, and/or a mutation or variance to a polynucleotide operable to encode the wild-type Mu-diguetoxin-Dc1a. The result of this variation is a non-naturally occurring polypeptide and/or polynucleotide sequence encoding the same that possesses insecticidal activity against one or more insect species, relative to the wild-type Mu-diguetoxin-Dc1a.

“DVP expression cassette” refers to one or more regulatory elements such as promoters; enhancer elements; mRNA stabilizing polyadenylation signal; an internal ribosome entry site (IRES); introns; post-transcriptional regulatory elements; and a polynucleotide operable to encode a DVP, e.g., a DVP ORF. For example, one example of a DVP expression cassette is one or more segments of DNA that contains a polynucleotide segment operable to express a DVP, a ADH1 promoter, a LAC4 terminator, and an alpha-MF secretory signal. A DVP expression cassette contains all of the nucleic acids necessary to encode a DVP or a DVP-insecticidal protein.

“DVP ORF” refers to a polynucleotide operable to encode a DVP, or a DVP-insecticidal protein.

“DVP ORF diagram” refers to the composition of one or more DVP ORFs, as written out in diagram or equation form. For example, a “DVP ORF diagram” can be written out as using acronyms or short-hand references to the DNA segments contained within the expression ORF. Accordingly, in one example, a “DVP ORF diagram” may describe the polynucleotide segments encoding the ERSP, LINKER, STA, and DVP, by diagramming in equation form the DNA segments as “ersp” (i.e., the polynucleotide sequence that encodes the ERSP polypeptide); “linker” or “L” (i.e., the polynucleotide sequence that encodes the LINKER polypeptide); “sta” (i.e., the polynucleotide sequence that encodes the STA polypeptide), and “dvp” (i.e., the polynucleotide sequence encoding a DVP), respectively. An example of a DVP ORF diagram is “ersp-sta-(linkerᵢ-dvpⱼ)_{N},” or “ersp-(dvpⱼ-linkerᵢ)_{N}-sta” and/or any combination of the DNA segments thereof.

“DVP-insecticidal protein” refers to any protein, peptide, polypeptide, amino acid sequence, configuration, or arrangement, consisting of: (1) at least one DVP, or two or more DVPs (wherein said two or more DVPs may be the same or different); and (2) additional non-toxin peptides, polypeptides, or proteins, wherein said additional non-toxin peptides, polypeptides, or proteins e.g., in some embodiments, have the ability to do one or more of the following: increase the mortality and/or inhibit the growth of insects when the insects are exposed to a DVP-insecticidal protein, relative to a DVP alone; increase the expression of said DVP-insecticidal protein, e.g., in a host cell or an expression system; and/or affect the post-translational processing of the DVP-insecticidal protein (e.g., allow for secreted expression of the DVP-insecticidal protein). In some embodiments, a DVP-insecticidal protein can be a polymer comprising two or more DVPs. In some embodiments, a DVP-insecticidal protein can be a polymer comprising two or more DVPs, wherein the DVPs are operably linked via a linker peptide, e.g., a cleavable and/or non-cleavable linker. In some embodiments, a DVP-insecticidal protein can refer to a one or more DVPs operably linked with one or more proteins such as a stabilizing domain (STA); an endoplasmic reticulum signaling protein (ERSP); an insect cleavable or insect non-cleavable linker (L); and/or any other combination thereof. In some embodiments, a DVP-insecticidal protein can be a non-naturally occurring protein comprising (1) a wild-type Dc1a protein; and (2) additional non-toxin peptides, polypeptides, or proteins, e.g., an ERSP; a linker; a STA; a UBI; or a histidine tag or similar marker. In some embodiments, the DVP-insecticidal protein can comprise: (1) a DVP; and (2) an alpha mating factor peptide. For example in some embodiments, a DVP-insecticidal protein can comprise: (1) a DVP; and (2) an alpha mating factor (alpha-MF) or α-mating factor (α-MF) secretion domain (for secreted expression). In some embodiments, a DVP-insecticidal protein can comprise: (1) a DVP; and (2) a K. lactis α-mating factor (α-MF) secretion domain (for secreted expression). In some embodiments, a DVP-insecticidal protein can comprise: (1) two or more DVPs, wherein the DVPs are operably linked via a linker peptide, e.g., a cleavable and/or non-cleavable linker; and wherein the DVPs are the same or different; and (2) an alpha-MF, e.g., a K. lactis α-mating factor (α-MF) secretion domain (for secreted expression).

“DVP construct” refers to the three-dimensional arrangement/orientation of peptides, polypeptides, and/or motifs of operably linked polypeptide segments (e.g., a DVP-insecticidal protein). For example, a DVP ORF can include one or more of the following components or motifs: a DVP; an endoplasmic reticulum signal peptide (ERSP); a linker peptide (L); a translational stabilizing protein (STA); or any combination thereof. And, as used herein, the term “DVP construct” is used to describe the designation and/or orientation of the structural motif. In other words, the DVP construct describes the arrangement and orientation of the components or motifs contained within a given DVP ORF. For example, in some embodiments, a DVP construct describes, without limitation, the orientation of one of the following DVP-insecticidal proteins: ERSP-DVP; ERSP-(DVP)_{N}; ERSP-DVP-L; ERSP-(DVP)_{N}-L; ERSP-(DVP-L)_{N}; ERSP-L-DVP; ERSP-L-(DVP)_{N}; ERSP-(L-DVP)_{N}; ERSP-STA-DVP; ERSP-STA-(DVP)_{N}; ERSP-DVP-STA; ERSP-(DVP)_{N}—STA; ERSP-(STA-DVP)_{N}; ERSP-(DVP-STA)_{N}; ERSP-L-DVP-STA; ERSP-L-STA-DVP; ERSP-L-(DVP-STA)_{N}; ERSP-L-(STA-DVP)_{N}; ERSP-L-(DVP)_{N}—STA; ERSP-(L-DVP)_{N}—STA; ERSP-(L-STA-DVP)_{N}; ERSP-(L-DVP-STA)_{N}; ERSP-(L-STA)_{N}-DVP; ERSP-(L-DVP)_{N}—STA; ERSP-STA-L-DVP; ERSP-STA-DVP-L; ERSP-STA-L-(DVP)_{N}; ERSP-(STA-L)_{N}-DVP; ERSP-STA-(L-DVP)_{N}; ERSP-(STA-L-DVP)_{N}; ERSP-STA-(DVP)_{N}-L; ERSP-STA-(DVP-L)_{N}; ERSP-(STA-DVP)_{N}-L; ERSP-(STA-DVP-L)_{N}; ERSP-DVP-L-STA; ERSP-DVP-STA-L; ERSP-(DVP)_{N}—STA-L ERSP-(DVP-L)_{N}-STA; ERSP-(DVP-STA)_{N}-L; ERSP-(DVP-L-STA)_{N}; or ERSP-(DVP-STA-L)_{N}; wherein N is an integer ranging from 1 to 200.

“ELISA” or “iELISA” means an assay protocol in which the samples are fixed to the surface of a plate and then detected as follows: a primary antibody is applied followed by a secondary antibody conjugated to an enzyme which converts a colorless substrate to colored substrate which can be detected and quantified across samples. During the protocol, antibodies are washed away such that only those that bind to their epitopes remain for detection. The samples, in our hands, are predominantly proteins, and ELISA allows for the quantification of the amount of protein recovered.

“Endogenous” refers to a polynucleotide, peptide, polypeptide, protein, or process that naturally occurs and/or exists in an organism, e.g., a molecule or activity that is already present in the host cell before a particular genetic manipulation.

“Enhancer element” refers to a DNA sequence operably linked to a promoter, which can exert increased transcription activity on the promoter relative to the transcription activity that results from the promoter in the absence of the enhancer element.

“ER” or “Endoplasmic reticulum” is a subcellular organelle common to all eukaryotes where some post translation modification processes occur.

“ERSP” or “Endoplasmic reticulum signal peptide” is an N-terminus sequence of amino acids that-during protein translation of the mRNA molecule encoding a DVP—is recognized and bound by a host cell signal-recognition particle, which moves the protein translation ribosome/mRNA complex to the ER in the cytoplasm. The result is the protein translation is paused until it docks with the ER where it continues and the resulting protein is injected into the ER.

“ersp” refers to a polynucleotide encoding the peptide, ERSP.

“ER trafficking” means transportation of a cell expressed protein into ER for post-translational modification, sorting and transportation.

“Expression cassette” refers to all the DNA elements necessary to complete transcription of a transgene or a heterologous polynucleotide—e.g., a polynucleotide operable to encode a DVP in a recombinant expression system. Thus, in some embodiments, an “expression cassette” refers to a (1) a DNA sequence of interest, e.g., a heterologous polynucleotide operable to encode a DVP; and one or more of the following: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements. The combination (1) with at least one of (2)-(6) is called an “expression cassette.”

For example, in some embodiments, an expression cassette can be (1) a heterologous polynucleotide operable to encode a DVP; and further comprising one or more: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements.

In some embodiments, an expression cassette can be (1) one or more heterologous polynucleotides operable to encode a DVP; and further comprising one or more: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements; wherein each of the one or more heterologous polynucleotides operable to encode a DVP, further comprises one or more of (2)-(6); wherein the DVP can be the same or different.

For example, in some embodiments, an expression cassette can refer to (1) a first heterologous polynucleotide operable to encode a DVP, and one or more additional heterologous polynucleotide operable to encode a DVP; further comprising one or more of: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements; wherein either the first heterologous polynucleotide operable to encode a DVP, and the one or more additional heterologous polynucleotide operable to encode a DVP further comprises one or more of (2)-(6); or wherein each of the first heterologous polynucleotide operable to encode a DVP, and each of the one or more additional heterologous polynucleotide operable to encode a DVP, each individually further comprises one or more of (2)-(6); wherein the DVP can be the same or different.

In alternative embodiments, there are two expression cassettes, each expression cassette comprising a heterologous polynucleotide operable to encode a DVP (i.e., a double expression cassette), wherein the DVP can be the same or different.

In other embodiments, there are three expression cassettes, each expression cassette comprising a heterologous polynucleotide operable to encode a DVP (i.e., a triple expression cassette); wherein the DVP can be the same or different.

In some embodiments, a double expression cassette can be generated by subcloning a second expression cassette into a vector containing a first expression cassette. In some embodiments, a triple expression cassette can be generated by subcloning a third expression cassette into a vector containing a first and a second expression cassette. Methods concerning expression cassettes and cloning techniques are well-known in the art and described herein.

“FECT” means a transient plant expression system using Foxtail mosaic virus with elimination of coating protein gene and triple gene block.

“GFP” means a green fluorescent protein from the jellyfish, Aequorea victoria.

“Growth medium” refers to a nutrient medium used for growing cells in vitro.

“Gut” as used herein can refer to any organ, structure, tissue, cell, extracellular matrix, and/or space comprising the gut, for example: the foregut, e.g., mouth, pharynx, esophagus, crop, proventriculus, or crop; the midgut, e.g., midgut caecum, ventriculus; the hindgut, e.g., pylorum, ileum, rectum or anus; the peritrophic membrane; microvilli; the basement membrane; the muscle layer; Malpighian tubules; or rectal ampulla.

“Homologous” refers to Homologous refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. Homologous refers to the sequence similarity between two polypeptide molecules or between two nucleic acid molecules. When a position in both of the two compared sequences is occupied by the same base or amino acid monomeric subunit, e.g., if a position in each of two DNA molecules is occupied by adenine, then the molecules are homologous at that position. The homology between two sequences is a function of the number of matching or homologous positions shared by the two sequences. For example, if 6 of 10 of the positions in two sequences are matched or homologous then the two sequences are 60% homologous. By way of example, the DNA sequences ATTGCC and TATGGC share 50% homology.

The term “homology,” when used in relation to nucleic acids, refers to a degree of complementarity. There may be partial homology, or complete homology and thus identical. “Sequence identity” refers to a measure of relatedness between two or more nucleic acids, and is given as a percentage with reference to the total comparison length. The identity calculation takes into account those nucleotide residues that are identical and in the same relative positions in their respective larger sequences.

“ICK motif” or “ICK motif protein” refers to a 16 to 60 amino acid peptide with at least 6 half-cystine core amino acids having three disulfide bridges. In some embodiments, the three disulfide bridges are covalent bonds and of the six half-cystine residues the covalent disulfide bonds are between the first and fourth, the second and fifth, and the third and sixth half-cystines, of the six core half-cystine amino acids starting from the N-terminal amino acid. In some embodiments, peptides possessing this motif comprise a beta-hairpin secondary structure, normally composed of residues situated between the fourth and sixth core half-cystines of the motif, wherein the hairpin is stabilized by the structural crosslinking provided by the motif's three disulfide bonds. In some embodiments, additional cysteine/cystine or half-cystine amino acids may be present within the inhibitor cystine knot motif.

“Identity” refers to a relationship between two or more polypeptide sequences or two or more polynucleotide sequences, as determined by comparing said sequences. The term “identity” also means the degree of sequence relatedness between polypeptide or polynucleotide sequences, as the case may be, as determined by the match between strings of such sequences. “Identity” and “similarity” can be readily calculated by any one of the myriad methods known to those having ordinary skill in the art, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), the disclosures of which are incorporated herein by reference in their entireties. Furthermore, methods to determine identity and similarity are codified in publicly available computer programs. For example in some embodiments, methods to determine identity and similarity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), the disclosures of which are incorporated herein by reference in their entireties.

“in vivo” refers to the natural environment (e.g., an animal or a cell) and to processes or reactions that occur within a natural environment.

“Inactive” refers to a condition wherein something is not in a state of use, e.g., lying dormant and/or not working. For example, when used in the context of a gene or when referring to a gene, the term inactive means said gene is no longer actively synthesizing a gene product, having said gene product translated into a protein, or otherwise having the gene perform its normal function. For example, in some embodiments, the term inactive can refer the failure of a gene to transcribe RNA, a failure of RNA processing (e.g., pre-mRNA processing; RNA splicing; or other post-transcriptional modifications); interference with non-coding RNA maturation; interference with RNA export (e.g., from the nucleus to the cytoplasm); interference with translation; protein folding; translocation; protein transport; and/or inhibition and/or interference with any of the molecules polynucleotides, peptides, polypeptides, proteins, transcription factors, regulators, inhibitors, or other factors that take part in any of the aforementioned processes.

“Increasing” or “increase” or “increased” or “increases” refers to making something (e.g., the expression of peptide, polypeptide, or protein) greater in size, amount, intensity, or degree. For example, in some embodiments, the removal of one or more disulfide bonds from a modifiable CRP not having a CK architecture according to Formula (II), can result in the creation of a recombinant CRP having a CK architecture according to Formula (II), wherein having a CK architecture according to Formula (II) results in the following effect: an increase in the level of expression of the recombinant CRP, and/or an increase in the yield of the recombinant CRP, relative to the modifiable CRP not having the CK architecture according to Formula (II).

Thus, in some embodiments, the terms “increased level of expression” or “an increase in the level of expression” or “increased yield” or “an increase in yield,” in a recombinant CRP having the CK architecture according to Formula (II), refers to an increase that is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 20.25%, at least about 2.5%, at least about 20.75%, at least about 3%, at least about 30.25%, at least about 3.5%, at least about 3.75%, at least about 4%, at least about 4 0.25%, at least about 4.5%, at least about 4.75%, at least about 5%, at least about 5.2 5%, at least about 5.5%, at least about 5.75%, at least about 6%, at least about 6 0.25%, at least about 6.5%, at least about 6.75%, at least about 7%, at least about 7.25%, at least about 7.5%, at least about 7.75%, at least about 8%, at least about 8.25%, at least about 8.5%, at least about 8.75%, at least about 9%, at least about 9 0.25%, at least about 9.5%, at least about 9 0.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, in the amount of protein, the level of expression of protein, and/or the yield of protein in the recombinant CRP having the CK architecture according to Formula (II), relative to the amount of protein, the level of expression of protein, and/or the yield of protein in the modifiable CRP that does not have the CK architecture according to Formula (II).

“Inoperable” refers to the condition of a thing not functioning, malfunctioning, or no longer able to function. For example, when used in the context of a gene or when referring to a gene, the term inoperable means said gene is no longer able to operate as it normally would, either permanently or transiently. For example, “inoperable,” in some embodiments, means that a gene is no longer able to synthesize a gene product, having said gene product translated into a protein, or is otherwise unable to gene perform its normal function. For example, in some embodiments, the term inoperable can refer the failure of a gene to transcribe RNA, a failure of RNA processing (e.g., pre-mRNA processing; RNA splicing; or other post-transcriptional modifications); interference with non-coding RNA maturation; interference with RNA export (e.g., from the nucleus to the cytoplasm); interference with translation; protein folding; translocation; protein transport; and/or inhibition and/or interference with any of the molecules polynucleotides, peptides, polypeptides, proteins, transcription factors, regulators, inhibitors, or other factors that take part in any of the aforementioned processes.

“Insect” includes all organisms in the class “Insecta.” The term “pre-adult” insects refers to any form of an organism prior to the adult stage, including, for example, eggs, larvae, and nymphs. As used herein, the term “insect refers to any arthropod and nematode, including acarids, and insects known to infest all crops, vegetables, and trees and includes insects that are considered pests in the fields of forestry, horticulture and agriculture. Examples of specific crops that might be protected with the methods disclosed herein are soybean, corn, cotton, alfalfa and the vegetable crops. A list of specific crops and insects is enclosed herein.

“Insect gut environment” or “gut environment” means the specific pH and proteinase conditions found within the fore, mid or hind gut of an insect or insect larva.

“Insect hemolymph environment” means the specific pH and proteinase conditions of found within an insect or insect larva.

As used herein, the term “insecticidal” is generally used to refer to the ability of a polypeptide or protein used herein, to increase mortality or inhibit growth rate of insects. As used herein, the term “nematicidal” refers to the ability of a polypeptide or protein used herein, to increase mortality or inhibit the growth rate of nematodes. In general, the term “nematode” comprises eggs, larvae, juvenile and mature forms of said organism.

“Insecticidal activity” means that upon or after exposing the insect to compounds, agents, or peptides, the insect either dies stops or slows its movement; stops or slows its feeding; stops or slows its growth; becomes confused (e.g., with regard to navigation, locating food, sleeping behaviors, and/or mating); fails to pupate; interferes with reproduction; and/or precludes the insect from producing offspring and/or precluding the insect from producing fertile offspring.

“Integrative expression vector” or “integrative vector” means a yeast expression vector which can insert itself into a specific locus of the yeast cell genome and stably becomes a part of the yeast genome.

“Intervening linker” refers to a short peptide sequence in the protein separating different parts of the protein, or a short DNA sequence that is placed in the reading frame in the ORF to separate the upstream and downstream DNA sequences. For example, in some embodiments, an intervening linker may be used allowing proteins to achieve their independent secondary and tertiary structure formation during translation. In some embodiments, the intervening linker can be either resistant or susceptible to cleavage in plant cellular environments, in the insect and/or lepidopteran gut environment, and in the insect hemolymph and lepidopteran hemolymph environment.

“Isolated” refers to separating a thing and/or a component from its natural environment, e.g., a toxin isolated from a given genus or species means that toxin is separated from its natural environment.

“Kappa-ACTX peptide” or “x-ACTX” (all used interchangeably) refers to a peptide belonging to a family of insecticidal inhibitor cystine knot (ICK) peptides that have been isolated from Australian funnel-web spiders belonging to the Atracinae subfamily. One such spider is the Australian Blue Mountains Funnel-web Spider, which has the scientific name Haydronyche versuta. An exemplary wild-type Kappa-ACTX peptide is provided herein, having the amino acid sequence: “AICTGADRPCAACCPCCPGTSCKAESNGVSYCRKDEP” (SEQ ID NO: 198) (UniProtKB/Swiss-Prot No. P82228.1).

“kb” refers to kilobase, i.e., 1000 bases. As used herein, the term “kb” means a length of nucleic acid molecules. For example, 1 kb refers to a nucleic acid molecule that is 1000 nucleotides long. A length of double-stranded DNA that is 1 kb long, contains two thousand nucleotides (i.e., one thousand on each strand). Alternatively, a length of single-stranded RNA that is 1 kb long, contains one thousand nucleotides.

“kDa” refers to kilodalton, a unit equaling 1,000 daltons; a “Dalton” or “dalton” is a unit of molecular weight (MW).

“Knock in” or “knock-in” or “knocks-in” or “knocking-in” refers to the replacement of an endogenous gene with an exogenous or heterologous gene, or part thereof. For example, in some embodiments, the term “knock-in” refers to the introduction of a nucleic acid sequence encoding a desired protein to a target gene locus by homologous recombination, thereby causing the expression of the desired protein. In some embodiments, a “knock-in” mutation can modify a gene sequence to create a loss-of-function or gain-of-function mutation. The term “knock-in” can refer to the procedure by which a exogenous or heterologous polynucleotide sequence or fragment thereof is introduced into the genome, (e.g., “they performed a knock-in” or “they knocked-in the heterologous gene”), or the resulting cell and/or organism (e.g., “the cell is a “knock-in” or “the animal is a “knock-in”).

“Knock out” or “knockout” or “knock-out” or “knocks-out” or “knocking-out” refers to a partial or complete suppression of the expression gene product (e.g., mRNA) of a protein encoded by an endogenous DNA sequence in a cell. In some embodiments, the “knock-out” can be effectuated by targeted deletion of a whole gene, or part of a gene encoding a peptide, polypeptide, or protein. As a result, the deletion may render a gene inactive, partially inactive, inoperable, partly inoperable, or otherwise reduce the expression of the gene or its products in any cell in the whole organism and/or cell in which it is normally expressed. The term “knock-out” can refer to the procedure by which an endogenous gene is made completely or partially inactive or inoperable (e.g., “they performed a knock-out” or “they knocked-out the endogenous gene”), or the resulting cell and/or organism (e.g., “the cell is a “knock-out” or “the animal is a “knock-out”).

“Knockdown dose 50” or “KD₅₀” refers to the median dose required to cause paralysis or cessation of movement in 50% of a population, for example a population of Musca domestica (common housefly) and/or Aedes aegypti (mosquito).

“I” or “linker” refers to a nucleotide encoding intervening linker peptide.

“L₁” refers to a peptide subunit located between the first cysteine and second cysteine residues that participate in the disulfide bond formation the cystine knot motif (i.e., C^{I} and C^{II}) in the CK architecture according to Formula (II).

“L₂” refers to a peptide subunit located between the second cysteine and third cysteine residues that participate in the disulfide bond formation the cystine knot motif (i.e., C^{I} and C^{III}) in the CK architecture according to Formula (II).

“L₃” refers to a peptide subunit located between the third cysteine and fourth cysteine residues that participate in the disulfide bond formation the cystine knot motif (i.e., C^{III} and C^{IV}) in the CK architecture according to Formula (II).

“L₄” refers to a peptide subunit located between the fourth cysteine and fifth cysteine residues that participate in the disulfide bond formation the cystine knot motif (i.e., C^{IV} and C^{V}) in the CK architecture according to Formula (II).

“L₅” refers to a peptide subunit located between the fifth cysteine and sixth cysteine residues that participate in the disulfide bond formation the cystine knot motif (i.e., C^{V} and C^{VI}) in the CK architecture according to Formula (II).

“L” in the proper context refers to an intervening linker peptide, which links a translational stabilizing protein (STA) with an additional polypeptide, e.g., a DVP, and/or multiple DVPs. When referring to amino acids, “L” can also mean leucine.

“LAC4 promoter” or “Lac4 promoter” or “pLac4” refers to a DNA segment comprised of the promoter sequence derived from the K. lactis β-galactosidase gene. The LAC4 promoters is strong and inducible reporter that is used to drive expression of exogenous genes transformed into yeast.

“LAC4 terminator” or “Lac4 terminator” refers to a DNA segment comprised of the transcriptional terminator sequence derived from the K. lactis β-galactosidase gene.

“Lepidopteran gut environment” means the specific pH and proteinase conditions of found within the fore, mid or hind gut of a lepidopteran insect or larva.

“Lepidopteran hemolymph environment” means the specific pH and proteinase conditions of found within lepidopteran insect or larva.

“LD₂₀” refers to a dose required to kill 20% of a population.

“LD₅₀” refers to lethal dose 50 which means the dose required to kill 50% of a population.

“Linker” or “LINKER” or “peptide linker” or “L” or “intervening linker” refers to a short peptide sequence operable to link two peptides together. Linker can also refer to a short DNA sequence that is placed in the reading frame of an ORF to separate an upstream and downstream DNA sequences. In some embodiments, a linker can be cleavable by an insect protease. In some embodiments, a linker may allow proteins to achieve their independent secondary and tertiary structure formation during translation. In some embodiments, the linker can be either resistant or susceptible to cleavage in plant cellular environments, in the insect and/or lepidopteran gut environment, and/or in the insect hemolymph and lepidopteran hemolymph environment. In some embodiments, a linker can be cleaved by a protease, e.g., in some embodiments, a linker can be cleaved by a plant protease (e.g., papain, bromelain, ficin, actinidin, zingibain, and/or cardosins), an insect protease, a fungal protease, a vertebrate protease, an invertebrate protease, a bacteria protease, a mammal protease, a reptile protease, or an avian protease. In some embodiments, a linker can be cleavable or non-cleavable. In some embodiments, a linker comprises a binary or tertiary region, wherein each region is cleavable by at least two types of proteases: one of which is an insect and/or nematode protease and the other one of which is a human protease. In some embodiments, a linker can have one of (at least) three roles: to cleave in the insect gut environment, to cleave in the plant cell, or to be designed not to intentionally cleave.

“Medium” (plural “media”) refers to a nutritive solution for culturing cells in cell culture.

“MOA” refers to mechanism of action.

“Modifiable CRP” refers to a cysteine rich protein having one or more non-CK disulfide bonds, in addition to a first disulfide bond, a second disulfide bond, and a third disulfide bond having a disulfide bond topology that forms a cystine knot motif, wherein the one or more non-CK disulfide bonds are not the first disulfide bond, the second disulfide bond, or the third disulfide bond, and wherein the one or more non-CK disulfide bonds do not form the CK motif. Examples of a modifiable CRP include an ApsIII protein having the amino acid sequence of a “CNSKGTPCTNADECCGGKCAYNVWNCIGGGCSKTCGY” (SEQ ID NO: 193; NCBI Accession No. P49268.1); a wild-type Kappa-ACTX peptide having the amino acid sequence: “AICTGADRPCAACCPCCPGTSCKAESNGVSYCRKDEP” (SEQ ID NO: 198; UniProtKB/Swiss-Prot No. P82228.1); and or any one of SEQ ID NOs: 1-2, or 195.

“Molecular weight (MW)” refers to the mass or weight of a molecule, and is typically measured in “daltons (Da)” or kilodaltons (kDa). In some embodiments, MW can be calculated using sodium dodecyl sulfate polyacrylamide gel electrophoresis (SDS-PAGE), analytical ultracentrifugation, or light scattering. In some embodiments, the SDS-PAGE method is as follows: the sample of interest is separated on a gel with a set of molecular weight standards. The sample is run, and the gel is then processed with a desired stain, followed by destaining for about 2 to 14 hours. The next step is to determine the relative migration distance (Rf) of the standards and protein of interest. The migration distance can be determined using the following equation:

$$Rf = \frac{Migration distance of the protein}{Migration distance of the dye front} \qquad Formula ( III )$$

Next, the logarithm of the MW can be determined based on the values obtained for the bands in the standard; e.g., in some embodiments, the logarithm of the molecular weight of an SDS-denatured polypeptide and its relative migration distance (Rf) is plotted into a graph. After plotting the graph, interpolating the value derived will provide the molecular weight of the unknown protein band.

“Motif” refers to a polynucleotide or polypeptide sequence that is implicated in having some biological significance and/or exerts some effect or is involved in some biological process.

“Multiple cloning site” or “MCS” refers to a segment of DNA found on a vector that contains numerous restriction sites in which a DNA sequence of interest can be inserted.

“Mutant” refers to an organism, DNA sequence, peptide sequence, or polypeptide sequence, that has an alteration (for example, in the DNA sequence), which causes said organism and/or sequence to be different from the naturally occurring or wild-type organism and/or sequence. For example, a wild-type Mu-diguetoxin-Dc1a polypeptide can be altered resulting in a non-naturally occurring DVP.

“N_{E}” refers to a peptide subunit having a C-terminus that is operably linked to the first cysteine residue that participates in the disulfide bond formation the cystine knot motif (i.e., C^{I}), in the CK architecture according to Formula (II).

“N-terminal” refers to the free amine group (i.e., —NH₂) that is positioned on beginning or start of a polypeptide.

“NCBI” refers to the National Center for Biotechnology Information.

“nm” refers to nanometers.

“Non-Polar amino acid” is an amino acid that is weakly hydrophobic and includes glycine, alanine, proline, valine, leucine, isoleucine, phenylalanine and methionine. Glycine or gly is the most preferred non-polar amino acid for the dipeptides of this invention.

“Normalized peptide yield” means the peptide yield in the conditioned medium divided by the corresponding cell density at the point the peptide yield is measured. The peptide yield can be represented by the mass of the produced peptide in a unit of volume, for example, mg per liter or mg/L, or by the UV absorbance peak area of the produced peptide in the HPLC chromatograph, for example, mAu·sec. The cell density can be represented by visible light absorbance of the culture at wavelength of 600 nm (OD600).

“OD” refers to optical density. Typically, OD is measured using a spectrophotometer.

“OD660 nm” or “OD₆₆₀ₙₘ” refers to optical densities at 660 nanometers (nm).

“One letter code” means the peptide sequence which is listed in its one letter code to distinguish the various amino acids in the primary structure of a protein: alanine=A, arginine=R, asparagine=N, aspartic acid=D, asparagine or aspartic acid=B, cysteine=C, glutamic acid=E, glutamine=Q, glutamine or glutamic acid=Z, glycine=G, histidine=H, isoleucine=I, leucine=L, lysine=K, methionine=M, phenylalanine=F, proline=P, serine=S, threonine=T, tryptophan=W, tyrosine=Y, and valine=V.

“Operable” refers to the ability to be used, the ability to do something, and/or the ability to accomplish some function or result. For example, in some embodiments, “operable” refers to the ability of a polynucleotide, DNA sequence, RNA sequence, or other nucleotide sequence or gene to encode a peptide, polypeptide, and/or protein. For example, in some embodiments, a polynucleotide may be operable to encode a protein, which means that the polynucleotide contains information that imbues it with the ability to create a protein (e.g., by transcribing mRNA, which is in turn translated to protein).

“Operably linked” refers to a juxtaposition wherein the components so described are in a relationship permitting them to function in their intended manner. For example, in some embodiments, operably linked can refer to two or more DNA, peptide, or polypeptide sequences. In other embodiments, operably linked can mean that the two adjacent DNA sequences are placed together such that the transcriptional activation of one DNA sequence can act on the other DNA sequence. In yet other embodiments, the term “operably linked” can refer to two or more peptides and/or polypeptides, wherein said two or more peptides and/or polypeptides are connected in such a way as to yield a single polypeptide chain; alternatively, the term operably linked can refer to two or more peptides that are connected in such a way that one peptide exerts some effect on the other. In yet other embodiments, operably linked can refer to two adjacent DNA sequences are placed together such that the transcriptional activation of one can act on the other.

“ORF” or “open reading frame” refers to a length of RNA or DNA sequence, between a translation start signal (e.g., AUG or ATG, respectively) and any one or more of the known termination codons, which encodes one or more polypeptide sequences. Put another way, the ORF describes the frame of reference as seen from the point of view of a ribosome translating the RNA code, insofar that the ribosome is able to keep reading (i.e., adding amino acids to the nascent protein) because it has not encountered a stop codon. Thus, “open reading frame” or “ORF” refers to the amino acid sequence encoded between translation initiation and termination codons of a coding sequence. Here, the terms “initiation codon” and “termination codon” refer to a unit of three adjacent nucleotides (i.e., a codon) in a coding sequence that specifies initiation and chain termination, respectively, of protein synthesis (mRNA translation).

In some embodiments, an ORF is a continuous stretch of codons that begins with a start codon (usually ATG for DNA, and AUG for RNA) and ends at a stop codon (usually UAA, UAG or UGA). In other embodiments, an ORF can be length of RNA or DNA sequence, between a translation start signal (e.g., AUG or ATG) and any one or more of the known termination codons, wherein said length of RNA or DNA sequence encodes one or more polypeptide sequences. In some other embodiments, an ORF can be a DNA sequence encoding a protein which begins with an ATG start codon and ends with a TGA, TAA or TAG stop codon. ORF can also mean the translated protein that the DNA encodes. Generally, those having ordinary skill in the art distinguish the terms “open reading frame” and “ORF,” from the term “coding sequence,” based upon the fact that the broadest definition of “open reading frame” simply contemplates a series of codons that does not contain a stop codon. Accordingly, while an ORF may contain introns, the coding sequence is distinguished by referring to those nucleotides (e.g., concatenated exons) that can be divided into codons that are actually translated into amino acids by the ribosomal translation machinery (i.e., a coding sequence does not contain introns); however, as used herein, the terms “coding sequence”; “CDS”; “open reading frame”; and “ORF,” are used interchangeably.

“Out-recombined” or “out-recombination” refers to the removal of a gene and/or polynucleotide sequence (e.g., an endogenous gene) that is flanked by two site-specific recombination sites (e.g., the 5′- and 3′-nucleotide sequence of a target gene that is homologous to the homology arms of a target vector) during in vivo homologous recombination. See “knockout.”

“Peptide expression vector” means a host organism expression vector which contains a heterologous peptide transgene.

“Peptide expression yeast strain”, “peptide expression strain” or “peptide production strain” means a yeast strain which can produce a heterologous peptide.

“Peptide Linker” see Linker.

“Peptide subunit” refers to an amino acid sequence upstream, downstream, and/or between one or more cysteine residues in a peptide, polypeptide, or protein. In some embodiments, a peptide subunit is upstream, downstream, and/or between cysteine residues in a recombinant CRP having a CK architecture according to Formula (II). In some embodiments, a peptide subunit can have a length of 1 to 13 amino acid residues. In yet other embodiments, a peptide subunit can have a length of 13 or more amino acid residues. In some embodiments, peptide subunits in a recombinant CRP comprising the CK architecture according to Formula (II) are designated as N_{E}, L₁, L₂, L₃, L₄, L₅, and C_{E}.

“Peptide transgene” or “insecticidal peptide transgene” or “insecticidal protein transgene” or “Mu-diguetoxin-Dc1a variant transgene” refers to a DNA sequence that encodes an DVP and can be translated in a biological expression system.

“Peptide yield” means the insecticidal peptide concentration in the conditioned medium which is produced from the cells of a peptide expression yeast strain. It can be represented by the mass of the produced peptide in a unit of volume, for example, mg per liter or mg/L, or by the UV absorbance peak area of the produced peptide in the HPLC chromatograph, for example, mAu·sec.

“Pest” includes, but is not limited to: insects, fungi, bacteria, nematodes, mites, ticks, and the like.

“Pesticidally-effective amount” refers to an amount of the pesticide that is able to bring about death to at least one pest, or to noticeably reduce pest growth, feeding, or normal physiological development. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective polypeptide composition. The formulations may also vary with respect to climatic conditions, environmental considerations, and/or frequency of application and/or severity of pest infestation.

“Pharmaceutically acceptable salt” refers to a compound that is modified by making acid or base salts thereof.

“Plant” shall mean whole plants, plant tissues, plant cells, plant parts, plant organs (e.g., leaves, stems, roots, etc.), seeds, propagules, embryos and progeny of the same. Plant cells can be differentiated or undifferentiated (e.g. callus, suspension culture cells, protoplasts, leaf cells, root cells, phloem cells, and pollen).

“Plant transgenic protein” means a protein from a heterologous species that is expressed in a plant after the DNA or RNA encoding it was delivered into one or more of the plant cells.

“Plant-incorporated protectant” or “PIP” means an insecticidal protein produced by transgenic plants, and the genetic material necessary for the plant to produce the protein.

“Plant cleavable linker” means a cleavable linker peptide, or a nucleotide encoding a cleavable linker peptide, which contains a plant protease recognition site and can be cleaved during the protein expression process in the plant cell.

“Plant regeneration media” means any media that contains the necessary elements and vitamins for plant growth and plant hormones necessary to promote regeneration of a cell into an embryo which can germinate and generate a plantlet derived from tissue culture. Often the media contains a selectable agent to which the transgenic cells express a selection gene that confers resistance to the agent.

“Plasmid” refers to a DNA segment that acts as a carrier for a gene of interest (e.g., dvp) and, when transformed or transfected into an organism, can replicate and express the DNA sequence contained within the plasmid independently of the host organism. Plasmids are a type of vector, and can be “cloning vectors” (i.e., simple plasmids used to clone a DNA fragment and/or select a host population carrying the plasmid via some selection indicator) or “expression plasmids” (i.e., plasmids used to produce large amounts of polynucleotides and/or polypeptides).

“Polar amino acid” is an amino acid that is polar and includes serine, threonine, cysteine, asparagine, glutamine, histidine, tryptophan and tyrosine; preferred polar amino acids are serine, threonine, cysteine, asparagine and glutamine; with serine being most highly preferred.

“Polynucleotide” refers to a polymeric-form of nucleotides (e.g., ribonucleotides, deoxyribonucleotides, or analogs thereof) of any length; e.g., a sequence of two or more ribonucleotides or deoxyribonucleotides. As used herein, the term “polynucleotide” includes double- and single-stranded DNA, as well as double- and single-stranded RNA; it also includes modified and unmodified forms of a polynucleotide (modifications to and of a polynucleotide, for example, can include methylation, phosphorylation, and/or capping). In some embodiments, a polynucleotide can be one of the following: a gene or gene fragment (for example, a probe, primer, EST, or SAGE tag); genomic DNA; genomic DNA fragment; exon; intron; messenger RNA (mRNA); transfer RNA; ribosomal RNA; ribozyme; cDNA; recombinant polynucleotide; branched polynucleotide; plasmid; vector; isolated DNA of any sequence; isolated RNA of any sequence; nucleic acid probe; primer or amplified copy of any of the foregoing.

In yet other embodiments, a polynucleotide can refer to a polymeric-form of nucleotides operable to encode the open reading frame of a gene.

In some embodiments, a polynucleotide can refer to cDNA.

In some embodiments, polynucleotides can have any three-dimensional structure and may perform any function, known or unknown. The structure of a polynucleotide can also be referenced to by its 5′- or 3′-end or terminus, which indicates the directionality of the polynucleotide. Adjacent nucleotides in a single-strand of polynucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the polynucleotide, which may be called the 5′ and 3′ ends or termini. The 5′ and 3′ ends can also be called the phosphoryl (PO₄) and hydroxyl (OH) ends, respectively, because of the chemical groups attached to those ends. The term polynucleotide also refers to both double- and single-stranded molecules. Unless otherwise specified or required, any embodiment that makes or uses a polynucleotide encompasses both the double-stranded form and each of two complementary single-stranded forms known or predicted to make up the double-stranded form.

In some embodiments, a polynucleotide can include modified nucleotides, such as methylated nucleotides and nucleotide analogs (including nucleotides with non-natural bases, nucleotides with modified natural bases such as aza- or deaza-purines, etc.). If present, modifications to the nucleotide structure can be imparted before or after assembly of the polynucleotide.

In some embodiments, a polynucleotide can also be further modified after polymerization, such as by conjugation with a labeling component. Additionally, the sequence of nucleotides in a polynucleotide can be interrupted by non-nucleotide components. One or more ends of the polynucleotide can be protected or otherwise modified to prevent that end from interacting in a particular way (e.g. forming a covalent bond) with other polynucleotides.

In some embodiments, a polynucleotide can be composed of a specific sequence of four nucleotide bases: adenine (A); cytosine (C); guanine (G); and thymine (T). Uracil (U) can also be present, for example, as a natural replacement for thymine when the polynucleotide is RNA. Uracil can also be used in DNA. Thus, the term “sequence” refers to the alphabetical representation of a polynucleotide or any nucleic acid molecule, including natural and non-natural bases.

The term “RNA molecule” or ribonucleic acid molecule refers to a polynucleotide having a ribose sugar rather than deoxyribose sugar and typically uracil rather than thymine as one of the pyrimidine bases. An RNA molecule of the invention is generally single-stranded, but can also be double-stranded. In the context of an RNA molecule from an RNA sample, the RNA molecule can include the single-stranded molecules transcribed from DNA in the cell nucleus, mitochondrion or chloroplast, which have a linear sequence of nucleotide bases that is complementary to the DNA strand from which it is transcribed.

In some embodiments, a polynucleotide can further comprise one or more heterologous regulatory elements. For example, in some embodiments, the regulatory element is one or more promoters; enhancers; silencers; operators; splicing signals; polyadenylation signals; termination signals; RNA export elements, internal ribosomal entry sites (IRES); poly-U sequences; or combinations thereof.

“Post-transcriptional regulatory elements” are DNA segments and/or mechanisms that affect mRNA after it has been transcribed. Mechanisms of post-transcriptional mechanisms include splicing events; capping, splicing, and addition of a Poly (A) tail, and other mechanisms known to those having ordinary skill in the art.

“Promoter” refers to a region of DNA to which RNA polymerase binds and initiates the transcription of a gene.

“Protein” has the same meaning as “peptide” and/or “polypeptide” in this document.

“Ratio” refers to the quantitative relation between two amounts or between two objects, which shows the relationship (in amount or quantity) between the two or more amounts, or between the two or more objects. Accordingly, in some embodiments, a ratio shows the number of times a first value contains, or is contained, within a second value.

“Reading frame” refers to one of the six possible reading frames, three in each direction, of the double stranded DNA molecule. The reading frame that is used determines which codons are used to encode amino acids within the coding sequence of a DNA molecule. In some embodiments, a reading frame is a way of dividing the sequence of nucleotides in a polynucleotide and/or nucleic acid (e.g., DNA or RNA) into a set of consecutive, non-overlapping triplets.

“Recombinant CRP” refers to refers to a non-naturally-occurring, recombinant peptide, polypeptide, or protein comprising a cystine knot (CK) architecture according to Formula (II), that is derived from a modifiable CRP that does not have the cystine knot (CK) architecture according to Formula (II). As used herein, the term “recombinant” encompasses, for example, a polypeptide that comprises one or more changes, including additions, deletions, and/or substitutions, relative to its naturally occurring counterpart, or relative to a non-naturally occurring protein that does not does not have the cystine knot (CK) architecture according to Formula (II) (e.g., a non-natural, modifiable CRP), wherein such changes were introduced, e.g., by recombinant DNA techniques. The term “recombinant” also encompasses a peptide, polypeptide, or protein that comprises, consists essentially of, or consists of: an amino acid sequence generated by humans; an artificial peptide, polypeptide, or protein; a fusion protein; and/or and a chimeric polypeptide; a nucleotide sequence generated by humans; an artificial nucleotide, polynucleotide, DNA, RNA, or gene; a polynucleotide encoding a fusion protein; and/or and a polynucleotide encoding a chimeric polypeptide. Once expressed, recombinant peptides, polypeptides, and/or proteins can be purified according to standard procedures known to one of ordinary skill in the art, e.g., including but not limited to: ammonium sulfate precipitation, affinity columns, column chromatography, gel electrophoresis and the like. In some embodiments, recombinant proteins may be produced by any means, including, for example, peptide, polypeptide, or protein synthesis.

“Recombinant DNA” or “rDNA” refers to DNA that is comprised of two or more different DNA segments.

“Recombinant vector” means a DNA plasmid vector into which foreign DNA has been inserted.

“Regulatory elements” refers to a genetic element that controls some aspect of the expression and/or processing of nucleic acid sequences. For example, in some embodiments, a regulatory element can be found at the transcriptional and post-transcriptional level. Regulatory elements can be cis-regulatory elements (CREs), or trans-regulatory elements (TREs). In some embodiments, a regulatory element can be one or more promoters; enhancers; silencers; operators; splicing signals; polyadenylation signals; termination signals; RNA export elements, internal ribosomal entry sites (IRES); poly-U sequences; and/or other elements that influence gene expression, for example, in a tissue-specific manner; temporal-dependent manner; to increase or decrease expression; and/or to cause constitutive expression.

“Restriction enzyme” or “restriction endonuclease” refers to an enzyme that cleaves DNA at a specified restriction site. For example, a restriction enzyme can cleave a plasmid at an EcoRI, SacII or BstXI restriction site allowing the plasmid to be linearized, and the DNA of interest to be ligated.

“Restriction site” refers to a location on DNA comprising a sequence of 4 to 8 nucleotides, and whose sequence is recognized by a particular restriction enzyme.

“Selection gene” means a gene which confers an advantage for a genetically modified organism to grow under the selective pressure.

“Serovar” or “serotype” refers to a group of closely related microorganisms distinguished by a characteristic set of antigens. In some embodiments, a serovar is an antigenically and serologically distinct variety of microorganism

“sp.” refers to species.

“ssp.” or “subsp.” refers to subspecies.

“Subcloning” or “subcloned” refers to the process of transferring DNA from one vector to another, usually advantageous vector. For example, polynucleotide encoding a mutant DVP can be subcloned into a pLB102 plasmid subsequent to selection of yeast colonies transformed with pKLAC1 plasmids.

“SSI” is an acronym that is context dependent. In some contexts, it can refer to “site-specific integration,” which is used to refer to a sequence that will permit in vivo homologous recombination to occur at a specific site within a host organism's genome. Thus, in some embodiments, the term “site-specific integration” refers to the process directing a transgene to a target site in a host-organism's genome, allowing the integration of genes of interest into pre-selected genome locations of a host-organism. However, in other contexts, SSI can refer to “surface spraying indoors,” which is a technique of applying a variable volume sprayable volume of an insecticide onto surfaces where vectors rest, such as on walls, windows, floors and ceilings.

“STA” or “Translational stabilizing protein” or “stabilizing domain” or “stabilizing protein” (used interchangeably herein) means a peptide or protein with sufficient tertiary structure that it can accumulate in a cell without being targeted by the cellular process of protein degradation. The protein can be between 5 and 50 amino acids long. The translational stabilizing protein is coded by a DNA sequence for a protein that is operably linked with a sequence encoding an insecticidal protein or a DVP in the ORF. The operably-linked STA can either be upstream or downstream of the DVP and can have any intervening sequence between the two sequences (STA and DVP) as long as the intervening sequence does not result in a frame shift of either DNA sequence. The translational stabilizing protein can also have an activity which increases delivery of the DVP across the gut wall and into the hemolymph of the insect. Examples of a STA include, without limitation, any of the translational stabilizing proteins described, or taught by this document including GFP (Green Fluorescent Protein; SEQ ID NO:57; NCBI Accession No. P42212); GNA (SEQ ID NO: 58; NCBI Accession No. AAL07474.1); or Jun a 3, (Juniperus ashei; SEQ ID NO:59; NCBI Accession No. P81295.1).

“sta” means a nucleotide encoding a translational stabilizing protein.

“Strain” refers to a genetic variant, an isolate, a subtype, a group thereof, or a culture thereof, exhibiting phenotypic and/or genotypic traits belonging to the same lineage, distinct from those of other members of the same species. For example, in some embodiments, the term “strain” can refer to one or more yeast cells having one or more characteristics that makes them differ in some way relative to other yeast cells of their species, wherein said other yeast cells do not possess the one or more characteristics.

“Structural motif” refers to the three-dimensional arrangement of peptides and/or polypeptides, and/or the arrangement of operably linked polypeptide segments. For example, the polypeptide comprising ERSP-STA-L-DVP has an ERSP motif, an STA motif, a LINKER motif, and a DVP polypeptide motif.

“Toxin” refers to a venom and/or a poison, especially a protein or conjugated protein produced by certain animals, higher plants, and pathogenic bacteria. Generally, the term “toxin” is reserved natural products, e.g., molecules and peptides found in scorpions, spiders, snakes, poisonous mushrooms, etc., whereas the term “toxicant” is reserved for man-made products and/or artificial products e.g., man-made chemical pesticides. However, as used herein, the terms “toxin” and “toxicant” are used synonymously

“Transfection” and “transformation” both refer to the process of introducing exogenous and/or heterologous DNA or RNA (e.g., a vector containing a polynucleotide that encodes a DVP) into a host organism (e.g., a prokaryote or a eukaryote). Generally, those having ordinary skill in the art sometimes reserve the term “transformation” to describe processes where exogenous and/or heterologous DNA or RNA are introduced into a bacterial cell; and reserve the term “transfection” for processes that describe the introduction of exogenous and/or heterologous DNA or RNA into eukaryotic cells. However, as used herein, the term “transformation” and “transfection” are used synonymously, regardless of whether a process describes the introduction exogenous and/or heterologous DNA or RNA into a prokaryote (e.g., bacteria) or a eukaryote (e.g., yeast, plants, or animals).

“Transgene” means a heterologous and/or exogenous DNA sequence encoding a protein which is transformed into a plant.

“Transgenic host cell” or “host cell” means a cell which is transformed with a gene and has been selected for its transgenic status via an additional selection gene.

“Transgenic plant” means a plant that has been derived from a single cell that was transformed with foreign DNA such that every cell in the plant contains that transgene.

“Transient expression system” means an Agrobacterium tumefaciens-based system which delivers DNA encoding a disarmed plant virus into a plant cell where it is expressed. The plant virus has been engineered to express a protein of interest at high concentrations, up to 40% of the TSP.

“Triple expression cassette” refers to three DVP expression cassettes contained on the same vector.

“TRBO” means a transient plant expression system using Tobacco mosaic virus with removal of the viral coating protein gene.

“Trypsin cleavage” means an in vitro assay that uses the protease enzyme trypsin (which recognizes exposed lysine and arginine amino acid residues) to separate a cleavable linker at that cleavage site. It also means the act of the trypsin enzyme cleaving that site.

“TSP” or “total soluble protein” means the total amount of protein that can be extracted from a plant tissue sample and solubilized into the extraction buffer.

“UBI” refers to ubiquitin. For example, in some embodiments, UBI can refer to a ubiquitin monomer isolated from Zea mays.

“var.” refers to varietas or variety. The term “var.” is used to indicate a taxonomic category that ranks below the species level and/or subspecies (where present). In some embodiments, the term “var.” represents members differing from others of the same subspecies or species in minor but permanent or heritable characteristics.

“Variant” or “variant sequence” or “variant peptide” refers to an amino acid sequence that possesses one or more conservative amino acid substitutions or conservative modifications. The conservative amino acid substitutions in a “variant” does not substantially diminish the activity of the variant in relation to its non-variant form. For example, in some embodiments, a “variant” possesses one or more conservative amino acid substitutions when compared to a peptide with a disclosed and/or claimed sequence, as indicated by a SEQ ID NO.

“Vector” refers to the DNA segment that accepts a heterologous polynucleotide of interest (e.g., dvp). The heterologous polynucleotide of interest is known as an “insert” or “transgene.”

“Wild type” or “WT” refer to the phenotype and/or genotype (i.e., the appearance or sequence) of an organism, polynucleotide sequence, and/or polypeptide sequence, as it is found and/or observed in its naturally occurring state or condition.

“Yeast expression vector” or “expression vector” or “vector” means a plasmid which can introduce a heterologous gene and/or expression cassette into yeast cells to be transcribed and translated.

“Yield” refers to the production of a peptide, and increased yields can mean increased amounts of production, increased rates of production, and an increased average or median yield and increased frequency at higher yields. The term “yield” when used in reference to plant crop growth and/or production, as in “yield of the plant” refers to the quality and/or quantity of biomass produced by the plant.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, and formulation. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000); each of these references are incorporated herein by reference in their entireties.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in the entireties, except for any definitions, subject matter disclaimers, or disavowals, and except to the extent that the incorporated material is inconsistent with the express disclosure herein, in which case the language in this disclosure controls.

Wild-Type Diguetoxins and DVPS

The American Desert Spider (Diguetia canities), also known as “the desert bush spider,” is a species of coneweb spider found in desert and semi-desert habitats in the United States. Diguetia canities produces toxins that have been shown to have an insecticidal effect, while having no effect on mammals. See Bende et al., A distinct sodium channel voltage-sensor locus determines insect selectivity of the spider toxin Dc1a. Nat Commun. 2014 Jul. 11; 5: 4350.

One of the toxins that Diguetia canities produces is, inter alia, Mu-diguetoxin-Dc1a, (also known as μ-DGTX-Dc1a, or simply “Dc1a”). An exemplary wild-type Mu-diguetoxin-Dc1a polypeptide sequence from Diguetia canities is provided herein, having the amino acid sequence of SEQ ID NO:1 (NCBI Accession No. P49126.1).

The wild-type Dc1a polypeptide exemplified in SEQ ID NO:1 includes a signal peptide region and a propeptide region. Following polypeptide processing, the mature wild-type Dc1a polypeptide possesses an amino acid sequence of “AKDGDVEGPAGCKKYDVECDSGECCQKQYLWYKWRPLDCRCLKSGFFSSKCVCRDV” (SEQ ID NO:2). Dc1a possesses an inhibitor cystine knot (ICK) motif, along with a three-strand beta-sheet that is derived from an extended N-terminal segment, and large inter-cystine loop between residues C25 and C39. Dc1a has disulfide bond connectivity between cysteines at C12 and C25; C19 and C39; C24 and C53; and C41 and C51.

Mu-diguetoxin-Dc1a Variant Polypeptides (DVPs), or pharmaceutically acceptable salts thereof, are mutants or variants that differ from the wild-type mature Mu-diguetoxin-Dc1a (SEQ ID NO:2), e.g., in some embodiments, this variance can be an amino acid substitution, amino acid deletion/insertion, or a change to the polynucleotide encoding the wild-type Mu-diguetoxin-Dc1a. The result of this variation is a non-naturally occurring polypeptide and/or polynucleotide sequence encoding the same that possesses insecticidal activity against one or more insect species relative to the wild-type Mu-diguetoxin-Dc1a.

In some embodiments, a DVP can comprise an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁I-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; Xₙ is C, F, A, T, S, M, or V; and X₁₂ is V, A, or T; or a pharmaceutically acceptable salt thereof.

In some embodiments, a DVP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219, or a pharmaceutically acceptable salt thereof.

In some embodiments, a DVP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a pharmaceutically acceptable salt thereof.

In some embodiments, a DVP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 47, 53, 136, 139-140, 144, 146-147, 187-191, 210-215, or 217-219, or a pharmaceutically acceptable salt thereof.

In some embodiments, a DVP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 213, or 217-219, or a pharmaceutically acceptable salt thereof.

In some embodiments, a DVP can be a homopolymer or heteropolymer of two or more DVPs, wherein the amino acid sequence of each DVP is the same or different.

In some embodiments, a DVP can be a fused protein comprising two or more DVPs separated by a cleavable or non-cleavable linker, and wherein the amino acid sequence of each DVP may be the same or different. And, in some embodiments, the linker is cleavable inside the gut or hemolymph of an insect.

In some embodiments, the DVP can be combined with one or more additional peptides and/or produces. For example, a DVP can be part of a composition comprising a DVP as described herein, and an excipient.

In some embodiments, a DVP can be encoded by a polynucleotide. For example, a polynucleotide operable to encode a DVP, said DVP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; Xₙ is C, F, A, T, S, M, or V; and X₁₂ is V, A, or T, or a complementary nucleotide sequence thereof. In other embodiments, if the polynucleotide encodes a DVP wherein if X₉ is G, T, A, S, M or V, or X_{II} is F, A, T, S, M or V, then a disulfide bond is removed.

In yet other embodiments, the polynucleotide encodes a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219, or a complementary nucleotide sequence thereof.

In yet other embodiments, the polynucleotide encodes a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a complementary nucleotide sequence thereof.

In yet other embodiments, the polynucleotide encodes a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 47, 53, 136, 139-140, 144, 146-147, 187-191, 210-215, or 217-219, or a complementary nucleotide sequence thereof.

In yet other embodiments, the polynucleotide encodes a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 213, or 217-219, or a complementary nucleotide sequence thereof.

In some embodiments, a plant, plant tissue, plant cell, plant seed, or part thereof can comprise one or more DVPs as described herein, or a polynucleotide encoding a DVP as described herein.

In some embodiments, a DVP can be produced by a method comprising: (a) preparing a vector comprising a first expression cassette comprising a polynucleotide operable to express a DVP or complementary nucleotide sequence thereof, said DVP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): A-X₁-D-G-D-V-E-G-P-A-G-C-K—K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T; or a pharmaceutically acceptable salt thereof; or a pharmaceutically acceptable salt thereof, (b) introducing the vector into a yeast cell; and (c) growing the yeast cell in a growth medium under conditions operable to enable expression of the DVP and secretion into the growth medium. In some embodiments, if X₉ is G, T, A, S, M or V, or X₁₁ is F, A, T, S, M or V, then a disulfide bond is removed.

In some embodiments, the vector is a plasmid comprising an alpha-MF signal. In other embodiments, the vector is transformed into a yeast strain. For example, in some embodiments, the yeast strain is selected from any species of the genera Saccharomyces, Pichia, Kluyveromyces, Hansenula, Yarrowia or Schizosaccharomyces. In some embodiments, the yeast strain is selected from the group consisting of Kluyveromyces lactis, Kluyveromyces marxianus, Saccharomyces cerevisiae, and Pichia pastoris. For example, in some embodiments, the yeast strain is Kluyveromyces lactis.

In some embodiments, expression of the DVP provides a yield of: at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of DVP per liter of medium. For example, in some embodiments, expression of the DVP provides a yield of at least 100 mg/L of DVP per liter of medium.

In some embodiments, expression of the DVP in the medium results in the expression of a single DVP in the medium.

In some embodiments, expression of the DVP in the medium results in the expression of a DVP polymer comprising two or more DVP polypeptides in the medium.

In some embodiments, the vector comprises two or three expression cassettes, each expression cassette operable to encode the DVP of the first expression cassette. In some embodiments, the vector comprises two or three expression cassettes, each expression cassette operable to encode the DVP of the first expression cassette, or a DVP of a different expression cassette. In some embodiments, the expression cassette is operable to encode a DVP as set forth in any one of SEQ ID NOs: 6-43, 45-51, 53, 128, 130, 136, 139-140, 144, 146-147, 187-191, 202-215, or 217-219.

Exemplary DVPs of the present invention are provided in Table 1, below.

TABLE 1
Exemplary Mu-diguetoxin-Dc1a Variant Polypeptides including
shorthand name, SEQ ID NO, and full amino acid sequence listing. Nucl. = Nucleotide.
While nucleotide sequences are provided here, the nucleic acid sequence of a
nucleic acid molecule that encodes a protein or polypeptide (e.g., a DVP) can vary
due to degeneracies.

| | Mu-diguetoxin-Dc1a | | Nucl. |
|---|---|---|---|
| SEQ | Variant Polypeptide | | SEQ ID |
| ID NO. | Name | Amino Acid Sequence | NO. |
| 5 | Disulfide Deletion | AKDGDVEGPAGCKKYDVECDSGECCQKQYLWYKWRP | 76 |
| | | LDCRGLKSGFFSSKFVCRDV | |
| 6 | C41T/C51A | AKDGDVEGPAGCKKYDVECDSGECCQKQYLWYKWRP | 77 |
| | | LDCRTLKSGFFSSKAVCRDV | |
| 7 | C41A/C51A | AKDGDVEGPAGCKKYDVECDSGECCQKQYLWYKWRP | 78 |
| | | LDCRALKSGFFSSKAVCRDV | |
| 8 | C41S/C51A | AKDGDVEGPAGCKKYDVECDSGECCQKQYLWYKWRP | 79 |
| | | LDCRSLKSGFFSSKAVCRDV | |
| 9 | C41V/C51A | AKDGDVEGPAGCKKYDVECDSGECCQKQYLWYKWRP | 80 |
| | | LDCRVLKSGFFSSKAVCRDV | |
| 10 | C41A/C51T | AKDGDVEGPAGCKKYDVECDSGECCQKQYLWYKWRP | 81 |
| | | LDCRALKSGFFSSKTVCRDV | |
| 11 | C41A/C51S | AKDGDVEGPAGCKKYDVECDSGECCQKQYLWYKWRP | 82 |
| | | LDCRALKSGFFSSKSVCRDV | |
| 12 | C41A/C51V | AKDGDVEGPAGCKKYDVECDSGECCQKQYLWYKWRP | 83 |
| | | LDCRALKSGFFSSKVVCRDV | |
| 13 | C41T/C51S | AKDGDVEGPAGCKKYDVECDSGECCQKQYLWYKWRP | 84 |
| | | LDCRTLKSGFFSSKSVCRDV | |
| 14 | C41S/C51S | AKDGDVEGPAGCKKYDVECDSGECCQKQYLWYKWRP | 85 |
| | | LDCRSLKSGFFSSKSVCRDV | |
| 15 | C41T/C51A/V17A | AKDGDVEGPAGCKKYDAECDSGECCQKQYLWYKWRP | 86 |
| | | LDCRTLKSGFFSSKAVCRDV | |
| 16 | C41T/C51A/D20A | AKDGDVEGPAGCKKYDVECASGECCQKQYLWYKWRP | 87 |
| | | LDCRTLKSGFFSSKAVCRDV | |
| 17 | C41T/C51A/S21A | AKDGDVEGPAGCKKYDVECDAGECCQKQYLWYKWRP | 88 |
| | | LDCRTLKSGFFSSKAVCRDV | |
| 18 | C41T/C51A/W31A | AKDGDVEGPAGCKKYDVECDSGECCQKQYLAYKWRP | 89 |
| | | LDCRTLKSGFFSSKAVCRDV | |
| 19 | C41T/C51A/Y32A | AKDGDVEGPAGCKKYDVECDSGECCQKQYLWAKWRP | 90 |
| | | LDCRTLKSGFFSSKAVCRDV | |
| 20 | C41T/C51A/P36A | AKDGDVEGPAGCKKYDVECDSGECCQKQYLWYKWRA | 91 |
| | | LDCRTLKSGFFSSKAVCRDV | |
| 21 | C41T/C51A/D38A | AKDGDVEGPAGCKKYDVECDSGECCQKQYLWYKWRP | 92 |
| | | LACRTLKSGFFSSKAVCRDV | |
| 22 | C41T/C51A/L42A | AKDGDVEGPAGCKKYDVECDSGECCQKQYLWYKWRP | 93 |
| | | LDCRTAKSGFFSSKAVCRDV | |
| 23 | C41T/C51A/V52A | AKDGDVEGPAGCKKYDVECDSGECCQKQYLWYKWRP | 94 |
| | | LDCRTLKSGFFSSKAACRDV | |
| 24 | C41T/C51A/W31F | AKDGDVEGPAGCKKYDVECDSGECCQKQYLFYKWRP | 95 |
| | | LDCRTLKSGFFSSKAVCRDV | |
| 25 | C41T/C51A/Y32S | AKDGDVEGPAGCKKYDVECDSGECCQKQYLWSKWRP | 96 |
| | | LDCRTLKSGFFSSKAVCRDV | |
| 26 | C41T/C51A/W31F/ | AKDGDVEGPAGCKKYDVECDSGECCQKQYLFSKWRA | 97 |
| | Y32S/P36A | LDCRTLKSGFFSSKAVCRDV | |
| 27 | C41T/C51A/D20A/ | AKDGDVEGPAGCKKYDVECASGECCQKQYLWYKWRP | 98 |
| | L42N | LDCRTNKSGFFSSKAVCRDV | |
| 28 | C41T/C51A/D20A/ | AKDGDVEGPAGCKKYDVECASGECCQKQYLWYKWRP | 99 |
| | L42V | LDCRTVKSGFFSSKAVCRDV | |
| 29 | C41T/C51A/D38A | AKDGDVEGPAGCKKYDVECDSGECCQKQYLWYKWRP | 100 |
| | | LACRTLKSGFFSSKAVCRDV | |
| 30 | C41T/C51A/D38K | AKDGDVEGPAGCKKYDVECDSGECCQKQYLWYKWRP | 101 |
| | | LKCRTLKSGFFSSKAVCRDV | |
| 31 | C41T/C51A/D38S | AKDGDVEGPAGCKKYDVECDSGECCQKQYLWYKWRP | 102 |
| | | LSCRTLKSGFFSSKAVCRDV | |
| 32 | C41T/C51A/D38A/ | AKDGDVEGPAGCKKYDVECDSGECCQKQYLWYKWRP | 103 |
| | V52T | LACRTLKSGFFSSKATCRDV | |
| 33 | C41T/C51A/D38A/ | AKDGDVEGPAGCKKYDVECDSGECCQKQYLWYKWRP | 104 |
| | V52A | LACRTLKSGFFSSKAACRDV | |
| 34 | C41T/C51A/D38A/ | AKDGDVEGPAGCKKYDEECDSGECCQKQYLWYKWRP | 105 |
| | V17E | LACRTLKSGFFSSKAVCRDV | |
| 35 | C41T/C51A/D38A/ | AKDGDVEGPAGCKKYDVECDSGECCQKQYLWYKWRP | 106 |
| | L42V | LACRTVKSGFFSSKAVCRDV | |
| 36 | C41T/C51A/D38A/ | AKDGDVEGPAGCKKYDVECDSGECCQKQYLWYKWRP | 107 |
| | L42S | LACRTSKSGFFSSKAVCRDV | |
| 37 | C41T/C51A/D38A/ | AKDGDVEGPAGCKKYDVECDSGECCQKQYLWYKWRP | 108 |
| | L42E | LACRTEKSGFFSSKAVCRDV | |
| 38 | C41T/C51A/D38A/ | AKDGDVEGPAGCKKYDVECDSGECCQKQYLWYKWRP | 109 |
| | L42Q | LACRTQKSGFFSSKAVCRDV | |
| 39 | C41T/C51A/D38A/ | AKDGDVEGPAGCKKYDVECASGECCQKQYLWYKWRP | 110 |
| | D20A | LACRTLKSGFFSSKAVCRDV | |
| 40 | C41T/C51A/D20A/ | AKDGDVEGPAGCKKYDVECASGECCQKQYLWSKWRP | 111 |
| | Y32S | LDCRTLKSGFFSSKAVCRDV | |
| 41 | C41T/C51A/D38A/ | AKDGDVEGPAGCKKYDVECDSGECCQKQYLWSKWRP | 112 |
| | Y32S | LACRTLKSGFFSSKAVCRDV | |
| 42 | C41T/C51A/D20A/ | AKDGDVEGPAGCKKYDVECASGECCQKQYLWSKWRP | 113 |
| | D38A/Y32S | LACRTLKSGFFSSKAVCRDV | |
| 43 | C41T/C51A/D20A/ | AKDGDVEGPAGCKKYDVECASGECCQKQYLFSKWRA | 114 |
| | W31F/Y32S/P36A | LDCRTLKSGFFSSKAVCRDV | |
| 44 | D38A | AKDGDVEGPAGCKKYDVECDSGECCQKQYLWYKWRP | 115 |
| | | LACRCLKSGFFSSKCVCRDV | |
| 45 | C41S/C51T/D38A | AKDGDVEGPAGCKKYDVECDSGECCQKQYLWYKWRP | 116 |
| | | LACRSLKSGFFSSKTVCRDV | |
| 46 | C41T/C51T/D38A | AKDGDVEGPAGCKKYDVECDSGECCQKQYLWYKWRP | 117 |
| | | LACRTLKSGFFSSKTVCRDV | |
| 47 | C41S/C51S/D38A | AKDGDVEGPAGCKKYDVECDSGECCQKQYLWYKWRP | 118 |
| | | LACRSLKSGFFSSKSVCRDV | |
| 48 | C41T/C51S/D38A | AKDGDVEGPAGCKKYDVECDSGECCQKQYLWYKWRP | 119 |
| | | LACRTLKSGFFSSKSVCRDV | |
| 49 | C41V/C51T/D38A | AKDGDVEGPAGCKKYDVECDSGECCQKQYLWYKWRP | 120 |
| | | LACRVLKSGFFSSKTVCRDV | |
| 50 | C41T/C51V/D38A | AKDGDVEGPAGCKKYDVECDSGECCQKQYLWYKWRP | 121 |
| | | LACRTLKSGFFSSKVVCRDV | |
| 51 | C41S/C51V/D38A | AKDGDVEGPAGCKKYDVECDSGECCQKQYLWYKWRP | 122 |
| | | LACRSLKSGFFSSKVVCRDV | |
| 52 | C41V/C51S/D38A | AKDGDVEGPAGCKKYDVECDSGECCQKQYLWYKWRP | 123 |
| | | LACRVLKSGFFSSKSVCRDV | |
| 53 | C41S/C51S/D38A/ | AKDGDVEGPAGCKKYDVECDSGECCQKQYLWYKWRP | 124 |
| | L42V | LACRSVKSGFFSSKSVCRDV | |
| 125 | C41N/C51A/D38A/ | AKDGDVEGPAGCKKYDVECDSGECCQKQYLWYKWRP | 153 |
| | L42V | LACRNVKSGFFSSKAVCRDV | |
| 126 | C41D/C51A/D38A/ | AKDGDVEGPAGCKKYDVECDSGECCQKQYLWYKWRP | 154 |
| | L42V | LACRDVKSGFFSSKAVCRDV | |
| 127 | C41S/C51A/D38A/ | AKDGDVEGPAGCKKYDVECDSGECCQKQYLWYKWRP | 155 |
| | L42V | LACRSVKSGFFSSKAVCRDV | |
| 128 | C41M/C51A/D38A/ | AKDGDVEGPAGCKKYDVECDSGECCQKQYLWYKWRP | 156 |
| | L42V | LACRMVKSGFFSSKAVCRDV | |
| 129 | C41T/C51G/D38A/ | AKDGDVEGPAGCKKYDVECDSGECCQKQYLWYKWRP | 157 |
| | L42V | LACRTVKSGFFSSKGVCRDV | |
| 130 | C41T/C51D/D38A/ | AKDGDVEGPAGCKKYDVECDSGECCQKQYLWYKWRP | 158 |
| | L42V | LACRTVKSGFFSSKDVCRDV | |
| 131 | C41T/C51N/D38A/ | AKDGDVEGPAGCKKYDVECDSGECCQKQYLWYKWRP | 159 |
| | L42V | LACRTVKSGFFSSKNVCRDV | |
| 132 | C41T/C51Q/D38A/ | AKDGDVEGPAGCKKYDVECDSGECCQKQYLWYKWRP | 160 |
| | L42V | LACRTVKSGFFSSKQVCRDV | |
| 133 | C41T/C51E/D38A/ | AKDGDVEGPAGCKKYDVECDSGECCQKQYLWYKWRP | 161 |
| | L42V | LACRTVKSGFFSSKEVCRDV | |
| 134 | C41T/C51V/D38A/ | AKDGDVEGPAGCKKYDVECDSGECCQKQYLWYKWRP | 162 |
| | L42V | LACRTVKSGFFSSKVVCRDV | |
| 135 | C41T/C51H/D38A/ | AKDGDVEGPAGCKKYDVECDSGECCQKQYLWYKWRP | 163 |
| | L42V | LACRTVKSGFFSSKHVCRDV | |
| 136 | C41T/C51M/D38A/ | AKDGDVEGPAGCKKYDVECDSGECCQKQYLWYKWRP | 164 |
| | L42V | LACRTVKSGFFSSKMVCRDV | |
| 137 | C41V/C51V/D38A/ | AKDGDVEGPAGCKKYDVECDSGECCQKQYLWYKWRP | 165 |
| | L42V | LACRVVKSGFFSSKVVCRDV | |
| 138 | C41M/C51M/D38A/ | AKDGDVEGPAGCKKYDVECDSGECCQKQYLWYKWRP | 166 |
| | L42V | LACRMVKSGFFSSKMVCRDV | |
| 139 | C41K/C51E/D38A/ | AKDGDVEGPAGCKKYDVECDSGECCQKQYLWYKWRP | 167 |
| | L42V | LACRDVKSGFFSSKEVCRDV | |
| 140 | C41E/C51K/D38A/ | AKDGDVEGPAGCKKYDVECDSGECCQKQYLWYKWRP | 168 |
| | L42V | LACREVKSGFFSSKKVCRDV | |
| 141 | C41T/C51A/D20V/ | AKDGDVEGPAGCKKYDVECVSGECCQKQYLWYKWRP | 169 |
| | D38A/L42V | LACRTVKSGFFSSKAVCRDV | |
| 142 | C41T/C51A/D20G/ | AKDGDVEGPAGCKKYDVECGSGECCQKQYLWYKWRP | 170 |
| | D38A/L42V | LACRTVKSGFFSSKAVCRDV | |
| 143 | C41T/C51A/D20K/ | AKDGDVEGPAGCKKYDVECKSGECCQKQYLWYKWRP | 171 |
| | D38A/L42V | LACRTVKSGFFSSKAVCRDV | |
| 144 | C41T/C51A/D20E/ | AKDGDVEGPAGCKKYDVECESGECCQKQYLWYKWRP | 172 |
| | D38A/L42V | LACRTVKSGFFSSKAVCRDV | |
| 145 | C41T/C51A/D20L/ | AKDGDVEGPAGCKKYDVECLSGECCQKQYLWYKWRP | 173 |
| | D38A/L42V | LACRTVKSGFFSSKAVCRDV | |
| 146 | C41T/C51A/D20N/ | AKDGDVEGPAGCKKYDVECNSGECCQKQYLWYKWRP | 174 |
| | D38A/L42V | LACRTVKSGFFSSKAVCRDV | |
| 147 | C41T/C51A/D20Y/ | AKDGDVEGPAGCKKYDVECYSGECCQKQYLWYKWRP | 175 |
| | D38A/L42V | LACRTVKSGFFSSKAVCRDV | |
| 148 | C41T/C51A/S21G/ | AKDGDVEGPAGCKKYDVECDGGECCQKQYLWYKWRP | 176 |
| | D38A/L42V | LACRTVKSGFFSSKAVCRDV | |
| 149 | C41T/C51A/E18P/ | AKDGDVEGPAGCKKYDVPCDSGECCQKQYLWYKWRP | 177 |
| | D38A/L42V | LACRTVKSGFFSSKAVCRDV | |
| 150 | C41T/C51A/E18K/ | AKDGDVEGPAGCKKYDVKCDSGECCQKQYLWYKWRP | 178 |
| | D38A/L42V | LACRTVKSGFFSSKAVCRDV | |
| 151 | C41T/C51A/E18S/ | AKDGDVEGPAGCKKYDVSCDSGECCQKQYLWYKWRP | 179 |
| | D38A/L42V | LACRTVKSGFFSSKAVCRDV | |
| 152 | C41T/C51A/E18D/ | AKDGDVEGPAGCKKYDVDCDSGECCQKQYLWYKWRP | 180 |
| | D38A/L42V | LACRTVKSGFFSSKAVCRDV | |
| 181 | C41V/C51T/D38A/ | AKDGDVEGPAGCKKYDVECDSGECCQKQYLWYKWRP | 230 |
| | L42V | LACRVLKSGFFSSKTVCRDV | |
| 182 | C41N/C51A | AKDGDVEGPAGCKKYDVECDSGECCQKQYLWYKWRP | 231 |
| | | LDCRNLKSGFFSSKAVCRDV | |
| 187 | Y32S/P36A | AKDGDVEGPAGCKKYDVECDSGECCQKQYLWSKWRA | 232 |
| | | LDCRCLKSGFFSSKCVCRDV | |
| 188 | Y32K/P36A | AKDGDVEGPAGCKKYDVECDSGECCQKQYLWKKWRA | 233 |
| | | LDCRCLKSGFFSSKCVCRDV | |
| 189 | Y32H/P36A | AKDGDVEGPAGCKKYDVECDSGECCQKQYLWHKWRA | 234 |
| | | LDCRCLKSGFFSSKCVCRDV | |
| 190 | W31F/Y32S | AKDGDVEGPAGCKKYDVECDSGECCQKQYLFSKWRP | 235 |
| | | LDCRCLKSGFFSSKCVCRDV | |
| 191 | W31F/Y32S/P36A | AKDGDVEGPAGCKKYDVECDSGECCQKQYLFSKWRA | 236 |
| | | LDCRCLKSGFFSSKCVCRDV | |
| 192 | Y32H/P36A/C41A/ | AKDGDVEGPAGCKKYDVECDSGECCQKQYLWHKWRA | 237 |
| | C51A | LDCRALKSGFFSSKAVCRDV | |
| 202 | C41T/C51A/Y29A | AKDGDVEGPAGCKKYDVECDSGECCQKQALWYKWRP | 238 |
| | | LDCRTLKSGFFSSKAVCRDV | |
| 203 | C41T/C51A/G45A | AKDGDVEGPAGCKKYDVECDSGECCQKQYLWYKWRP | 239 |
| | | LDCRTLKSAFFSSKAVCRDV | |
| 204 | C41T/C51A/F47A | AKDGDVEGPAGCKKYDVECDSGECCQKQYLWYKWRP | 240 |
| | | LDCRTLKSGFASSKAVCRDV | |
| 205 | C41T/C51A/R54A | AKDGDVEGPAGCKKYDVECDSGECCQKQYLWYKWRP | 241 |
| | | LDCRTLKSGFFSSKAVCADV | |
| 206 | C41T/C51A/Y32A | AKDGDVEGPAGCKKYDVECDSGECCQKQYLWAKWRP | 242 |
| | | LDCRTLKSGFFSSKAVCRDV | |
| 207 | C41T/C51A/P36A | AKDGDVEGPAGCKKYDVECDSGECCQKQYLWYKWRA | 243 |
| | | LDCRTLKSGFFSSKAVCRDV | |
| 208 | C41T/C51A/D38A/ | AKDGDVEGPAGCKKYDVECASGECCQKQYLWYKWRP | 244 |
| | L42H | LDCRTHKSGFFSSKAVCRDV | |
| 209 | Y32S/D38A/C41S/ | AKDGDVEGPAGCKKYDVECDSGECCQKQYLWSKWRP | 245 |
| | L42I/C51S | LACRSIKSGFFSSKSVCRDV | |
| 210 | D38A/L42I/C41S/ | AKDGDVEGPAGCKKYDVECDSGECCQKQYLWYKWRP | 220 |
| | C51S | LACRSIKSGFFSSKSVCRDV | |
| 211 | K2L/D38A/C41S/ | ALDGDVEGPAGCKKYDVECDSGECCQKQYLWYKWRP | 221 |
| | C51S | LACRSLKSGFFSSKSVCRDV | |
| 212 | Y32S/D38A/ | AKDGDVEGPAGCKKYDVECDSGECCQKQYLWSKWRP | 222 |
| | C41S/C51S | LACRSLKSGFFSSKSVCRDV | |
| 213 | K2L/Y32S/D38A/ | ALDGDVEGPAGCKKYDVECDSGECCQKQYLWSKWRP | 223 |
| | C41S/C51S | LACRSLKSGFFSSKSVCRDV | |
| 214 | D38T/C41S/C51S | AKDGDVEGPAGCKKYDVECDSGECCQKQYLWYKWRP | 224 |
| | | LTCRSLKSGFFSSKSVCRDV | |
| 215 | D38S/C41S/C51S | AKDGDVEGPAGCKKYDVECDSGECCQKQYLWYKWRP | 225 |
| | | LSCRSLKSGFFSSKSVCRDV | |
| 216 | D38M/C41S/C51S | AKDGDVEGPAGCKKYDVECDSGECCQKQYLWYKWRP | 226 |
| | | LMCRSLKSGFFSSKSVCRDV | |
| 217 | K2L/Y32S/L42I | ALDGDVEGPAGCKKYDVECDSGECCQKQYLWSKWRP | 227 |
| | | LDCRCIKSGFFSSKCVCRDV | |
| 218 | K2L/Y32S/D38A/ | ALDGDVEGPAGCKKYDVECDSGECCQKQYLWSKWRP | 228 |
| | L42I/C41S/C51S | LACRSIKSGFFSSKSVCRDV | |
| 219 | K2L/D38A/L42I/ | ALDGDVEGPAGCKKYDVECDSGECCQKQYLWYKWRP | 229 |
| | C41S/C51S | LACRSIKSGFFSSKSVCRDV | |

In some embodiments, a DVP can have a disulfide deletion. For example, in some embodiments, a DVP can have amino acid substitutions at residues C41 and C51, resulting in the deletion of a disulfide bond. In some embodiments, a DVP with a disulfide deletion can have an amino acid substitution of C51G, C51IF, and/or both, relative to SEQ TD NO:2. In some embodiments, a DVP with a disulfide deletion can have an amino acid sequence of SEQ TD NO: 5. In some embodiments, the term “Disulfide deletion” refers to those embodiments that have an amino acid substitution of C51G, C51IF, and/or both, relative to SEQ TD NO:2.

In some embodiments, a DVP can have amino acid substitutions of C41T and C51A relative to SEQ TD NO:2. For example, in some embodiments, a DVP can have an amino acid sequence of SEQ TD NO:6. In some embodiments, the term “C41T/C51A” refers to those embodiments that have an amino acid substitution of C51G, C51F, and/or both, relative to SEQ ID NO: 2.

In some embodiments, a DVP can have amino acid substitutions of C41A and C51A relative to SEQ TD NO:2. For example, in some embodiments, a DVP can have an amino acid sequence of SEQ ID NO:7. In some embodiments, the term “C41A/C51A” refers to those embodiments that have an amino acid substitution of C41A and C51A relative to SEQ ID NO:2.

In some embodiments, a DVP can have amino acid substitutions of C41S and C51A relative to SEQ ID NO:2. For example, in some embodiments, a DVP can have an amino acid sequence of SEQ ID NO:8. In some embodiments, the term “C41S/C51A” refers to those embodiments that have an amino acid substitution of C41S and C51A relative to SEQ ID NO:2.

In some embodiments, a DVP can have amino acid substitutions of C41V and C51A relative to SEQ ID NO:2. For example, in some embodiments, a DVP can have an amino acid sequence of SEQ ID NO:9. In some embodiments, the term “C41V/C51A” refers to those embodiments that have an amino acid substitution of C41V and C51A relative to SEQ ID NO:2.

In some embodiments, a DVP can have amino acid substitutions of C41A and C51T relative to SEQ ID NO:2. For example, in some embodiments, a DVP can have an amino acid sequence of SEQ ID NO:10. In some embodiments, the term “C41A/C51T” refers to those embodiments that have an amino acid substitution of C41A and C51T relative to SEQ ID NO:2.

In some embodiments, a DVP can have amino acid substitutions of C41A and C51S relative to SEQ ID NO:2. For example, in some embodiments, a DVP can have an amino acid sequence of SEQ ID NO:11. In some embodiments, the term “C41A/C51S” refers to those embodiments that have an amino acid substitution of C41A and C51S relative to SEQ ID NO:2.

In some embodiments, a DVP can have amino acid substitutions of C41A and C51V relative to SEQ ID NO:2. For example, in some embodiments, a DVP can have an amino acid sequence of SEQ ID NO:12. In some embodiments, the term “C41A/C51V” refers to those embodiments that have an amino acid substitution of C41A and C51V relative to SEQ ID NO:2.

In some embodiments, a DVP can have amino acid substitutions of C41T and C51S relative to SEQ ID NO:2. For example, in some embodiments, a DVP can have an amino acid sequence of SEQ ID NO:13. In some embodiments, the term “C41T/C51S” refers to those embodiments that have an amino acid substitution of C41T and C51S relative to SEQ ID NO:2.

In some embodiments, a DVP can have amino acid substitutions of C41S and C51S relative to SEQ ID NO:2. For example, in some embodiments, a DVP can have an amino acid sequence of SEQ ID NO:14. In some embodiments, the term “C41S/C51S” refers to those embodiments that have an amino acid substitution of C41S and C51S relative to SEQ ID NO:2.

In some embodiments, a DVP can have amino acid substitutions of C41T, C51A, and V17A relative to SEQ ID NO:2. For example, in some embodiments, a DVP can have an amino acid sequence of SEQ ID NO:15. In some embodiments, the term “C41T/C51A/V17A” refers to those embodiments that have an amino acid substitution of C41T, C51A, and V17A relative to SEQ ID NO:2.

In some embodiments, a DVP can have amino acid substitutions of C41T, C51A, and D20A relative to SEQ ID NO:2. For example, in some embodiments, a DVP can have an amino acid sequence of SEQ ID NO:16. In some embodiments, the term “C41T/C51A/D20A” refers to those embodiments that have an amino acid substitution of C41T, C51A, and D20A relative to SEQ ID NO:2.

In some embodiments, a DVP can have amino acid substitutions of C41T, C51A, and S21A relative to SEQ ID NO:2. For example, in some embodiments, a DVP can have an amino acid sequence of SEQ ID NO:17. In some embodiments, the term “C41T/C51A/S21A” refers to those embodiments that have an amino acid substitution of C41T, C51A, and S21A relative to SEQ ID NO:2.

In some embodiments, a DVP can have amino acid substitutions of C41T, C51A, and W31A relative to SEQ ID NO:2. For example, in some embodiments, a DVP can have an amino acid sequence of SEQ ID NO:18. In some embodiments, the term “C41T/C51A/W31A” refers to those embodiments that have an amino acid substitution of C41T, C51A, and W31A relative to SEQ ID NO:2.

In some embodiments, a DVP can have amino acid substitutions of C41T, C51A, and Y32A relative to SEQ ID NO:2. For example, in some embodiments, a DVP can have an amino acid sequence of SEQ ID NO: 19. In some embodiments, the term “C41T/C51A/Y32A” refers to those embodiments that have an amino acid substitution of C41T, C51A, and Y32A relative to SEQ ID NO:2.

In some embodiments, a DVP can have amino acid substitutions of C41T, C51A, and P36A relative to SEQ ID NO:2. For example, in some embodiments, a DVP can have an amino acid sequence of SEQ ID NO:20. In some embodiments, the term “C41T/C51A/P36A” refers to those embodiments that have an amino acid substitution of C41T, C51A, and P36A relative to SEQ ID NO:2.

In some embodiments, a DVP can have amino acid substitutions of C41T, C51A, and D38A relative to SEQ ID NO:2. For example, in some embodiments, a DVP can have an amino acid sequence of SEQ ID NO:21. In some embodiments, the term “C41T/C51A/D38A” refers to those embodiments that have an amino acid substitution of C41T, C51A, and D38A relative to SEQ ID NO:2.

In some embodiments, a DVP can have amino acid substitutions of C41T, C51A, and L42A relative to SEQ ID NO:2. For example, in some embodiments, a DVP can have an amino acid sequence of SEQ ID NO:22. In some embodiments, the term “C41T/C51A/L42A” refers to those embodiments that have an amino acid substitution of C41T, C51A, and L42A relative to SEQ ID NO:2.

In some embodiments, a DVP can have amino acid substitutions of C41T, C51A, and V52A relative to SEQ ID NO:2. For example, in some embodiments, a DVP can have an amino acid sequence of SEQ ID NO:23. In some embodiments, the term “C41T/C51A/V52A” refers to those embodiments that have an amino acid substitution of C41T, C51A, and V52A relative to SEQ ID NO:2.

In some embodiments, a DVP can have amino acid substitutions of C41T, C51A, and W31F relative to SEQ ID NO:2. For example, in some embodiments, a DVP can have an amino acid sequence of SEQ ID NO:24. In some embodiments, the term “C41T/C51A/W31F” refers to those embodiments that have an amino acid substitution of C41T, C51A, and W31F relative to SEQ ID NO:2.

In some embodiments, a DVP can have amino acid substitutions of C41T, C51A, and Y32S relative to SEQ ID NO:2. For example, in some embodiments, a DVP can have an amino acid sequence of SEQ ID NO:25. In some embodiments, the term “C41T/C51A/Y32S” refers to those embodiments that have an amino acid substitution of C41T, C51A, and Y32S relative to SEQ ID NO:2.

In some embodiments, a DVP can have amino acid substitutions of C41T, C51A, W31F, Y32S, and P36A relative to SEQ ID NO:2. For example, in some embodiments, a DVP can have an amino acid sequence of SEQ ID NO:26. In some embodiments, the term “C41T/C51A/W31F/Y32S/P36A” refers to those embodiments that have an amino acid substitution of C41T, C51A, W31F, Y32S, and P36A relative to SEQ ID NO:2.

In some embodiments, a DVP can have amino acid substitutions of C41T, C51A, D20A, and L42N relative to SEQ ID NO:2. For example, in some embodiments, a DVP can have an amino acid sequence of SEQ ID NO:27. In some embodiments, the term “C41T/C51A/D20A/L42N” refers to those embodiments that have an amino acid substitution of C41T, C51A, D20A, and L42N relative to SEQ ID NO:2.

In some embodiments, a DVP can have amino acid substitutions of C41T, C51A, D20A, and L42V relative to SEQ ID NO:2. For example, in some embodiments, a DVP can have an amino acid sequence of SEQ ID NO:28. In some embodiments, the term “C41T/C51A/D20A/L42V” refers to those embodiments that have an amino acid substitution of C41T, C51A, D20A, and L42V relative to SEQ ID NO:2.

In some embodiments, a DVP can have amino acid substitutions of C41T, C51A, and D38A relative to SEQ ID NO:2. For example, in some embodiments, a DVP can have an amino acid sequence of SEQ ID NO:29. In some embodiments, the term “C41T/C51A/D38A” refers to those embodiments that have an amino acid substitution of C41T, C51A, and D38A relative to SEQ ID NO:2.

In some embodiments, a DVP can have amino acid substitutions of C41T, C51A, and D38K relative to SEQ ID NO:2. For example, in some embodiments, a DVP can have an amino acid sequence of SEQ ID NO:30. In some embodiments, the term “C41T/C51A/D38K” refers to those embodiments that have an amino acid substitution of C41T, C51A, and D38K relative to SEQ ID NO:2.

In some embodiments, a DVP can have amino acid substitutions of C41T, C51A, and D38S relative to SEQ ID NO:2. For example, in some embodiments, a DVP can have an amino acid sequence of SEQ ID NO:31. In some embodiments, the term “C41T/C51A/D38S” refers to those embodiments that have an amino acid substitution of C41T, C51A, and D38S relative to SEQ ID NO:2.

In some embodiments, a DVP can have amino acid substitutions of C41T, C51A, D38A, and V52T relative to SEQ ID NO:2. For example, in some embodiments, a DVP can have an amino acid sequence of SEQ ID NO:32. In some embodiments, the term “C41T/C51A/D38A/V52T” refers to those embodiments that have an amino acid substitution of C41T, C51A, D38A, and V52T relative to SEQ ID NO:2.

In some embodiments, a DVP can have amino acid substitutions of C41T, C51A, D38A, and V52A relative to SEQ ID NO:2. For example, in some embodiments, a DVP can have an amino acid sequence of SEQ ID NO:33. In some embodiments, the term “C41T/C51A/D38A/V52A” refers to those embodiments that have an amino acid substitution of C41T, C51A, D38A, and V52A relative to SEQ ID NO:2.

In some embodiments, a DVP can have amino acid substitutions of C41T, C51A, D38A, and V17E relative to SEQ ID NO:2. For example, in some embodiments, a DVP can have an amino acid sequence of SEQ ID NO:34. In some embodiments, the term “C41T/C51A/D38A/V17E” refers to those embodiments that have an amino acid substitution of C41T, C51A, D38A, and V17E relative to SEQ ID NO:2.

In some embodiments, a DVP can have amino acid substitutions of C41T, C51A, D38A, and L42V relative to SEQ ID NO:2. For example, in some embodiments, a DVP can have an amino acid sequence of SEQ ID NO:35. In some embodiments, the term “C41T/C51A/D38A/L42V” refers to those embodiments that have an amino acid substitution of C41T, C51A, D38A, and L42V relative to SEQ ID NO:2.

In some embodiments, a DVP can have amino acid substitutions of C41T, C51A, D38A, and L42S relative to SEQ ID NO:2. For example, in some embodiments, a DVP can have an amino acid sequence of SEQ ID NO:36. In some embodiments, the term “C41T/C51A/D38A/L42S” refers to those embodiments that have an amino acid substitution of C41T, C51A, D38A, and L42S relative to SEQ ID NO:2.

In some embodiments, a DVP can have amino acid substitutions of C41T, C51A, D38A, and L42E relative to SEQ ID NO:2. For example, in some embodiments, a DVP can have an amino acid sequence of SEQ ID NO:37. In some embodiments, the term “C41T/C51A/D38A/L42E” refers to those embodiments that have an amino acid substitution of C41T, C51A, D38A, and L42E relative to SEQ ID NO:2.

In some embodiments, a DVP can have amino acid substitutions of C41T, C51A, D38A, and L42Q relative to SEQ ID NO:2. For example, in some embodiments, a DVP can have an amino acid sequence of SEQ ID NO:38. In some embodiments, the term “C41T/C51A/D38A/L42Q” refers to those embodiments that have an amino acid substitution of C41T, C51A, D38A, and L42Q relative to SEQ ID NO:2.

In some embodiments, a DVP can have amino acid substitutions of C41T, C51A, D38A, and D20A relative to SEQ ID NO:2. For example, in some embodiments, a DVP can have an amino acid sequence of SEQ ID NO:39. In some embodiments, the term “C41T/C51A/D38A/D20A” refers to those embodiments that have an amino acid substitution of C41T, C51A, D38A, and D20A relative to SEQ ID NO:2.

In some embodiments, a DVP can have amino acid substitutions of C41T, C51A, D20A, and Y32S relative to SEQ ID NO:2. For example, in some embodiments, a DVP can have an amino acid sequence of SEQ ID NO:40. In some embodiments, the term “C41T/C51A/D20A/Y32S” refers to those embodiments that have an amino acid substitution of C41T, C51A, D20A, and Y32S relative to SEQ ID NO:2.

In some embodiments, a DVP can have amino acid substitutions of C41T, C51A, D38A, and Y32S relative to SEQ ID NO:2. For example, in some embodiments, a DVP can have an amino acid sequence of SEQ ID NO:41. In some embodiments, the term “C41T/C51A/D38A/Y32S” refers to those embodiments that have an amino acid substitution of C41T, C51A, D38A, and Y32S relative to SEQ ID NO:2.

In some embodiments, a DVP can have amino acid substitutions of C41T, C51A, D20A, D38A, and Y32S relative to SEQ ID NO:2. For example, in some embodiments, a DVP can have an amino acid sequence of SEQ ID NO:42. In some embodiments, the term “C41T/C51A/D20A/D38A/Y32S” refers to those embodiments that have an amino acid substitution of C41T, C51A, D20A, D38A, and Y32S relative to SEQ ID NO:2.

In some embodiments, a DVP can have amino acid substitutions of C41T, C51A, D20A, W31F, Y32S, and P36A relative to SEQ ID NO:2. For example, in some embodiments, a DVP can have an amino acid sequence of SEQ ID NO:43. In some embodiments, the term “C41T/C51A/D20A/W31F/Y32S/P36A” refers to those embodiments that have an amino acid substitution of C41T, C51A, D20A, W31F, Y32S, and P36A relative to SEQ ID NO:2.

In some embodiments, a DVP can have an amino acid substitution of D38A relative to SEQ ID NO:2. For example, in some embodiments, a DVP can have an amino acid sequence of SEQ ID NO:44. In some embodiments, the term “D38A” refers to those embodiments that have an amino acid substitution of D38A relative to SEQ ID NO:2.

In some embodiments, a DVP can have an amino acid substitution of C41S, C51T, and D38A relative to SEQ ID NO:2. For example, in some embodiments, a DVP can have an amino acid sequence of SEQ ID NO:45. In some embodiments, the term “C41S/C51T/D38A” refers to those embodiments that have an amino acid substitution of C41S, C51T, and D38A relative to SEQ ID NO:2.

In some embodiments, a DVP can have an amino acid substitution of C41T, C51T, and D38A relative to SEQ ID NO:2. For example, in some embodiments, a DVP can have an amino acid sequence of SEQ ID NO:46. In some embodiments, the term “C41T/C51T/D38A” refers to those embodiments that have an amino acid substitution of C41T, C51T, and D38A relative to SEQ ID NO:2.

In some embodiments, a DVP can have an amino acid substitution of C41S, C51S, and D38A relative to SEQ ID NO:2. For example, in some embodiments, a DVP can have an amino acid sequence of SEQ ID NO:47. In some embodiments, the term “C41S/C51S/D38A” refers to those embodiments that have an amino acid substitution of C41S, C51S, and D38A relative to SEQ ID NO:2.

In some embodiments, a DVP can have an amino acid substitution of C41T, C51S, and D38A relative to SEQ ID NO:2. For example, in some embodiments, a DVP can have an amino acid sequence of SEQ ID NO:48. In some embodiments, the term “C41T/C51S/D38A” refers to those embodiments that have an amino acid substitution of C41T, C51S, and D38A relative to SEQ ID NO:2.

In some embodiments, a DVP can have an amino acid substitution of C41V, C51T, and D38A relative to SEQ ID NO:2. For example, in some embodiments, a DVP can have an amino acid sequence of SEQ ID NO:49. In some embodiments, the term “C41V/C51T/D38A” refers to those embodiments that have an amino acid substitution of C41V, C51T, and D38A relative to SEQ ID NO:2.

In some embodiments, a DVP can have an amino acid substitution of C41T, C51V, and D38A relative to SEQ ID NO:2. For example, in some embodiments, a DVP can have an amino acid sequence of SEQ ID NO:50. In some embodiments, the term “C41T/C51V/D38A” refers to those embodiments that have an amino acid substitution of C41T, C51V, and D38A relative to SEQ ID NO:2.

In some embodiments, a DVP can have an amino acid substitution of C41S, C51V, and D38A relative to SEQ ID NO:2. For example, in some embodiments, a DVP can have an amino acid sequence of SEQ ID NO:51. In some embodiments, the term “C41S/C51V/D38A” refers to those embodiments that have an amino acid substitution of C41S, C51V, and D38A relative to SEQ ID NO:2.

In some embodiments, a DVP can have an amino acid substitution of C41V, C51S, and D38A relative to SEQ ID NO:2. For example, in some embodiments, a DVP can have an amino acid sequence of SEQ ID NO:52. In some embodiments, the term “C41V/C51S/D38A” refers to those embodiments that have an amino acid substitution of C41V, C51S, and D38A relative to SEQ ID NO:2.

In some embodiments, a DVP can have an amino acid substitution of C41S, C51iS, D38A, and L42V relative to SEQ ID NO:2. For example, in some embodiments, a DVP can have an amino acid sequence of SEQ ID NO:53. In some embodiments, the term “C41S/C51S/D38A/L42V” refers to those embodiments that have an amino acid substitution of C41S, C51S, D38A, and L42V relative to SEQ ID NO:2.

In some embodiments, a DVP can have an amino acid substitution of C41S, C51iS, D38A, and L42V relative to SEQ ID NO:2. For example, in some embodiments, a DVP can have an amino acid sequence of SEQ ID NO: 53. In some embodiments, the term “C41S/C51S/D38A/L42V” refers to those embodiments that have an amino acid substitution of C41S, C51S, D38A, and L42V relative to SEQ ID NO:2.

In some embodiments, a DVP can have an amino acid substitution of D38A, L42I, C41S, and C51S relative to SEQ ID NO:2. For example, in some embodiments, a DVP can have an amino acid sequence of SEQ ID NO: 210. In some embodiments, the term “D38A/L42I/C41S/C51S” refers to those embodiments that have an amino acid substitution of D38A, L42I, C41S, and C51S relative to SEQ ID NO:2.

In some embodiments, a DVP can have an amino acid substitution of K2L, D38A, C41S, and C51S relative to SEQ ID NO:2. For example, in some embodiments, a DVP can have an amino acid sequence of SEQ ID NO: 211. In some embodiments, the term “K2L/D38A/C41S/C51S” can refer to those embodiments that have an amino acid substitution of K2L, D38A, C41S, and C51S relative to SEQ ID NO:2.

In some embodiments, a DVP can have an amino acid substitution of Y32S, D38A, C41S, and C51S relative to SEQ ID NO:2. For example, in some embodiments, a DVP can have an amino acid sequence of SEQ ID NO: 212. In some embodiments, the term “Y32S/D38A/C41S/C51S” can refer to those embodiments that have an amino acid substitution of Y32S, D38A, C41S, and C51S relative to SEQ ID NO:2.

In some embodiments, a DVP can have an amino acid substitution of K2L, Y32S, D38A, C41S, and C51S relative to SEQ ID NO:2. For example, in some embodiments, a DVP can have an amino acid sequence of SEQ ID NO: 213. In some embodiments, the term “K2L/Y32S/D38A/C41S/C51S” can refer to those embodiments that have an amino acid substitution of K2L, Y32S, D38A, C41S, and C51S relative to SEQ ID NO:2.

In some embodiments, a DVP can have an amino acid substitution of D38T, C41S, and C51S relative to SEQ ID NO:2. For example, in some embodiments, a DVP can have an amino acid sequence of SEQ ID NO: 214. In some embodiments, the term “D38T/C41S/C51S” can refer to those embodiments that have an amino acid substitution of D38T, C41S, and C51S relative to SEQ ID NO:2.

In some embodiments, a DVP can have an amino acid substitution of D38S, C41S, and C51S relative to SEQ ID NO:2. For example, in some embodiments, a DVP can have an amino acid sequence of SEQ ID NO: 215. In some embodiments, the term “D38S/C41S/C51S” can refer to those embodiments that have an amino acid substitution of D38S, C41S, and C51S relative to SEQ ID NO:2.

In some embodiments, a DVP can have an amino acid substitution of K2L, Y32S, and L42I relative to SEQ ID NO:2. For example, in some embodiments, a DVP can have an amino acid sequence of SEQ ID NO: 217. In some embodiments, the term “K2L/Y32S/L42I” can refer to those embodiments that have an amino acid substitution of K2L, Y32S, and L42I relative to SEQ ID NO:2.

In some embodiments, a DVP can have an amino acid substitution of K2L, Y32S, L42I, C41S, and C51S relative to SEQ ID NO:2. For example, in some embodiments, a DVP can have an amino acid sequence of SEQ ID NO: 217. In some embodiments, the term “K2L/Y32S/L42I/C41S/C51S” can refer to those embodiments that have an amino acid substitution of K2L, Y32S, L42I, C41S, and C51S relative to SEQ ID NO:2.

In various embodiments, polynucleotides encoding DVPs can be used to transform plant cells, yeast cells, or bacteria cells. In some embodiments, the insecticidal DVP transgenic proteins may be formulated into compositions that can be sprayed or otherwise applied in any manner known to those skilled in the art to the surface of plants or parts thereof. Accordingly, DNA constructs are provided herein, operable to encode one or more DVPs under the appropriate conditions in a host cell, for example, a plant cell. Methods for controlling a pest infection by a parasitic insect of a plant cell comprises administering or introducing a polynucleotide encoding an DVP as described herein to a plant, plant tissue, or a plant cell by recombinant techniques and growing said recombinantly altered plant, plant tissue or plant cell in a field exposed to the pest. Alternatively, DVPs can be formulated into a sprayable composition consisting of a DVP and an excipient, and applied directly to susceptible plants by direct application, such that upon ingestion of the DVP by the infectious insect results in a deleterious effect.

In some embodiments, the DVP may comprise an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 6-43, 45-51, 53, 128, 130, 136, 139-140, 144, 146-147, 187-191, 202-215, or 217-219, or a pharmaceutically acceptable salt thereof.

In some embodiments, the DVP may comprise an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 6-11, 15-16, 20-22, 24-26, 29, 35, 45-48, 53, 128, 136, 139-140, 144, 146-147, 187-191, 207, 210-215, or 217-219, or a pharmaceutically acceptable salt thereof.

In some embodiments, the DVP may comprise an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 47, 53, 136, 139-140, 144, 146-147, 187-191, 210-215, or 217-219, or a pharmaceutically acceptable salt thereof.

In some embodiments, the DVP may comprise an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 213, or 217-219, or a pharmaceutically acceptable salt thereof.

In some embodiments, the DVP may comprise an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 128 or 147, or a pharmaceutically acceptable salt thereof.

In some embodiments, a polynucleotide operable to encode a DVP may have an nucleic acid sequence of any one of SEQ ID NOs: 77-114, 116-122, 124, 156, 158, 164, 167-168, 172, 174-175, 220-225, or 227-219. In some embodiments, the polynucleotide operable to encode a DVP may comprise a nucleic acid sequence having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% nucleotide sequence identity to of SEQ ID NOs: 77-114, 116-122, 124, 156, 158, 164, 167-168, 172, 174-175,220-225, or 227-219.

In some embodiments, a polynucleotide operable to encode a DVP may comprise an nucleic acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 77-114, 116-122, 124, 156, 158, 164, 167-168, 172, 174-175,220-225, or 227-219.

In some embodiments, a polynucleotide encoding a DVP can encode a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 6-43, 45-51, 53, 128, 130, 136, 139-140, 144, 146-147, 187-191,202-215, or 217-219.

In some embodiments, a polynucleotide of the present invention comprises a polynucleotide operable to encode a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 6-11, 15-16, 20-22, 24-26, 29, 35, 45-48, 53, 128, 136, 139-140, 144, 146-147, 187-191, 207, 210-215, or 217-219, or a complementary sequence thereof.

In some embodiments, a polynucleotide of the present invention comprises a polynucleotide operable to encode a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 47, 53, 136, 139-140, 144, 146-147, 187-191, 210-215, or 217-219, or a complementary sequence thereof.

In some embodiments, a polynucleotide of the present invention comprises a polynucleotide operable to encode a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in SEQ ID NOs: 213, or 217-219, or a complementary sequence thereof.

In some embodiments, a polynucleotide of the present invention comprises a polynucleotide operable to encode a DVP having an amino sequence as set forth in any one of SEQ ID NOs: 6-43, 45-51, 53, 128, 130, 136, 139-140, 144, 146-147, 187-191, 202-215, or 217-219, or a complementary sequence thereof.

In some embodiments, a polynucleotide of the present invention comprises a polynucleotide operable to encode a DVP having an amino sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a complementary sequence thereof.

In some embodiments, a polynucleotide of the present invention comprises a polynucleotide operable to encode a DVP having an amino sequence as set forth in any one of SEQ ID NOs: 47, 53, 136, 139-140, 144, 146-147, 187-191, 210-215, or 217-219, or a complementary sequence thereof.

In some embodiments, a polynucleotide of the present invention comprises a polynucleotide operable to encode a DVP having an amino sequence as set forth in any one of SEQ ID NOs: 213, or 217-218, or a complementary sequence thereof.

DVP-Insecticidal Proteins

In some embodiments, a DVP-insecticidal protein can be any protein, peptide, polypeptide, amino acid sequence, configuration, construct or arrangement, comprising: (1) at least one DVP, or two or more DVPs; and (2) additional non-toxin peptides, polypeptides, or proteins. For example, in some embodiments, these additional peptides, polypeptides, or proteins may have the ability to increase the mortality and/or inhibit the growth of insects exposed to the DVP-insecticidal protein, relative to the DVP alone; increase the expression of the DVP-insecticidal protein, e.g., in a host cell; and/or affect the post-translational processing of the DVP-insecticidal protein.

In some embodiments, a DVP-insecticidal protein can be a polymer comprising two or more DVPs. In yet other embodiments, a DVP-insecticidal protein can be a polymer comprising two or more DVPs, wherein the DVPs are operably linked via a linker peptide, e.g., a cleavable and/or non-cleavable linker.

In some embodiments, a DVP-insecticidal protein can refer to a one or more DVPs operably linked with one or more proteins such as a stabilizing domain (STA); an endoplasmic reticulum signaling protein (ERSP); an insect cleavable or insect non-cleavable linker (L); and/or any other combination thereof.

In some embodiments, a DVP-insecticidal protein can be a polymer of amino acids that when properly folded or in its most natural thermodynamic state exerts an insecticidal activity against one or more insects. For example, in some embodiments, a DVP-insecticidal protein can be a polymer comprising two or more DVPs that are different. In other embodiments, an insecticidal protein can be a polymer of two or more DVPs that are the same.

In yet other embodiments, a DVP-insecticidal protein can comprise one or more DVPs, and one or more peptides, polypeptides, or proteins, that may assist in the DVP-insecticidal protein's folding.

In some embodiments, a DVP-insecticidal protein can comprise one or more DVPs, and one or more peptides, polypeptides, or proteins, wherein the one or more peptides, polypeptides, or proteins are protein tags that help stability or solubility. In other embodiments, the peptides, polypeptides, or proteins can be protein tags that aid in affinity purification.

In some embodiments, a DVP-insecticidal protein can refer to a one or more DVPs operably linked with one or more proteins such as a stabilizing domain (STA); an endoplasmic reticulum signaling protein (ERSP); an insect cleavable or insect non-cleavable linker; one or more heterologous peptides; one or more additional polypeptides; and/or any other combination thereof. In some embodiments, an insecticidal protein can comprise a one or more DVPs as disclosed herein.

In some embodiments, a DVP-insecticidal protein can comprise a DVP homopolymer, e.g., two or more DVP monomers that are the same DVP. In some embodiments, the insecticidal protein can comprise a DVP heteropolymer, e.g., two or more DVP monomers, wherein the DVP monomers are different.

In some embodiments, the DVP-insecticidal protein may comprise a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191,202-215, or 217-219, or a pharmaceutically acceptable salt thereof.

In some embodiments, a DVP-insecticidal protein can comprise one or more DVPs having an amino acid sequence set forth in SEQ ID NOs: 6-43, 45-51, 53, 128, 130, 136, 139-140, 144, 146-147, 187-191, 202-215, or 217-219, or a pharmaceutically acceptable salt thereof.

In some embodiments, the DVP-insecticidal protein may comprise a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a pharmaceutically acceptable salt thereof.

In some embodiments, the DVP-insecticidal protein may comprise a DVP having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 47, 53, 136, 139-140, 144, 146-147, 187-191, 210-215, or 217-219, or a pharmaceutically acceptable salt thereof.

In some embodiments, a DVP-insecticidal protein can comprise one or more DVPs having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191,202-215, or 217-219, or a pharmaceutically acceptable salt thereof.

In some embodiments, a DVP-insecticidal protein can comprise one or more DVPs having an amino acid sequence set forth in SEQ ID NOs: 6-43, 45-51, 53, 128, 130, 136, 139-140, 144, 146-147, 187-191, 202-215, or 217-219, or a pharmaceutically acceptable salt thereof.

In some embodiments, the DVP-insecticidal protein can comprise one or more DVPs, wherein the DVPs are the same or different.

Exemplary methods for the generation of cleavable and non-cleavable linkers can be found in U.S. patent application Ser. No. 15/727,277; and PCT Application No. PCT/US2013/030042, the disclosure of which are incorporated herein by reference in their entireties.

In some embodiments, a DVP-insecticidal protein can be a fusion protein comprising one or more DVPs as described herein, operably linked to an alpha mating factor (alpha-MF) peptide.

An “alpha mating factor (alpha-MF) peptide” or “alpha-MF signal” or “alpha-MF” or “alpha mating factor secretion signal” or “αMF secretion signal” (all used interchangeably) refers to a signal peptide that allows for secreted expression in a recombinant expression system, when the alpha-MF peptide is operably linked to a recombinant peptide of interest (e.g., a DVP). The Alpha-MF peptide directs nascent recombinant polypeptides to the secretory pathway of the recombinant expression system (e.g., a yeast recombinant expression system.

Alpha-MF peptides are well known in the art. Exemplary alpha-MF peptides are provided herein, including, without limitation: Kluyveromyces lactis alpha mating factor pre-pro secretion leader of the pKLAC1 vector (SEQ ID NO: 246); NCBI Accession No. XP_454814 (SEQ ID NO: 247); Mf(alpha)1/Mf(alpha)2 (SEQ ID NO: 248; NCBI Accession No. QEU61411.1); Mating factor alpha precursor N-terminus (SEQ ID NO: 249; NCBI Accession No. KAG0674310); and the like.

In some embodiments, a fusion protein can comprise one or more DVPs operably linked to an alpha mating factor (alpha-MF) peptide; wherein said one or more DVPs have an amino acid sequence that is at least 80%, 85%, 90%, or at least 95% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁I-X₁₂-C-R-D-V, wherein the DVP comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; Xₙ is C, F, A, T, S, M, or V; and X₁₂ is V, A, or T, or a pharmaceutically acceptable salt thereof.

In some embodiments, a fusion protein can comprise one or more DVPs operably linked to an alpha mating factor (alpha-MF) peptide; wherein said one or more DVPs have an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): A-X₁-D-G-D-V-E-G-P-A-G-C-K—K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the DVP comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T, or a pharmaceutically acceptable salt thereof, wherein if X₉ is G, T, A, S, M or V, or X₁₁ is F, A, T, S, M or V, then a disulfide bond is removed.

In some embodiments, a fusion protein can comprise one or more DVPs operably linked to an alpha mating factor (alpha-MF) peptide; wherein the one or more DVPs comprise an amino sequence as set forth in any one of SEQ ID NOs: 6-43, 45-51, 53, 128, 130, 136, 139-140, 144, 146-147, 187-191, 202-215, or 217-219.

In some embodiments, a fusion protein can comprise one or more DVPs operably linked to an alpha mating factor (alpha-MF) peptide; wherein the one or more DVPs comprise an amino sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219.

In some embodiments, a fusion protein can comprise one or more DVPs operably linked to an alpha mating factor (alpha-MF) peptide; wherein the one or more DVPs comprise an amino sequence as set forth in any one of SEQ ID NOs: 47, 53, 136, 139-140, 144, 146-147, 187-191, 210-215, or 217-219.

In some embodiments, a fusion protein can comprise one or more DVPs operably linked to an alpha mating factor (alpha-MF) peptide; the one or more DVPs is a homopolymer or heteropolymer of two or more DVPs, wherein the amino acid sequence of each DVP is the same or different.

In some embodiments, a fusion protein can comprise one or more DVPs operably linked to an alpha mating factor (alpha-MF) peptide; wherein the one or more DVPs, the alpha-MF, or a combination thereof, are separated by a cleavable linker or non-cleavable linker.

In some embodiments, a fusion protein can comprise one or more DVPs operably linked to an alpha mating factor (alpha-MF) peptide; wherein the cleavable linker is cleavable inside the gut or hemolymph of an insect.

In some embodiments, a fusion protein can comprise one or more DVPs operably linked to an alpha mating factor (alpha-MF) peptide; wherein the alpha-MF peptide is an alpha-MF peptide derived from a yeast species.

In some embodiments, a fusion protein can comprise one or more DVPs operably linked to an alpha mating factor (alpha-MF) peptide; wherein the alpha-MF peptide is derived from a yeast species selected from any species of the genera Saccharomyces, Pichia, Kluyveromyces, Hansenula, Yarrowia or Schizosaccharomyces.

In some embodiments, a fusion protein can comprise one or more DVPs operably linked to an alpha mating factor (alpha-MF) peptide; wherein the alpha-MF peptide is derived from a yeast species that is selected from the group consisting of Kluyveromyces lactis, Kluyveromyces marxianus, Saccharomyces cerevisiae, and Pichia pastoris.

In some embodiments, a fusion protein can comprise one or more DVPs operably linked to an alpha mating factor (alpha-MF) peptide; wherein the alpha-MF peptide is derived from a Kluyveromyces lactis or Kluyveromyces marxianus.

In some embodiments, the alpha-MF peptide can be an alpha-MF peptide derived from a Kluyveromyces lactis.

In some embodiments, the alpha-MF peptide can be a K. lactis α-mating factor (a-MF) secretion domain (for secreted expression).

In some embodiments, the alpha-MF peptide can having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 246-249.

In some embodiments, the alpha-MF peptide can having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in SEQ ID NO: 246.

In some embodiments, the alpha-MF peptide can having an amino acid sequence as set forth in any one of SEQ ID NOs: 246-249.

In some embodiments, the alpha-MF peptide can having an amino acid sequence as set forth in SEQ ID NO: 246.

In some embodiments, a fusion protein can comprise one or more DVPs having an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219; wherein the one or more DVPs are operably linked to an alpha-MF peptide having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 246-249.

In some embodiments, a fusion protein can comprise one or more DVPs having an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219; wherein the one or more DVPs are operably linked to an alpha-MF peptide having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 246-249; and further comprising additional non-toxin peptides, polypeptides, or proteins, wherein said additional non-toxin peptides, polypeptides, or proteins e.g., in some embodiments, have the ability to do one or more of the following: increase the mortality and/or inhibit the growth of insects when the insects are exposed to a DVP-insecticidal protein, relative to a DVP alone; increase the expression of said DVP-insecticidal protein, e.g., in a host cell or an expression system; and/or affect the post-translational processing of the DVP-insecticidal protein (e.g., allow for secreted expression of the DVP-insecticidal protein).

In some embodiments, a fusion protein can comprise one or more DVPs operably linked to an alpha mating factor (alpha-MF) peptide; wherein there are two or more DVPs.

In some embodiments, a fusion protein can comprise one or more DVPs operably linked to an alpha mating factor (alpha-MF) peptide; wherein there are two or more DVPs, wherein the DVPs and/or the alpha-MF peptide are operably linked via a linker peptide, e.g., a cleavable and/or non-cleavable linker.

In some embodiments, a DVP-insecticidal protein can be a fusion protein comprising one or more DVPs operably linked to an alpha mating factor (alpha-MF) peptide; and further operably linked with one or more proteins such as a stabilizing domain (STA); an endoplasmic reticulum signaling protein (ERSP); an insect cleavable or insect non-cleavable linker (L); and/or any other combination thereof.

Any of the DVPs described herein, can be used to produce a fusion protein comprising one or more DVPs operably linked to an alpha mating factor (alpha-MF) peptide. For example, any of the DVPs described herein can be used to produce a fusion protein comprising one or more DVPs operably linked to an alpha mating factor (alpha-MF) peptide, e.g., wherein the one or more DVPs has an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219, which are likewise described herein.

Exemplary DVPs and DVP-Insecticidal Proteins

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AKDGDVEGPAGCKKYDVECDSGECCQKQYLWYKWRPLACRSLKSGFFSSKSVCRDV” (SEQ ID NO: 47), or a pharmaceutically acceptable salt thereof.

In some embodiments, a DVP or a DVP-insecticidal protein comprises, consists essentially of, or consists of, the amino acid sequence: “AKDGDVEGPAGCKKYDVECDSGECCQKQYLWYKWRPLACRSLKSGFFSSKSVCRDV” (SEQ ID NO: 47), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AKDGDVEGPAGCKKYDVECDSGECCQKQYLWYKWRPLACRSVKSGFFSSKSVCRDV” (SEQ ID NO: 53), or a pharmaceutically acceptable salt thereof.

In some embodiments, a DVP or a DVP-insecticidal protein comprises, consists essentially of, or consists of, the amino acid sequence: “AKDGDVEGPAGCKKYDVECDSGECCQKQYLWYKWRPLACRSVKSGFFSSKSVCRDV” (SEQ ID NO: 53), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence: “AKDGDVEGPAGCKKYDVECDSGECCQKQYLWYKWRPLACRTVKSGFFSSKMVCRDV” (SEQ ID NO: 136), or a pharmaceutically acceptable salt thereof.

In some embodiments, a DVP or a DVP-insecticidal protein comprises, consists essentially of, or consists of, the amino acid sequence: “AKDGDVEGPAGCKKYDVECDSGECCQKQYLWYKWRPLACRTVKSGFFSSKMVCRDV” (SEQ ID NO: 136), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWYKWRPLACRDVKSGFFSSKEVCRDV”(SEQ ID NO: 139), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWYKWRPLACRDVKSGFFSSKEVCRDV” (SEQ ID NO: 139), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWYKWRPLACREVKSGFFSSKKVCRDV” (SEQ ID NO: 140), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWYKWRPLACREVKSGFFSSKKVCRDV” (SEQ ID NO: 140), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECESGECCQKQYLWYKWRPLACRTVKSGFFSSKAVCRDV” (SEQ ID NO: 144), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECESGECCQKQYLWYKWRPLACRTVKSGFFSSKAVCRDV” (SEQ ID NO: 144), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECNSGECCQKQYLWYKWRPLACRTVKSGFFSSKAVCRDV” (SEQ ID NO: 146), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECNSGECCQKQYLWYKWRPLACRTVKSGFFSSKAVCRDV” (SEQ ID NO: 146), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECYSGECCQKQYLWYKWRPLACRTVKSGFFSSKAVCRDV” (SEQ ID NO: 147), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECYSGECCQKQYLWYKWRPLACRTVKSGFFSSKAVCRDV” (SEQ ID NO: 147), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWSKWRALDCRCLKSGFFSSKCVCRDV” (SEQ ID NO: 187), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWSKWRALDCRCLKSGFFSSKCVCRDV” (SEQ ID NO: 187), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWKKWRALDCRCLKSGFFSSKCVCRDV” (SEQ ID NO: 188), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWKKWRALDCRCLKSGFFSSKCVCRDV” (SEQ ID NO: 188), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWHKWRALDCRCLKSGFFSSKCVCRDV” (SEQ ID NO: 189), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWHKWRALDCRCLKSGFFSSKCVCRDV” (SEQ ID NO: 189), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLFSKWRPLDCRCLKSGFFSSKCVCRDV” (SEQ ID NO: 190), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLFSKWRPLDCRCLKSGFFSSKCVCRDV” (SEQ ID NO: 190), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLFSKWRALDCRCLKSGFFSSKCVCRDV” (SEQ ID NO: 191), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLFSKWRALDCRCLKSGFFSSKCVCRDV” (SEQ ID NO: 191), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWSKWRPLACRSIKSGFFSSKSVCRDV” (SEQ ID NO: 209), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWSKWRPLACRSIKSGFFSSKSVCRDV” (SEQ ID NO: 209), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWYKWRPLACRSIKSGFFSSKSVCRDV” (SEQ ID NO: 210), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWYKWRPLACRSIKSGFFSSKSVCRDV” (SEQ ID NO: 210), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“ALDGDVEGPAGCKKYDVECDSGECCQKQYLWYKWRPLACRSLKSGFFSSKSVCRDV” (SEQ ID NO: 211), or a pharmaceutically acceptable salt thereof.

In some embodiments, a DVP or a DVP-insecticidal protein comprises, consists essentially of, or consists of, the amino acid sequence: “ALDGDVEGPAGCKKYDVECDSGECCQKQYLWYKWRPLACRSLKSGFFSSKSVCRDV” (SEQ ID NO: 211), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWSKWRPLACRSLKSGFFSSKSVCRDV” (SEQ ID NO: 212), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWSKWRPLACRSLKSGFFSSKSVCRDV” (SEQ ID NO: 212), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“ALDGDVEGPAGCKKYDVECDSGECCQKQYLWSKWRPLACRSLKSGFFSSKSVCRDV” (SEQ ID NO: 213), or a pharmaceutically acceptable salt thereof.

In some embodiments, a DVP or a DVP-insecticidal protein comprises, consists essentially of, or consists of, the amino acid sequence:

“ALDGDVEGPAGCKKYDVECDSGECCQKQYLWSKWRPLACRSLKSGFFSSKSVCRDV” (SEQ ID NO: 213), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWYKWRPLTCRSLKSGFFSSKSVCRDV” (SEQ ID NO: 214), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWYKWRPLTCRSLKSGFFSSKSVCRDV” (SEQ ID NO: 214), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“AKDGDVEGPAGCKKYDVECDSGECCQKQYLWYKWRPLSCRSLKSGFFSSKSVCRDV” (SEQ ID NO: 215), or a pharmaceutically acceptable salt thereof.

In some embodiments, a DVP or a DVP-insecticidal protein comprises, consists essentially of, or consists of, the amino acid sequence:

“AKDGDVEGPAGCKKYDVECDSGECCQKQYLWYKWRPLSCRSLKSGFFSSKSVCRDV” (SEQ ID NO: 215), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“ALDGDVEGPAGCKKYDVECDSGECCQKQYLWSKWRPLDCRCIKSGFFSSKCVCRDV” (SEQ ID NO: 217), or a pharmaceutically acceptable salt thereof.

In some embodiments, a DVP or a DVP-insecticidal protein comprises, consists essentially of, or consists of, the amino acid sequence:

“ALDGDVEGPAGCKKYDVECDSGECCQKQYLWSKWRPLDCRCIKSGFFSSKCVCRDV” (SEQ ID NO: 217), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“ALDGDVEGPAGCKKYDVECDSGECCQKQYLWSKWRPLACRSIKSGFFSSKSVCRDV” (SEQ ID NO: 218), or a pharmaceutically acceptable salt thereof.

In some embodiments, a DVP or a DVP-insecticidal protein comprises, consists essentially of, or consists of, the amino acid sequence:

“ALDGDVEGPAGCKKYDVECDSGECCQKQYLWSKWRPLACRSIKSGFFSSKSVCRDV” (SEQ ID NO: 218), or a pharmaceutically acceptable salt thereof.

In some embodiments, a DVP or a DVP-insecticidal protein comprises, consists essentially of, or consists of, an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence:

“ALDGDVEGPAGCKKYDVECDSGECCQKQYLWYKWRPLACRSIKSGFFSSKSVCRDV” (SEQ ID NO: 219), or a pharmaceutically acceptable salt thereof.

In some embodiments, a DVP or a DVP-insecticidal protein comprises, consists essentially of, or consists of, the amino acid sequence:

“ALDGDVEGPAGCKKYDVECDSGECCQKQYLWYKWRPLACRSIKSGFFSSKSVCRDV” (SEQ ID NO: 219), or a pharmaceutically acceptable salt thereof.

Methods for Producing a DVP

Methods of producing proteins are well known in the art, and there are a variety of techniques available. For example, in some embodiments, proteins can be produced using recombinant methods, or chemically synthesized.

In some embodiments, a DVP of the present invention can be created using any known method for producing a protein. For example, in some embodiments, and without limitation, a DVP can be created using a recombinant expression system, such as yeast expression system or a bacterial expression system. However, those having ordinary skill in the art will recognize that other methods of protein production are available.

In some embodiments, the present invention provides a method of producing a DVP using a recombinant expression system.

In some embodiments, the present invention comprises, consists essentially of, or consists of, a method of producing a DVP, said method comprising: (a) preparing a vector comprising a first expression cassette comprising, consisting essentially of, or consisting of, a polynucleotide operable to encode a DVP, or a complementary nucleotide sequence thereof, (b) introducing the vector into a host cell, for example a bacteria or a yeast, or an insect, or a plant cell, or an animal cell; and (c) growing the yeast strain in a growth medium under conditions operable to enable expression of the DVP and secretion into the growth medium. In some related embodiments, the host cell, is a yeast cell.

The invention is practicable in a wide variety of host cells (see host cell section below). Indeed, an end-user of the invention can practice the teachings thereof in any host cell of his or her choosing. Thus, in some embodiments, the host cell can be any host cell that satisfies the requirements of the end-user; i.e., in some embodiments, the expression of a DVP may be accomplished using a variety of host cells, and pursuant to the teachings herein. For example, in some embodiments, a user may desire to use one specific type of host cell (e.g., a yeast cell or a bacteria cell) as opposed to another; the preference of a given host cell can range from availability to cost.

For example, in some embodiments, in some embodiments, the present invention comprises, consists essentially of, or consists of, a method of producing a DVP, said method comprising: (a) preparing a vector comprising a first expression cassette comprising, consisting essentially of, or consisting of, a polynucleotide operable to encode a DVP, or a complementary nucleotide sequence thereof, (b) introducing the vector into a host cell, for example a bacteria or a yeast, or an insect, or a plant cell, or an animal cell; and (c) growing the yeast strain in a growth medium under conditions operable to enable expression of the DVP and secretion into the growth medium. In some related embodiments, the host cell, is a yeast cell.

Isolating and Mutating Wild-Type Mu-Diguetoxin-Dc1a

In various illustrative embodiments, an DVP can be obtained by creating a mutation in the wild-type Mu-diguetoxin-Dc1a polynucleotide sequence; inserting that Mu-diguetoxin-Dc1a variant polynucleotide (dvp) sequence into the appropriate vector; transforming a host organism in such a way that the polynucleotide encoding a DVP is expressed; culturing the host organism to generate the desired amount of DVP; and then purifying the DVP from in and/or around host organism.

Wild-type Mu-diguetoxin-Dc 1a toxins can be isolated from venom, which in turn can be isolated from the venom glands of spiders, e.g., Diguetia canities, using any of the techniques known to those having ordinary skill in the art. For example, in some embodiments, venom can be isolated according to the methods described in U.S. Pat. No. 5,688,764, the disclosure of which is incorporated herein by reference in its entirety.

A wild-type Mu-diguetoxin-Dc1a polynucleotide sequence can be obtained by screening a genomic library using primer probes directed to the Mu-diguetoxin-Dc1a polynucleotide sequence. Alternatively, wild-type Mu-diguetoxin-Dc1a polynucleotide sequence and/or DVP polynucleotide sequences can be chemically synthesized. For example, a wild-type Mu-diguetoxin-Dc1a polynucleotide sequence and/or DVP polynucleotide sequence can be generated using the oligonucleotide synthesis methods such as the phosphoramidite; triester, phosphite, or H-Phosphonate methods (see Engels, J. W. and Uhlmann, E. (1989), Gene Synthesis [New Synthetic Methods (77)]. Angew. Chem. Int. Ed. Engl., 28: 716-734, the disclosure of which is incorporated herein by reference in its entirety).

Producing a mutation in wild-type Mu-diguetoxin-Dc1a polynucleotide sequence can be achieved by various means that are well known to those having ordinary skill in the art. Methods of mutagenesis include Kunkel's method; cassette mutagenesis; PCR site-directed mutagenesis; the “perfect murder” technique (delitto perfetto); direct gene deletion and site-specific mutagenesis with PCR and one recyclable marker; direct gene deletion and site-specific mutagenesis with PCR and one recyclable marker using long homologous regions; transplacement “pop-in pop-out” method; and CRISPR-Cas 9. Exemplary methods of site-directed mutagenesis can be found in Ruvkun & Ausubel, A general method for site-directed mutagenesis in prokaryotes. Nature. 1981 Jan. 1; 289(5793):85-8; Wallace et al., Oligonucleotide directed mutagenesis of the human beta-globin gene: a general method for producing specific point mutations in cloned DNA. Nucleic Acids Res. 1981 Aug. 11; 9(15):3647-56; Dalbadie-McFarland et al., Oligonucleotide-directed mutagenesis as a general and powerful method for studies of protein function. Proc Natl Acad Sci USA. 1982 November; 79(21):6409-13; Bachman. Site-directed mutagenesis. Methods Enzymol. 2013; 529:241-8; Carey et al., PCR-mediated site-directed mutagenesis. Cold Spring Harb Protoc. 2013 Aug. 1; 2013(8):738-42; and Cong et al., Multiplex genome engineering using CRISPR/Cas systems. Science. 2013 Feb. 15; 339(6121):819-23, the disclosures of all of the aforementioned references are incorporated herein by reference in their entireties.

Chemically Synthesizing DVP Polynucleotides

In some embodiments, the polynucleotide sequence encoding the DVP can be chemically synthesized using commercially available polynucleotide synthesis services such as those offered by Genewiz® (e.g., TurboGENE™; PriorityGENE; and FragmentGENE), or Sigma-Aldrich® (e.g., Custom DNA and RNA Oligos Design and Order Custom DNA Oligos). Exemplary method for generating DNA and or custom chemically synthesized polynucleotides are well known in the art, and are illustratively provided in U.S. Pat. No. 5,736,135, Ser. No. 08/389,615, filed on Feb. 13, 1995, the disclosure of which is incorporated herein by reference in its entirety. See also Agarwal, et al., Chemical synthesis of polynucleotides. Angew Chem Int Ed Engl. 1972 June; 11(6):451-9; Ohtsuka et al., Recent developments in the chemical synthesis of polynucleotides. Nucleic Acids Res. 1982 Nov. 11; 10(21): 6553-6570; Sondek & Shortle. A general strategy for random insertion and substitution mutagenesis: substoichiometric coupling of trinucleotide phosphoramidites. Proc Natl Acad Sci USA. 1992 Apr. 15; 89(8): 3581-3585; Beaucage S. L., et al., Advances in the Synthesis of Oligonucleotides by the Phosphoramidite Approach. Tetrahedron, Elsevier Science Publishers, Amsterdam, NL, vol. 48, No. 12, 1992, pp. 2223-2311; Agrawal (1993) Protocols for Oligonucleotides and Analogs: Synthesis and Properties; Methods in Molecular Biology Vol. 20, the disclosure of which is incorporated herein by reference in its entirety.

Chemically synthesizing polynucleotides allows for a DNA sequence to be generated that is tailored to produce a desired polypeptide based on the arrangement of nucleotides within said sequence (i.e., the arrangement of cytosine [C], guanine [G], adenine [A] or thymine [T] molecules); the mRNA sequence that is transcribed from the chemically synthesized DNA polynucleotide can be translated to a sequence of amino acids, each amino acid corresponding to a codon in the mRNA sequence. Accordingly, the amino acid composition of a polypeptide chain that is translated from an mRNA sequence can be altered by changing the underlying codon that determines which of the 20 amino acids will be added to the growing polypeptide; thus, mutations in the DNA such as insertions, substitutions, deletions, and frameshifts may cause amino acid insertions, substitutions, or deletions, depending on the underlying codon.

In some embodiments, a polynucleotide can be chemically synthesized, wherein said polynucleotide harbors one or more mutations. In some embodiments, an mRNA can be created from the template DNA sequence. In yet other embodiments, the mRNA can be cloned and transformed into a competent cell.

Vectors and Transformation

A vector of the present invention refers to a means for introducing one or more heterologous polynucleotides into a host cell (e.g., a yeast cell). There are a variety of vectors available and cloning strategies known to those having ordinary skill in the art.

As used herein, the term “vector” refers to a carrier nucleic acid molecule into which a polynucleotide can be inserted for introduction into a cell (e.g., transformation), and where it can be replicated. In some embodiments, a vector may contain “vector elements,” e.g., and without limitation: an origin of replication (ORI); a gene or nucleotide sequence that allows for selection (e.g., a gene that confers antibiotic resistance or a nucleotide sequence that allows growth in defined media); multiple cloning sites; a promoter region; a primer binding site; and/or a combination thereof.

In some embodiments, some of the polynucleotides or nucleotide sequences inserted into a vector can be “heterologous” or “exogenous,” which means that it is foreign to the cell into which the vector is being introduced, or that the sequence is homologous to a sequence in the cell but in a position within the host cell nucleic acid in which the sequence is ordinarily not found. For example, in some embodiments, a recombinant yeast cell can be transformed with a vector comprising a heterologous polynucleotide comprising an endogenous nucleotide sequence, but is in a position within the host cell nucleic acid in which the endogenous nucleotide sequence is ordinarily not found.

Vectors can be used both as a means to prepare the heterologous polynucleotides of the present invention, or to ultimately transform the cells used to generate a recombinant yeast cell and/or as a method to increase expression of a heterologous polypeptide.

In some embodiments, vectors include plasmids, cosmids, viruses (bacteriophage, animal viruses, and plant viruses), and artificial chromosomes (e.g., YACs). For example, in some embodiments, a vector can be a plasmid, which can introduce a heterologous polynucleotide and/or expression cassette into host cells to be transcribed and translated.

One having ordinary skill in the art would be well equipped to construct a vector through standard recombinant techniques, which are described in Sambrook et al., 1989 and Ausubel et al., 1996, both incorporated herein by reference in their entireties.

In some embodiments, in addition to encoding heterologous polynucleotide, a vector may also encode a targeting molecule. A targeting molecule is one that directs the desired polynucleotide to a particular location.

In some embodiments, a heterologous polynucleotide operable to encode a DVP, can be inserted into any suitable vector, e.g., a plasmid, bacteriophage, or viral vector for amplification, and may thereby be propagated using methods known in the art, such as those described in Molecular Cloning A Laboratory Manual, 2nd Ed., ed. by Sambrook, Fritsch and Maniatis (Cold Spring Harbor Laboratory Press: 1989), the disclosure of which is incorporated herein by reference in its entirety.

Obtaining a DVP from a chemically synthesized DNA polynucleotide sequence and/or a wild-type DNA polynucleotide sequence that has been altered via mutagenesis can be achieved by cloning the DNA sequence into an appropriate vector. There are a variety of expression vectors available, host organisms, and cloning strategies known to those having ordinary skill in the art. For example, the vector can be a plasmid, which can introduce a heterologous gene and/or expression cassette into yeast cells to be transcribed and translated. The term “vector” is used to refer to a carrier nucleic acid molecule into which a nucleic acid sequence can be inserted for introduction into a cell where it can be replicated. A vector may contain “vector elements” such as an origin of replication (ORI); a gene that confers antibiotic resistance to allow for selection; multiple cloning sites; a promoter region; a selection marker for non-bacterial transfection; and a primer binding site. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to encoding an Dc1a variant polynucleotide, a vector may encode a targeting molecule. A targeting molecule is one that directs the desired nucleic acid to a particular tissue, cell, or other location.

In some embodiments, a polynucleotide operable to encode a DVP or a DVP-insecticidal protein can be transformed into a host cell.

In some embodiments, a polynucleotide operable to encode a DVP or a DVP-insecticidal protein can be cloned into a vector, and transformed into a host cell.

In some embodiments, a DVP ORF can be transformed into a host cell.

In addition to a polynucleotide sequence operable to encode a DVP (e.g., a DVP ORF) or a DVP-insecticidal protein, additional DNA segments known as regulatory elements can be cloned into a vector that allow for enhanced expression of the foreign DNA or transgene; examples of such additional DNA segments include (1) promoters, terminators, and/or enhancer elements; (2) an appropriate mRNA stabilizing polyadenylation signal; (3) an internal ribosome entry site (IRES); (4) introns; and (5) post-transcriptional regulatory elements. The combination of a DNA segment of interest (e.g., dvp) with any one of the foregoing cis-acting elements is called an “expression cassette.”

In some embodiments, an expression cassette or DVP expression cassette can contain one or more DVPs, and/or one or more DVP-insecticidal proteins.

In some embodiments, an expression cassette or DVP expression cassette can contain one or more DVPs, and/or one or more DVP-insecticidal proteins, and one or more additional regulatory elements such as: (1) promoters, terminators, and/or enhancer elements; (2) an appropriate mRNA stabilizing polyadenylation signal; (3) an internal ribosome entry site (IRES); (4) introns; and (5) post-transcriptional regulatory elements.

In some embodiments, a single expression cassette can contain one or more of the aforementioned regulatory elements, and a polynucleotide operable to express a DVP. For example, in some embodiments, a DVP expression cassette can comprise polynucleotide operable to express an DVP, and an α-MF signal; Kex2 site; LAC4 terminator; ADN1 promoter; and an acetamidase (amdS) selection marker-flanked by LAC4 promoters on the 5′-end and 3′-end.

In some embodiments, there can be numerous expression cassettes cloned into a vector. For example, in some embodiments, there can be a first expression cassette comprising a polynucleotide operable to express a DVP. In alternative embodiments, there are two expression cassettes operable to encode a DVP (i.e., a double expression cassette). In other embodiments, there are three expression cassettes operable to encode a DVP (i.e., a triple expression cassette).

In some embodiments, a double expression cassette can be generated by subcloning a second DVP expression cassette into a vector containing a first DVP expression cassette.

In some embodiments, a triple expression cassette can be generated by subcloning a third DVP expression cassette into a vector containing a first and a second DVP expression cassette.

In some embodiments, a DVP polynucleotide can be cloned into a vector using a variety of cloning strategies, and commercial cloning kits and materials readily available to those having ordinary skill in the art. For example, the DVP polynucleotide can be cloned into a vector using such strategies as the SnapFast; Gateway; TOPO; Gibson; LIC; InFusionHD; or Electra strategies. There are numerous commercially available vectors that can be used to produce DVP. For example, a DVP polynucleotide can be generated using polymerase chain reaction (PCR), and combined with a pCR™II-TOPO vector, or a PCR™2.1-TOPO® vector (commercially available as the TOPO® TA Cloning® Kit from Invitrogen) for 5 minutes at room temperature; the TOPO® reaction can then be transformed into competent cells, which can subsequently be selected based on color change (see Janke et al., A versatile toolbox for PCR-based tagging of yeast genes: new fluorescent proteins, more markers and promoter substitution cassettes. Yeast. 2004 August; 21(11):947-62; see also, Adams et al. Methods in Yeast Genetics. Cold Spring Harbor, N Y, 1997, the disclosure of which is incorporated herein by reference in its entirety).

In some embodiments, a polynucleotide encoding a DVP can be cloned into a vector such as a plasmid, cosmid, virus (bacteriophage, animal viruses, and plant viruses), and/or artificial chromosome (e.g., YACs).

In some embodiments, a polynucleotide encoding a DVP can be inserted into a vector, for example, a plasmid vector using E. coli as a host, by performing the following: digesting about 2 to 5 μg of vector DNA using the restriction enzymes necessary to allow the DNA segment of interest to be inserted, followed by overnight incubation to accomplish complete digestion (alkaline phosphatase may be used to dephosphorylate the 5′-end in order to avoid self-ligation/recircularization); gel purify the digested vector. Next, amplify the DNA segment of interest, for example, a polynucleotide encoding an DVP, via PCR, and remove any excess enzymes, primers, unincorporated dNTPs, short-failed PCR products, and/or salts from the PCR reaction using techniques known to those having ordinary skill in the art (e.g., by using a PCR clean-up kit). Ligate the DNA segment of interest to the vector by creating a mixture comprising: about 20 ng of vector; about 100 to 1,000 ng or DNA segment of interest; 2 μL 10× buffer (i.e., 30 mM Tris-HCl 4 mM MgCl₂, 26 μM NAD, 1 mM DTT, 50 μg/ml BSA, pH 8, stored at 25° C.); 1 μL T4 DNA ligase; all brought to a total volume of 20 μL by adding H₂O. The ligation reaction mixture can then be incubated at room temperature for 2 hours, or at 16° C. for an overnight incubation. The ligation reaction (i.e., about 1 μL) can then be transformed to competent cell, for example, by using electroporation or chemical methods, and a colony PCR can then be performed to identify vectors containing the DNA segment of interest.

In some embodiments a polynucleotide encoding a DVP (e.g., a DVP ORF), along with other DNA segments together composing a DVP expression cassette can be designed for secretion from host yeast cells. An illustrative method of designing a DVP expression cassette is as follows: the cassette can begin with a signal peptide sequence, followed by a DNA sequence encoding a Kex2 cleavage site (Lysine-Arginine), and subsequently followed by the DVP polynucleotide transgene (DVP ORF), with the addition of glycine-serine codons at the 5′-end, and finally a stop codon at the 3′-end. All these elements will then be expressed to a fusion peptide in yeast cells as a single open reading frame (ORF). An α-mating factor (αMF) signal sequence is most frequently used to facilitate metabolic processing of the recombinant insecticidal peptides through the endogenous secretion pathway of the recombinant yeast, i.e. the expressed fusion peptide will typically enter the Endoplasmic Reticulum, wherein the α-mating factor signal sequence is removed by signal peptidase activity, and then the resulting pro-insecticidal peptide will be trafficked to the Golgi Apparatus, in which the Lysine-Arginine dipeptide mentioned above is completely removed by Kex2 endoprotease, after which the mature, polypeptide (i.e., DVP), is secreted out of the cells.

In some embodiments, polypeptide expression levels in recombinant yeast cells can be enhanced by optimizing the codons based on the specific host yeast species. Naturally occurring frequencies of codons observed in endogenous open reading frames of a given host organism need not necessarily be optimized for high efficiency expression. Furthermore, different yeast species (for example, Kluyveromyces lactis, Pichia pastoris, Saccharomyces cerevisiae, etc.) have different optimal codons for high efficiency expression. Hence, codon optimization should be considered for the DVP expression cassette, including the sequence elements encoding the signal sequence, the Kex2 cleavage site and the DVP, because they are initially translated as one fusion peptide in the recombinant yeast cells.

In some embodiments, a codon-optimized DVP expression cassette can be ligated into a yeast-specific expression vectors for yeast expression. There are many expression vectors available for yeast expression, including episomal vectors and integrative vectors, and they are usually designed for specific yeast strains. One should carefully choose the appropriate expression vector in view of the specific yeast expression system which will be used for the peptide production. In some embodiments, integrative vectors can be used, which integrate into chromosomes of the transformed yeast cells and remain stable through cycles of cell division and proliferation. The integrative DNA sequences are homologous to targeted genomic DNA loci in the transformed yeast species, and such integrative sequences include pLAC4, 25S rDNA, pAOX1, and TRP2, etc. The locations of insecticidal peptide transgenes can be adjacent to the integrative DNA sequence (Insertion vectors) or within the integrative DNA sequence (replacement vectors).

In some embodiments, the expression vectors or cloning vectors can contain E. coli elements for DNA preparation in E. coli, for example, E. coli replication origin, antibiotic selection marker, etc. In some embodiments, vectors can contain an array of the sequence elements needed for expression of the transgene of interest, for example, transcriptional promoters, terminators, yeast selection markers, integrative DNA sequences homologous to host yeast DNA, etc. There are many suitable yeast promoters available, including natural and engineered promoters, for example, yeast promoters such as pLAC4, pAOX1, pUPP, pADH1, pTEF, pGal1, etc., and others, can be used in some embodiments.

In some embodiments, selection methods such as acetamide prototrophy selection; zeocin-resistance selection; geneticin-resistance selection; nourseothricin-resistance selection; uracil deficiency selection; and/or other selection methods may be used. For example, in some embodiments, the Aspergillus nidulans amdS gene can be used as selectable marker. Exemplary methods for the use of selectable markers can be found in U.S. Pat. No. 6,548,285 (filed Apr. 3, 1997); U.S. Pat. No. 6,165,715 (filed Jun. 22, 1998); and 6,110,707 (filed Jan. 17, 1997), the disclosures of which are incorporated herein by reference in its entirety.

In some embodiments, a polynucleotide encoding a DVP can be inserted into a pKLAC1 vector. The pKLAC1 is commercially available from New England Biolabs® Inc., (item no. (NEB #E1000). The pKLAC1 is designed to accomplish high-level expression of recombinant protein (e.g., DVP) in the yeast Kluyveromyces lactis. The pKLAC1 plasmid can be ordered alone, or as part of a K. lactis Protein Expression Kit. The pKLAC1 plasmid can be linearized using the SacII or BstXI restriction enzymes, and possesses a MCS downstream of an αMF secretion signal. The αMF secretion signal directs recombinant proteins to the secretory pathway, which is then subsequently cleaved via Kex2 resulting in peptide of interest, for example, a DVP. Kex2 is a calcium-dependent serine protease, which is involved in activating proproteins of the secretory pathway, and is commercially available (PeproTech®; item no. 450-45).

In some embodiments, a polynucleotide encoding a DVP can be inserted into a pLB102 plasmid, or subcloned into a pLB102 plasmid subsequent to selection of yeast colonies transformed with pKLAC1 plasmids ligated with polynucleotide encoding a DVP. Yeast, for example K. lactis, transformed with a pKLAC1 plasmids ligated with polynucleotide encoding a DVP can be selected based on acetamidase (amdS), which allows transformed yeast cells to grow in YCB medium containing acetamide as its only nitrogen source. Once positive yeast colonies transformed with a pKLAC1 plasmids ligated with polynucleotide encoding a DVP are identified.

In some embodiments, a polynucleotide encoding a DVP can be inserted into other commercially available plasmids and/or vectors that are readily available to those having skill in the art, e.g., plasmids are available from Addgene (a non-profit plasmid repository); GenScript®; Takara®; Qiagen®; and Promega™.

In some embodiments, a yeast cell transformed with one or more DVP expression cassettes can produce DVP in a yeast culture with a yield of: at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of DVP per liter of medium.

In some embodiments, a culture of K. lactis transformed with one or more DVP expressions cassettes, can produce DVP in a yeast culture with a yield of: at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of DVP per liter of growth medium containing: (1) MSM media recipe: 2 g/L sodium citrate dihydrate; 1 g/L calcium sulfate dihydrate (0.79 g/L anhydrous calcium sulfate); 42.9 g/L potassium phosphate monobasic; 5.17 g/L ammonium sulfate; 14.33 g/L potassium sulfate; 11.7 g/L magnesium sulfate heptahydrate; 2 mL/L PTM1trace salt solution; 0.4 ppm biotin (from 500X, 200 ppm stock); 1-2% pure glycerol or other carbon source. (2) PTM1 trace salts solution: Cupric sulfate-5H2O 6.0 g; Sodium iodide 0.08 g; Manganese sulfate-H2O 3.0 g; Sodium molybdate-2H₂O, 0.2 g; Boric Acid 0.02 g; Cobalt chloride 0.5 g; Zinc chloride 20.0 g; Ferrous sulfate-7H₂O, 65.0 g; Biotin 0.2 g; Sulfuric Acid 5.0 ml; add Water to a final volume of 1 liter. An illustrative composition for K. lactis defined medium (DMSor) is as follows: 11.83 g/L KH₂PO₄, 2.299 g/L K₂IPO₄, 20 g/L of a fermentable sugar, e.g., galactose, maltose, latotriose, sucrose, fructose or glucose and/or a sugar alcohol, for example, erythritol, hydrogenated starch hydrolysates, isomalt, lactitol, maltitol, mannitol, and xylitol, 1 g/L MgSO₄·7H₂O, 10 g/L (NH₄)SO₄, 0.33 g/L CaCl₂.2H₂O, 1 g/L NaCl, 1 g/L KCl, 5 mg/L CuSO₄·5H₂O, 30 mg/L MnSO₄·H₂O, 10 mg/L, ZnCl₂, 1 mg/L KI, 2 mg/L COC1₂.6H₂O, 8 mg/L Na₂MoO₄.2H₂O, 0.4 mg/L H₃BO₃,15 mg/L FeCl₃.6H₂O, 0.8 mg/L biotin, 20 mg/L Ca-pantothenate, 15 mg/L thiamine, 16 mg/L myo-inositol, 10 mg/L nicotinic acid, and 4 mg/L pyridoxine; a selection marker, and culturing under conditions that enable optimum expression.

In some embodiments, one or more expression cassettes comprising a polynucleotide operable to express a DVP can be inserted into a vector, resulting in a yield of about 100 mg/L of DVP (supernatant of yeast fermentation broth). For example, in some embodiments, two expression cassettes comprising a polynucleotide operable to express a DVP can be inserted into a vector, for example a pKS022 plasmid, resulting in a yield of about 2 g/L of DVP (supernatant of yeast fermentation broth). Alternatively, in some embodiments, three expression cassettes comprising a polynucleotide operable to express a DVP can be inserted into a vector, for example a pLB103bT plasmid.

In some embodiments, multiple DVP expression cassettes can be transfected into yeast in order to enable integration of one or more copies of the optimized DVP transgene into the K. lactis genome. An exemplary method of introducing multiple DVP expression cassettes into a K. lactis genome is as follows: a DVP expression cassette DNA sequence is synthesized, comprising an intact LAC4 promoter element, a codon-optimized DVP ORF element and a pLAC4 terminator element; the intact expression cassette is ligated into the pLB103b vector between Sal I and Kpn I restriction sites, downstream of the pLAC4 terminator of pLB10V5, resulting in the double transgene DVP expression vector, pKS022; the double transgene vectors, pKS022, are then linearized using Sac II restriction endonuclease and transformed into YCT306 strain of K. lactis by electroporation. The resulting yeast colonies are then grown on YCB agar plate supplemented with 5 mM acetamide, which only the acetamidase-expressing cells could use efficiently as a metabolic source of nitrogen. To evaluate the yeast colonies, about 100 to 400 colonies can be picked from the pKS022 yeast plates. Inoculates from the colonies are each cultured in 2.2 mL of the defined K. lactis media with 2% sugar alcohol added as a carbon source. Cultures are incubated at 23.5° C., with shaking at 280 rpm, for six days, at which point cell densities in the cultures will reach their maximum levels as indicated by light absorbance at 600 nm (OD600). Cells are then removed from the cultures by centrifugation at 4,000 rpm for 10 minutes, and the resulting supernatants (conditioned media) are filtered through 0.2 μM membranes for HPLC yield analysis.

Expression Cassettes

In addition to a heterologous polynucleotide operable to encode a DVP or a DVP-insecticidal protein, additional DNA segments known as regulatory elements can be cloned into a vector that allow for enhanced expression of the heterologous polynucleotide. Examples of such regulatory elements include (1) promoters, terminators, and/or enhancer elements; (2) an appropriate mRNA stabilizing polyadenylation signal; (3) an internal ribosome entry site (IRES); (4) introns; and (5) post-transcriptional regulatory elements.

As described above, the combination of a DNA segment of interest (e.g., a heterologous polynucleotide operable to encode a DVP or a DVP-insecticidal protein) with any one of the foregoing cis-acting elements is called an “expression cassette.”

Thus, in some embodiments, these additional DNA segments known as regulatory elements can be operably linked and in any orientation with regard to a heterologous polynucleotide operable to encode a DVP or a DVP-insecticidal protein.

For example, in some embodiments, a vector can comprise an expression cassette, wherein the expression cassette comprises one or more (1) promoters, terminators, and/or enhancer elements; (2) an appropriate mRNA stabilizing polyadenylation signal; (3) an internal ribosome entry site (IRES); (4) introns; and (5) post-transcriptional regulatory elements, that allow for enhanced expression of the heterologous polynucleotide operable to encode a DVP or a DVP-insecticidal protein.

And, in some embodiments, the vector can comprise multiple heterologous polynucleotides operable to encode a DVP or a DVP-insecticidal protein, wherein each of the individual heterologous polynucleotides operable to encode a DVP or a DVP-insecticidal protein, has its own expression cassette comprising one or more (1) promoters, terminators, and/or enhancer elements; (2) an appropriate mRNA stabilizing polyadenylation signal; (3) an internal ribosome entry site (IRES); (4) introns; and (5) post-transcriptional regulatory elements, that allow for enhanced expression each of the heterologous polynucleotide operable to encode a DVP or a DVP-insecticidal protein, respectively.

In some embodiments, a heterologous polynucleotide can comprise one or more expression cassettes.

In some embodiments, a vector can comprise one or more expression cassettes.

Cloning Strategies

Insertion of the appropriate polynucleotide into a vector can be performed by a variety of procedures.

In general, the DNA sequence is ligated to the desired position in the vector following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley & Sons, Inc. 1997 and Sambrook et al., Molecular Cloning: A Laboratory Manual 2nd Ed., Cold Spring Harbor Laboratory Press (1989); the disclosures of which are incorporated herein by reference in their entireties. Such procedures and others are deemed to be within the scope of those skilled in the art.

In some embodiments, a heterologous polynucleotide operable to encode a DVP or a DVP-insecticidal protein can be inserted into other commercially available plasmids and/or vectors that are readily available to those having skill in the art, e.g., plasmids are available from Addgene (a non-profit plasmid repository); GenScript®; Takara®; Qiagen®; and Promega™.

In some embodiments, a vector can be, for example, in the form of a plasmid, a viral particle, or a phage. In other embodiments, a vector can include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus and pseudorabies.

In some embodiments, vectors compatible with eukaryotic cells, such as vertebrate cells, can be used. Eukaryotic cell vectors are well known in the art and are available from commercial sources. Contemplated vectors may contain both prokaryotic sequences (to facilitate the propagation of the vector in bacteria), and one or more eukaryotic transcription units that are functional in non-bacterial cells. Typically, such vectors provide convenient restriction sites for insertion of the desired recombinant DNA molecule. The pcDNAI, pSV2, pSVK, pMSG, pSVL, pPVV-1/PML2d and pTDT1 (ATCC No. 31255) derived vectors are examples of mammalian vectors suitable for transfection of non-human cells. In some embodiments, some of the foregoing vectors may be modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) may be used for expression of proteins in swine cells. The various methods employed in the preparation of the plasmids and the transformation of host cells are well known in the art.

In some embodiments, and in addition to a heterologous polynucleotide operable to encode a DVP or a DVP-insecticidal protein, a vector may include a signal sequence or a leader sequence for targeting membranes or secretion as well as expression regulatory elements, such as a promoter, an operator, an initiation codon, a stop codon, a polyadenylation signal, and/or an enhancer; and can be constructed in various forms depending on the purpose thereof. The initiation codon and stop codons are generally considered to be a portion of a nucleotide sequence coding for a target protein, are necessary to be functional in a subject to which a genetic construct has been administered, and must be in frame with the coding sequence.

In some embodiments, the promoter of the vector may be constitutive or inducible. In addition, expression vectors may include a selectable marker that allows the selection of host cells containing the vector, and replicable expression vectors include a replication origin. The vector may be self-replicable, or may be integrated into the host DNA.

Use of promoters may not be required in cases in which transcriptionally active genes are targeted, if the design of the construct results in the marker being transcribed as directed by an endogenous promoter. Exemplary constructs and vectors for carrying out such targeted modification are described herein. However, other vectors that can be used in such approaches are known in the art and can readily be adapted for use in the invention.

In some embodiments, a targeting vector can be used. A basic targeting vector comprises a site-specific integration (SSI) sequence, e.g., 5′- and 3′-homology arms of sequence that is homologous to an endogenous DNA segment that is being targeted.

In some embodiments, a targeting vector can also optionally include one or more positive and/or negative selection markers. In some embodiments, the selection markers can be used to disrupt gene function and/or to identify cells that have integrated targeting vector nucleotide sequences following transformation.

In some embodiments, the use of a targeting vector may utilize a heterologous polynucleotide comprising one or more mutations, in order to create restriction patterns that are distinguishable from the endogenous gene (if the transgene and endogenous gene are similar).

Homology Arms

Those having ordinary skill in the art will recognize that targeted gene modification requires the use of nucleic acid molecule vectors comprising regions of homology with a targeted gene (or flanking regions thereof), such that integration of the vector into the genome can be facilitated. Thus, a targeting vector is generally designed to contain three main regions: (1) a first region that is homologous to the locus to be targeted; (2) a second region that is a heterologous polynucleotide sequence (e.g., comprising a polynucleotide operable to encode a protein of interest and/or encoding a selectable marker, such as an antibiotic resistance protein) that is to be inserted at a target locus and/or to specifically replace a portion of the targeted locus; and (3) a third region that, like the first region, is homologous to the targeted locus, but typically is not contiguous with the first region of the genome.

Homologous recombination between the targeting vector and the targeted endogenous or wild-type locus results in deletion of any locus sequences between the two regions of homology represented in the targeting vector and replacement of that sequence with, or insertion into that sequence of, a heterologous sequence that, for example, encodes the polynucleotide of interest and optionally one or more additional regulatory elements.

In order to facilitate homologous recombination, the first and third regions of the targeting vectors (see above) include sequences that exhibit substantial identity to the genes to be targeted (or flanking regions). By “substantially identical” is meant having a sequence that is at least 80%, preferably at least 85%, preferably at least 90%, more preferably at least 95%, even more preferably at least 98%, and even more preferably 100% identical to that of another sequence. Sequence identity is typically measured using BLAST® (Basic Local Alignment Search Tool) or BLAST® 2 with the default parameters specified therein (see, Altschul et al., J. Mol. Biol. 215: 403-410, 1990; Tatiana et al., FEMS Microbiol. Lett. 174: 247-250, 1999). These software programs match similar sequences by assigning degrees of homology to various substitutions, deletions, and other modifications. Thus, sequences having at least 80%, preferably at least 85%, preferably at least 90%, more preferably at least 95%, even more preferably at least 98%, and even more preferably 100% sequence identity with the targeted gene loci can be used in the invention to facilitate homologous recombination.

The total size of the two regions of homology (i.e., the first and third regions noted above) can be, for example, approximately between 1-25 kilobases (kb) (for example, approximately between 2-20 kb, approximately between 5-15 kb, or approximately between 6-10 kb), and the size of the second region that replaces a portion of the targeted locus can be, for example, approximately between 0.5-5 kb (for example, approximately between 1-4 kb, approximately between 1-3 kb, approximately between 1-2 kb, or approximately between 3-4 kb).

In some embodiments, a targeting vector generally can comprise a selection marker and a site-specific integration (SSI) sequence. The SSI sequence can comprise a transgene of interest, e.g., a heterologous polynucleotide operable to encode a DVP or a DVP-insecticidal protein; which is flanked with two genomic DNA fragments called “5′- and 3′-homology arms” or “5′ and 3′ arms” or “left and right arms” or “homology arms.” These homology arms recombine with the target genome sequence and/or endogenous gene of interest in the host organism in order to achieve successful genetic modification of the host organism's chromosomal locus.

When designing the homology arms for a targeting vector, both the 5′- and 3′-arms should possess sufficient sequence homology with the endogenous sequence to be targeted in order to engender efficient in vivo pairing of the sequences, and cross-over formation. And, while homology arm length is variable, a homology covering at least 5-8 kb in total for both arms (with the shorter arm having no less than 1 kb in length), is a general guideline that can be followed to help ensure successful recombination.

In some embodiments, the 5′- and/or 3′-homology arms may vary. For example, in some embodiments, different loci could be targeted by the 5′- and/or 3′-homology arms, e.g., either upstream and/or downstream from a homology arm described herein to exchange the sequence of interest at a different location.

Additional exemplary methods of vector design and in vivo homologous recombination can be found in U.S. Pat. No. 5,464,764, entitled “Positive-negative selection methods and vectors” (filed Feb. 4, 1993; assignee University of Utah Research Foundation, Salt Lake City, UT); U.S. Pat. No. 5,733,761, entitled “Protein production and protein delivery” (filed May 26, 1995; assignee Transkaryotic Therapies, Inc., Cambridge, MA); U.S. Pat. No. 5,789,215, entitled “Gene targeting in animal cells using isogenic DNA constructs” (filed Aug. 7, 1997; assignee GenPharm International, San Jose, CA); U.S. Pat. No. 6,090,554, entitled “Efficient construction of gene targeting vectors” (filed Oct. 31, 1997; assignee Amgen, Inc., Thousand Oaks, CA); U.S. Pat. No. 6,528,314, entitled “Procedure for specific replacement of a copy of a gene present in the recipient genome by the integration of a gene different from that where the integration is made” (filed Jun. 6, 1995; assignee Institut, Pasteur); U.S. Pat. No. 6,537,542, entitled “Targeted introduction of DNA into primary or secondary cells and their use for gene therapy and protein production (filed Apr. 14, 2000; assignee Transkaryotic Therapies, Inc., Cambridge, MA); U.S. Pat. No. 8,048,645, entitled “Method of producing functional protein domains (filed Aug. 1, 2001; assignee Merck Serono SA); and U.S. Pat. No. 8,173,394, entitled “Systems and methods for protein production” (filed Apr. 6, 2009; assignee Wyeth LLC, Madison, NJ); the disclosures of which are incorporated herein by reference in their entirety.

Exemplary descriptions and methods concerning selection markers are provided in Wigler et al., Cell 11:223 (1977); Szybalska & Szybalski, Proc. Natl. Acad. Sci. USA 48:202 (1992); Lowy et al., Cell 22:817 (1980); Wigler et al., Natl. Acad. Sci. USA 77:357 (1980); O'Hare et al., Proc. Natl. Acad. Sci. USA 78:1527 (1981); Mulligan & Berg, Proc. Natl. Acad. Sci. USA 78:2072 (1981); Wu and Wu, Biotherapy 3:87-95 (1991); Tolstoshev, Ann. Rev. Pharmacol. Toxicol. 32:573-596 (1993); Mulligan, Science 260:926-932 (1993); Morgan and Anderson, Ann. Rev. Biochem. 62:191-217 (1993); Santerre et al., Gene 30:147 (1984); Ausubel et al. (eds.), Current Protocols in Molecular Biology, John Wiley & Sons, N Y (1993); Kriegler, Gene Transfer and Expression, A Laboratory Manual, Stockton Press, N Y (1990); in Chapters 12 and 13, Dracopoli et al. (eds), Current Protocols in Human Genetics, John Wiley & Sons, N Y (1994); Colberre-Garapin et al., J. Mol. Biol. 150:1 (1981); U.S. Pat. No. 6,548,285 (filed Apr. 3, 1997); U.S. Pat. No. 6,165,715 (filed Jun. 22, 1998); and 6,110,707 (filed Jan. 17, 1997), the disclosures of which are incorporated by reference herein in their entireties.

Exemplary Vectors

In some embodiments, the present invention comprises, consists essentially of, or consists of, a vector comprising: (a) a heterologous polynucleotide, or a complementary nucleotide sequence thereof, comprising: (i) a nucleotide sequence operable to encode a DVP or a DVP-insecticidal protein; (b) a 5′-homology arm, and a 3′-homology arm, wherein said 5′-homology arm and said 3′-homology arm are located upstream and downstream of the heterologous polynucleotide, respectively; wherein said vector is operable to allow a homologous-recombination-mediated integration of the heterologous polynucleotide into an endogenous host cell locus; and wherein said homologous-recombination-mediated integration results in a replacement of an endogenous host cell DNA segment with the heterologous polynucleotide.

In some embodiments, a heterologous polynucleotide, or a complementary nucleotide sequence thereof, comprising: (i) a nucleotide sequence operable to encode a DVP or a DVP-insecticidal protein can be cloned or inserted into a vector (e.g., a plasmid). In other embodiments, any of the components of the heterologous polynucleotide, or a complementary nucleotide sequence thereof, i.e., (i) a nucleotide sequence operable to encode a DVP or a DVP-insecticidal protein, can be cloned or inserted into a vector.

In some embodiments, a recombinant host cell is transformed with a vector comprising, consisting essentially of, or consisting of, a heterologous polynucleotide operable to encode a DVP or a DVP-insecticidal protein, or a complementary nucleotide sequence thereof, said heterologous polynucleotide comprising the following nucleotide sequences, operably linked and in any orientation: (i) at least one nucleotide sequence operable to encode a DVP or a DVP-insecticidal protein.

In some embodiments, a heterologous polynucleotide operable to encode a DVP or a DVP-insecticidal protein; can be cloned into a vector using a variety of cloning strategies, and commercial cloning kits and materials readily available to those having ordinary skill in the art.

For example, a heterologous polynucleotide and/or a nucleotide sequence operable to encode a DVP or a DVP-insecticidal protein, can be cloned into a vector using such strategies as the SnapFast; Gateway; TOPO; Gibson; LIC; InFusionHD; or Electra strategies.

There are numerous commercially available vectors that can be used to produce a vector of the present invention. For example, a heterologous polynucleotide operable to encode a DVP or a DVP-insecticidal protein can be generated using polymerase chain reaction (PCR), and combined with a pCRTJII-TOPO vector, or a PCR™2.1-TOPO® vector (commercially available as the TOPO® TA Cloning® Kit from Invitrogen) for 5 minutes at room temperature; the TOPO® reaction can then be transformed into competent cells, which can subsequently be selected based on color change (see Janke et al., A versatile toolbox for PCR-based tagging of yeast genes: new fluorescent proteins, more markers and promoter substitution cassettes. Yeast. 2004 August; 21(11):947-62; see also, Adams et al. Methods in Yeast Genetics. Cold Spring Harbor, N Y, 1997, the disclosure of which is incorporated herein by reference in its entirety).

In some embodiments, a heterologous polynucleotide operable to encode a DVP or a DVP-insecticidal protein, can be cloned into a vector such as a plasmid, cosmid, virus (bacteriophage, animal viruses, and plant viruses), and/or artificial chromosome (e.g., YACs).

In some embodiments, a heterologous polynucleotide operable to encode a DVP or a DVP-insecticidal protein, can be inserted into a vector, for example, a plasmid vector using E. coli as a host, by performing the following: digesting about 2 to 5 μg of vector DNA using the restriction enzymes necessary to allow the DNA segment of interest to be inserted, followed by overnight incubation to accomplish complete digestion (alkaline phosphatase may be used to dephosphorylate the 5′-end in order to avoid self-ligation/recircularization); gel purify the digested vector. Next, amplify the DNA segment of interest, for example, a heterologous polynucleotide operable to encode a DVP or a DVP-insecticidal protein, via PCR, and remove any excess enzymes, primers, unincorporated dNTPs, short-failed PCR products, and/or salts from the PCR reaction using techniques known to those having ordinary skill in the art (e.g., by using a PCR clean-up kit). Ligate the DNA segment of interest to the vector by creating a mixture comprising: about 20 ng of vector; about 100 to 1,000 ng or DNA segment of interest; 2 L 10× buffer (i.e., 30 mM Tris-HCl 4 mM MgCl₂, 26 μM NAD, 1 mM DTT, 50 μg/ml BSA, pH 8, stored at 25° C.); 1 μL T4 DNA ligase; all brought to a total volume of 20 μL by adding H₂O. The ligation reaction mixture can then be incubated at room temperature for 2 hours, or at 16° C. for an overnight incubation. The ligation reaction (i.e., about 1 μL) can then be transformed to competent cell, for example, by using electroporation or chemical methods, and a colony PCR can then be performed to identify vectors containing the DNA segment of interest.

In some embodiments, a heterologous polynucleotide operable to encode a DVP or a DVP-insecticidal protein, along with other DNA segments together composing an expression ORF can be designed for secretion from host yeast cells. An illustrative method of designing an expression ORF is as follows: the ORF can begin with a signal peptide sequence, followed by a DNA sequence encoding a Kex2 cleavage site (Lysine-Arginine), and subsequently followed by the heterologous polynucleotide transgene, with the addition of glycine-serine codons at the 5′-end, and finally a stop codon at the 3′-end. All these elements will then be expressed to a fusion peptide in yeast cells as a single open reading frame (ORF). An α-mating factor (αMF) signal sequence is most frequently used to facilitate metabolic processing of the recombinant insecticidal peptides through the endogenous secretion pathway of the recombinant yeast, i.e. the expressed fusion peptide will typically enter the Endoplasmic Reticulum, wherein the α-mating factor signal sequence is removed by signal peptidase activity, and then the resulting pro-insecticidal peptide will be trafficked to the Golgi Apparatus, in which the Lysine-Arginine dipeptide mentioned above is completely removed by Kex2 endoprotease, after which the mature, DVP or DVP-insecticidal protein is secreted out of the cells.

In some embodiments, polypeptide expression levels in recombinant cells can be enhanced by optimizing the codons based on the specific host yeast species. Naturally occurring frequencies of codons observed in endogenous open reading frames of a given host organism need not necessarily be optimized for high efficiency expression. Furthermore, different yeast species (for example, Kluyveromyces lactis, Pichia pastoris, Saccharomyces cerevisiae, etc.) have different optimal codons for high efficiency expression. Hence, codon optimization should be considered for the expression ORF, including the sequence elements encoding the signal sequence, the Kex2 cleavage site and the heterologous polypeptide, because they are initially translated as one fusion peptide in the recombinant yeast cells.

In some embodiments, a codon-optimized expression ORF can be ligated into a yeast-specific expression vectors for yeast expression. There are many expression vectors available for yeast expression, including episomal vectors and integrative vectors, and they are usually designed for specific yeast cells. One should carefully choose the appropriate expression vector in view of the specific yeast expression system which will be used for the peptide production. In some embodiments, integrative vectors can be used, which integrate into chromosomes of the transformed yeast cells and remain stable through cycles of cell division and proliferation. The integrative DNA sequences are homologous to targeted genomic DNA loci in the transformed yeast species, and such integrative sequences include pLAC4, 25S rDNA, pAOX1, and TRP2, etc. The locations of insecticidal peptide transgenes can be adjacent to the integrative DNA sequence (Insertion vectors) or within the integrative DNA sequence (replacement vectors).

In some embodiments, the expression vectors can contain E. coli elements for DNA preparation in E. coli, for example, E. coli replication origin, antibiotic selection marker, etc. In some embodiments, vectors can contain an array of the sequence elements needed for expression of the transgene of interest, for example, transcriptional promoters, terminators, yeast selection markers, integrative DNA sequences homologous to host yeast DNA, etc. There are many suitable yeast promoters available, including natural and engineered promoters, for example, yeast promoters such as pLAC4, pAOX1, pUPP, pADH1, pTEF, pGal1, etc., and others, can be used in some embodiments.

In some embodiments, a heterologous polynucleotide operable to encode a DVP or a DVP-insecticidal protein can be inserted into other commercially available plasmids and/or vectors that are readily available to those having skill in the art, e.g., plasmids are available from Addgene (a non-profit plasmid repository); GenScript®; Takara®; Qiagen®; and Promega™.

Following the preparation of a vector comprising a heterologous polynucleotide operable to encode a DVP or a DVP-insecticidal protein, the vector is transformed into the yeast cell to produce a recombinant yeast cell of the present invention.

In some embodiments, a vector of the present invention comprises: (a) a heterologous polynucleotide, or a complementary nucleotide sequence thereof, comprising: (i) a heterologous polynucleotide operable to encode a DVP or a DVP-insecticidal protein; (b) a 5′-homology arm, and a 3′-homology arm, wherein said 5′-homology arm and said 3′-homology arm are located upstream and downstream of the heterologous polynucleotide, respectively; wherein said vector is operable to allow a homologous-recombination-mediated integration of the heterologous polynucleotide into an endogenous yeast host cell gene locus; and wherein said homologous-recombination-mediated integration results in a replacement of an endogenous yeast host cell gene DNA segment with the heterologous polynucleotide.

In some embodiments, a vector can comprise a polynucleotide operable to encode a DVP, or a complementary sequence thereof.

In some embodiments, a vector can comprise a polynucleotide operable to encode a DVP having an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191,202-215, or 217-219, or a complementary sequence thereof.

In some embodiments, a vector can comprise a polynucleotide operable to encode a DVP having an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219, or a complementary sequence thereof.

In some embodiments, a vector can comprise a polynucleotide operable to encode a DVP an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 47, 53, 136, 139-140, 144, 146-147, 187-191, 210-215, or 217-219, or a complementary sequence thereof.

In some embodiments, a vector can comprise a polynucleotide operable to encode a DVP an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 213, or 217-219, or a complementary sequence thereof.

Transformation and Cell Culture Methods

The terms “transformation” and “transfection” both describe the process of introducing exogenous and/or heterologous DNA or RNA to a host organism. Generally, those having ordinary skill in the art sometimes reserve the term “transformation” to describe processes where exogenous and/or heterologous DNA or RNA are introduced into a bacterial cell; and reserve the term “transfection” for processes that describe the introduction of exogenous and/or heterologous DNA or RNA into eukaryotic cells. However, as used herein, the term “transformation” and “transfection” are used synonymously, regardless of whether a process describes the introduction exogenous and/or heterologous DNA or RNA into a prokaryote (e.g., bacteria) or a eukaryote (e.g., yeast, plants, or animals).

In some embodiments, a host cell can be transformed with a polynucleotide operable to encode a DVP.

In some embodiments, a vector containing a DVP expression cassette can be cloned into an expression plasmid and transformed into a host cell. In some embodiments, the yeast cell can any one of those yeast cells described herein.

In some embodiments, a host cell can be transformed using the following methods: electroporation; cell squeezing; microinjection; impalefection; the use of hydrostatic pressure; sonoporation; optical transfection; continuous infusion; lipofection; through the use of viruses such as adenovirus, adeno-associated virus, lentivirus, herpes simplex virus, and retrovirus; the chemical phosphate method; endocytosis via DEAE-dextran or polyethylenimine (PEI); protoplast fusion; hydrodynamic deliver; magnetofection; nucleoinfection; and/or others. Exemplary methods regarding transfection and/or transformation techniques can be found in Makrides (2003), Gene Transfer and Expression in Mammalian Cells, Elvesier; Wong, TK & Neumann, E. Electric field mediated gene transfer. Biochem. Biophys. Res. Commun. 107, 584-587 (1982); Potter & Heller, Transfection by Electroporation. Curr Protoc Mol Biol. 2003 May; CHAPTER: Unit-9.3; Kim & Eberwine, Mammalian cell transfection: the present and the future. Anal Bioanal Chem. 2010 August; 397(8): 3173-3178, each of these references are incorporated herein by reference in their entireties.

In some embodiments, electroporation can be used transform a cell with one or more DVP expression cassettes, which can produce DVP in a yeast culture with a yield of: at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of DVP per liter of medium.

Electroporation is a technique in which electricity is applied to cells causing the cell membrane to become permeable; this in turn allows exogenous DNA to be introduced into the cells. Electroporation is readily known to those having ordinary skill in the art, and the tools and devices required to achieve electroporation are commercially available (e.g., Gene Pulser Xcell™ Electroporation Systems, Bio-Rad®; Neon® Transfection System for Electroporation, Thermo-Fisher Scientific; and other tools and/or devices). Exemplary methods of electroporation are illustrated in Potter & Heller, Transfection by Electroporation. Curr Protoc Mol Biol. 2003 May; CHAPTER: Unit-9.3; Saito (2015) Electroporation Methods in Neuroscience. Springer press; Pakhomov et al., (2017) Advanced Electroporation Techniques in Biology and Medicine. Taylor & Francis; the disclosure of which is incorporated herein by reference in its entirety.

In some embodiments, electroporation can be used to introduce a vector containing a polynucleotide encoding a DVP into yeast, for example, in some embodiments, a DVP expression cassette cloned into a plasmid, and transformed into yeast cells via electroporation.

In some embodiments, a DVP expression cassette cloned into a plasmid, and transformed a yeast cell via electroporation can be accomplished by inoculating about 10-200 mL of yeast extract peptone dextrose (YEPD) with a suitable yeast species, for example, Kluyveromyces lactis, Kluyveromyces marxianus, Saccharomyces cerevisiae, Pichia pastoris, etc., and incubate on a shaker at 30° C. until the early exponential phase of yeast culture (e.g. about 0.6 to 2×10⁸ cells/mL); harvesting the yeast in sterile centrifuge tube and centrifuging at 3000 rpm for 5 minutes at 4° C. (note: keep cells chilled during the procedure) washing cells with 40 mL of ice cold, sterile deionized water, and pelleting the cells a 23,000 rpm for 5 minutes; repeating the wash step, and the resuspending the cells in 20 mL of 1M fermentable sugar, e.g. galactose, maltose, latotriose, sucrose, fructose or glucose and/or sugar alcohol, for example, erythritol, hydrogenated starch hydrolysates, isomalt, lactitol, maltitol, mannitol, and xylitol, followed by spinning down at 3,000 rpm for 5 minutes; resuspending the cells with proper volume of ice cold 1M fermentable sugar, e.g. galactose, maltose, latotriose, sucrose, fructose or glucose and/or a sugar alcohol, for example, erythritol, hydrogenated starch hydrolysates, isomalt, lactitol, maltitol, mannitol, and xylitol to final cell density of 3×10⁹ cell/mL; (1.5×10⁹ cell/mL to 6×10⁹ cell/mL are acceptable cell densities); mixing 40 μl of the yeast suspension with about 1-4 μl (at a concentration of 100-300 ng/μl) of the vector containing a linear polynucleotide encoding a DVP (˜1 μg) in a prechilled 0.2 cm electroporation cuvette (note: ensure the sample is in contact with both sides of the aluminum cuvette); providing a single pulse at 2000 V, for optimal time constant of 5 ms of the RC circuit, the cells was then let recovered in 0.5 ml YED and 0.5 mL 1M fermentable sugar, e.g. galactose, maltose, latotriose, sucrose, fructose or glucose and/or a sugar alcohol, for example, erythritol, hydrogenated starch hydrolysates, isomalt, lactitol, maltitol, mannitol, and xylitol mixture, and then spreading onto selective plates.

In some embodiments, electroporation can be used to introduce a vector containing a polynucleotide encoding a DVP into yeast, for example, a DVP cloned into a plasmid, and transformed into K. lactis cells via electroporation, can be accomplished by inoculating about 10-200 mL of yeast extract peptone dextrose (YEPD) incubating on a shaker at 30° C. until the early exponential phase of yeast culture (e.g. about 0.6 to 2×10⁸ cells/mL); harvesting the yeast in sterile centrifuge tube and centrifuging at 3000 rpm for 5 minutes at 4° C. (note: keep cells chilled during the procedure) washing cells with 40 mL of ice cold, sterile deionized water, and pelleting the cells a 23,000 rpm for 5 minutes; repeating the wash step, and the resuspending the cells in 20 mL of 1M fermentable sugar, e.g. galactose, maltose, latotriose, sucrose, fructose or glucose and/or sugar alcohol, for example, erythritol, hydrogenated starch hydrolysates, isomalt, lactitol, maltitol, mannitol, and xylitol, followed by spinning down at 3,000 rpm for 5 minutes; resuspending the cells with proper volume of ice cold 1M fermentable sugar, e.g. galactose, maltose, latotriose, sucrose, fructose or glucose and/or a sugar alcohol, for example, erythritol, hydrogenated starch hydrolysates, isomalt, lactitol, maltitol, mannitol, and xylitol to final cell density of 3×10⁹ cell/mL; mixing 40 μl of the yeast suspension with about 1-4 μl of the vector containing a linear polynucleotide encoding a DVP (˜1 μg) in a prechilled 0.2 cm electroporation cuvette (note: ensure the sample is in contact with both sides of the aluminum cuvette); providing a single pulse at 2000 V, for optimal time constant of 5 ms of the RC circuit, the cells was then let recovered in 0.5 ml YED and 0.5 mL 1M fermentable sugar, e.g. galactose, maltose, latotriose, sucrose, fructose or glucose and/or a sugar alcohol, for example, erythritol, hydrogenated starch hydrolysates, isomalt, lactitol, maltitol, mannitol, and xylitol mixture, and then spreading onto selective plates.

In some embodiments, using the illustrated methods described herein, i.e., vectors of the present invention utilizing yeast, and methods transformation and fermentation, may result in production of DVP in amounts of: at least 70 mg/L, at least 80 mg/L, at least 90 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, at least 190 mg/L 200 mg/L, at least 500 mg/L, at least 750 mg/L, at least 1,000 mg/L, at least 1,250 mg/L, at least 1,500 mg/L, at least 1,750 mg/L, at least 2,000 mg/L, at least 2,500 mg/L, at least 3,000 mg/L, at least 3,500 mg/L, at least 4,000 mg/L, at least 4,500 mg/L, at least 5,000 mg/L, at least 5,500 mg/L, at least at least 6,000 mg/L, at least 6,500 mg/L, at least 7,000 mg/L, at least 7,500 mg/L, at least 8,000 mg/L, at least 8,500 mg/L, at least 9,000 mg/L, at least 9,500 mg/L, at least 10,000 mg/L, at least 11,000 mg/L, at least 12,000 mg/L, at least 12,500 mg/L, at least 13,000 mg/L, at least 14,000 mg/L, at least 15,000 mg/L, at least 16,000 mg/L, at least 17,000 mg/L, at least 17,500 mg/L, at least 18,000 mg/L, at least 19,000 mg/L, at least 20,000 mg/L, at least 25,000 mg/L, at least 30,000 mg/L, at least 40,000 mg/L, at least 50,000 mg/L, at least 60,000 mg/L, at least 70,000 mg/L, at least 80,000 mg/L, at least 90,000 mg/L, or at least 100,000 mg/L of DVP per liter of medium.

In some embodiments, electroporation can be used to introduce a vector containing a polynucleotide encoding a DVP into plant protoplasts by incubating sterile plant material in a protoplast solution (e.g., around 8 mL of 10 mM 2-[N-morpholino]ethanesulfonic acid (MES), pH 5.5; 0.01% (w/v) pectylase; 1% (w/v) macerozyme; 40 mM CaCl₂; and 0.4 M mannitol) and adding the mixture to a rotary shaker for about 3 to 6 hours at 30° C. to produce protoplasts; removing debris via 80-μm-mesh nylon screen filtration; rinsing the screen with about 4 ml plant electroporation buffer (e.g., 5 mM CaCl₂; 0.4 M mannitol; and PBS); combining the protoplasts in a sterile 15 mL conical centrifuge tube, and then centrifuging at about 300×g for about 5 minutes; subsequent to centrifugation, discarding the supernatant and washing with 5 mL of plant electroporation buffer; resuspending the protoplasts in plant electroporation buffer at about 1.5×10⁶ to 2×10⁶ protoplasts per mL of liquid; transferring about 0.5-mL of the protoplast suspension into one or more electroporation cuvettes, set on ice, and adding the vector (note: for stable transformation, the vector should be linearized using anyone of the restriction methods described above, and about 1 to 10 μg of vector may be used; for transient expression, the vector may be retained in its supercoiled state, and about 10 to 40 μg of vector may be used); mixing the vector and protoplast suspension; placing the cuvette into the electroporation apparatus, and shocking for one or more times at about 1 to 2 kV (a 3- to 25-μF capacitance may be used initially while optimizing the reaction); returning the cuvette to ice; diluting the transformed cells 20-fold in complete medium; and harvesting the protoplasts after about 48 hours.

Heterologous Polynucleotide Incorporation Analysis

Incorporation of a heterologous polynucleotide operable to encode a DVP or a DVP-insecticidal protein, can be analyzed by methods known in the art. For example, in some embodiments, quantitative PCR (qPCR) and paralog ratio test (PRT) can be used to determine if the heterologous polynucleotide has been incorporated. In some embodiments, qPCR is used to confirm the integration of the heterologous polynucleotide operable to encode a DVP or a DVP-insecticidal protein, into the recombinant host cell.

Quantitative PCR (qPCR) has been utilized for the analysis of gene expression and quantification of copy number variation by real-time PCR. qPCR involves amplification of a test locus with unknown copy number and a reference locus with known copy number. There are two approaches to the assay: fluorescent dyes and intercalating dyes. In either approach, fluorescence doubles with every cycle of PCR, and the amount of starting template can be determined from the number of cycles required to achieve a specified threshold level of fluorescence. The actual qPCR experiment takes half a day after sample preparation. Commonly used methods for qPCR data analysis are absolute quantification by relating the PCR signal to a standard curve and relative quantification that relates the PCR signal of the target transcript in one group to another.

To measure DNA copy number, the amplicon should be located either within an exon or intron with sequences unique to that gene. A control gene with two copies should also be included. A master mix containing all of the components is prepared and distributed in 96 or 384-well plate. Template and/or primers are added for each reaction. The assay is performed on a qPCR instrument and data are collected in real time.

Chemically Synthesizing DVPs

Peptide synthesis or the chemical synthesis or peptides and/or polypeptides can be used to generate DVPs: these methods can be performed by those having ordinary skill in the art, and/or through the use of commercial vendors (e.g., GenScript®; Piscataway, New Jersey). For example, in some embodiments, chemical peptide synthesis can be achieved using Liquid phase peptide synthesis (LPPS), or solid phase peptide synthesis (SPPS).

In some embodiments, peptide synthesis can generally be achieved by using a strategy wherein the coupling the carboxyl group of a subsequent amino acid to the N-terminus of a preceding amino acid generates the nascent polypeptide chain—a process that is opposite to the type of polypeptide synthesis that occurs in nature.

Peptide deprotection is an important first step in the chemical synthesis of polypeptides. Peptide deprotection is the process in which the reactive groups of amino acids are blocked through the use of chemicals in order to prevent said amino acid's functional group from taking part in an unwanted or non-specific reaction or side reaction; in other words, the amino acids are “protected” from taking part in these undesirable reactions.

Prior to synthesizing the peptide chain, the amino acids must be “deprotected” to allow the chain to form (i.e., amino acids to bind). Chemicals used to protect the N-termini include 9-fluorenylmethoxycarbonyl (Fmoc), and tert-butoxycarbonyl (Boc), each of which can be removed via the use of a mild base (e.g., piperidine) and a moderately strong acid (e.g., trifluoracetic acid (TFA)), respectively.

The C-terminus protectant required is dependent on the type of chemical peptide synthesis strategy used: e.g., LPPS requires protection of the C-terminal amino acid, whereas SPPS does not owing to the solid support which acts as the protecting group. Side chain amino acids require the use of several different protecting groups that vary based on the individual peptide sequence and N-terminal protection strategy; typically, however, the protecting group used for side chain amino acids are based on the tert-butyl (tBu) or benzyl (Bzl) protecting groups.

Amino acid coupling is the next step in a peptide synthesis procedure. To effectuate amino acid coupling, the incoming amino acid's C-terminal carboxylic acid must be activated: this can be accomplished using carbodiimides such as diisopropylcarbodiimide (DIC), or dicyclohexylcarbodiimide (DCC), which react with the incoming amino acid's carboxyl group to form an O-acylisourea intermediate. The O-acylisourea intermediate is subsequently displaced via nucleophilic attack via the primary amino group on the N-terminus of the growing peptide chain. The reactive intermediate generated by carbodiimides can result in the racemization of amino acids. To avoid racemization of the amino acids, reagents such as 1-hydroxybenzotriazole (HOBt) are added in order to react with the O-acylisourea intermediate. Other couple agents that may be used include 2-(1H-benzotriazol-1-yl)-1,1,3,3-tetramethyluronium hexafluorophosphate (HBTU), and benzotriazol-1-yl-oxy-tris(dimethylamino)phosphonium hexafluorophosphate (BOP), with the additional activating bases. Finally, following amino acid deprotection and coupling,

At the end of the synthesis process, removal of the protecting groups from the polypeptide must occur—a process that usually occurs through acidolysis. Determining which reagent is required for peptide cleavage is a function of the protection scheme used and overall synthesis method. For example, in some embodiments, hydrogen bromide (HBr); hydrogen fluoride (HF); or trifluoromethane sulfonic acid (TFMSA) can be used to cleave Bzl and Boc groups. Alternatively, in other embodiments, a less strong acid such as TFA can effectuate acidolysis of tBut and Fmoc groups. Finally, peptides can be purified based on the peptide's physiochemical characteristics (e.g., charge, size, hydrophobicity, etc.). Techniques that can be used to purify peptides include Purification techniques include Reverse-phase chromatography (RPC); Size-exclusion chromatography; Partition chromatography; High-performance liquid chromatography (HPLC); and Ion exchange chromatography (IEC).

Exemplary methods of peptide synthesis can be found in Anderson G. W. and McGregor A. C. (1957) T-butyloxycarbonylamino acids and their use in peptide synthesis. Journal of the American Chemical Society. 79, 6180-3; Carpino L. A. (1957) Oxidative reactions of hydrazines. Iv. Elimination of nitrogen from 1, 1-disubstituted-2-arenesulfonhydrazides1-4. Journal of the American Chemical Society. 79, 4427-31; McKay F. C. and Albertson N. F. (1957) New amine-masking groups for peptide synthesis. Journal of the American Chemical Society. 79, 4686-90; Merrifield R. B. (1963) Solid phase peptide synthesis. I. The synthesis of a tetrapeptide. Journal of the American Chemical Society. 85, 2149-54; Carpino L. A. and Han G. Y. (1972) 9-fluorenylmethoxycarbonyl amino-protecting group. The Journal of Organic Chemistry. 37, 3404-9; and A Lloyd-Williams P. et al. (1997) Chemical approaches to the synthesis of peptides and proteins. Boca Raton: CRC Press. 278; U.S. Pat. No. 3,714,140 (filed Mar. 16, 1971); U.S. Pat. No. 4,411,994 (filed Jun. 8, 1978); U.S. Pat. No. 7,785,832 (filed Jan. 20, 2006); U.S. Pat. No. 8,314,208 (filed Feb. 10, 2006); and 10,442,834 (filed Oct., 2, 2015); and United States Patent Application 2005/0165215 (filed Dec. 23, 2004), the disclosures of which are incorporated herein by reference in their entirety.

Cell Culture and Fermentation Techniques

Cell culture techniques are well-known in the art. In some embodiments, the culture method and/or materials will necessarily require adaption based on the host cell selected (e.g., modifying pH, temperature, medium contents, and the like). In some embodiments, the medium culture contains a sole carbon source (e.g., sorbitol). In some embodiments, any known culture technique may be employed to produce a recombinant yeast cell of the present invention.

Exemplary culture methods are provided in U.S. Pat. Nos. 3,933,590; 3,946,780; 4,988,623; 5,153,131; 5,153,133; 5,155,034; 5,316,905; 5,330,908; 6,159,724; 7,419,801; 9,320,816; 9,714,408; and 10,563,169; the disclosures of which are incorporated herein by reference in their entireties.

Host Cells

The methods, compositions, DVPs, and DVP-insecticidal proteins of the present invention may be implemented in any cell type, e.g., a eukaryotic or prokaryotic cell.

In some embodiments, the host cell used to produce a DVP or DVP-insecticidal protein is a prokaryote. For example, in some embodiments, the host cell may be an Archaebacteria or Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus.

In some embodiments, the host cell used to produce a DVP or DVP-insecticidal protein may be a unicellular cell. For example, in some embodiments, the host cell may be bacterial cells such as gram positive bacteria.

In some embodiments, the host cell may be a bacteria selected from the following genera consisting of: Candidatus Chloracidobacterium, Arthrobacter, Corynebacterium, Frankia, Micrococcus, Mycobacterium, Propionibacterium, Streptomyces, Aquifex Bacteroides, Porphyromonas, Bacteroides, Porphyromonas, Flavobacterium, Chlamydia, Prosthecobacter, Verrucomicrobium, Chloroflexus, Chroococcus, Merismopedia, Synechococcus, Anabaena, Nostoc, Spirulina, Trichodesmium, Pleurocapsa, Prochlorococcus, Prochloron, Bacillus, Listeria, Staphylococcus, Clostridium, Dehalobacter, Epulopiscium, Ruminococcus, Enterococcus, Lactobacillus, Streptococcus, Erysipelothrix, Mycoplasma, Leptospirillum, Nitrospira, Thermodesulfobacterium, Gemmata, Pirellula, Planctomyces, Caulobacter, Agrobacterium, Bradyrhizobium, Brucella, Methylobacterium, Prosthecomicrobium, Rhizobium, Rhodopseudomonas, Sinorhizobium, Rhodobacter, Roseobacter, Acetobacter, Rhodospirillum, Rickettsia, Rickettsia conorii, Mitochondria, Wolbachia, Erythrobacter, Erythromicrobium, Sphingomonas, Alcaligenes, Burkholderia, Leptothrix, Sphaerotilus, Thiobacillus, Neisseria, Nitrosomonas, Gallionella, Spirillum, Azoarcus, Aeromonas, Succinomonas, Succinivibrio, Ruminobacter, Nitrosococcus, Thiocapsa, Enterobacter, Escherichia, Klebsiella, Salmonella, Shigella, Wigglesworthia, Yersinia, Coxiella, Legionella, Halomonas, Pasteurella, Acinetobacter, Azotobacter, Pseudomonas, Psychrobacter, Beggiatoa, Thiomargarita, Vibrio, Xanthomonas, Bdellovibrio, Campylobacter, Helicobacter, Myxococcus, Desulfosarcina, Geobacter, Desulfuromonas, Borrelia, Leptospira, Treponema, Petrotoga, Thermotoga, Deinococcus, or Thermus.

In some embodiments, the host cell used to produce a DVP or DVP-insecticidal protein may be selected from one of the following bacteria species: Bacillus alkalophilus, Bacillus amyloliquefaciens, Bacillus brevis, Bacillus circulans, Bacillus coagulans, Bacillus lautus, Bacillus lentus, Bacillus lichenformis, Bacillus megaterium, Bacillus stearothermophilus, Bacillus subtilis, Bacillus thuringiensis, Streptomyces lividans, Streptomyces murinus, Streptomyces coelicolor, Streptomyces albicans, Streptomyces griseus, Streptomyces plicatosporus, Escherichia albertii, Escherichia blattae, Escherichia coli, Escherichia fergusonii, Escherichia hermannii, Escherichia senegalensis, Escherichia vulneris, Pseudomonas abietaniphila, Pseudomonas agarici, Pseudomonas agarolyticus, Pseudomonas alcaliphila, Pseudomonas alginovora, Pseudomonas andersonii, Pseudomonas antarctica, Pseudomonas asplenii, Pseudomonas azelaica, Pseudomonas batumici, Pseudomonas borealis, Pseudomonas brassicacearum, Pseudomonas chloritidismutans, Pseudomonas cremoricolorata, Pseudomonas diterpeniphila, Pseudomonas filiscindens, Pseudomonas frederiksbergensis, Pseudomonas gingeri, Pseudomonas graminis, Pseudomonas grimontii, Pseudomonas halodenitrificans, Pseudomonas halophila, Pseudomonas hibiscicola, Pseudomonas hydrogenovora, Pseudomonas indica, Pseudomonas japonica, Pseudomonas jessenii, Pseudomonas kilonensis, Pseudomonas koreensis, Pseudomonas lini, Pseudomonas lurida, Pseudomonas lutea, Pseudomonas marginata, Pseudomonas meridiana, Pseudomonas mesoacidophila, Pseudomonas pachastrellae, Pseudomonas palleroniana, Pseudomonas parafulva, Pseudomonas pavonanceae, Pseudomonas proteolyica, Pseudomonas psychrophila, Pseudomonas psychrotolerans, Pseudomonas pudica, Pseudomonas rathonis, Pseudomonas reactans, Pseudomonas rhizosphaerae, Pseudomonas salmononii, Pseudomonas thermaerum, Pseudomonas thermocarboxydovorans, Pseudomonas thermotolerans, Pseudomonas thivervalensis, Pseudomonas umsongensis, Pseudomonas vancouverensis, Pseudomonas wisconsinensis, Pseudomonas xanthomarina Pseudomonas xiamenensis, Pseudomonas aeruginosa, Pseudomonas alcaligenes, Pseudomonas anguilliseptica, Pseudomonas citronellolis, Pseudomonas flavescens, Pseudomonas jinjuensis, Pseudomonas mendocina, Pseudomonas nitroreducens, Pseudomonas oleovorans, Pseudomonas pseudoalcaligenes, Pseudomonas resinovorans, Pseudomonas straminae, Pseudomonas aurantiaca, Pseudomonas chlororaphis, Pseudomonas fragi, Pseudomonas lundensis, Pseudomonas taetrolens Pseudomonas azotoformans, Pseudomonas brenneri, Pseudomonas cedrina, Pseudomonas congelans, Pseudomonas corrugata, Pseudomonas costantinii, Pseudomonas extremorientalis, Pseudomonas fluorescens, Pseudomonas fulgida, Pseudomonas gessardii, Pseudomonas libanensis, Pseudomonas mandelii, Pseudomonas marginalis, Pseudomonas mediterranea, Pseudomonas migulae, Pseudomonas mucidolens, Pseudomonas orientalis, Pseudomonas poae, Pseudomonas rhodesiae, Pseudomonas synxantha, Pseudomonas tolaasii, Pseudomonas trivialis, Pseudomonas veronii Pseudomonas denitrificans, Pseudomonas pertucinogena, Pseudomonas fulva, Pseudomonas monteilii, Pseudomonas mosselii, Pseudomonas oryzihabitans, Pseudomonas plecoglossicida, Pseudomonas putida, Pseudomonas balearica, Pseudomonas luteola, or Pseudomonas stutzeri. Pseudomonas avellanae, Pseudomonas cannabina, Pseudomonas caricapapyae, Pseudomonas cichorii, Pseudomonas coronafaciens, Pseudomonas fuscovaginae, Pseudomonas tremae, or Pseudomonas viridiflava

In some embodiments, the host cell used to produce a DVP or DVP-insecticidal protein can be eukaryote.

In some embodiments, the host cell used to produce a DVP or DVP-insecticidal protein may be a cell belonging to the clades: Opisthokonta; Viridiplantae (e.g., algae and plant); Amebozoa; Cercozoa; Alveolata; Marine flagellates; Heterokonta; Discicristata; or Excavata.

In some embodiments, the procedures and methods described here can be accomplished using a host cell that is, e.g., a Metazoan, a Choanoflagellata, or a fungi.

In some embodiments, the procedures and methods described here can be accomplished using a host cell that is a fungi. For example, in some embodiments, the host cell may be a cell belonging to the eukaryote phyla: Ascomycota, Basidiomycota, Chytridiomycota, Microsporidia, or Zygomycota

In some embodiments, the procedures and methods described here can be accomplished using a host cell that is a fungi belonging to one of the following genera: Aspergillus, Cladosporium, Magnaporthe, Morchella, Neurospora, Penicillium, Saccharomyces, Cryptococcus, or Ustilago.

In some embodiments, the procedures and methods described here can be accomplished using a host cell that is a fungi belonging to one of the following species: Saccharomyces cerevisiae, Saccharomyces boulardi, Saccharomyces uvarum; Aspergillus flavus, A. terreus, A. awamori; Cladosporium elatum, Cladosporium Herbarum, Cladosporium Sphaerospermum, and Cladosporium cladosporioides; Magnaporthe grise, Magnaporthe oryzae, Magnaporthe rhizophila; Morchella deliciosa, Morchella esculenta, Morchella conica; Neurospora crassa, Neurospora intermedia, Neurospora tetrasperma; Penicillium notatum, Penicillium chrysogenum, Penicillium roquefortii, or Penicillium simplicissimum.

In some embodiments, the procedures and methods described here can be accomplished using a host cell that is a Kluyveromyces lactis, Kluyveromyces marxianus, Saccharomyces cerevisiae, or Pichia pastoris.

In some embodiments, the host cell used to produce a DVP or DVP-insecticidal protein may be a fungi belonging to one of the following genera: Aspergillus, Cladosporium, Magnaporthe, Morchella, Neurospora, Penicillium, Saccharomyces, Cryptococcus, or Ustilago.

In some embodiments, the host cell used to produce a DVP or DVP-insecticidal protein may be a member of the Saccharomycetaceae family. For example, in some embodiments, the host cell may be one of the following genera within the Saccharomycetaceae family: Brettanomyces, Candida, Citeromyces, Cyniclomyces, Debaryomyces, Issatchenkia, Kazachstania, Kluyveromyces, Komagataella, Kuraishia, Lachancea, Lodderomyces, Nakaseomyces, Pachysolen, Pichia, Saccharomyces, Spathaspora, Tetrapisispora, Vanderwaltozyma, Torulaspora, Williopsis, Zygosaccharomyces, or Zygotorulaspora.

In some embodiments, the host cell used to produce a DVP or DVP-insecticidal protein may be one of the following: Aspergillus flavus, Aspergillus terreus, Aspergillus awamori, Cladosporium elatum, Cladosporium Herbarum, Cladosporium Sphaerospermum, Cladosporium cladosporioides, Magnaporthe grisea, Magnaporthe oryzae, Magnaporthe rhizophila, Morchella deliciosa, Morchella esculenta, Morchella conica, Neurospora crassa, Neurospora intermedia, Neurospora tetrasperma, Penicillium notatum, Penicillium chrysogenum, Penicillium roquefortii, or Penicillium simplicissimum.

In some embodiments, the host cell used to produce a DVP or DVP-insecticidal protein may be a species within the Candida genus. For example, the host cell may be one of the following: Candida albicans, Candida ascalaphidarum, Candida amphixiae, Candida antarctica, Candida argentea, Candida atlantica, Candida atmosphaerica, Candida auris, Candida blankii, Candida blattae, Candida bracarensis, Candida bromeliacearum, Candida carpophila, Candida carvajalis, Candida cerambycidarum, Candida chauliodes, Candida corydalis, Candida dosseyi, Candida dubliniensis, Candida ergatensis, Candida fructus, Candida glabrata, Candida fermentati, Candida guilliermondii, Candida haemulonii, Candida humilis, Candida insectamens, Candida insectorum, Candida intermedia, Candida jeffresii, or Candida kefyr.

In some embodiments, the host cell used to produce a DVP or DVP-insecticidal protein may be a species within the Kluyveromyces genus. For example, the host cell may be one of the following: Kluyveromyces aestuarii, Kluyveromyces dobzhanskii, Kluyveromyces lactis, Kluyveromyces marxianus, Kluyveromyces nonfermentans, or Kluyveromyces wickerhamii.

In some embodiments, the host cell used to produce a DVP or DVP-insecticidal protein may be a species within the Pichia genus. For example, the host cell may be one of the following: Pichia farinose, Pichia anomala, Pichia heedii, Pichia guilliermondii, Pichia kluyveri, Pichia membranfaciens, Pichia norvegensis, Pichia ohmeri, Pichia pastoris, Pichia methanolica, or Pichia subpelliculosa.

In some embodiments, the host cell used to produce a DVP or DVP-insecticidal protein may be a species within the Saccharomyces genus. For example, the host cell may be one of the following: Saccharomyces arboricolus, Saccharomyces bayanus, Saccharomyces bulderi, Saccharomyces cariocanus, Saccharomyces cariocus, Saccharomyces cerevisiae, Saccharomyces cerevisiae var boulardii, Saccharomyces chevalieri, Saccharomyces dairenensis, Saccharomyces ellipsoideus, Saccharomyces eubayanus, Saccharomyces exiguous, Saccharomyces florentinus, Saccharomyces fragilis, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, or Saccharomyces zonatus.

In some embodiments, the host cell used to produce a DVP or DVP-insecticidal protein may be one of the following: Saccharomyces cerevisiae, Pichia pastoris, Pichia methanolica, Schizosaccharomyces pombe, or Hansenula anomala.

The use of yeast cells as a host organism to generate recombinant DVP is an exceptional method, well known to those having ordinary skill in the art. In some embodiments, the methods and compositions described herein can be performed with any species of yeast, including but not limited to any species of the genus Saccharomyces, Pichia, Kluyveromyces, Hansenula, Yarrowia or Schizosaccharomyces and the species Saccharomyces includes any species of Saccharomyces, for example Saccharomyces cerevisiae species selected from following strains: INVSc1, YNN27, S150-2B, W303-1B, CG25, W3124, JRY188, BJ5464, AH22, GRF18, W303-1A and BJ3505. In some embodiments, members of the Pichia species including any species of Pichia, for example the Pichia species, Pichia pastoris, for example, the Pichia pastoris is selected from following strains: Bg08, Y-11430, X-33, GS 115, GS190, JC220, JC254, GS200, JC227, JC300, JC301, JC302, JC303, JC304, JC305, JC306, JC307, JC308, YJN165, KM71, MC100-3, SMD1163, SMD1165, SMD1168, GS241, MS105, any pep4 knock-out strain and any prbl knock-out strain, as well as Pichia pastoris selected from following strains: Bg08, X-33, SMD1168 and KM71. In some embodiments, any Kluyveromyces species can be used to accomplish the methods described here, including any species of Kluyveromyces, for example, Kluyveromyces lactis, and we teach that the stain of Kluyveromyces lactis can be but is not required to be selected from following strains: GG799, YCT306, YCT284, YCT389, YCT390, YCT569, YCT598, NRRL Y-1140, MW98-8C, MS1, CBS293.91, Y721, MD2/1, PM6-7A, WM37, K6, K7, 22AR1, 22A295-1, SD11, MG1/2, MSK110, JA6, CMK5, HP101, HP108 and PM6-3C, in addition to Kluyveromyces lactis species is selected from GG799, YCT306 and NRRL Y-1140.

In some embodiments, the host cell used to produce a DVP or a DVP-insecticidal protein can be an Aspergillus oryzae.

In some embodiments, the host cell used to produce a DVP or a DVP-insecticidal protein can be an Aspergillus japonicas.

In some embodiments, the host cell used to produce a DVP or a DVP-insecticidal protein can be an Aspergillus niger.

In some embodiments, the host cell used to produce a DVP or a DVP-insecticidal protein can be a Bacillus lichenformis.

In some embodiments, the host cell used to produce a DVP or a DVP-insecticidal protein can be a Bacillus subtilis.

In some embodiments, the host cell used to produce a DVP or a DVP-insecticidal protein can be a Trichoderma reesei.

In some embodiments, the procedures and methods described here can be accomplished with any species of yeast, including but not limited to any species of Hansenula species including any species of Hansenula and preferably Hansenula polymorpha. In some embodiments, the procedures and methods described here can be accomplished with any species of yeast, including but not limited to any species of Yarrowia species for example, Yarrowia lipolytica. In some embodiments, the procedures and methods described here can be accomplished with any species of yeast, including but not limited to any species of Schizosaccharomyces species including any species of Schizosaccharomyces and preferably Schizosaccharomyces pombe.

In some embodiments, yeast species such as Kluyveromyces lactis, Saccharomyces cerevisiae, Pichia pastoris, and others, can be used as a host organism. Yeast cell culture techniques are well known to those having ordinary skill in the art. Exemplary methods of yeast cell culture can be found in Evans, Yeast Protocols. Springer (1996); Bill, Recombinant Protein Production in Yeast. Springer (2012); Hagan et al., Fission Yeast: A Laboratory Manual, CSH Press (2016); Konishi et al., Improvement of the transformation efficiency of Saccharomyces cerevisiae by altering carbon sources in pre-culture. Biosci Biotechnol Biochem. 2014; 78(6):1090-3; Dymond, Saccharomyces cerevisiae growth media. Methods Enzymol. 2013; 533:191-204; Looke et al., Extraction of genomic DNA from yeasts for PCR-based applications. Biotechniques. 2011 May; 50(5):325-8; and Romanos et al., Culture of yeast for the production of heterologous proteins. Curr Protoc Cell Biol. 2014 Sep. 2; 64:20.9.1-16, the disclosure of which is incorporated herein by reference in its entirety.

Recipes for yeast cell fermentation media and stocks are described as follows: (1) MSM media recipe: 2 g/L sodium citrate dihydrate; 1 g/L calcium sulfate dihydrate (0.79 g/L anhydrous calcium sulfate); 42.9 g/L potassium phosphate monobasic; 5.17 g/L ammonium sulfate; 14.33 g/L potassium sulfate; 11.7 g/L magnesium sulfate heptahydrate; 2 mL/L PTM1trace salt solution; 0.4 ppm biotin (from 500X, 200 ppm stock); 1-2% pure glycerol or other carbon source. (2) PTM1 trace salts solution: Cupric sulfate-5H2O 6.0 g; Sodium iodide 0.08 g; Manganese sulfate-H2O 3.0 g; Sodium molybdate-2H₂O, 0.2 g; Boric Acid 0.02 g; Cobalt chloride 0.5 g; Zinc chloride 20.0 g; Ferrous sulfate-7H₂O, 65.0 g; Biotin 0.2 g; Sulfuric Acid 5.0 ml; add Water to a final volume of 1 liter. An illustrative composition for K. lactis defined medium (DMSor) is as follows: 11.83 g/L KH₂PO₄, 2.299 g/L K₂IPO₄, 20 g/L of a fermentable sugar, e.g., galactose, maltose, latotriose, sucrose, fructose or glucose and/or a sugar alcohol, for example, erythritol, hydrogenated starch hydrolysates, isomalt, lactitol, maltitol, mannitol, and xylitol, 1 g/L MgSO₄·7H₂O, 10 g/L (NH₄)SO₄, 0.33 g/L CaCl₂.2H₂O, 1 g/L NaCl, 1 g/L KCl, 5 mg/L CuSO₄·5H₂O, 30 mg/L MnSO₄·H₂O, 10 mg/L, ZnCl₂, 1 mg/L KI, 2 mg/L COC1₂.6H₂O, , 8 mg/L Na₂MoO₄.2H₂O, 0.4 mg/L H₃BO₃,15 mg/L FeCl₃.6H₂O, 0.8 mg/L biotin, 20 mg/L Ca-pantothenate, 15 mg/L thiamine, 16 mg/L myo-inositol, 10 mg/L nicotinic acid, and 4 mg/L pyridoxine.

Yeast cells can be cultured in 48-well Deep-well plates, sealed after inoculation with sterile, air-permeable cover. Colonies of yeast, for example, K. lactis cultured on plates can be picked and inoculated the deep-well plates with 2.2 mL media per well, composed of DMSor. Inoculated deep-well plates can be grown for 6 days at 23.5° C. with 280 rpm shaking in a refrigerated incubator-shaker. On day 6 post-inoculation, conditioned media should be harvested by centrifugation at 4000 rpm for 10 minutes, followed by filtration using filter plate with 0.22 μM membrane, with filtered media are subject to HPLC analyses.

In some embodiments, a yeast cell can be produced by (a) preparing a vector comprising a first expression cassette comprising a polynucleotide operable to express a DVP or complementary nucleotide sequence thereof, said DVP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; Xₙ is C, F, A, T, S, M, or V; and X₁₂ is V, A, or T; or a pharmaceutically acceptable salt thereof, (b) introducing the vector into a yeast cell; and (c) growing the yeast cell in a growth medium under conditions operable to enable expression of the DVP and secretion into the growth medium.

In some embodiments, a yeast cell can be produced by (a) preparing a vector comprising a first expression cassette comprising a polynucleotide operable to express a DVP or complementary nucleotide sequence thereof, said DVP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; Xₙ is C, F, A, T, S, M, or V; and X₁₂ is V, A, or T; or a pharmaceutically acceptable salt thereof, (b) introducing the vector into a yeast cell; and (c) growing the yeast cell in a growth medium under conditions operable to enable expression of the DVP and secretion into the growth medium; wherein if X₉ is G, T, A, S, M or V, or X₁₁ is F, A, T, S, M or V, then a disulfide bond is removed.

In some embodiments, a yeast cell can be produced by (a) preparing a vector comprising a first expression cassette comprising a polynucleotide operable to express a DVP or complementary nucleotide sequence thereof, said DVP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; Xₙ is C, F, A, T, S, M, or V; and X₁₂ is V, A, or T; or a pharmaceutically acceptable salt thereof, (b) introducing the vector into a yeast cell; and (c) growing the yeast cell in a growth medium under conditions operable to enable expression of the DVP and secretion into the growth medium; wherein if X₉ is G, T, A, S, M or V, or X₁₁ is F, A, T, S, M or V, then a disulfide bond is removed.

In some embodiments, a yeast cell can be operable to express a DVP or DVP-insecticidal protein, wherein the DVP comprises an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219.

In some embodiments, a yeast cell can be operable to express a DVP or DVP-insecticidal protein, wherein the DVP comprises an amino sequence as set forth in any one of SEQ ID NOs: 6-43, 45-51, 53, 128, 130, 136, 139-140, 144, 146-147, 187-191, 202-215, or 217-219.

In some embodiments, a yeast cell can be operable to express a DVP or DVP-insecticidal protein, wherein the DVP comprises an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219.

In some embodiments, a yeast cell can be operable to express a DVP or DVP-insecticidal protein, wherein the DVP comprises an amino sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219.

In some embodiments, a yeast cell can be operable to express a DVP or DVP-insecticidal protein, wherein the DVP comprises an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 47, 53, 136, 139-140, 144, 146-147, 187-191, 210-215, or 217-219.

In some embodiments, a yeast cell can be operable to express a DVP or DVP-insecticidal protein, wherein the DVP comprises an amino sequence as set forth in any one of SEQ ID NOs: 47, 53, 136, 139-140, 144, 146-147, 187-191, 210-215, or 217-219.

In some embodiments, a yeast cell can be operable to express a DVP or DVP-insecticidal protein, wherein the DVP comprises an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 213, or 217-219.

In some embodiments, a yeast cell can be operable to express a DVP or DVP-insecticidal protein, wherein the DVP comprises an amino sequence as set forth in any one of SEQ ID NOs: 213, or 217-219.

In some embodiments, a yeast cell can be operable to express a DVP or DVP-insecticidal protein, wherein the DVP is a homopolymer or heteropolymer of two or more DVPs, wherein the amino acid sequence of each DVP is the same or different.

In some embodiments, a yeast cell can be operable to express a DVP or DVP-insecticidal protein, wherein the DVP is a fused protein comprising two or more DVPs separated by a cleavable or non-cleavable linker, and wherein the amino acid sequence of each DVP may be the same or different.

In some embodiments, a yeast cell can be operable to express a DVP or DVP-insecticidal protein, wherein the linker is cleavable inside the gut or hemolymph of an insect.

In some embodiments, a yeast cell can be operable to express a DVP or DVP-insecticidal protein, wherein the vector is a plasmid comprising an alpha-MF signal.

In some embodiments, a yeast cell can be operable to express a DVP or DVP-insecticidal protein, wherein the vector is transformed into a yeast cell.

In some embodiments, a yeast cell can be operable to express a DVP or DVP-insecticidal protein, wherein the yeast cell is selected from any species of the genera Saccharomyces, Pichia, Kluyveromyces, Hansenula, Yarrowia or Schizosaccharomyces.

In some embodiments, a yeast cell can be operable to express a DVP or DVP-insecticidal protein, wherein the yeast cell is selected from the group consisting of Kluyveromyces lactis, Kluyveromyces marxianus, Saccharomyces cerevisiae, and Pichia pastoris.

In some embodiments, a yeast cell can be operable to express a DVP or DVP-insecticidal protein, wherein the yeast cell is Kluyveromyces lactis.

In some embodiments, a yeast cell can be operable to express a DVP or DVP-insecticidal protein, wherein expression of the DVP provides a yield of at least: 70 mg/L, 80 mg/L, 90 mg/L, 100 mg/L, 110 mg/L, 120 mg/L, 130 mg/L, 140 mg/L, 150 mg/L, 160 mg/L, 170 mg/L, 180 mg/L, 190 mg/L 200 mg/L, 500 mg/L, 750 mg/L, 1,000 mg/L, 1,250 mg/L, 1,500 mg/L, 1,750 mg/L or at least 20,000 mg/L of DVP per liter of medium.

In some embodiments, a yeast cell can be operable to express a DVP or DVP-insecticidal protein, wherein expression of the DVP provides a yield of at least 100 mg/L of DVP per liter of medium.

In some embodiments, a yeast cell can be operable to express a DVP or DVP-insecticidal protein, wherein expression of the DVP in the medium results in the expression of a single DVP in the medium.

In some embodiments, a yeast cell can be operable to express a DVP or DVP-insecticidal protein, wherein expression of the DVP in the medium results in the expression of a DVP polymer comprising two or more DVP polypeptides in the medium.

In some embodiments, a yeast cell can be operable to express a DVP or DVP-insecticidal protein, wherein the vector comprises two or three expression cassettes, each expression cassette operable to encode the DVP of the first expression cassette.

In some embodiments, a yeast cell can be operable to express a DVP or DVP-insecticidal protein, wherein the vector comprises two or three expression cassettes, each expression cassette operable to encode the DVP of the first expression cassette, or a DVP of a different expression cassette.

In some embodiments, a yeast cell can be operable to express a DVP or DVP-insecticidal protein, wherein the expression cassette is operable to encode a DVP an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191,202-215, or 217-219.

Any of the aforementioned methods, and/or any of the methods described herein, can be used to produce one or more of the DVPs or DVP-insecticidal proteins as described herein. For example, any of the methods described herein can be used to produce one or more of the DVPs described in the present disclosure, e.g., DVPs an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219, which are likewise described herein.

Yeast Transformation, DVP Purification, and Analysis

An exemplary method of yeast transformation is as follows: the expression vectors carrying a DVP ORF are transformed into yeast cells. First, the expression vectors are usually linearized by specific restriction enzyme cleavage to facilitate chromosomal integration via homologous recombination. The linear expression vector is then transformed into yeast cells by a chemical or electroporation method of transformation and integrated into the targeted locus of the yeast genome by homologous recombination. The integration can happen at the same chromosomal locus multiple times; therefore, the genome of a transformed yeast cell can contain multiple copies of DVP expression cassettes. The successfully transformed yeast cells can be identified using growth conditions that favor a selective marker engineered into the expression vector and co-integrated into yeast chromosomes with the DVP ORF; examples of such markers include, but are not limited to, acetamide prototrophy, zeocin resistance, geneticin resistance, nourseothricin resistance, and uracil prototrophy.

Due to the influence of unpredictable and variable factors-such as epigenetic modification of genes and networks of genes, and variation in the number of integration events that occur in individual cells in a population undergoing a transformation procedure-individual yeast colonies of a given transformation process will differ in their capacities to produce a DVP ORF. Therefore, transgenic yeast colonies carrying the DVP transgenes should be screened for high yield strains. Two effective methods for such screening—each dependent on growth of small-scale cultures of the transgenic yeast to provide conditioned media samples for subsequent analysis-use reverse-phase HPLC or housefly injection procedures to analyze conditioned media samples from the positive transgenic yeast colonies.

The transgenic yeast cultures can be performed using 14 mL round bottom polypropylene culture tubes with 5 to 10 mL defined medium added to each tube, or in 48-well deep well culture plates with 2.2 mL defined medium added to each well. The defined medium, not containing crude proteinaceous extracts or by-products such as yeast extract or peptone, is used for the cultures to reduce the protein background in the conditioned media harvested for the later screening steps. The cultures are performed at the optimal temperature, for example, 23.5° C. for K. lactis, for about 5-6 days, until the maximum cell density is reached. DVPs will now be produced by the transformed yeast cells and secreted out of cells to the growth medium. To prepare samples for the screening, cells are removed from the cultures by centrifugation and the supernatants are collected as the conditioned media, which are then cleaned by filtration through 0.22 μm filter membrane and then made ready for strain screening.

In some embodiments, positive yeast colonies transformed with DVP can be screened via reverse-phase HPLC (rpHPLC) screening of putative yeast colonies. In this screening method, an HPLC analytic column with bonded phase of C18 can be used. Acetonitrile and water are used as mobile phase solvents, and a UV absorbance detector set at 220 nm is used for the peptide detection. Appropriate amounts of the conditioned medium samples are loaded into the rpHPLC system and eluted with a linear gradient of mobile phase solvents. The corresponding peak area of the insecticidal peptide in the HPLC chromatograph is used to quantify the DVP concentrations in the conditioned media. Known amounts of pure DVP are run through the same rpHPLC column with the same HPLC protocol to confirm the retention time of the peptide and to produce a standard peptide HPLC curve for the quantification.

An exemplary reverse-phase HPLC screening process of positive K. lactis cells is as follows: a DVP ORF can be inserted into the expression vector, pKLAC1, and transformed into the K. lactis strain, YCT306, from New England Biolabs, Ipswich, MA, USA. pKLAC1 vector is an integrative expression vector. Once the DVP transgenes were cloned into pKLAC1 and transformed into YCT306, their expression was controlled by the LAC4 promoter. The resulting transformed colonies produced pre-propeptides comprising an α-mating factor signal peptide, a Kex2 cleavage site and mature DVPs. The α-Mating factor signal peptide guides the pre-propeptides to enter the endogenous secretion pathway, and mature DVPs are released into the growth media.

In some embodiments, codon optimization for DVP expression can be performed in two rounds, for example, in the first round, based on some common features of high expression DNA sequences, multiple variants of the DVP ORF, expressing an α-Mating factor signal peptide, a Kex2 cleavage site and the DVP, are designed and their expression levels are evaluated in the YCT306 strain of K. lactis, resulting in an initial K. lactis expression algorithm; in a second round of optimization, additional variant DVP ORFs can be designed based on the initial K. lactis expression algorithm to further fine-tuned the K. lactis expression algorithm, and identify the best ORF for DVP expression in K. lactis. In some embodiments, the resulting DNA sequence from the foregoing optimization can have an open reading frame encoding an α-MF signal peptide, a Kex2 cleavage site and a DVP, which can be cloned into the pKLAC1 vector using Hind III and Not I restriction sites, resulting in DVP expression vectors.

In some embodiments, the yeast, Pichia pastoris, can be transformed with a DVP expression cassette. An exemplary method for transforming P. pastoris is as follows: yeast vectors can be used to transform a DVP expression cassette into P. pastoris. The vectors can be obtained from commercial vendors known to those having ordinary skill in the art. In some embodiments, the vectors can be integrative vectors, and may use the uracil phosphoribosyltransferase promoter (pUPP) to enhance the heterologous transgene expression. In some embodiments, the vectors may offer different selection strategies; e.g., in some embodiments, the only difference between the vectors can be that one vector may provide G418 resistance to the host yeast, while the other vector may provide Zeocin resistance. In some embodiments, pairs of complementary oligonucleotides, encoding the DVP may be designed and synthesized for subcloning into the two yeast expression vectors. Hybridization reactions can be performed by mixing the corresponding complementary oligonucleotides to a final concentration of 20 μM in 30 mM NaCl, 10 mM Tris-Cl (all final concentrations), pH 8, and then incubating at 95° C. for 20 min, followed by a 9-hour incubation starting at 92° C. and ending at 17° C., with 3° C. drops in temperature every 20 min. The hybridization reactions will result in DNA fragments encoding DVP. The two P. pastoris vectors can be digested with BsaI-HF restriction enzymes, and the double stranded DNA products of the reactions are then subcloned into the linearized P. pastoris vectors using standard procedures. Following verification of the sequences of the subclones, plasmid aliquots can be transfected by electroporation into a P. pastoris strain (e.g., Bg08). The resulting transformed yeast, can be selected based on resistance (e.g., in this example, to Zeocin or G418) conferred by elements engineered into the vectors.

Peptide Yield Screening and Evaluation

In some embodiments, DVP or DVP-insecticidal protein yield can be evaluated using an Agilent 1100 HPLC system equipped with an Onyx monolithic 4.5×100 mm, C18 reverse-phase analytical HPLC column and an auto-injector. An illustrative use of the Agilent 1100 HPLC system equipped with an Onyx monolithic 4.5×100 mm, C18 reverse-phase analytical HPLC column and an auto-injector is as follows: filtered conditioned media samples from transformed K. lactis cells are analyzed using Agilent 1100 HPLC system equipped with an Onyx monolithic 4.5×100 mm, C18 reverse-phase analytical HPLC column and an auto-injector by analyzing HPLC grade water and acetonitrile containing 0.1% trifluoroacetic acid, constituting the two mobile phase solvents used for the HPLC analyses; the peak areas of both the DVP or Dvp-insecticidal protein are analyzed using HPLC chromatographs, and then used to calculate the peptide concentration in the conditioned media, which can be further normalized to the corresponding final cell densities (as determined by OD600 measurements) as normalized peptide yield.

In some embodiments, positive yeast colonies transformed with DVP or DVP-insecticidal protein can be screened using a housefly injection assay. DVP or DVP-insecticidal protein can paralyze/kill houseflies when injected in measured doses through the body wall of the dorsal thorax. The efficacy of the DVP or DVP-insecticidal protein can be defined by the median paralysis/lethal dose of the peptide (PD₅₀/LD₅₀), which causes 50% knock-down ratio or mortality of the injected houseflies respectively. The pure DVP or DVP-insecticidal protein is normally used in the housefly injection assay to generate a standard dose-response curve, from which a PD₅₀/LD₅₀ value can be determined. Using a PD₅₀/LD₅₀ value from the analysis of a standard dose-response curve of the pure DVP or DVP-insecticidal protein, quantification of the DVP or DVP-insecticidal protein produced by the transformed yeast can be achieved using a housefly injection assay performed with serial dilutions of the corresponding conditioned media.

An exemplary housefly injection bioassay is as follows: conditioned media is serially diluted to generate full dose-response curves from the housefly injection bioassay. Before injection, adult houseflies (Musca domestica) are immobilized with CO₂, and 12-18 mg houseflies are selected for injection. A microapplicator, loaded with a 1 cc syringe and 30-gauge needle, is used to inject 0.5 μL per fly, doses of serially diluted conditioned media samples into houseflies through the body wall of the dorsal thorax. The injected houseflies are placed into closed containers with moist filter paper and breathing holes on the lids, and they are examined by knock-down ratio or by mortality scoring at 24 hours post-injection. Normalized yields are calculated. Peptide yield means the peptide concentration in the conditioned media in units of mg/L. However, peptide yields are not always sufficient to accurately compare the strain production rate. Individual strains may have different growth rates, hence when a culture is harvested, different cultures may vary in cell density. A culture with a high cell density may produce a higher concentration of the peptide in the media, even though the peptide production rate of the strain is lower than another strain which has a higher production rate. Accordingly, the term “normalized yield” is created by dividing the peptide yield with the cell density in the corresponding culture and this allows a better comparison of the peptide production rate between strains. The cell density is represented by the light absorbance at 600 nm with a unit of “A” (Absorbance unit).

Screening yeast colonies that have undergone a transformation with DVP or DVP-insecticidal protein can identify the high yield yeast strains from hundreds of potential colonies. These strains can be fermented in bioreactor to achieve at least up to 4 g/L or at least up to 3 g/L or at least up to 2 g/L yield of the DVP or DVP-insecticidal protein when using optimized fermentation media and fermentation conditions described herein. The higher rates of production (expressed in mg/L) can be anywhere from about 100 mg/L to about 100,000 mg/L; or from about 100 mg/L to about 90,000 mg/L; or from about 100 mg/L to about 80,000 mg/L; or from about 100 mg/L to about 70,000 mg/L; or from about 100 mg/L to about 60,000 mg/L; or from about 100 mg/L to about 50,000 mg/L; or from about 100 mg/L to about 40,000 mg/L; or from about 100 mg/L to about 30,000 mg/L; or from about 100 mg/L to about 20,000 mg/L; or from about 100 mg/L to about 17,500 mg/L; or from about 100 mg/L to about 15,000 mg/L; or from about 100 mg/L to about 12,500 mg/L; or from about 100 mg/L to about 10,000 mg/L; or from about 100 mg/L to about 9,000 mg/L; or from about 100 mg/L to about 8,000 mg/L; or from about 100 mg/L to about 7,000 mg/L; or from about 100 mg/L to about 6,000 mg/L; or from about 100 mg/L to about 5,000 mg/L; or from about 100 mg/L to about 3,000 mg/L; or from about 100 mg/L to 2,000 mg/L; or from about 100 mg/L to 1,500 mg/L; or from about 100 mg/L to 1,000 mg/L; or from about 100 mg/L to 750 mg/L; or from about 100 mg/L to 500 mg/L; or from about 150 mg/L to 100,000 mg/L; or from about 200 mg/L to 100,000 mg/L; or from about 300 mg/L to 100,000 mg/L; or from about 400 mg/L to 100,000 mg/L; or from about 500 mg/L to 100,000 mg/L; or from about 750 mg/L to 100,000 mg/L; or from about 1,000 mg/L to 100,000 mg/L; or from about 1,250 mg/L to 100,000 mg/L; or from about 1,500 mg/L to 100,000 mg/L; or from about 2,000 mg/L to 100,000 mg/L; or from about 2,500 mg/L to 100,000 mg/L; or from about 3,000 mg/L to 100,000 mg/L; or from about 3,500 mg/L to 100,000 mg/L; or from about 4,000 mg/L to 100,000 mg/L; or from about 4,500 mg/L to 100,000 mg/L; or from about 5,000 mg/L to 100,000 mg/L; or from about 6,000 mg/L to 100,000 mg/L; or from about 7,000 mg/L to 100,000 mg/L; or from about 8,000 mg/L to 100,000 mg/L; or from about 9,000 mg/L to 100,000 mg/L; or from about 10,000 mg/L to 100,000 mg/L; or from about 12,500 mg/L to 100,000 mg/L; or from about 15,000 mg/L to 100,000 mg/L; or from about 17,500 mg/L to 100,000 mg/L; or from about 20,000 mg/L to 100,000 mg/L; or from about 30,000 mg/L to 100,000 mg/L; or from about 40,000 mg/L to 100,000 mg/L; or from about 50,000 mg/L to 100,000 mg/L; or from about 60,000 mg/L to 100,000 mg/L; or from about 70,000 mg/L to 100,000 mg/L; or from about 80,000 mg/L to 100,000 mg/L; or from about 90,000 mg/L to 100,000 mg/L; or any range of any value provided or even greater yields than can be achieved with a peptide before conversion, using the same or similar production methods that were used to produce the peptide before conversion.

Pharmaceutically Acceptable Salts

As used herein, the term “pharmaceutically acceptable salt” and “agriculturally acceptable salt” are synonymous. In some embodiments, pharmaceutically acceptable salts, hydrates, solvates, crystal forms and individual isomers, enantiomers, tautomers, diastereomers and prodrugs of the DVP described herein can be utilized.

In some embodiments, a pharmaceutically acceptable salt of the present invention possesses the desired pharmacological activity of the parent compound. Such salts include: acid addition salts, formed with inorganic acids; acid addition salts formed with organic acids; or salts formed when an acidic proton present in the parent compound is replaced by a metal ion, e.g., an alkali metal ion, aluminum ion; or coordinates with an organic base such as ethanolamine, and the like.

In some embodiments, pharmaceutically acceptable salts include conventional toxic or non-toxic salts. For example, in some embodiments, convention non-toxic salts include those such as fumarate, phosphate, citrate, chlorydrate, and the like. In some embodiments, the pharmaceutically acceptable salts of the present invention can be synthesized from a parent compound by conventional chemical methods. In some embodiments, such salts can be prepared by reacting the free acid or base forms of these compounds with a stoichiometric amount of the appropriate base or acid in water or in an organic solvent, or in a mixture of the two. In some embodiments, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is incorporated herein by reference in its entirety.

In some embodiments, a pharmaceutically acceptable salt can be one of the following: hydrochloride; sodium; sulfate; acetate; phosphate or diphosphate; chloride; potassium; maleate; calcium; citrate; mesylate; nitrate; tartrate; aluminum; or gluconate.

In some embodiments, a list of pharmaceutically acceptable acids that can be used to form salts can be: glycolic acid; hippuric acid; hydrobromic acid; hydrochloric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); undecylenic acid; a 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; or glycerophosphoric acid.

In some embodiments, pharmaceutically acceptable salt can be any organic or inorganic addition salt.

In some embodiments, the salt may use an inorganic acid and an organic acid as a free acid. The inorganic acid may be hydrochloric acid, bromic acid, nitric acid, sulfuric acid, perchloric acid, phosphoric acid, etc. The organic acid may be citric acid, acetic acid, lactic acid, maleic acid, fumaric acid, gluconic acid, methane sulfonic acid, gluconic acid, succinic acid, tartaric acid, galacturonic acid, embonic acid, glutamic acid, aspartic acid, oxalic acid, (D) or (L) malic acid, maleic acid, methane sulfonic acid, ethane sulfonic acid, 4-toluene sulfonic acid, salicylic acid, citric acid, benzoic acid, malonic acid, etc.

In some embodiments, the salts include alkali metal salts (sodium salts, potassium salts, etc.) and alkaline earth metal salts (calcium salts, magnesium salts, etc.). For example, the acid addition salt may include acetate, aspartate, benzoate, besylate, bicarbonate/carbonate, bisulfate/sulfate, borate, camsylate, citrate, edisilate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthalate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salt, etc., and among them, hydrochloride or trifluoroacetate may be used.

In yet other embodiments, the pharmaceutically acceptable salt can be a salt with an acid such as acetic acid, propionic acid, butyric acid, formic acid, trifluoroacetic acid, maleic acid, tartaric acid, citric acid, stearic acid, succinic acid, ethylsuccinic acid, lactobionic acid, gluconic acid, glucoheptonic acid, benzoic acid, methanesulfonic acid, ethanesulfonic acid, 2-hydroxyethanesulfonic acid, benzenesulfonic acid, p-toluenesulfonic acid, laurylsulfuric acid, malic acid, aspartic acid, glutaminic acid, adipic acid, cysteine, N-acetylcysteine, hydrochloric acid, hydrobromic acid, phosphoric acid, sulfuric acid, hydroiodic acid, nicotinic acid, oxalic acid, picric acid, thiocyanic acid, undecanoic acid, polyacrylate or carboxyvinyl polymer.

In some embodiments, the pharmaceutically acceptable salt can be prepared from either inorganic or organic bases. Salts derived from inorganic bases include, but are not limited to, the sodium, potassium, lithium, ammonium, calcium, magnesium, ferrous, zinc, copper, manganous, aluminum, ferric, manganic salts, and the like. Preferred inorganic salts are the ammonium, sodium, potassium, calcium, and magnesium salts. Salts derived from organic bases include, but are not limited to, salts of primary, secondary, and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Preferred organic bases are isopropylamine, diethylamine, ethanolamine, piperidine, tromethamine, and choline.

In some embodiments, pharmaceutically acceptable salt refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), the disclosure of which is incorporated herein by reference in its entirety.

In some embodiments, the salts of the present invention can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Exemplary descriptions of pharmaceutically acceptable salts is provided in P. H. Stahl and C. G. Wermuth, (editors), Handbook of Pharmaceutical Salts: Properties, Selection and Use, John Wiley & Sons, Aug. 23, (2002), the disclosure of which is incorporated herein by reference in its entirety.

DVP Incorporation into Plants or Parts Thereof

The DVPs described herein, and/or an insecticidal protein comprising at least one DVP as described herein, can be incorporated into plants, plant tissues, plant cells, plant seeds, and/or plant parts thereof, for either the stable, or transient expression of a DVP or a DVP-insecticidal protein, and/or a polynucleotide sequence encoding the same.

In some embodiments, the DVP or DVP-insecticidal protein can be incorporated into a plant using recombinant techniques known in the art. In some embodiments, the DVP or DVP-insecticidal protein may be in the form of an insecticidal protein which may comprise one or more DVP monomers.

As used herein, with respect to transgenic plants, plant tissues, plant cells, and plant seeds, the term “DVP” also encompasses a DVP-insecticidal protein, and a “DVP polynucleotide” is similarly also used to encompass a polynucleotide or group of polynucleotides operable to express and/or encode an insecticidal protein comprising one or more DVPs.

The goal of incorporating a DVP into plants is to deliver DVPs and/or DVP-insecticidal proteins to the pest via the insect's consumption of the transgenic DVP expressed in a plant tissue consumed by the insect. Upon the consumption of the DVP by the insect from its food (e.g., via an insect feeding upon a transgenic plant transformed with a DVP), the consumed DVP may have the ability to inhibit the growth, impair the movement, or even kill an insect. Accordingly, transgenic plants expressing a DVP polynucleotide and/or a DVP polypeptide may express said DVP polynucleotide/polypeptide in a variety of plant tissues, including but not limited to: the epidermis (e.g., mesophyll); periderm; phloem; xylem; parenchyma; collenchyma; sclerenchyma; and primary and secondary meristematic tissues. For example, in some embodiments, a polynucleotide sequence encoding a DVP can be operably linked to a regulatory region containing a phosphoenolpyruvate carboxylase promoter, resulting in the expression of a DVP in a plant's mesophyll tissue.

Transgenic plants expressing a DVP and/or a polynucleotide operable to express DVP can be generated by any one of the various methods and protocols well known to those having ordinary skill in the art; such methods of the invention do not require that a particular method for introducing a nucleotide construct to a plant be used, only that the nucleotide construct gains access to the interior of at least one cell of the plant. Methods for introducing nucleotide constructs into plants are known in the art including, but not limited to, stable transformation methods, transient transformation methods, and virus-mediated methods. “Transgenic plants” or “transformed plants” or “stably transformed” plants or cells or tissues refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell. “Heterologous” generally refers to the nucleic acid sequences that are not endogenous to the cell or part of the native genome in which they are present, and have been added to the cell by infection, transfection, microinjection, electroporation, microprojection, or the like.

Transformation of plant cells can be accomplished by one of several techniques known in the art. Typically, a construct that expresses an exogenous or heterologous peptide or polypeptide of interest (e.g., a DVP), would contain a promoter to drive transcription of the gene, as well as a 3′ untranslated region to allow transcription termination and polyadenylation. The design and organization of such constructs is well known in the art. In some embodiments, a gene can be engineered such that the resulting peptide is secreted, or otherwise targeted within the plant cell to a specific region and/or organelle. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression.

Typically, a plant expression cassette can be inserted into a plant transformation vector. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors.” Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the DVP are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Because the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Transformation protocols as well as protocols for introducing nucleotide sequences into plants may vary depending on the type of plant or plant cell, i.e., monocot or dicot, targeted for transformation. Generation of transgenic plants may be performed by one of several methods, including, but not limited to, microinjection, electroporation, direct gene transfer, introduction of heterologous DNA by Agrobacterium into plant cells (Agrobacterium-mediated transformation), bombardment of plant cells with heterologous foreign DNA adhered to particles, ballistic particle acceleration, aerosol beam transformation, Lec1 transformation, and various other non-particle direct-mediated methods to transfer DNA. Exemplary transformation protocols are disclosed in U.S. Published Application No. 20010026941; U.S. Pat. No. 4,945,050; International Publication No. WO 91/00915; and U.S. Published Application No. 2002015066, the disclosures of which are incorporated herein by reference in their entireties.

Chloroplasts can also be readily transformed, and methods concerning the transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606, the disclosure of which is incorporated herein by reference in its entirety. The method of chloroplast transformation relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

Following integration of heterologous foreign DNA into plant cells, one having ordinary skill may then apply a maximum threshold level of appropriate selection chemical/reagent (e.g., an antibiotic) in the medium to kill the untransformed cells, and separate and grow the putatively transformed cells that survive from this selection treatment by transferring said surviving cells regularly to a fresh medium. By continuous passage and challenge with appropriate selection, an artisan identifies and proliferates the cells that are transformed with the plasmid vector. Molecular and biochemical methods can then be used to confirm the presence of the integrated heterologous gene of interest into the genome of the transgenic plant.

The cells that have been transformed may be grown into plants in accordance with conventional methods known to those having ordinary skill in the art. See, for example, McCormick et al. (1986) Plant Cell Reports 5:81-84, the disclosure of which is incorporated herein by reference in its entirety. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct of the invention, for example, an expression cassette of the invention, stably incorporated into their genome.

In various embodiments, the present disclosure provides a DVP-insecticidal protein, that act as substrates for insect proteinases, proteases and peptidases (collectively referred to herein as “proteases”) as described above.

In some embodiments, transgenic plants or parts thereof, that may be receptive to the expression of DVPs can include: alfalfa, banana, barley, bean, broccoli, cabbage, canola, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and a wheat plant.

In some embodiments the transgenic plant may be grown from cells that were initially transformed with the DNA constructs described herein. In other embodiments, the transgenic plant may express the encoded DVP in a specific tissue, or plant part, for example, a leaf, a stem a flower, a sepal, a fruit, a root, a seed, or combinations thereof.

In some embodiments, the plant, plant tissue, plant cell, or plant seed can be transformed with a DVP wherein the DVP has an amino acid sequence of any of the DVPs of the present invention (e.g., one or more the DVPs described herein), or a polynucleotide encoding the same.

In some embodiments, the plant, plant tissue, plant cell, or plant seed can be transformed with a DVP having an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 187-191, or a polynucleotide encoding the same.

In some embodiments, the plant, plant tissue, plant cell, or plant seed can be transformed with a DVP wherein the DVP is a homopolymer or heteropolymer of two or more DVP polypeptides, wherein the amino acid sequence of each DVP is the same or different, or a polynucleotide encoding the same.

Polynucleotide Incorporation into Plants, the Proteins Expressed Therefrom

A challenge regarding the expression of heterogeneous polypeptides in transgenic plants is maintaining the desired effect (e.g., insecticidal activity) of the introduced polypeptide upon expression in the host organism; one way to maintain such an effect is to increase the chance of proper protein folding through the use of an operably linked Endoplasmic Reticulum Signal Peptide (ERSP). Another method to maintain the effect of a transgenic protein is to incorporate a Translational Stabilizing Protein (STA).

Plants can be transiently or stably transfected with the DNA sequence that encodes a DVP or a DVP-insecticidal protein comprising one or more DVPs, using any of the transfection methods described above. Alternatively, plants can be transfected with a polynucleotide that encodes a DVP, wherein said DVP is operably linked to a polynucleotide operable to encode an Endoplasmic Reticulum Signal Peptide (ERSP); linker, Translational Stabilizing Protein (STA); or combination thereof. For example, in some embodiments, a transgenic plant or plant genome can be transformed with a polynucleotide sequence that encodes the Endoplasmic Reticulum Signal Peptide (ERSP); DVP; and/or intervening linker peptide (LINKER or L), thus causing mRNA transcribed from the heterogeneous DNA to be expressed in the transformed plant, and subsequently, said mRNA to be translated into a peptide.

Endoplasmic Reticulum Signal Peptide (ERSP)

The subcellular targeting of a recombinant protein to the ER can be achieved through the use of an ERSP operably linked to said recombinant protein; this allows for the correct assembly and/or folding of such proteins, and the high level accumulation of these recombinant proteins in plants. Exemplary methods concerning the compartmentalization of host proteins into intracellular storage are disclosed in McCormick et al., Proc. Natl. Acad. Sci. USA 96(2):703-708, 1999; Staub et al., Nature Biotechnology 18:333-338, 2000; Conrad et al., Plant Mol. Biol. 38:101-109, 1998; and Stoger et al., Plant Mol. Biol. 42:583-590, 2000, the disclosures of which are incorporated herein by reference in their entireties. Accordingly, one way to achieve the correct assembly and/or folding of recombinant proteins, is to operably link an endoplasmic reticulum signal peptide (ERSP) to the recombinant protein of interest.

In some embodiments, a peptide comprising an Endoplasmic Reticulum Signal Peptide (ERSP) can be operably linked to a DVP (designated as ERSP-DVP), wherein said ERSP is the N-terminal of said peptide. In some embodiments, the ERSP peptide is between 3 to 60 amino acids in length, between 5 to 50 amino acids in length, between 20 to 30 amino acids in length.

In some embodiments, DVP ORF starts with an ersp at its 5′-end. For the DVP to be properly folded and functional when it is expressed from a transgenic plant, it must have an ersp nucleotide fused in frame with the polynucleotide encoding a DVP. During the cellular translation process, translated ERSP can direct the DVP being translated to insert into the Endoplasmic Reticulum (ER) of the plant cell by binding with a cellular component called a signal-recognition particle. Within the ER the ERSP peptide is cleaved by signal peptidase and the DVP is released into the ER, where the DVP is properly folded during the post-translation modification process, for example, the formation of disulfide bonds. Without any additional retention protein signals, the protein is transported through the ER to the Golgi apparatus, where it is finally secreted outside the plasma membrane and into the apoplastic space. DVP can accumulate at apoplastic space efficiently to reach the insecticidal dose in plants.

The ERSP peptide is at the N-terminal region of the plant-translated DVP complex and the ERSP portion is composed of about 3 to 60 amino acids. In some embodiments it is 5 to 50 amino acids. In some embodiments it is 10 to 40 amino acids but most often is composed of 15 to 20; 20 to 25; or 25 to 30 amino acids. The ERSP is a signal peptide so called because it directs the transportation of a protein. Signal peptides may also be called targeting signals, signal sequences, transit peptides, or localization signals. The signal peptides for ER trafficking are often 15 to 30 amino acid residues in length and have a tripartite organization, comprised of a core of hydrophobic residues flanked by a positively charged amino terminal and a polar, but uncharged carboxyterminal region. (Zimmermann, et al, “Protein translocation across the ER membrane,” Biochimica et Biohysica Acta, 2011, 1808: 912-924).

Many ERSPs are known. It is NOT required that the ERSP be derived from a plant ERSP, non-plant ERSPs will work with the procedures described herein. Many plant ERSPs are however well known and we describe some plant derived ERSPs here. For example, ins some embodiments, the ERSP can be a barley alpha-amylase signal peptide (BAAS), which is derived from the plant, Hordeum vulgare, and has an amino acid sequence as follows: “MANKHLSLSLFLVLLGLSASLASG” (SEQ ID NO:60).

Plant ERSPs, which are selected from the genomic sequence for proteins that are known to be expressed and released into the apoplastic space of plants, include examples such as BAAS, carrot extensin, and tobacco PR1. The following references provide further descriptions, and are incorporated by reference herein in their entirety: De Loose, M. et al. “The extensin signal peptide allows secretion of a heterologous protein from protoplasts” Gene, 99 (1991) 95-100; De Loose, M. et al. described the structural analysis of an extension-encoding gene from Nicotiana plumbaginfolia, the sequence of which contains a typical signal peptide for translocation of the protein to the endoplasmic reticulum; Chen, M. H. et al. “Signal peptide-dependent targeting of a rice alpha-amylase and cargo proteins to plastids and extracellular compartments of plant cells” Plant Physiology, 2004 July; 135(3): 1367-77. Epub 2004 Jul. 2. Chen, M. H. et al. studied the subcellular localization of α-amylases in plant cells by analyzing the expression of α-amylase, with and without its signal peptide, in transgenic tobacco. These references and others teach and disclose the signal peptide that can be used in the methods, procedures and peptide, protein and nucleotide complexes and constructs described herein.

In some embodiments, the ERSP can include, but is not limited to, one of the following: a BAAS; a tobacco extensin signal peptide; a modified tobacco extensin signal peptide; or a Jun a 3 signal peptide from Juniperus ashei. For example, in some embodiments, a plant can be transformed with a nucleotide that encodes any of the peptides that are described herein as Endoplasmic Reticulum Signal Peptides (ERSP), and a DVP.

The tobacco extensin signal peptide motif is another exemplary type of ERSP. See Memelink et al, the Plant Journal, 1993, V4: 1011-1022; Pogue G P et al, Plant Biotechnology Journal, 2010, V8: 638-654, the disclosures of which are incorporated herein by reference in their entireties.

In some embodiments, a DVP ORF can have a nucleotide sequence operable to encode a tobacco extensin signal peptide motif. In one embodiment, the DVP ORF can encode an extensin motif according to SEQ ID NO:61. In another embodiment, the DVP ORF can encode an extensin motif according to SEQ ID NO:62.

An illustrative example of how to generate an embodiment with an extensin signal motif is as follows: A DNA sequence encoding an extensin motif is designed (for example, the DNA sequence shown in SEQ ID NO:63 or SEQ ID NO:64) using oligo extension PCR with four synthetic DNA primers; ends sites such as a restriction site, for example, a Pac I restriction site at the 5′-end, and a 5′-end of a GFP sequence at the 3′-end, can be added using PCR with the extensin DNA sequence serving as a template, and resulting in a fragment; the fragment is used as the forward PCR primer to amplify the DNA sequence encoding a DVP ORF, for example “gfp-1-dvp” contained in a pFECT vector, thus producing a DVP ORF encoding (from N′ to C′ terminal) “ERSP-GFP-L-DVP” wherein the ERSP is extensin. The resulting DNA sequence can then be cloned into Pac I and Avr II restriction sites of a FECT vector to generate the pFECT-DVP vector for transient plant expression of GFP fused DVP.

In some embodiments, an illustrative expression system can include the FECT expression vectors containing DVP ORF is transformed into Agrobacterium, GV3101, and the transformed GV3101 is injected into tobacco leaves for transient expression of DVP ORF.

Translational Stabilizing Protein (STA)

A Translational stabilizing protein (STA) can increase the amount of DVP in plant tissues. One of the DVP ORFs, ERSP-DVP, is sufficient to express a properly folded DVP in the transfected plant, but in some embodiments, effective protection of a plant from pest damage may require that the plant expressed DVP accumulate. With transfection of a properly constructed DVP ORF, a transgenic plant can express and accumulate greater amounts of the correctly folded DVP. When a plant accumulates greater amounts of properly folded DVP, it can more easily resist, inhibit, and/or kill the pests that attack and eat the plants. One method of increasing the accumulation of a polypeptide in transgenic tissues is through the use of a translational stabilizing protein (STA). The translational stabilizing protein can be used to significantly increase the accumulation of DVP in plant tissue, and thus increase the efficacy of a plant transfected with DVP with regard to pest resistance. The translational stabilizing protein is a protein with sufficient tertiary structure that it can accumulate in a cell without being targeted by the cellular process of protein degradation.

In some embodiments, the translational stabilizing protein can be a domain of another protein, or it can comprise an entire protein sequence. In some embodiments, the translational stabilizing protein can be between 5 and 50 amino acids, 50 to 250 amino acids (e.g., GNA), 250 to 750 amino acids (e.g., chitinase) and 750 to 1500 amino acids (e.g., enhancin).

One embodiment of the translational stabilizing protein can be a polymer of fusion proteins comprising at least one DVP. A specific example of a translational stabilizing protein is provided here to illustrate the use of a translational stabilizing protein. The example is not intended to limit the disclosure or claims in any way. Useful translational stabilizing proteins are well known in the art, and any proteins of this type could be used as disclosed herein. Procedures for evaluating and testing production of peptides are both known in the art and described herein. One example of one translational stabilizing protein is Green-Fluorescent Protein (GFP) (SEQ ID NO:57; NCBI Accession No. P42212.1).

In some embodiments, a protein comprising an Endoplasmic Reticulum Signal Peptide (ERSP) can be operably linked to a DVP, which is in turn operably linked to a Translational Stabilizing Protein (STA). Here, this configuration is designated as ERSP-STA-DVP or ERSP-DVP-STA, wherein said ERSP is the N-terminal of said protein and said STA may be either on the N-terminal side (upstream) of the DVP, or of the C-terminal side (downstream) of the DVP. In some embodiments, a protein designated as ERSP-STA-DVP or ERSP-DVP-STA, comprising any of the ERSPs or DVPs described herein, can be operably linked to a STA, for example, any of the translational stabilizing proteins described, or taught by this document including GFP (Green Fluorescent Protein; SEQ ID NO:57; NCBI Accession No. P42212), or Jun a 3, (Juniperus ashei; SEQ ID NO:59; NCBI Accession No. P81295.1).

Additional examples of translational stabilizing proteins can be found in the following references, the disclosures of which are incorporated herein by reference in their entirety: Kramer, K. J. et al. “Sequence of a cDNA and expression of the gene encoding epidermal and gut chitinases of Manduca sexta” Insect Biochemistry and Molecular Biology, Vol. 23, Issue 6, September 1993, pp. 691-701. Kramer, K. J. et al. isolated and sequenced a chitinase-encoding cDNA from the tobacco hornworm, Manduca sexta. Hashimoto, Y. et al. “Location and nucleotide sequence of the gene encoding the viral enhancing factor of the Trichoplusia ni granulosis virus” Journal of General Virology, (1991), 72, 2645-2651. These references and others teach and disclose translational stabilizing proteins that can be used in the methods, procedures and peptide, protein and nucleotide complexes and constructs described herein.

In some embodiments, a DVP ORF can be transformed into a plant, for example, in the tobacco plant, Nicotiana benthamiana, using a DVP ORF that contains a STA. For example, in some embodiments, the STA can be Jun a 3. The mature Jun a 3 is a ˜30 kDa plant defending protein that is also an allergen for some people. Jun a 3 is produced by Juniperus ashei trees and can be used in some embodiments as a translational stabilizing protein (STA). In some embodiments, the Jun a 3 amino acid sequence can be the sequence shown in SEQ ID NO:65. In other embodiments, the Jun a 3 amino acid sequence can be the sequence shown in SEQ ID NO:59.

Linkers

Linker proteins assist in the proper folding of the different motifs composing a DVP ORF. The DVP ORF described in this invention also incorporates polynucleotide sequences encoding intervening linker peptides between the polynucleotide sequences encoding the DVP (dvp) and the translational stabilizing protein (sta), or between polynucleotide sequence encoding multiple polynucleotide sequences encoding DVP, i.e., (l-dvp)_{N} or (dvp-l)_{N}, if the expression ORF involves multiple DVP domain expression. The intervening linker peptides (LINKERS or L) separate the different parts of the expressed DVP construct, and help proper folding of the different parts of the complex during the expression process. In the expressed DVP construct, different intervening linker peptides can be involved to separate different functional domains. In some embodiments, the LINKER is attached to a DVP and this bivalent group can be repeated up to 10 (N=1-10) and possibly even more than 10 times (e.g., N=200) in order to facilitate the accumulation of properly folded DVP in the plant that is to be protected.

In some embodiments the intervening linker peptide can be between 1 and 30 amino acids in length. However, it is not necessarily an essential component in the expressed DVP in plants.

In some embodiments, the DVP-insecticidal protein comprises at least one DVP operably linked to a cleavable peptide. In other embodiments, the DVP-insecticidal protein comprises at least one DVP operably linked to a non-cleavable peptide.

A cleavable linker peptide can be designed to the DVP ORF to release the properly DVP from the expressed DVP complex in the transformed plant to improve the protection the DVP affords the plant with regard to pest damage. One type of the intervening linker peptide is the plant cleavable linker peptide. This type of linker peptides can be completely removed from the expressed DVP ORF complex during plant post-translational modification. Therefore, in some embodiments, the properly folded DVP linked by this type of intervening linker peptides can be released in the plant cells from the expressed DVP ORF complex during post-translational modification in the plant.

Another type of the cleavable intervening linker peptide is not cleavable during the expression process in plants. However, it has a protease cleavage site specific to serine, threonine, cysteine, aspartate proteases or metalloproteases. The type of cleavable linker peptide can be digested by proteases found in the insect and lepidopteran gut environment and/or the insect hemolymph and lepidopteran hemolymph environment to release the DVP in the insect gut or hemolymph. Using the information taught by this disclosure it should be a matter of routine for one skilled in the art to make or find other examples of LINKERS that will be useful in this invention.

In some embodiments, the DVP ORF can contain a cleavable type of intervening linker, for example, the type listed in SEQ ID NO:54, having the amino acid code of “IGER” (SEQ ID NO:54). The molecular weight of this intervening linker or LINKER is 473.53 Daltons. In other embodiments, the intervening linker peptide (LINKER) can also be one without any type of protease cleavage site, i.e., an uncleavable intervening linker peptide, for example, the linker “ETMFKHGL” (SEQ ID NO:56).

In some embodiments, the DVP-insecticidal protein can have two or more cleavable peptides, wherein the insecticidal protein comprises an insect cleavable linker (L), the insect cleavable linker being fused in frame with a construct comprising (DVP-L)ₙ, wherein “n” is an integer ranging from 1 to 200, or from 1 to 100, or from 1 to 10. In another embodiment, the DVP-insecticidal protein, and described herein, comprises an endoplasmic reticulum signal peptide (ERSP) operably linked with a DVP, which is operably linked with an insect cleavable linker (L) and/or a repeat construct (L-DVP)ₙ or (DVP-L)ₙ, wherein n is an integer ranging from 1 to 200, or from 1 to 100, or from 1 to 10.

In some embodiments, a protein comprising an Endoplasmic Reticulum Signal Peptide (ERSP) can be operably linked to a DVP and an intervening linker peptide (L or Linker); such a construct is designated as ERSP-L-DVP, or ERSP-DVP-L, wherein said ERSP is the N-terminal of said protein, and said L or Linker may be either on the N-terminal side (upstream) of the DVP, or the C-terminal side (downstream) of the DVP. A protein designated as ERSP-L-DVP, or ERSP-DVP-L, comprising any of the ERSPs or DVPs described herein, can have a Linker “L” that can be an uncleavable linker peptide, or a cleavable linker peptide, and which may be cleavable in a plant cells during protein expression process, or may be cleavable in an insect gut environment and/or hemolymph environment.

In some embodiments, a DVP-insecticidal protein can comprise any of the intervening linker peptides (LINKER or L) described herein, or taught by this document, including but not limited to following sequences: IGER (SEQ ID NO:54), EEKKN, (SEQ ID NO:55), and ETMFKHGL (SEQ ID NO:56), or combinations thereof.

In various embodiments, an exemplary insecticidal protein can include a protein construct comprising: (ERSP)-(DVP-L)ₙ; (ERSP)-(L)-(DVP-L)ₙ; (ERSP)-(L-DVP)ₙ; (ERSP)-(L-DVP)ₙ-(L); wherein n is an integer ranging from 1 to 200 or from 1 to 100, or from 1 to 10. In various related embodiments described above, a DVP is the aforementioned Mu-diguetoxin-Dc1a Variant Polypeptides, L is a non-cleavable or cleavable peptide, and n is an integer ranging from 1 to 200, preferably an integer ranging from 1 to 100, and more preferably an integer ranging from 1 to 10. In some embodiments, the DVP-insecticidal protein may contain DVP peptides that are the same or different, and insect cleavable peptides that are the same or different. In some embodiments, the C-terminal DVP is operably linked at its C-terminus with a cleavable peptide that is operable to be cleaved in an insect gut environment. In some embodiments, the N-terminal DVP is operably linked at its N-terminus with a cleavable peptide that is operable to be cleaved in an insect gut environment.

Some of the available proteases and peptidases found in the insect gut environment are dependent on the life-stage of the insect, as these enzymes are often spatially and temporally expressed. The digestive system of the insect is composed of the alimentary canal and associated glands. Food enters the mouth and is mixed with secretions that may or may not contain digestive proteases and peptidases. The foregut and the hind gut are ectodermal in origin. The foregut serves generally as a storage depot for raw food. From the foregut, discrete boluses of food pass into the midgut (mesenteron or ventriculus). The midgut is the site of digestion and absorption of food nutrients. Generally, the presence of certain proteases and peptidases in the midgut follow the pH of the gut. Certain proteases and peptidases in the human gastrointestinal system may include: pepsin, trypsin, chymotrypsin, elastase, carboxypeptidase, aminopeptidase, and dipeptidase.

The insect gut environment includes the regions of the digestive system in the herbivore species where peptides and proteins are degraded during digestion. Some of the available proteases and peptidases found in insect gut environments may include: (1) serine proteases; (2) cysteine proteases; (3) aspartic proteases, and (4) metalloproteases.

The two predominant protease classes in the digestive systems of phytophagous insects are the serine and cysteine proteases. Murdock et al. (1987) carried out an elaborate study of the midgut enzymes of various pests belonging to Coleoptera, while Srinivasan et al. (2008) have reported on the midgut enzymes of various pests belonging to Lepidoptera. Serine proteases are known to dominate the larval gut environment and contribute to about 95% of the total digestive activity in Lepidoptera, whereas the Coleopteran species have a wider range of dominant gut proteases, including cysteine proteases.

The papain family contains peptidases with a wide variety of activities, including endopeptidases with broad specificity (such as papain), endopeptidases with very narrow specificity (such as glycyl endopeptidases), aminopeptidases, dipeptidyl-peptidase, and peptidases with both endopeptidase and exopeptidase activities (such as cathepsins B and H). Other exemplary proteinases found in the midgut of various insects include trypsin-like enzymes, e.g. trypsin and chymotrypsin, pepsin, carboxypeptidase-B and aminotripeptidases.

Serine proteases are widely distributed in nearly all animals and microorganisms (Joanitti et al., 2006). In higher organisms, nearly 2% of genes code for these enzymes (Barrette-Ng et al., 2003). Being essentially indispensable to the maintenance and survival of their host organism, serine proteases play key roles in many biological processes. Serine proteases are classically categorized by their substrate specificity, notably by whether the residue at P1: trypsin-like (Lys/Arg preferred at P1), chymotrypsin-like (large hydrophobic residues such as Phe/Tyr/Leu at P1), or elastase-like (small hydrophobic residues such as Ala/Val at P1) (revised by Tyndall et. al.., 2005). Serine proteases are a class of proteolytic enzymes whose central catalytic machinery is composed of three invariant residues, an aspartic acid, a histidine and a uniquely reactive serine, the latter giving rise to their name, the “catalytic triad”. The Asp-His-Ser triad can be found in at least four different structural contexts (Hedstrom, 2002). These four clans of serine proteases are typified by chymotrypsin, subtilisin, carboxypeptidase Y, and Clp protease. The three serine proteases of the chymotrypsin-like clan that have been studied in greatest detail are chymotrypsin, trypsin, and elastase. More recently, serine proteases with novel catalytic triads and dyads have been discovered for their roles in digestion, including Ser-His-Glu, Ser-Lys/His, His-Ser-His, and N-terminal Ser.

One class of well-studied digestive enzymes found in the gut environment of insects is the class of cysteine proteases. The term “cysteine protease” is intended to describe a protease that possesses a highly reactive thiol group of a cysteine residue at the catalytic site of the enzyme. There is evidence that many phytophagous insects and plant parasitic nematodes rely, at least in part, on midgut cysteine proteases for protein digestion. These include but are not limited to Hemiptera, especially squash bugs (Anasa tristis); green stink bug (Acrosternum hilare); Riptortus clavatus; and almost all Coleoptera examined to date, especially, Colorado potato beetle (Leptinotarsa deaemlineata); three-lined potato beetle (Lema trilineata); asparagus beetle (Crioceris asparagi); Mexican bean beetle (Epilachna varivestis); red flour beetle (Triolium castaneum); confused flour beetle (Tribolium confusum); the flea beetles (Chaetocnema spp., haltica spp., and Epitrix spp.); corn rootworm (Diabrotica Spp.); cowpea weevil (Callosobruchus aculatue); boll weevil (Antonomus grandis); rice weevil (Sitophilus oryza); maize weevil (Sitophilus zeamais); granary weevil (Sitophilus granarius); Egyptian alfalfa weevil (Hypera postica); bean weevil (Acanthoseelides obtectus); lesser grain borer (Rhyzopertha dominica); yellow meal worm (Tenebrio molitor); Thysanoptera, especially, western flower thrips (Franklini ella occidentalis); Diptera, especially, leafminer spp. (Liriomyza trifolii); plant parasitic nematodes especially the potato cyst nematodes (Globodera spp.), the beet cyst nematode (Heterodera schachtii) and root knot nematodes (Meloidogyne spp.).

Another class of digestive enzymes is the aspartic proteases. The term “aspartic protease” is intended to describe a protease that possesses two highly reactive aspartic acid residues at the catalytic site of the enzyme and which is most often characterized by its specific inhibition with pepstatin, a low molecular weight inhibitor of nearly all known aspartic proteases. There is evidence that many phytophagous insects rely, in part, on midgut aspartic proteases for protein digestion most often in conjunction with cysteine proteases. These include but are not limited to Hemiptera especially (Rhodnius prolixus) and bedbug (Cimex spp.) and members of the families Phymatidae, Pentatomidae, Lygaeidae and Belostomatidae; Coleoptera, in the families of the Meloidae, Chrysomelidae, Coccinelidae and Bruchidae all belonging to the series Cucujiformia, especially, Colorado potato beetle (Leptinotarsa decemlineata) three-lined potato beetle (Lematri lineata); southern and western corn rootworm (Diabrotica undecimpunctata and D. virgifera), boll weevil (Anthonomus grandis), squash bug (Anasatristis); flea beetle (Phyllotreta crucifera), bruchid beetle (Callosobruchus maculatus), Mexican bean beetle (Epilachna varivestis), soybean leafminer (odontota horni), margined blister beetle (Epicauta pestifera) and the red flour beetle (Triolium castaneum); Diptera, especially housefly (Musca domestica). See Terra and Ferreira (1994) Comn. Biochem. Physiol. 109B: 1-62; Wolfson and Murdock (1990) J. Chem. Ecol. 16: 1089-1102.

Other examples of intervening linker peptides can be found in the following references, which are incorporated by reference herein in their entirety: a plant expressed serine proteinase inhibitor precursor was found to contain five homogeneous protein inhibitors separated by six same linker peptides, as disclosed in Heath et al. “Characterization of the protease processing sites in a multidomain proteinase inhibitor precursor from Nicotiana alata” European Journal of Biochemistry, 1995; 230: 250-257. A comparison of the folding behavior of green fluorescent proteins through six different linkers is explored in Chang, H. C. et al. “De novo folding of GFP fusion proteins: high efficiency in eukaryotes but not in bacteria” Journal of Molecular Biology, 2005 Oct. 21; 353(2): 397-409. An isoform of the human GalNAc-Ts family, GalNAc-T2, was shown to retain its localization and functionality upon expression in N. benthamiana plants by Daskalova, S. M. et al. “Engineering of N. benthamiana L. plants for production of N-acetylgalactosamine-glycosylated proteins” BMC Biotechnology, 2010 Aug. 24; 10: 62. The ability of endogenous plastid proteins to travel through stromules was shown in Kwok, E. Y. et al. “GFP-labelled Rubisco and aspartate aminotransferase are present in plastid stromules and traffic between plastids” Journal of Experimental Botany, 2004 March; 55(397): 595-604. Epub 2004 Jan. 30. A report on the engineering of the surface of the tobacco mosaic virus (TMV), virion, with a mosquito decapeptide hormone, trypsin-modulating oostatic factor (TMOF) was made by Borovsky, D. et al. “Expression of Aedes trypsin-modulating oostatic factor on the virion of TMV: A potential larvicide” Proc Natl Acad Sci, 2006 Dec. 12; 103(50): 18963-18968. These references and others teach and disclose the intervening linkers that can be used in the methods, procedures and peptide, protein and nucleotide complexes and constructs described herein.

The DVP ORF and DVP Constructs

A “DVP ORF” refers to a nucleotide encoding a DVP, and/or one or more stabilizing proteins, secretory signals, or target directing signals, for example, ERSP or STA, and is defined as the nucleotides in the ORF that has the ability to be translated. Thus, a “DVP ORF diagram” refers to the composition of one or more DVP ORFs, as written out in diagram or equation form. For example, a “DVP ORF diagram” can be written out as using acronyms or short-hand references to the DNA segments contained within the expression ORF. Accordingly, in one example, a “DVP ORF diagram” may describe the polynucleotide segments encoding the ERSP, LINKER, STA, and DVP, by diagramming in equation form the DNA segments as “ersp” (i.e., the polynucleotide sequence that encodes the ERSP polypeptide); “linker” or “L” (i.e., the polynucleotide sequence that encodes the LINKER polypeptide); “sta” (i.e., the polynucleotide sequence that encodes the STA polypeptide), and “dvp” (i.e., the polynucleotide sequence encoding a DVP), respectively. An example of a DVP ORF diagram is “ersp-sta-(linkerᵢ-dvpⱼ)_{N},” or “ersp-(dvpⱼ-linkerᵢ)_{N}-sta” and/or any combination of the DNA segments thereof.

The following equations describe two examples of a DVP ORF that encodes an ERSP, a STA, a linker, and a DVP:

ersp-sta-l-dvp or ersp-dvp-l-sta

In some embodiments, the DVP expression open reading frame (ORF) described herein is a polynucleotide sequence that will enable the plant to express mRNA, which in turn will be translated into peptides be expressed, folded properly, and/or accumulated to such an extent that said proteins provide a dose sufficient to inhibit and/or kill one or more pests. In one embodiment, an example of a protein DVP ORF can be a Mu-diguetoxin-Dc1a variant polynucleotide (dvp), an “ersp” (i.e., the polynucleotide sequence that encodes the ERSP polypeptide) a “linker” (i.e., the polynucleotide sequence that encodes the LINKER polypeptide), a “sta” (i.e., the polynucleotide sequence that encodes the STA polypeptide), or any combination thereof, and can be described in the following equation format:

ersp-sta-(linkerᵢ-dvpⱼ)ₙ, or ersp-(dvpⱼ-linkerᵢ)ₙ-sta

The foregoing illustrative embodiment of a polynucleotide equation would result in the following protein complex being expressed: ERSP-STA-(LINKER₁-DVP_{J})_{N}, containing four possible peptide components with dash signs to separate each component. The nucleotide component of ersp is a polynucleotide segment encoding a plant endoplasmic reticulum trafficking signal peptide (ERSP). The component of sta is a polynucleotide segment encoding a translation stabilizing protein (STA), which helps the accumulation of the DVP expressed in plants, however, in some embodiments, the inclusion of sta may not be necessary in the DVP ORF. The component of linkerᵢ is a polynucleotide segment encoding an intervening linker peptide (L OR LINKER) to separate the DVP from other components contained in ORF, and from the translation stabilizing protein. The subscript letter “i” indicates that in some embodiments, different types of linker peptides can be used in the DVP ORF. The component “dvp” indicates the polynucleotide segment encoding the DVP (also known as the Mu-diguetoxin-Dc1a variant polynucleotide sequence). The subscript “j” indicates different Mu-diguetoxin-Dc1a variant polynucleotides may be included in the DVP ORF. For example, in some embodiments, the Mu-diguetoxin-Dc1a variant polynucleotide sequence can encode a DVP with an amino acid substitution, or an amino acid deletion. The subscript “n” as shown in “(linkerᵢ-dvpⱼ)ₙ” indicates that the structure of the nucleotide encoding an intervening linker peptide and a DVP can be repeated “n” times in the same open reading frame in the same DVP ORF, where “n” can be any integrate number from 1 to 10; “n” can be from 1 to 10, specifically “n” can be 1, 2, 3, 4, or 5, and in some embodiments “n” is 6, 7, 8, 9 or 10. The repeats may contain polynucleotide segments encoding different intervening linkers (LINKER) and different DVPs. The different polynucleotide segments including the repeats within the same DVP ORF are all within the same translation frame. In some embodiments, the inclusion of a sta polynucleotide in the DVP ORF may not be required. For example, an ersp polynucleotide sequence can be directly be linked to the polynucleotide encoding a DVP variant polynucleotide without a linker.

In the foregoing exemplary equation, the polynucleotide “dvp” encoding the polypeptide “DVP” can be the polynucleotide sequence that encodes any DVP as described herein.

In the foregoing exemplary equation, the polynucleotide “dvp” encoding the polypeptide “DVP” can be the polynucleotide sequence that encodes any DVP as described herein, e.g., a DVP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 187-191.

Any of the aforementioned methods, and/or any of the methods described herein, can be used to incorporate into a plant or a plant part thereof, one or more polynucleotides operable to express any one or more of the DVPs or DVP-insecticidal proteins as described herein.

In some embodiments, a polynucleotide is operable to encode a DVP-insecticidal protein having the following DVP construct orientation and/or arrangement: ERSP-DVP; ERSP-(DVP)_{N}; ERSP-DVP-L; ERSP-(DVP)_{N}-L; ERSP-(DVP-L)_{N}; ERSP-L-DVP; ERSP-L-(DVP)_{N}; ERSP-(L-DVP)_{N}; ERSP-STA-DVP; ERSP-STA-(DVP)_{N}; ERSP-DVP-STA; ERSP-(DVP)_{N}-STA; ERSP-(STA-DVP)_{N}; ERSP-(DVP-STA)_{N}; ERSP-L-DVP-STA; ERSP-L-STA-DVP; ERSP-L-(DVP-STA)_{N}; ERSP-L-(STA-DVP)_{N}; ERSP-L-(DVP)_{N}—STA; ERSP-(L-DVP)_{N}—STA; ERSP-(L-STA-DVP)_{N}; ERSP-(L-DVP-STA)_{N}; ERSP-(L-STA)_{N}-DVP; ERSP-(L-DVP)_{N}—STA; ERSP-STA-L-DVP; ERSP-STA-DVP-L; ERSP-STA-L-(DVP)_{N}; ERSP-(STA-L)_{N}-DVP; ERSP-STA-(L-DVP)_{N}; ERSP-(STA-L-DVP)_{N}; ERSP-STA-(DVP)_{N}-L; ERSP-STA-(DVP-L)_{N}; ERSP-(STA-DVP)_{N}-L; ERSP-(STA-DVP-L)_{N}; ERSP-DVP-L-STA; ERSP-DVP-STA-L; ERSP-(DVP)_{N}—STA-L ERSP-(DVP-L)_{N}-STA; ERSP-(DVP-STA)_{N}-L; ERSP-(DVP-L-STA)_{N}; or ERSP-(DVP-STA-L)_{N}; wherein N is an integer ranging from 1 to 200.

The present disclosure may be used for transformation of any plant species, including, but not limited to, monocots and dicots. Crops for which a transgenic approach or PEP would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

Transforming Plants with Polynucleotides

In some embodiments, the DVP ORFs and DVP constructs described above and herein can be cloned into any plant expression vector for DVP to be expressed in plants, either transiently or stably.

Transient plant expression systems can be used to promptly optimize the structure of the DVP ORF for some specific DVP expression in plants, including the necessity of some components, codon optimization of some components, optimization of the order of each component, etc. A transient plant expression vector is often derived from a plant virus genome. Plant virus vectors provide advantages in quick and high level of foreign gene expression in plant due to the infection nature of plant viruses. The full length of the plant viral genome can be used as a vector, but often a viral component is deleted, for example the coat protein, and transgenic ORFs are subcloned in that place. The DVP ORF can be subcloned into such a site to create a viral vector. These viral vectors can be introduced into plant mechanically since they are infectious themselves, for example through plant wound, spray-on etc. They can also be transfected into plants via agroinfection, by cloning the virus vector into the T-DNA of the crown gall bacterium, Agrobacterium tumefaciens, or the hairy root bacterium, Agrobacterium rhizogenes. The expression of the DVP in this vector is controlled by the replication of the RNA virus, and the virus translation to mRNA for replication is controlled by a strong viral promoter, for example, 35S promoter from Cauliflower mosaic virus. Viral vectors with DVP ORF are usually cloned into T-DNA region in a binary vector that can replicate itself in both E. coli strains and Agrobacterium strains. The transient transfection of a plant can be done by infiltration of the plant leaves with the Agrobacterium cells which contain the viral vector for DVP expression. In the transient transformed plant, it is common for the foreign protein expression to be ceased in a short period of time due to the post-transcriptional gene silencing (PTGS). Sometimes a PTGS suppressing protein gene is necessary to be co-transformed into the plant transiently with the same type of viral vector that drives the expression of with the DVP ORF. This improves and extends the expression of the DVP in the plant. The most commonly used PTGS suppressing protein is P19 protein discovered from tomato bushy stunt virus (TBSV).

In some embodiments, transient transfection of plants can be achieved by recombining a polynucleotide encoding a DVP with any one of the readily available vectors (see above and described herein), and confirmed, using a marker or signal (e.g., GFP emission). In some embodiments, a transiently transfected plant can be created by recombining a polynucleotide encoding a DVP with a DNA encoding a GFP-Hybrid fusion protein in a vector, and transfection said vector into a plant (e.g., tobacco) using different FECT vectors designed for targeted expression. In some embodiments, a polynucleotide encoding a DVP can be recombined with a pFECT vector for APO (apoplast localization) accumulation; a pFECT vector for CYTO (cytoplasm localization) accumulation; or pFECT with ersp vector for ER (endoplasm reticulum localization) accumulation.

An exemplary transient plant transformation strategy is agroinfection using a plant viral vector due to its high efficiency, ease, and low cost. In some embodiments, a tobacco mosaic virus overexpression system can be used to transiently transform plants with DVP. See TRBO, Lindbo J A, Plant Physiology, 2007, V145: 1232-1240, the disclosure of which is incorporated herein by reference in its entirety.

The TRBO DNA vector has a T-DNA region for agroinfection, which contains a CaMV 35S promoter that drives expression of the tobacco mosaic virus RNA without the gene encoding the viral coating protein. Moreover, this system uses the “disarmed” virus genome, therefore viral plant to plant transmission can be effectively prevented.

In another embodiment, the FECT viral transient plant expression system can be used to transiently transform plants with DVP. See Liu Z & Kearney C M, BMC Biotechnology, 2010, 10:88, the disclosure of which is incorporated herein by reference in its entirety. The FECT vector contains a T-DNA region for agroinfection, which contains a CaMV 35S promoter that drives the expression of the foxtail mosaic virus RNA without the genes encoding the viral coating protein and the triple gene block. Moreover, this system uses the “disarmed” virus genome, therefore viral plant to plant transmission can be effectively prevented. To efficiently express the introduced heterologous gene, the FECT expression system additionally needs to co-express P19, a RNA silencing suppressor protein from tomato bushy stunt virus, to prevent the post-transcriptional gene silencing (PTGS) of the introduced T-DNA (the TRBO expression system does not need co-expression of P19).

In some embodiments, the DVP ORF can be designed to encode a series of translationally fused structural motifs that can be described as follows: N′-ERSP-STA-L-DVP-C′ wherein the “N″” and “C′” indicating the N-terminal and C-terminal amino acids, respectively, and the ERSP motif can be the Barley Alpha-Amylase Signal peptide (BAAS) (SEQ ID NO:60); the stabilizing protein (STA) can be GFP (SEQ ID NO:57); the linker peptide “L” can be IGER (SEQ ID NO:54) In some embodiments, the ersp-sta-1-dvp ORF can chemically synthesized to include restrictions sites, for example a Pac I restriction site at its 5′-end, and an Avr II restriction site at its 3′-end. In some embodiments, the DVP ORF can be cloned into the Pac I and Avr II restriction sites of a FECT expression vector (pFECT) to create a Mu-diguetoxin-Dc1a variant expression vector for the FECT transient plant expression system (pFECT-DVP). To maximize expression in the FECT expression system, some embodiments may have a FECT vector expressing the RNA silencing suppressor protein P19 (pFECT-P19) generated for co-transformation.

In some embodiments, a Mu-diguetoxin-Dc1a variant expression vector can be recombined for use in a TRBO transient plant expression system, for example, by performing a routine PCR procedure and adding a Not I restriction site to the 3′-end of the DVP ORF described above, and then cloning the DVP ORF into Pac I and Not I restriction sites of the TRBO expression vector (pTRBO-DVP).

In some embodiments, an Agrobacterium tumefaciens strain, for example, commercially available GV3101 cells, can be used for the transient expression of a DVP ORF in a plant tissue (e.g., tobacco leaves) using one or more transient expression systems, for example, the FECT and TRBO expression systems. An exemplary illustration of such a transient transfection protocol includes the following: an overnight culture of GV3101 can be used to inoculate 200 mL Luria-Bertani (LB) medium; the cells can be allowed to grow to log phase with OD600 between 0.5 and 0.8; the cells can then be pelleted by centrifugation at 5000 rpm for 10 minutes at 4° C.; cells can then be washed once with 10 mL prechilled TE buffer (Tris-HCl 10 mM, EDTA 1 mM, pH8.0), and then resuspended into 20 mL LB medium; GV3101 cell resuspension can then be aliquoted in 250 μL fractions into 1.5 mL microtubes; aliquots can then be snap-frozen in liquid nitrogen and stored at −80° C. freezer for future transformation. The pFECT-DVP and pTRBO-DVP vectors can then transformed into the competent GV3101 cells using a freeze-thaw method as follows: the stored competent GV3101 cells are thawed on ice and mixed with 1 to 5 μg pure DNA (pFECT-DVP or pTRBO-DVP vector). The cell-DNA mixture is kept on ice for 5 minutes, transferred to −80° C. for 5 minutes, and incubated in a 37° C. water bath for 5 minutes. The freeze-thaw treated cells are then diluted into 1 mL LB medium and shaken on a rocking table for 2 to 4 hours at room temperature. A 200 μL aliquot of the cell-DNA mixture is then spread onto LB agar plates with the appropriate antibiotics (10 μg/mL rifampicin, 25 μg/mL gentamycin, and 50 μg/mL kanamycin can be used for both pFECT-DVP transformation and pTRBO-DVP transformation) and incubated at 28° C. for two days. Resulting transformed colonies are then picked and cultured in 6 mL aliquots of LB medium with the appropriate antibiotics for transformed DNA analysis and making glycerol stocks of the transformed GV3101 cells.

In some embodiments, the transient transformation of plant tissues, for example, tobacco leaves, can be performed using leaf injection with a 3-mL syringe without needle. In one illustrative example, the transformed GV3101 cells are streaked onto an LB plate with the appropriate antibiotics (as described above) and incubated at 28° C. for two days. A colony of transformed GV3101 cells are inoculated to 5 ml of LB-MESA medium (LB media supplemented with 10 mM MES, and 20 μM acetosyringone) and the same antibiotics described above, and grown overnight at 28° C. The cells of the overnight culture are collected by centrifugation at 5000 rpm for 10 minutes and resuspended in the induction medium (10 mM MES, 10 mM MgCl₂, 100 μM acetosyringone) at a final OD600 of 1.0. The cells are then incubated in the induction medium for 2 hours to overnight at room temperature and are then ready for transient transformation of tobacco leaves. The treated cells can be infiltrated into the underside of attached leaves of Nicotiana benthamiana plants by injection, using a 3-mL syringe without a needle attached.

In some embodiments, the transient transformation can be accomplished by transfecting one population of GV3101 cells with pFECT-DVP or pTRBO-DVP and another population with pFECT-P19, mixing the two cell populations together in equal amounts for infiltration of tobacco leaves by injection with a 3-mL syringe.

Stable integration of polynucleotide operable to encode DVP is also possible with the present disclosure, for example, the DVP ORF can also be integrated into plant genome using stable plant transformation technology, and therefore DVPs can be stably expressed in plants and protect the transformed plants from generation to generation. For the stable transformation of plants, the DVP expression vector can be circular or linear. The DVP ORF, the DVP expression cassette, and/or the vector with polynucleotide encoding an DVP for stable plant transformation should be carefully designed for optimal expression in plants based on what is known to those having ordinary skill in the art, and/or by using predictive vector design tools such as Gene Designer 2.0 (Atum Bio); VectorBuilder (Cyagen); SnapGene® viewer; GeneArt™ Plasmid Construction Service (Thermo-Fisher Scientific); and/or other commercially available plasmid design services. See Tolmachov, Designing plasmid vectors. Methods Mol Biol. 2009; 542:117-29. The expression of DVP is usually controlled by a promoter that promotes transcription in some, or all the cells of the transgenic plant. The promoter can be a strong plant viral promoter, for example, the constitutive 35S promoter from Cauliflower Mosaic Virus (CaMV); it also can be a strong plant promoter, for example, the hydroperoxide lyase promoter (pHPL) from Arabidopsis thaliana; the Glycine max polyubiquitin (Gmubi) promoter from soybean; the ubiquitin promoters from different plant species (rice, corn, potato, etc.), etc. A plant transcriptional terminator often occurs after the stop codon of the ORF to halt the RNA polymerase and transcription of the mRNA. To evaluate the DVPs expression, a reporter gene can be included in the DVP expression vector, for example, beta-glucuronidase gene (GUS) for GUS straining assay, green fluorescent protein (GFP) gene for green fluorescence detection under UV light, etc. For selection of transformed plants, a selection marker gene is usually included in the DVP expression vector. In some embodiments, the marker gene expression product can provide the transformed plant with resistance to specific antibiotics, for example, kanamycin, hygromycin, etc., or specific herbicide, for example, glyphosate etc. If agroinfection technology is adopted for plant transformation, T-DNA left border and right border sequences are also included in the DVP expression vector to transport the T-DNA portion into the plant.

The constructed DVP expression vector can be transfected into plant cells or tissues using many transfection technologies. Agroinfection is a very popular way to transform a plant using an Agrobacterium tumefaciens strain or an Agrobacterium rhizogenes strain. Particle bombardment (also called Gene Gun, or Biolistics) technology is also very common method of plant transfection. Other less common transfection methods include tissue electroporation, silicon carbide whiskers, direct injection of DNA, etc. After transfection, the transfected plant cells or tissues placed on plant regeneration media to regenerate successfully transfected plant cells or tissues into transgenic plants.

Evaluation of a transformed plant can be accomplished at the DNA level, RNA level and protein level. A stably transformed plant can be evaluated at all of these levels and a transiently transformed plant is usually only evaluated at protein level. To ensure that the DVP ORF integrates into the genome of a stably transformed plant, the genomic DNA can be extracted from the stably transformed plant tissues for and analyzed using PCR or Southern blot. The expression of the DVP in the stably transformed plant can be evaluated at the RNA level, for example, by analyzing total mRNA extracted from the transformed plant tissues using northern blot or RT-PCR. The expression of the DVP in the transformed plant can also be evaluated in protein level directly. There are many ways to evaluate expression of DVP in a transformed plant. If a reporter gene included in the DVP ORF, a reporter gene assay can be performed, for example, in some embodiments a GUS straining assay for GUS reporter gene expression, a green fluorescence detection assay for GFP reporter gene expression, a luciferase assay for luciferase reporter gene expression, and/or other reporter techniques may be employed.

In some embodiments total protein can be extracted from the transformed plant tissues for the direct evaluation of the expression of the DVP using a Bradford assay to evaluate the total protein level in the sample.

In some embodiments, analytical HPLC chromatography technology, Western blot technique, or iELISA assay can be adopted to qualitatively or quantitatively evaluate the DVP in the extracted total protein sample from the transformed plant tissues. DVP expression can also be evaluated by using the extracted total protein sample from the transformed plant tissues in an insect bioassay, for example, in some embodiments, the transformed plant tissue or the whole transformed plant itself can be used in insect bioassays to evaluate DVP expression and its ability to provide protection for the plant.

In some embodiments, a plant, plant tissue, plant cell, plant seed, or part thereof of the present invention, can comprise one or more DVPs, or a polynucleotide encoding the same, said DVP comprising an amino acid sequence that is at least

Confirming Successful Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual. Cold Spring Harbor Laboratory Press, Cold Spring Harbor, N.Y.). PCR is carried out using oligonucleotide primers specific to the gene of interest or Agrobacterium vector background, etc.

Plant transformation may be confirmed by Southern blot analysis of genomic DNA (Sambrook and Russell, 2001, supra). In general, total DNA is extracted from the transformed plant, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced gene into the plant genome according to standard techniques (Sambrook and Russell, 2001, supra).

In Northern blot analysis, RNA is isolated from specific tissues of transformed plant, fractionated in a formaldehyde agarose gel, and blotted onto a nylon filter according to standard procedures that are routinely used in the art (Sambrook and Russell, 2001, supra). Expression of RNA encoded by the polynucleotide encoding a DVP is then tested by hybridizing the filter to a radioactive probe derived from a DVP, by methods known in the art (Sambrook and Russell, 2001, supra).

Western blot, biochemical assays and the like may be carried out on the transgenic plants to confirm the presence of protein encoded by the DVP gene by standard procedures (Sambrook and Russell, 2001, supra) using antibodies that bind to one or more epitopes present on the DVP.

A number of markers have been developed to determine the success of plant transformation, for example, resistance to chloramphenicol, the aminoglycoside G418, hygromycin, or the like. Other genes that encode a product involved in chloroplast metabolism may also be used as selectable markers. For example, genes that provide resistance to plant herbicides such as glyphosate, bromoxynil, or imidazolinone may find particular use. Such genes have been reported (Stalker et al. (1985) J. Biol. Chem. 263:6310-6314 (bromoxynil resistance nitrilase gene); and Sathasivan et al. (1990) Nucl. Acids Res. 18:2188 (AHAS imidazolinone resistance gene). Additionally, the genes disclosed herein are useful as markers to assess transformation of bacterial, yeast, or plant cells. Methods for detecting the presence of a transgene in a plant, plant organ (e.g., leaves, stems, roots, etc.), seed, plant cell, propagule, embryo or progeny of the same are well known in the art. In one embodiment, the presence of the transgene is detected by testing for pesticidal activity.

Fertile plants expressing a DVP and/or Mu-diguetoxin-Dc1a variant polynucleotide may be tested for pesticidal activity, and the plants showing optimal activity selected for further breeding. Methods are available in the art to assay for pest activity. Generally, the protein is mixed and used in feeding assays. See, for example Marrone et al. (1985) J. of Economic Entomology 78:290-293.

In some embodiments, evaluating the success of a transient transfection procedure can be determined based on the expression of a reporter gene, for example, GFP. In some embodiments, GFP can be detected under U.V. light in tobacco leaves transformed with the FECT and/or TRBO vectors.

In some embodiments, DVP expression can be quantitatively evaluated in a plant (e.g., tobacco). An exemplary procedure that illustrates DVP quantification in a tobacco plant is as follows: 100 mg disks of transformed leaf tissue is collected by punching leaves with the large opening of a 1000 μL pipette tip. The collected leaf tissue is place into a 2 mL microtube with 5/32″ diameter stainless steel grinding balls, and frozen in −80° C. for 1 hour, and then homogenized using a Troemner-Talboys High Throughput Homogenizer. Next, 750 μL ice-cold TSP-SE1 extraction solutions (sodium phosphate solution 50 mM, 1:100 diluted protease inhibitor cocktail, EDTA 1 mM, DIECA 10 mM, PVPP 8%, pH 7.0) is added into the tube and vortexed. The microtube is then left still at room temperature for 15 minutes and then centrifuged at 16,000 g for 15 minutes at 4° C.; 100 μL of the resulting supernatant is taken and loaded into pre-Sephadex G-50-packed column in 0.45 μm Millipore MultiScreen filter microtiter plate with empty receiving Costar microtiter plate on bottom. The microtiter plates are then centrifuged at 800 g for 2 minutes at 4° C. The resulting filtrate solution, herein called total soluble protein extract (TSP extract) of the tobacco leaves, is then ready for the quantitative analysis.

In some embodiments, the total soluble protein concentration of the TSP extract can be estimated using Pierce Coomassie Plus protein assay. BSA protein standards with known concentrations can be used to generate a protein quantification standard curve. For example, 2 μL of each TSP extract can be mixed into 200 μL of the chromogenic reagent (CPPA reagent) of the Coomassie Plus protein assay kits and incubated for 10 minutes. The chromogenic reaction can then be evaluated by reading OD595 using a SpectroMax-M2 plate reader using SoftMax Pro as control software. The concentrations of total soluble proteins can be about 0.788±0.20 μg/μL or about 0.533±0.03 μg/μL in the TSP extract from plants transformed via FECT and TRBO, respectively, and the results can be used to calculate the percentage of the expressed Mu-diguetoxin-Dc1a Variant peptide in the TSP (% TSP) for the iELISA assay

In some embodiments, an indirect ELISA (iELISA) assay can be used to quantitatively evaluate the DVP content in the tobacco leaves transiently transformed with the FECT and/or TRBO expression systems. An illustrative example of using iELISA to quantify DVP is as follows: 5 μL of the leaf TSP extract is diluted with 95 μL of CB2 solution (Immunochemistry Technologies) in the well of an Immulon 2HD 96-well plate, with serial dilutions performed as necessary; leaf proteins obtained from extract samples are then allowed to coat the well walls for 3 hours in the dark, at room temperature, and the CB2 solution is then subsequently removed; each well is washed twice with 200 μL PBS (Gibco); 150 μL blocking solution (Block BSA in PBS with 5% non-fat dry milk) is added into each well and incubated for 1 hour, in the dark, at room temperature; after the removal of the blocking solution, a PBS wash of the wells, 100 μL of primary antibodies directed against DVP (custom antibodies are commercially available from ProMab Biotechnologies, Inc.; GenScript®; or raised using the knowledge readily available to those having ordinary skill in the art); the antibodies diluted at 1:250 dilution in blocking solution are added to each well and incubated for 1 hour in the dark at room temperature; the primary antibody is removed and each well is washed with PBS 4 times; 100 μL of HRP-conjugated secondary antibody (i.e., antibody directed against host species used to generate primary antibody, used at 1:1000 dilution in the blocking solution) is added into each well and incubated for 1 hour in the dark at room temperature.; the secondary antibody is removed and the wells are washed with PBS, 100 μL; substrate solution (a 1: 1 mixture of ABTS peroxidase substrate solution A and solution B, KPL) is added to each well, and the chromogenic reaction proceeds until sufficient color development is apparent; 100 μL of peroxidase stop solution is added to each well to stop the reaction; light absorbance of each reaction mixture in the plate is read at 405 nm using a SpectroMax-M2 plate reader, with SoftMax Pro used as control software; serially diluted known concentrations of pure DVPs samples can be treated in the same manner as described above in the iELISA assay to generate a mass-absorbance standard curve for quantities analysis. The expressed DVP can be detected by iELISA at about 3.09±1.83 ng/μL in the leaf TSP extracts from the FECT transformed tobacco; and about 3.56±0.74 ng/μL in the leaf TSP extract from the TRBO transformed tobacco. Alternatively, the expressed DVP can be about 0.40% total soluble protein (% TSP) for FECT transformed plants and about 0.67% TSP in TRBO transformed plants.

Mixtures, Compositions, and Formulations

As used herein, the terms “composition” and “formulations” are used interchangeably.

As used herein, “v/v” or “% v/v” or “volume per volume” refers to the volume concentration of a solution (“v/v” stands for volume per volume). Here, v/v can be used when both components of a solution are liquids. For example, when 50 mL of ingredient X is diluted with 50 mL of water, there will be 50 mL of ingredient X in a total volume of 100 mL; therefore, this can be expressed as “ingredient X 50% v/v.” Percent volume per volume (% v/v) is calculated as follows: (volume of solute (mL)/volume of solution (100 mL)); e.g., % v/v=mL of solute/100 mL of solution.

As used herein, “w/w” or “% w/w” or “weight per weight” refers to the weight concentration of a solution, i.e., percent weight in weight (“w/w” stands for weight per weight). Here, w/w expresses the number of grams (g) of a constituent in 100 g of solution or mixture. For example, a mixture consisting of 30 g of ingredient X, and 70 g of water would be expressed as “ingredient X 30% w/w.” Percent weight per weight (% w/w) is calculated as follows: (weight of solute (g)/weight of solution (g))×100; or (mass of solute (g)/mass of solution (g))×100.

As used herein, “w/v” or “% w/v” or “weight per volume” refers to the mass concentration of a solution, i.e., percent weight in volume (“w/v” stands for weight per volume). Here, w/v expresses the number of grams (g) of a constituent in 100 mL of solution. For example, if 1 g of ingredient X is used to make up a total volume of 100 mL, then a “1% w/v solution of ingredient X” has been made. Percent weight per volume (% w/v) is calculated as follows: (Mass of solute (g)/Volume of solution (mL))×100.

Any of the DVPs or DVP-insecticidal proteins described herein (e.g., a DVP having an amino acid sequence as set forth in SEQ ID NOs: 6-43, 45-51, 53, 128, 130, 136, 139-140, 144, 146-147, 187-191, 202-215, or 217-219, or a pharmaceutically acceptable salt thereof) can be used to create a mixture and/or composition, wherein said mixture and/or composition consists of at least one DVP.

Any of the compositions, products, polypeptides and/or plants transformed with polynucleotides operable to express a DVP, and described herein, can be used to control pests, their growth, and/or the damage caused by their actions, especially their damage to plants.

Compositions comprising a DVP, a DVP-insecticidal protein, or a pharmaceutically acceptable salt thereof, for example, agrochemical compositions, can include, but are not limited to, aerosols and/or aerosolized products, e.g., sprays, fumigants, powders, dusts, and/or gases; seed dressings; oral preparations (e.g., insect food, etc.); transgenic organisms expressing and/or producing a DVP, a DVP-insecticidal protein, and/or a DVP ORF (either transiently and/or stably), e.g., a plant or an animal.

The composition may be formulated as a powder, dust, pellet, granule, spray, emulsion, colloid, solution, or such like, and may be prepared by such conventional means as desiccation, lyophilization, homogenization, extraction, filtration, centrifugation, sedimentation, or concentration of a culture of cells comprising the polypeptide. In all such compositions that contain at least one such pesticidal polypeptide, the polypeptide may be present in a concentration of from about 1% to about 99% by weight.

In some embodiments, the pesticide compositions described herein may be made by formulating either the DVP, DVP-insecticidal protein, or pharmaceutically acceptable salt thereof, with the desired agriculturally-acceptable carrier. The compositions may be formulated prior to administration in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. In some embodiments, the formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, the disclosure of which is incorporated by reference herein in its entirety.

In some embodiments, a composition can comprise, consist essentially of, or consist of, a DVP and an excipient.

In some embodiments, a composition can comprise, consist essentially of, or consist of, a DVP-insecticidal protein and an excipient.

In some embodiments, a composition can comprise, consist essentially of, or consist of, DVP, DVP-insecticidal protein, or a pharmaceutically acceptable salt thereof, and an excipient.

Sprayable Compositions

Examples of spray products of the present invention can include field sprayable formulations for agricultural usage and indoor sprays for use in interior spaces in a residential or commercial space. In some embodiments, residual sprays or space sprays comprising a DVP, a DVP-insecticidal protein, or a pharmaceutically acceptable salt thereof can be used to reduce or eliminate insect pests in an interior space.

Surface spraying indoors (SSI) is the technique of applying a variable volume sprayable volume of an insecticide onto indoor surfaces where vectors rest, such as on walls, windows, floors and ceilings. The primary goal of variable volume sprayable volume is to reduce the lifespan of the insect pest, (for example, a fly, a flea, a tick, or a mosquito vector) and thereby reduce or interrupt disease transmission. The secondary impact is to reduce the density of insect pests within the treatment area. SSI can be used as a method for the control of insect pest vector diseases, such as Lyme disease, Salmonella, Chikungunya virus, Zika virus, and malaria, and can also be used in the management of parasites carried by insect vectors, such as Leishmaniasis and Chagas disease. Many mosquito vectors that harbor Zika virus, Chikungunya virus, and malaria include endophilic mosquito vectors, resting inside houses after taking a blood meal. These mosquitoes are particularly susceptible to control through surface spraying indoors (SSI) with a sprayable composition comprising a DVP, a DVP-insecticidal protein, or a pharmaceutically acceptable salt thereof, and an excipient. As its name implies, SSI involves applying the composition onto the walls and other surfaces of a house with a residual insecticide.

In one embodiment, the composition comprising a DVP, a DVP-insecticidal protein, or a pharmaceutically acceptable salt thereof, and an excipient will knock down insect pests that come in contact with these surfaces. SSI does not directly prevent people from being bitten by mosquitoes. Rather, it usually controls insect pests after they have blood fed, if they come to rest on the sprayed surface. SSI thus prevents transmission of infection to other persons. To be effective, SSI must be applied to a very high proportion of households in an area (usually greater than 40-80 percent). Therefore, sprays in accordance with the invention having good residual efficacy and acceptable odor are particularly suited as a component of integrated insect pest vector management or control solutions.

In contrast to SSI, which requires that the active DVP or DVP-insecticidal protein be bound to surfaces of dwellings, such as walls or ceilings, as with a paint, for example, space spray products of the invention rely on the production of a large number of small insecticidal droplets intended to be distributed through a volume of air over a given period of time. When these droplets impact on a target insect pest, they deliver a knockdown effective dose of the DVP or DVP-insecticidal protein effective to control the insect pest. The traditional methods for generating a space-spray include thermal fogging (whereby a dense cloud of a composition comprising a DVP, a DVP-insecticidal protein, or a pharmaceutically acceptable salt thereof is produced giving the appearance of a thick fog) and Ultra Low Volume (ULV), whereby droplets are produced by a cold, mechanical aerosol-generating machine. Ready-to-use aerosols such as aerosol cans may also be used.

Because large areas can be treated at any one time, the foregoing method is a very effective way to rapidly reduce the population of flying insect pests in a specific area. And, because there is very limited residual activity from the application, it must be repeated at intervals of 5-7 days in order to be fully effective. This method can be particularly effective in epidemic situations where rapid reduction in insect pest numbers is required. As such, it can be used in urban dengue control campaigns.

Effective space-spraying is generally dependent upon the following specific principles. Target insects are usually flying through the spray cloud (or are sometimes impacted whilst resting on exposed surfaces). The efficiency of contact between the spray droplets and target insects is therefore crucial. This is achieved by ensuring that spray droplets remain airborne for the optimum period of time and that they contain the right dose of insecticide. These two issues are largely addressed through optimizing the droplet size. If droplets are too big they drop to the ground too quickly and don't penetrate vegetation or other obstacles encountered during application (limiting the effective area of application). If one of these big droplets impacts an individual insect then it is also “overkill,” because a high dose will be delivered per individual insect. If droplets are too small then they may either not deposit on a target insect (no impaction) due to aerodynamics or they can be carried upwards into the atmosphere by convection currents. The optimum size of droplets for space-spray application are droplets with a Volume Median Diameter (VMD) of 10-25 microns.

In some embodiments, a sprayable composition may contain an amount of a DVP, or a pharmaceutically acceptable salt thereof, ranging from about 0.005 wt % to about 99 wt %.

In some embodiments, a sprayable composition may contain an amount of a DVP-insecticidal protein, or a pharmaceutically acceptable salt thereof, ranging from about 0.005 wt % to about 99 wt %.

Foams

The active compositions of the present invention comprising a DVP, a DVP-insecticidal protein, or a pharmaceutically acceptable salt thereof, and an excipient, may be made available in a spray product as an aerosol-based application, including aerosolized foam applications. Pressurized cans are the typical vehicle for the formation of aerosols. An aerosol propellant that is compatible with the DVP or DVP-insecticidal protein used. Preferably, a liquefied-gas type propellant is used.

Suitable propellants include compressed air, carbon dioxide, butane and nitrogen. The concentration of the propellant in the active compound composition is from about 5 percent to about 40 percent by weight of the pyridine composition, preferably from about 15 percent to about 30 percent by weight of the comprising a DVP, a DVP-insecticidal protein, or a pharmaceutically acceptable salt thereof, and an excipient.

In one embodiment, formulations comprising a DVP, a DVP-insecticidal protein, or a pharmaceutically acceptable salt thereof can also include one or more foaming agents. Foaming agents that can be used include sodium laureth sulfate, cocamide DEA, and cocamidopropyl betaine. Preferably, the sodium laureth sulfate, cocamide DEA and cocamidopropyl are used in combination. The concentration of the foaming agent(s) in the active compound composition is from about 10 percent to about 25 percent by weight, more preferably 15 percent to 20 percent by weight of the composition.

When such formulations are used in an aerosol application not containing foaming agents, the active compositions of the present invention can be used without the need for mixing directly prior to use. However, aerosol formulations containing the foaming agents do require mixing (i.e., shaking) immediately prior to use. In addition, if the formulations containing foaming agents are used for an extended time, they may require additional mixing at periodic intervals during use.

In some embodiments, an aerosolized foam may contain an amount of a DVP, or a pharmaceutically acceptable salt thereof, ranging from about 0.005 wt % to about 99 wt %.

In some embodiments, an aerosolized foam may contain an amount of a DVP-insecticidal protein, or a pharmaceutically acceptable salt thereof, ranging from about 0.005 wt % to about 99 wt %.

Burning Formulations

In some embodiments, a dwelling area may also be treated with an active DVP or DVP-insecticidal protein composition by using a burning formulation, such as a candle, a smoke coil or a piece of incense containing the composition. For example, the composition may be formulated into household products such as “heated” air fresheners in which insecticidal compositions are released upon heating, e.g., electrically, or by burning. The active compound compositions of the present invention comprising a DVP, a DVP-insecticidal protein, or a pharmaceutically acceptable salt thereof may be made available in a spray product as an aerosol, a mosquito coil, and/or a vaporizer or fogger.

In some embodiments, a burning formulation may contain an amount of a DVP, or a pharmaceutically acceptable salt thereof, ranging from about 0.005 wt % to about 99 wt %.

In some embodiments, a burning formulation may contain an amount of a DVP-insecticidal protein, or a pharmaceutically acceptable salt thereof, ranging from about 0.005 wt % to about 99 wt %.

Fabric Treatments

In some embodiments, fabrics and garments may be made containing a pesticidal effective composition comprising a DVP, a DVP-insecticidal protein, or a pharmaceutically acceptable salt thereof, and an excipient. In some embodiments, the concentration of the DVP or DVP-insecticidal protein in the polymeric material, fiber, yarn, weave, net, or substrate described herein, can be varied within a relatively wide concentration range from, for example, 0.05 to 15 percent by weight, preferably 0.2 to 10 percent by weight, more preferably 0.4 to 8 percent by weight, especially 0.5 to 5, such as 1 to 3, percent by weight.

Similarly, the concentration of the composition comprising a DVP, a DVP-insecticidal protein, or a pharmaceutically acceptable salt thereof, and an excipient (whether for treating surfaces or for coating a fiber, yarn, net, weave) can be varied within a relatively wide concentration range from, for example 0.1 to 70 percent by weight, such as 0.5 to 50 percent by weight, preferably 1 to 40 percent by weight, more preferably 5 to 30 percent by weight, especially 10 to 20 percent by weight.

The concentration of the DVP or DVP-insecticidal protein may be chosen according to the field of application such that the requirements concerning knockdown efficacy, durability and toxicity are met. Adapting the properties of the material can also be accomplished and so custom-tailored textile fabrics are obtainable in this way.

Accordingly, an effective amount of a DVP, a DVP-insecticidal protein, or a pharmaceutically acceptable salt thereof can depend on the specific use pattern, the insect pest against which control is most desired and the environment in which the DVP or DVP-insecticidal protein will be used. Therefore, an effective amount of a DVP, a DVP-insecticidal protein, or a pharmaceutically acceptable salt thereof is sufficient that control of an insect pest is achieved.

In some embodiments, a fabric treatment may contain an amount of a DVP, or a pharmaceutically acceptable salt thereof, ranging from about 0.005 wt % to about 99 wt %.

In some embodiments, a fabric treatment may contain an amount of a DVP-insecticidal protein, or a pharmaceutically acceptable salt thereof, ranging from about 0.005 wt % to about 99 wt %.

Surface-Treatment Compositions

In some embodiments, the present disclosure provides compositions or formulations comprising a DVP and an excipient, or comprising a DVP-insecticidal protein and an excipient, for coating walls, floors and ceilings inside of buildings, and for coating a substrate or non-living material. The inventive compositions comprising a DVP, a DVP-insecticidal protein, or a pharmaceutically acceptable salt thereof, and an excipient, can be prepared using known techniques for the purpose in mind. Preparations of compositions comprising a DVP-insecticidal protein and an excipient, could be so formulated to also contain a binder to facilitate the binding of the compound to the surface or other substrate. Agents useful for binding are known in the art and tend to be polymeric in form. The type of binder suitable for a compositions to be applied to a wall surface having particular porosities and/or binding characteristics would be different compared to a fiber, yarn, weave or net-thus, a skilled person, based on known teachings, would select a suitable binder based on the desired surface and/or substrate.

Typical binders are poly vinyl alcohol, modified starch, poly vinyl acrylate, polyacrylic, polyvinyl acetate co polymer, polyurethane, and modified vegetable oils. Suitable binders can include latex dispersions derived from a wide variety of polymers and co-polymers and combinations thereof. Suitable latexes for use as binders in the inventive compositions comprise polymers and copolymers of styrene, alkyl styrenes, isoprene, butadiene, acrylonitrile lower alkyl acrylates, vinyl chloride, vinylidene chloride, vinyl esters of lower carboxylic acids and alpha, beta-ethylenically unsaturated carboxylic acids, including polymers containing three or more different monomer species copolymerized therein, as well as post-dispersed suspensions of silicones or polyurethanes. Also suitable may be a polytetrafluoroethylene (PTFE) polymer for binding the active ingredient to other surfaces.

In some embodiments, a surface-treatment composition may contain an amount of a DVP, or a pharmaceutically acceptable salt thereof, ranging from about 0.005 wt % to about 99 wt %.

In some embodiments, a surface-treatment composition may contain an amount of a DVP-insecticidal protein, or a pharmaceutically acceptable salt thereof, ranging from about 0.005 wt % to about 99 wt %.

Dispersants

In some exemplary embodiments, an insecticidal formulation according to the present disclosure may consist of a DVP, a DVP-insecticidal protein, or a pharmaceutically acceptable salt thereof, and an excipient, diluent or carrier (e.g., such as water), a polymeric binder, and/or additional components such as a dispersing agent, a polymerizing agent, an emulsifying agent, a thickener, an alcohol, a fragrance, or any other inert excipients used in the preparation of sprayable insecticides known in the art.

In some embodiments, a composition comprising a DVP, a DVP-insecticidal protein, or a pharmaceutically acceptable salt thereof, and an excipient, can be prepared in a number of different forms or formulation types, such as suspensions or capsules suspensions. And a person skilled in the art can prepare the relevant composition based on the properties of the particular DVP or DVP-insecticidal protein, its uses, and also its application type. For example, the DVP or DVP-insecticidal protein used in the methods, embodiments, and other aspects of the present disclosure, may be encapsulated in a suspension or capsule suspension formulation. An encapsulated DVP or DVP-insecticidal protein can provide improved wash-fastness, and also a longer period of activity. The formulation can be organic based or aqueous based, preferably aqueous based.

In some embodiments, a dispersant may contain an amount of a DVP, or a pharmaceutically acceptable salt thereof, ranging from about 0.005 wt % to about 99 wt %.

In some embodiments, a dispersant may contain an amount of a DVP-insecticidal protein, or a pharmaceutically acceptable salt thereof, ranging from about 0.005 wt % to about 99 wt %.

Microencapsulation

Microencapsulated DVP or DVP-insecticidal protein suitable for use in the compositions and methods according to the present disclosure may be prepared with any suitable technique known in the art. For example, various processes for microencapsulating material have been previously developed. These processes can be divided into three categories: physical methods, phase separation, and interfacial reaction. In the physical methods category, microcapsule wall material and core particles are physically brought together and the wall material flows around the core particle to form the microcapsule. In the phase separation category, microcapsules are formed by emulsifying or dispersing the core material in an immiscible continuous phase in which the wall material is dissolved and caused to physically separate from the continuous phase, such as by coacervation, and deposit around the core particles. In the interfacial reaction category, microcapsules are formed by emulsifying or dispersing the core material in an immiscible continuous phase and then an interfacial polymerization reaction is caused to take place at the surface of the core particles. The concentration of the DVP or DVP-insecticidal protein present in the microcapsules can vary from 0.1 to 60% by weight of the microcapsule.

In some embodiments, a microencapsulation may contain an amount of a DVP, or a pharmaceutically acceptable salt thereof, ranging from about 0.005 wt % to about 99 wt %.

In some embodiments, a microencapsulation may contain an amount of a DVP-insecticidal protein, or a pharmaceutically acceptable salt thereof, ranging from about 0.005 wt % to about 99 wt %.

Kits, Formulations, Dispersants, and the Ingredients Thereof

The formulation used in the compositions (comprising a DVP, a DVP-insecticidal protein, or a pharmaceutically acceptable salt thereof, and an excipient), methods, embodiments and other aspects according to the present disclosure, may be formed by mixing all ingredients together with water, and optionally using suitable mixing and/or dispersing aggregates. In general, such a formulation is formed at a temperature of from 10 to 70° C., preferably 15 to 50° C., more preferably 20 to 40° C. Generally, a formulation comprising one or more of (A), (B), (C), and/or (D) is possible, wherein it is possible to use: a DVP, a DVP-insecticidal protein, or a pharmaceutically acceptable salt thereof (as pesticide) (A); solid polymer (B); optional additional additives (D); and to disperse them in the aqueous component (C). If a binder is present in a composition of the present invention (comprising a DVP, a DVP-insecticidal protein, or a pharmaceutically acceptable salt thereof, and an excipient), it is preferred to use dispersions of the polymeric binder (B) in water as well as aqueous formulations of the DVP or DVP-insecticidal protein (A) in water which have been separately prepared before. Such separate formulations may contain additional additives for stabilizing (A) and/or (B) in the respective formulations and are commercially available. In a second process step, such raw formulations and optionally additional water (component (C)) are added. Also, combinations of the abovementioned ingredients based on the foregoing scheme are likewise possible, e.g., using a pre-formed dispersion of (A) and/or (B) and mixing it with solid (A) and/or (B). A dispersion of the polymeric binder (B) may be a pre-manufactured dispersion already made by a chemicals manufacturer.

Moreover, it is also within the scope of the present invention to use “hand-made” dispersions, i.e., dispersions made in small-scale by an end-user. Such dispersions may be made by providing a mixture of about 20 percent of the binder (B) in water, heating the mixture to temperature of 90° C. to 100° C. and intensively stirring the mixture for several hours. It is possible to manufacture the formulation as a final product so that it can be readily used by the end-user for the process according to the present invention. And, it is of course similarly possible to manufacture a concentrate, which may be diluted by the end-user with additional water (C) to the desired concentration for use.

In an embodiment, a composition (comprising a DVP, a DVP-insecticidal protein, or a pharmaceutically acceptable salt thereof, and an excipient) suitable for SSI application or a coating formulation (comprising a DVP, a DVP-insecticidal protein, or a pharmaceutically acceptable salt thereof, and an excipient), contains the active ingredient and a carrier, such as water, and may also one or more co-formulants selected from a dispersant, a wetter, an anti-freeze, a thickener, a preservative, an emulsifier and a binder or sticker.

In some embodiments, an exemplary solid formulation of a DVP, a DVP-insecticidal protein, or a pharmaceutically acceptable salt thereof, is generally milled to a desired particle size, such as the particle size distribution d(0.5) is generally from 3 to 20, preferably 5 to 15, especially 7 to 12, μm.

Furthermore, it may be possible to ship the formulation to the end-user as a kit comprising at least a first component comprising a DVP, a DVP-insecticidal protein, or a pharmaceutically acceptable salt thereof (A); and a second component comprising at least one polymeric binder (B). Further additives (D) may be a third separate component of the kit, or may be already mixed with components (A) and/or (B). The end-user may prepare the formulation for use by just adding water (C) to the components of the kit and mixing. The components of the kit may also be formulations in water. Of course it is possible to combine an aqueous formulation of one of the components with a dry formulation of the other component(s). As an example, the kit can consist of one formulation of a DVP, a DVP-insecticidal protein, or a pharmaceutically acceptable salt thereof (A) and optionally water (C); and a second, separate formulation of at least one polymeric binder (B), water as component (C) and optionally components (D).

The concentrations of the components (A), (B), (C) and optionally (D) will be selected by the skilled artisan depending of the technique to be used for coating/treating. In general, the amount of a DVP, a DVP-insecticidal protein, or a pharmaceutically acceptable salt thereof (A) may be up to 50, preferably 1 to 50, such as 10 to 40, especially 15 to 30, percent by weight, based on weight of the composition. The amount of polymeric binder (B) may be in the range of 0.01 to 30, preferably 0.5 to 15, more preferably 1 to 10, especially 1 to 5, percent by weight, based on weight of the composition. If present, in general the amount of additional components (D) is from 0.1 to 20, preferably 0.5 to 15, percent by weight, based on weight of the composition. If present, suitable amounts of pigments and/or dyestuffs and/or fragrances are in general 0.01 to 5, preferably 0.1 to 3, more preferably 0.2 to 2, percent by weight, based on weight of the composition. A typical formulation ready for use comprises 0.1 to 40, preferably 1 to 30, percent of components (A), (B), and optionally (D), the residual amount being water (C). A typical concentration of a concentrate to be diluted by the end-user may comprise 5 to 70, preferably 10 to 60, percent of components (A), (B), and optionally (D), the residual amount being water (C).

Illustrative Mixtures, Compositions, Products, And Transgenic Organisms

The present disclosure contemplates mixtures, compositions, products, and transgenic organisms that contain—or, in the case of transgenic organisms, express or otherwise produce-one or more DVPs, or one or more DVP-insecticidal proteins.

In some embodiments, the illustrative mixtures consists of: (1) a DVP, or a DVP-insecticidal proteins; or a pharmaceutically acceptable salt thereof, and (2) an excipient (e.g., any of the excipients described herein).

In some embodiments, the mixtures of the present invention consist of: (1) one or more DVPs, or one or more DVP-insecticidal proteins, or a pharmaceutically acceptable salt thereof; and (2) one or more excipients (e.g., any of the excipients described herein).

In some embodiments, the mixtures of the present invention consist of: (1) one or more DVPs, or one or more DVP-insecticidal proteins, or a pharmaceutically acceptable salt thereof; and (2) one or more excipients (e.g., any of the excipients described herein); wherein either of the foregoing (1) or (2) can be used concomitantly, or sequentially.

Any of the combinations, mixtures, products, polypeptides and/or plants utilizing a DVP, or a DVP-insecticidal protein (as described herein), can be used to control pests, their growth, and/or the damage caused by their actions, especially their damage to plants.

Compositions comprising a DVP or a DVP-insecticidal protein, or a pharmaceutically acceptable salt thereof, and an excipient, can include agrochemical compositions. For example, in some embodiments, agrochemical compositions can include, but is not limited to, aerosols and/or aerosolized products (e.g., sprays, fumigants, powders, dusts, and/or gases); seed dressings; oral preparations (e.g., insect food, etc.); or a transgenic organisms (e.g., a cell, a plant, or an animal) expressing and/or producing a DVP or a DVP-insecticidal protein, either transiently and/or stably.

In some embodiments, the active ingredients of the present disclosure can be applied in the form of compositions and can be applied to the crop area or plant to be treated, simultaneously or in succession, with other non-active compounds. These compounds can be fertilizers, weed killers, cryoprotectants, surfactants, detergents, soaps, dormant oils, polymers, and/or time-release or biodegradable carrier formulations that permit long-term dosing of a target area following a single application of the formulation. One or more of these non-active compounds can be prepared, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise, the formulations may be prepared into edible “baits” or fashioned into pest “traps” to permit feeding or ingestion by a target pest of the pesticidal formulation.

Methods of applying an active ingredient of the present disclosure or an agrochemical composition of the present disclosure that consists of a DVP or DVP-insecticidal protein or a pharmaceutically acceptable salt thereof, and an excipient, as produced by the methods described herein of the present disclosure, include leaf application, seed coating and soil application. In some embodiments, the number of applications and the rate of application depend on the intensity of infestation by the corresponding pest.

The composition comprising a DVP or a DVP-insecticidal protein or a pharmaceutically acceptable salt thereof and an excipient may be formulated as a powder, dust, pellet, granule, spray, emulsion, colloid, solution, or such like, and may be prepared by such conventional means as desiccation, lyophilization, homogenization, extraction, filtration, centrifugation, sedimentation, or concentration of a culture of cells comprising the polypeptide. In all such compositions that contain at least one such pesticidal polypeptide, the polypeptide may be present in a concentration of from about 1% to about 99% by weight.

In some embodiments, compositions containing DVPs or DVP-insecticidal proteins (or a pharmaceutically acceptable salt thereof) may be prophylactically applied to an environmental area to prevent infestation by a susceptible pest, for example, a lepidopteran and/or coleopteran pest, which may be killed or reduced in numbers in a given area by the methods of the invention. In some embodiments, the pest ingests, or comes into contact with, a pesticidally-effective amount of the polypeptide.

In some embodiments, the pesticide compositions described herein may be made by formulating either the DVP or DVP-insecticidal-protein or a pharmaceutically acceptable salt thereof transformed bacterial, yeast, or other cell, crystal and/or spore suspension, or isolated protein component with the desired agriculturally-acceptable carrier. The compositions may be formulated prior to administration in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. In some embodiments, the formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, the disclosure of which is incorporated herein by reference in its entirety.

Methods of Using the Present Invention

Methods for Protecting Plants, Plant Parts, and Seeds

In some embodiments, the present invention provides a method of protecting a plant from insects comprising, providing a plant that expresses a DVP, or polynucleotide encoding the same.

In some embodiments, the present invention provides a method of protecting a plant from insects comprising, providing a plant that expresses a DVP, or polynucleotide encoding the same, wherein said DVP is a DVP as described herein.

In some embodiments, the present invention provides a method of protecting a plant from insects comprising, providing a plant that expresses a DVP, or polynucleotide encoding the same, wherein the DVP has an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 187-191.

In some embodiments, the present invention provides a method of protecting a plant from insects comprising, providing a plant that expresses a DVP, or polynucleotide encoding the same, wherein the DVP has an amino acid sequence as set forth in any one of SEQ ID NOs: 187-191.

In some embodiments, the present invention provides a method of protecting a plant from insects comprising, providing a plant that expresses a DVP, or polynucleotide encoding the same, wherein the DVP further comprises a homopolymer or heteropolymer of two or more DVPs, wherein the amino acid sequence of each DVP is the same or different.

In some embodiments, the present invention provides a method of protecting a plant from insects comprising, providing a plant that expresses a DVP, or polynucleotide encoding the same, wherein the DVP is a fused protein comprising two or more DVPs separated by a cleavable or non-cleavable linker, and wherein the amino acid sequence of each DVP may be the same or different.

In some embodiments, the present invention provides a method of protecting a plant from insects comprising, providing a plant that expresses a DVP, or polynucleotide encoding the same, wherein the linker is cleavable inside the gut or hemolymph of an insect.

In some embodiments, the present invention provides a method for controlling insects comprising, providing to said insect a transgenic plant that comprises in its genome a stably incorporated expression cassette, wherein said stably incorporated expression cassette comprises polynucleotide operable to encode a DVP.

In some embodiments, the present disclosure provides a method for controlling an invertebrate pest in agronomic and/or nonagronomic applications, comprising contacting the invertebrate pest or its environment, a solid surface, including a plant surface or part thereof, with a biologically effective amount of one or more of the DVPs of the invention, or with a DVP-insecticidal protein, or a pharmaceutically acceptable salt thereof.

In some embodiments, the present disclosure provides a method for controlling an invertebrate pest in agronomic and/or nonagronomic applications, comprising contacting the invertebrate pest or its environment, a solid surface, including a plant surface or part thereof, with a biologically effective amount of a composition comprising at least one DVP of the invention and an excipient.

Methods for Controlling an Invertebrate Pest

In some embodiments, the present disclosure provides a method for controlling an invertebrate pest in agronomic and/or nonagronomic applications, comprising contacting the invertebrate pest or its environment, a solid surface, including a plant surface or part thereof, with a biologically effective amount of a composition comprising at least one DVP-insecticidal protein of the invention and an excipient.

Examples of suitable compositions comprising: (1) at least one DVP of the invention; two or more of the DVPs of the present invention; a DVP-insecticidal protein; two or more DVP-insecticidal proteins; or a pharmaceutically acceptable salt thereof; and (2) an excipient; include said compositions formulated win inactive ingredients to be delivered in the form of: a liquid solution, an emulsion, a powder, a granule, a nanoparticle, a microparticle, or a combination thereof.

In some embodiments, to achieve contact with a compound, mixture, or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a DVP, a DVP-insecticidal protein, or a pharmaceutically acceptable salt thereof, and an excipient, can be applied to the plant foliage or the soil. Compounds of this invention can also be effectively delivered through plant uptake by contacting the plant with a composition comprising a compound of this invention applied as a soil drench of a liquid formulation, a granular formulation to the soil, a nursery box treatment or a dip of transplants. Of note is a composition of the present disclosure in the form of a soil drench liquid formulation. Also of note is a method for controlling an invertebrate pest comprising contacting the invertebrate pest or its environment with a biologically effective amount of a DVP or DVP-insecticidal protein. Of further note, in some illustrative embodiments, the illustrative method contemplates a soil environment, wherein the composition is applied to the soil as a soil drench formulation. Of further note is that a DVP, a DVP-insecticidal protein, or a pharmaceutically acceptable salt thereof, is also effective by localized application to the locus of infestation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting, application onto clothing, application into candle formulations and the like).

In some embodiments, a DVP, a DVP-insecticidal protein, or a pharmaceutically acceptable salt thereof, is also useful in seed treatments for protecting seeds from invertebrate pests. In the context of the present disclosure and claims, treating a seed means contacting the seed with a biologically effective amount of a DVP, a DVP-insecticidal protein, or a pharmaceutically acceptable salt thereof, which is typically formulated as a composition of the invention. This seed treatment protects the seed from invertebrate soil pests and generally can also protect roots and other plant parts in contact with the soil of the seedling developing from the germinating seed. The seed treatment may also provide protection of foliage by translocation of the DVP or DVP-insecticidal protein within the developing plant. Seed treatments can be applied to all types of seeds, including those from which plants genetically transformed to express specialized traits will germinate. In addition, a DVP or a DVP-insecticidal protein can be transformed into a plant or part thereof, for example a plant cell, or plant seed, that is already transformed, e.g., those expressing herbicide resistance such as glyphosate acetyltransferase, which provides resistance to glyphosate.

One method of seed treatment is by spraying or dusting the seed with a DVP, a DVP-insecticidal protein, or a pharmaceutically acceptable salt thereof, (i.e. as a formulated composition or a mixture comprising a DVP, a DVP-insecticidal protein, or a pharmaceutically acceptable salt thereof and an excipient) before sowing the seeds. Compositions formulated for seed treatment generally consist of a DVP, a DVP-insecticidal protein, or a pharmaceutically acceptable salt thereof, and a film former or adhesive agent. Therefore, typically, a seed coating composition of the present disclosure consists of a biologically effective amount of a DVP, a DVP-insecticidal protein, or a pharmaceutically acceptable salt thereof, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Monograph No. 57, and references listed therein, the disclosures of which are incorporated herein by reference in their entireties.

The treated seed typically comprises a DVP, a DVP-insecticidal protein, or a pharmaceutically acceptable salt thereof, in an amount ranging from about 0.01 g to 1 kg per 100 kg of seed (i.e. from about 0.00001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

Methods of Using Compositions

In some embodiments, the present invention provides a method of using a mixture comprising: (1) a DVP, a DVP-insecticidal protein, or a pharmaceutically acceptable salt thereof; and (2) an excipient; to control insects, wherein the DVP is selected from one or any combination of the DVPs described herein, e.g., a DVP having insecticidal activity against one or more insect species, said DVP comprising an amino acid sequence that is at least 95% identical to the amino acid sequence according to Formula (I): A-X₁-D-G-D-V-E-G-P-A-G-C-K-K-Y-D-X₂-E-C-X₃—X₄-G-E-C-C-Q-K-Q-Y-L-X₅-X₆-K-W-R-X₇-L-X₈-C-R-X₉-X₁₀-K-S-G-F-F-S-S-K-X₁₁—X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T; or a pharmaceutically acceptable salt thereof; wherein said method comprises, preparing the mixture and then applying said mixture to the locus of an insect.

In some embodiments, the present invention provides a method of using a mixture to control insects, said mixture comprising: (1) a DVP, a DVP-insecticidal protein, or a pharmaceutically acceptable salt thereof, and (2) an excipient; wherein the insects are selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola; Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana) (ground only); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Jo Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex; Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popilla japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and/or Xanthogaleruca luteola.

In some embodiments, the present invention provides a method of protecting a plant from insects comprising, providing a plant which expresses one or more DVPs, or polynucleotides encoding the same.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP is selected from one or any combination of the DVPs described herein, e.g., an insecticidal Mu-diguetoxin-Dc1a variant polypeptide (DVP), said DVP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence according to Formula (I): A-X₁-D-G-D-V-E-G-P-A-G-C—K-K-Y-D-X₂-E-C-X₃-X₄-G-E-C-C-Q-K-Q-Y-L-X₅-X₆-K-W-R-X₇-L-X₈-C—R—X₉-X₁₀-K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T; or a pharmaceutically acceptable salt thereof, wherein the mixture is applied to the locus of the pest, or to a plant or animal susceptible to an attack by the pest.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence that is at least 90% identical to the amino acid sequence according to Formula (I): A-X₁-D-G-D-V-E-G-P-A-G-C—K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F—F-S-S-K-X₁₁-X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T; or a pharmaceutically acceptable salt thereof; wherein if X₉ is G, T, A, S, M or V, or X₁₁ is F, A, T, S, M or V, then a disulfide bond is removed.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-43, 45-51, 53, 128, 130, 136, 139-140, 144, 146-147, 187-191, 202-215, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino acid sequence as set forth in any one of SEQ ID NOs: 6-43, 45-51, 53, 128, 130, 136, 139-140, 144, 146-147, 187-191, 202-215, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino acid sequence as set forth in any one of SEQ ID NOs: 6-11, 15-16, 20-22, 24-26, 29, 35, 45-48, 53, 128, 136, 139-140, 144, 146-147, 187-191, 207, 210-215, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 47, 53, 136, 139-140, 144, 146-147, 187-191, 210-215, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino acid sequence as set forth in any one of SEQ ID NOs: 47, 53, 136, 139-140, 144, 146-147, 187-191, 210-215, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 213, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; wherein the DVP has an amino acid sequence as set forth in any one of SEQ ID NOs: 213, or 217-219.

In some embodiments, the present invention provides a method of combating, controlling, or inhibiting a pest comprising, applying a pesticidally effective amount of a mixture comprising: (1) a DVP, a DVP-insecticidal protein, or a pharmaceutically acceptable salt thereof; and (2) an excipient; to the locus of a pest, wherein the pest is selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola; Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana) (ground only); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Jo Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex; Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popilla japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and/or Xanthogaleruca luteola.

Crops and Pests

Specific crop pests and insects that may be controlled by these methods include the following: Dictyoptera (cockroaches); Isoptera (termites); Orthoptera (locusts, grasshoppers and crickets); Diptera (house flies, mosquito, tsetse fly, crane-flies and fruit flies); Hymenoptera (ants, wasps, bees, saw-flies, ichneumon flies and gall-wasps); Anoplura (biting and sucking lice); Siphonaptera (fleas); and Hemiptera (bugs and aphids), as well as arachnids such as Acari (ticks and mites), and the parasites that each of these organisms harbor.

“Pest” includes, but is not limited to: insects, fungi, bacteria, nematodes, mites, ticks, and the like.

Insect pests include, but are not limited to, insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, and the like. More particularly, insect pests include Coleoptera, Lepidoptera, and Diptera.

Insects of suitable agricultural, household and/or medical/veterinary importance for treatment with the insecticidal polypeptides include, but are not limited to, members of the following classes and orders:

The order Coleoptera includes the suborders Adephaga and Polyphaga. Suborder Adephaga includes the superfamilies Caraboidea and Gyrinoidea. Suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

Examples of Coleoptera include, but are not limited to: the American bean weevil Acanthoscelides obtectus, the leaf beetle Agelastica alni, click beetles (Agriotes lineatus, Agriotes obscurus, Agriotes bicolor), the grain beetle ahasverus advena, the summer schafer Amphimallon solstitialis, the furniture beetle Anobium punctatum, Anthonomus spp. (weevils), the Pygmy mangold beetle Atomaria linearis, carpet beetles (anthrenus spp., Attagenus spp.), the cowpea weevil Callosobruchus maculates, the fried fruit beetle carpophilus hemipterus, the cabbage seedpod weevil Ceutorhynchus assimilis, the rape winter stem weevil Ceutorhynchus picitarsis, the wireworms Conoderus vespertinus and Conoderus falli, the banana weevil Cosmopolites sordidus, the New Zealand grass grub Costelytra zealandica, the June beetle Cotinis nitida, the sunflower stem weevil Cylindrocopturus adspersus, the larder beetle Dermestes lardarius, the corn rootworms Diabrotica virgifera, Diabrotica virgifera virgifera, and Diabrotica barberi, the Mexican bean beetle Epilachna varivestis, the old house borer Hylotropes bajulus, the lucerne weevil Hypera postica, the shiny spider beetle Gibbium psylloides, the cigarette beetle Lasioderma serricorne, the Colorado potato beetle Leptinotarsa decemlineata, Lyctus beetles (Lyctus spp.), the pollen beetle Meligethes aeneus, the common cockshafer Melolontha melolontha, the American spider beetle Mezium americanum, the golden spider beetle Niptus hololeucus, the grain beetles Oryzaephilus surinamensis and Oryzaephilus mercator, the black vine weevil Otiorhynchus sulcatus, the mustard beetle Phaedon cochleariae, the crucifer flea beetle Phyllotreta cruciferae, the striped flea beetle Phyllotreta striolata, the cabbage steam flea beetle psylliodes chrysocephala, Ptinus spp. (spider beetles), the lesser grain borer Rhizopertha dominica, the pea and been weevil Sitona lineatus, the rice and granary beetles Sitophilus oryzae and Sitophilus granaries, the red sunflower seed weevil Smicronyx fulvus, the drugstore beetle Stegobium paniceum, the yellow mealworm beetle Tenebrio molitor, the flour beetles Tribolium castaneum and Tribolium confusum, warehouse and cabinet beetles (Trogoderma spp.), and the sunflower beetle Zygogramma exclamationis.

Examples of Dermaptera (earwigs) include, but are not limited to: the European earwig, Forficula auricularia, and the striped earwig, Labidura riparia.

Examples of Dictvontera include, but are not limited to: the oriental cockroach, Blatta orientalis, the German cockroach, Blatella germanica, the Madeira cockroach, Leucophaea maderae, the American cockroach, Periplaneta americana, and the smokybrown cockroach Periplaneta fuliginosa.

Examples of Diplonoda include, but are not limited to: the spotted snake millipede Blaniulus guttulatus, the flat-back millipede Brachydesmus superus, and the greenhouse millipede Oxidus gracilis.

The order Diptera includes the Suborders nematocera, brachycera, and Cyclorrhapha. Suborder nematocera includes the families Tipulidae, Psychodidae, Culicidae, Ceratopogonidae, Chironomidae, Simuliidae, Bibionidae, and Cecidomyiidae. Suborder brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and calyptratae. Section Acalyptratae includes the families Otitidae, Tephritidae, Agromyzidae, and Drosophilidae. Section calyptratae includes the families Hippoboscidae, Oestridae, Tachinidae, Anthomyiidae, Muscidae, Calliphoridae, and Sarcophagidae.

Examples of Diptera include, but are not limited to: the house fly (Musca domestica), the African tumbu fly (Cordylobia anthropophaga), biting midges (Culicoides spp.), bee louse (Braula spp.), the beet fly Pegomyia betae, blackflies (Cnephia spp., Eusimulium spp., Simulium spp.), bot flies (Cuterebra spp., Gastrophilus spp., Oestrus spp.), craneflies (Tipula spp.), eye gnats (Hippelates spp.), filth-breeding flies (Calliphora spp., Fannia spp., Hermetia spp., Lucilia spp., Musca spp., Muscina spp., Phaenicia spp., Phormia spp.), flesh flies (Sarcophaga spp., Wohlfahrtia spp.); the flit fly Oscinella frit, fruitflies (Dacus spp., Drosophila spp.), head and canon flies (Hydrotea spp.), the hessian fly Mayetiola destructor, horn and buffalo flies (Haematobia spp.), horse and deer flies (Chrysops spp., Haematopota spp., Tabanus spp.), louse flies (Lipoptena spp., Lynchia spp., and Pseudolynchia spp.), medflies (Ceratitus spp.), mosquitoes (Aedes spp., Anopheles spp., Culex spp., Psorophora spp.), sandflies (Phlebotomus spp., Lutzomyia-spp.), screw-worm flies (Chtysomya bezziana and Cochlomyia hominivorax), sheep keds (Melophagus spp.); stable flies (Stomoxys spp.), tsetse flies (Glossina spp.), and warble flies (Hypoderma spp.).

Examples of Isontera (termites) include, but are not limited to: species from the families Hodotennitidae, Kalotermitidae, Mastotermitidae, Rhinotennitidae, Serritermitidae, Termitidae, and Termopsidae.

Examples of heteroptera include, but are not limited to: the bed bug Cimex lectularius, the cotton stainer Dysdercus intermedius, the Sunn pest Eurygaster integriceps, the tarnished plant bug Lygus lineolaris, the green stink bug Nezara antennata, the southern green stink bug Nezara viridula, and the triatomid bugs Panstrogylus megistus, Rhodnius ecuadoriensis, Rhodnius pallescans, Rhodnius prolixus, Rhodnius robustus, Triatoma dimidiata, Triatoma infestans, and Triatoma sordida.

Examples of Homoptera include, but are not limited to: the California red scale Aonidiella aurantii, the black bean aphid Aphis fabae, the cotton or melon aphid Aphis gossypii, the green apple aphid Aphis pomi, the citrus spiny whitefly Aleurocanthus spiniferus, the oleander scale Aspidiotus hederae, the sweet potato whitefly Bemesia tabaci, the cabbage aphid Brevicoryne brassicae, the pear psylla Cacopsylla pyricola, the currant aphid Cryptomyzus ribis, the grape phylloxera Daktulosphaira vitifoliae, the citrus psylla Diaphorina citri, the potato leafhopper Empoasca fabae, the bean leafhopper Empoasca solana, the vine leafhopper Empoasca vitis, the woolly aphid Eriosoma lanigerum, the European fruit scale Eulecanium corni, the mealy plum aphid Hyalopterus arundinis, the small brown planthopper Laodelphax striatellus, the potato aphid Macrosiphum euphorbiae, the green peach aphid Myzus persicae, the green rice leafhopper Nephotettix cinticeps, the brown planthopper Nilaparvata lugens, gall-forming aphids (Pemphigus spp.), the hop aphid Phorodon humuli, the bird-cherry aphid Rhopalosiphum padi, the black scale Saissetia oleae, the greenbug Schizaphis graminum, the grain aphid Sitobion avenae, and the greenhouse whitefly Trialeurodes vaporariorum.

Examples of isopoda include, but are not limited to: the common pillbug Armadillidium vulgare and the common woodlouse Oniscus asellus.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Examples of Lepidoptera include, but are not limited to: Adoxophyes orana (summer fruit tortrix moth), Agrotis ipsolon (black cutworm), Archips podana (fruit tree tortrix moth), Bucculatrix pyrivorella (pear leafminer), Bucculatrix thurberiella (cotton leaf perforator), Bupalus piniarius (pine looper), Carpocapsa pomonella (codling moth), Chilo suppressalis (striped rice borer), Choristoneura fumiferana (eastern spruce budworm), Cochylis hospes (banded sunflower moth), Diatraea grandiosella (southwestern corn borer), Earls insulana (Egyptian bollworm), Euphestia kuehniella (Mediterranean flour moth), Eupoecilia ambiguella (European grape berry moth), Euproctis chrysorrhoea (brown-tail moth), Euproctis subflava (oriental tussock moth), Galleria mellonella (greater wax moth), Helicoverpa armigera (cotton bollworm), Helicoverpa zea (cotton bollworm), Heliothis virescens (tobacco budworm), Hofmannophila pseudopretella (brown house moth), Homeosoma electellum (sunflower moth), Homona magnanima (oriental tea tree tortrix moth), Lithocolletis blancardella (spotted tentiform leafminer), Lymantria dispar (gypsy moth), Malacosoma neustria (tent caterpillar), Mamestra brassicae (cabbage armyworm), Mamestra configurata (Bertha armyworm), the hornworms Manduca sexta and Manuduca quinquemaculata, Operophtera brumata (winter moth), Ostrinia nubilalis (European corn borer), Panolis flammea (pine beauty moth), Pectinophora gossypiella (pink bollworm), Phyllocnistis citrella (citrus leafminer), Pieris brassicae (cabbage white butterfly), Plutella xylostella (diamondback moth), Rachiplusia ni (soybean looper), Spilosoma virginica (yellow bear moth), Spodoptera exigua (beet armyworm), Spodoptera frugiperda (fall armyworm), Spodoptera littoralis (cotton leafworin), Spodoptera litura (common cutworm), Spodoptera praefica (yellowstriped armyworm), Sylepta derogata (cotton leaf roller), Tineola bisselliella (webbing clothes moth), Tineola pellionella (case-making clothes moth), Tortrix viridana (European oak leafroller), Trichoplusia ni (cabbage looper), and Yponomeuta padella (small ermine moth).

Examples of Orthoptera include, but are not limited to: the common cricket Acheta domesticus, tree locusts (Anacridium spp.), the migratory locust Locusta migratoria, the twostriped grasshopper Melanoplus bivittatus, the differential grasshopper Melanoplus dfferentialis, the redlegged grasshopper Melanoplus femurrubrum, the migratory grasshopper Melanoplus sanguinipes, the northern mole cricket Neocurtilla hexadectyla, the red locust Nomadacris septemfasciata, the shortwinged mole cricket Scapteriscus abbreviatus, the southern mole cricket Scapteriscus borellii, the tawny mole cricket Scapteriscus vicinus, and the desert locust Schistocerca gregaria.

Examples of Phthiraptera include, but are not limited to: the cattle biting louse Bovicola bovis, biting lice (Damalinia spp.), the cat louse Felicola subrostrata, the shortnosed cattle louse Haematopinus eloysternus, the tail-switch louse Haematopinus quadriperiussus, the hog louse Haematopinus suis, the face louse Linognathus ovillus, the foot louse Linognathus pedalis, the dog sucking louse Linognathus setosus, the long-nosed cattle louse Linognathus vituli, the chicken body louse Menacanthus stramineus, the poultry shaft louse Menopon gallinae, the human body louse Pediculus humanus, the pubic louse Phthiruspubis, the little blue cattle louse Solenopotes capillatus, and the dog biting louse Trichodectes canis.

Examples of Psocoptera include, but are not limited to: the booklice Liposcelis bostrychophila, Liposcelis decolor, Liposcelis entomophila, and Trogium pulsator um. Examples of Siphonaptera include, but are not limited to: the bird flea Ceratophyllus gallinae, the dog flea Ctenocephalides canis, the cat flea Ctenocephalides fells, the human flea Pulex irritans, and the oriental rat flea Xenopsylla cheopis.

Examples of symphyla include, but are not limited to: the garden symphylan Scutigerella immaculate.

Examples of Thysanura include, but are not limited to: the gray silverfish Ctenolepisma longicaudata, the four-lined silverfish Ctenolepisma quadriseriata, the common silverfish Lepisma saccharina, and the firebrat Thennobia domestica;

Examples of Thysanoptera include, but are not limited to: the tobacco thrips Frankliniella fusca, the flower thrips Frankliniella intonsa, the western flower thrips Frankliniella occidentalis, the cotton bud thrips Frankliniella schultzei, the banded greenhouse thrips Hercinothrips femoralis, the soybean thrips Neohydatothrips variabilis, Kelly's citrus thrips Pezothrips kellyanus, the avocado thrips Scirtothrips perseae, the melon thrips, Thrips palmi, and the onion thrips, Thrips tabaci.

Examples of Nematodes include, but are not limited to: parasitic nematodes such as root-knot, cyst, and lesion nematodes, including Heterodera spp., Meloidogyne spp., and Globodera spp.; particularly members of the cyst nematodes, including, but not limited to: Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include, but are not limited to: Pratylenchus spp.

Other insect species susceptible to the present invention include: athropod pests that cause public and animal health concerns, for example, mosquitos for example, mosquitoes from the genera Aedes, Anopheles and Culex, from ticks, flea, and flies etc.

In one embodiment, a DVP, a DVP-insecticidal protein, or a pharmaceutically acceptable salt thereof can be employed to treat ectoparasites. Ectoparasites include, but are not limited to: fleas, ticks, mange, mites, mosquitoes, nuisance and biting flies, lice, and combinations comprising one or more of the foregoing ectoparasites. The term “fleas” includes the usual or accidental species of parasitic flea of the order Siphonaptera, and in particular the species Ctenocephalides, in particular C. fells and C. cams, rat fleas (Xenopsylla cheopis) and human fleas (Pulex irritans).

The present invention may be used to control, inhibit, and/or kill insect pests of major crops, e.g., in some embodiments, the major crops and corresponding insect pest include, but are not limited to: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis, corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis Orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, banded winged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Soybean: Pseudoplusia includens, soybean looper; Anticarsia gemmatalis, velvet bean caterpillar; Plathypena scabra, green clover worm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoascafabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

In some embodiments, a DVP, a DVP-insecticidal protein, or a pharmaceutically acceptable salt thereof can be employed to treat any one or more of the foregoing insects.

The insects that are susceptible to present invention include but are not limited to the following: families such as: Blattaria, Coleoptera, Collembola, Diptera, Echinostomida, Hemiptera, Hymenoptera, Isoptera, Lepidoptera, neuroptera, Orthoptera, Rhabditida, Siphonoptera, and Thysanoptera. Genus Species are indicated as follows: Actebiafennica, Agrotis ipsilon, A. segetum, Anticarsia gemmatalis, Argyrotaenia citrana, Artogeia rapae, Bombyx mori, Busseola fusca, Cacyreus marshall, Chilo suppressalis, Christoneura fumiferana, C. occidentalis, C. pinus pinus, C. rosacena, Cnaphalocrocis medinalis, Conopomorpha cramerella, Ctenopsuestis obliquana, Cydia pomonella, Danaus plexippus, Diatraea saccharallis, D. grandiosella, Earias vittella, Elasmolpalpus lignoselius, Eldana saccharina, Ephestia kuehniella, Epinotia aporema, Epiphyas postvittana, Galleria mellonella, Genus Species, Helicoverpa zea, H. punctigera, H. armigera, Heliothis virescens, Hyphantria cunea, Lambdina fiscellaria, Leguminivora glycinivorella, Lobesia botrana, Lymantria dispar, Malacosoma disstria, Mamestra brassicae, M configurata, Manduca sexta, Marasmia patnalis, Maruca vitrata, Orgyia leucostigma, Ostrinia nubilalis, O. furnacalis, Pandemis pyrusana, Pectinophora gossypiella, Perileucoptera coffeella, Phthorimaea opercullela, Pianotortrix octo, Piatynota stultana, Pieris brassicae, Plodia interpunctala, Plutella xylostella, Pseudoplusia includens, Rachiplusia nu, Sciropophaga incertulas, Sesamia calamistis, Spilosoma virginica, Spodoptera exigua, Spodoptera frugiperda, Spodoptera littoralis, Spodoptera exempta, Spodoptera litura, Tecia solanivora, Thaumetopoea pityocampa, Trichoplusia ni, Wiseana cervinata, Wiseana copularis, Wiseana jocosa, Blattaria blattella, Collembola xenylla, Collembola folsomia, Folsomia candida, Echinostomida fasciola, Hemiptera oncopeltrus, Hemiptera bemisia, Hemiptera macrosiphum, Hemiptera rhopalosiphum, Hemiptera myzus, Hymenoptera diprion, Hymenoptera apis, Hymenoptera Macrocentrus, Hymenoptera Meteorus, Hymenoptera Nasonia, Hymenoptera Solenopsis, isopoda porcellio, Isoptera reticulitermes, Orthoptera Achta, Prostigmata tetranychus, Rhabitida acrobeloides, Rhabitida caenorhabditis, Rhabitida distolabrellus, Rhabitidapanagrellus, Rhabitidapristionchus, Rhabitidapratylenchus, Rhabitida ancylostoma, Rhabitida nippostrongylus, Rhabitida panagrellus, Rhabitida haemonchus, Rhabitida meloidogyne, and Siphonaptera ctenocephalides.

The present disclosure provides methods for plant transformation, which may be used for transformation of any plant species, including, but not limited to, monocots and dicots. Crops for which a transgenic approach would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

The present disclosure provides methods for plant transformation, which may be used for transformation of any plant species, including, but not limited to, monocots and dicots. Crops for which a transgenic approach or plaint incorporated protectants (PIP) would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

In some embodiments, the compositions, mixtures, and/or methods of the present invention can be applied to the locus of an insect and/or pest selected from the group consisting of: Loopers; Omnivorous Leafroller; Hornworms; Imported Cabbageworm; Diamondback Moth; Green Cloverworm; Webworm; Saltmarsh Caterpillar; Armyworms; Cutworms; Cross-Striped Cabbageworm; Podworms; Velvetbean Caterpillar; Soybean Looper; Tomato Fruitworm; Variegated Cutworm; Melonworms; Rindworm complex; Fruittree Leafroller; Citrus Cutworm; Heliothis; Orangedog; Citrus Cutworm; Redhumped Caterpillar; Tent Caterpillars; Fall Webworm; Walnut Caterpillar; Cankerworms; Gypsy Moth; Variegated Leafroller; Redbanded Leafroller; Tufted Apple Budmoth; Oriental Fruit Moth); Filbert Leafroller; Obliquebanded Leafroller; Codling Moth; Twig Borer; Grapeleaf Skeletonizer; Grape Leafroller; Achema Sphinx Moth (Hornworm); Orange Tortrix; Tobacco Budworm); Grape Berry Moth; Spanworm; Alfalfa Caterpillar; Cotton Bollworm; Head Moth; Amorbia Moth; Omnivorous Looper; Ello Moth (Hornworm); Jo Moth; Oleander Moth; Azalea Caterpillar; Hornworm; Leafrollers; Banana Skipper; Batrachedra comosae (Hodges); Thecla Moth; Artichoke Plume Moth; Thistle Butterfly; Bagworm; Spring & Fall Cankerworm; Elm Spanworm; California Oakworm; Pine Butterfly; Spruce Budworms; Saddle Prominent Caterpillar; Douglas Fir Tussock Moth; Western Tussock Moth; Blackheaded Budworm; Mimosa Webworm; Jack Pine Budworm; Saddleback Caterpillar; Greenstriped Mapleworm; or Hemlock Looper.

In some embodiments, the compositions, mixtures, and/or methods of the present invention can be applied to the locus of an insect and/or pest selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola) (Essex Skipper); Fall Webworm (Melissopus latiferreanus); Filbert Leafroller (Archips rosanus); Fruittree Leafroller (Archips argyrospilia); Grape Berry Moth (Paralobesia viteana); Grape Leafroller (Platynota stultana); Grapeleaf Skeletonizer (Harrisina americana) (ground only); Green Cloverworm (Plathypena scabra); Greenstriped Mapleworm (Dryocampa rubicunda); Gummosos-Batrachedra Comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Redbanded Leafroller (Argyrotaenia velutinana); Redhumped Caterpillar (Schizura concinna); Rindworm Complex (Various Leps.); Saddleback Caterpillar (Sibine stimulea); Saddle Prominent Caterpillar (Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and/or Xanthogaleruca luteola.

In some embodiments, the compositions, mixtures, and/or methods of the present invention can be applied to the locus of an adult beetle selected from the group consisting of: Asiatic garden beetle (Maladera castanea); Gold spotted oak borer (Agrilus coxalis auroguttatus); Green June beetle (Cotinis nitida); Japanese beetle (Popillia japonica); May or June beetle (Phyllophaga sp.); Oriental beetle (Anomala orientalis); and/or Soap berry-borer (Agrilus prionurus).

In some embodiments, the compositions, mixtures, and/or methods of the present invention can be applied to the locus of an insect and/or pest that is a larvae (annual white grub) selected from the group consisting of: Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.); Northern masked chafer (Cyclocephala borealis); Oriental beetle (Anomala orientalis); Southern masked chafer (Cyclocephala lurida); and Billbug (Curculionoidea).

Cystine Knot Architecture

Cysteine Rich Proteins (CRPs) are peptides rich in cysteine residues that, in some embodiments, are operable to form disulfide bonds between such cysteine residues. In some embodiments, CRPs contain 4, 5, 6, 7, 8, 9, 10, or more cysteine amino acids. And, in some embodiments, the cysteine residues present in a CRP may form 3 or more disulfide bonds. In some embodiments, the disulfide bonds contribute to the folding, three-dimensional structure, and activity of the insecticidal peptide.

CRPs, by virtue of their cysteine-cysteine disulfide bonds, can have remarkable stability when exposed to the environment. In some embodiments, a CRP can have insecticidal properties. For example, in some embodiments, a CRP can be a cysteine rich insecticidal protein (CRIP). And, in some embodiments, the cysteine-cysteine disulfide bonds, and the three dimensional structure formed therefrom, play a significant role in the insecticidal nature of these proteins.

In some embodiments, the 3 disulfide bonds present in a CRP can have a disulfide bond topology that forms a cystine knot (CK) motif. A cystine knot (CK) motif is a protein structural motif containing at least three disulfide bridges or bonds (formed between pairs of cysteine molecules). The cystine knot is built from two disulfide bonds and their connecting backbone segments forming an internal ring in the structure that is threaded by the third disulfide bond to form an interlocking and cross braced structure, forming a rotaxane substructure.

In some embodiments, the 3 disulfide bonds have a disulfide bond topology that creates one of the following CK motifs: an inhibitor cystine knot (ICK) motif, a growth factor cystine knot (GFCK) motif, or a cyclic cystine knot (CCK) motif.

And inhibitor cystine knot (ICK), or “knottin,” is a protein structural motif containing at least three disulfide bonds. Along with the peptide subunits between the bonds, two disulfides (linking the first and fourth cysteine and the second and fifth cysteine, respectively) form a loop through which the third disulfide bond (linking the third and sixth cysteine in the sequence) passes, forming a knot. The motif is common in invertebrate toxins such as those from arachnids and mollusks. The motif is also found in some inhibitor proteins found in plants.

Proteins comprising an ICK motif can be 16 to 60 amino acids long, with at least 6 half-cystine core amino acids having at least three disulfide bridges, wherein the 3 disulfide bridges are covalent bonds, and of the six half-cystine residues the covalent disulfide bonds are between the first (C^{I}) and fourth (C^{IV}), the second (C^{II}) and fifth (C^{V}), and the third (C^{III}) and sixth (C^{VI}), half-cystines, of the six core half-cystine amino acids starting from the N-terminal amino acid. In general this type of protein comprises a beta-hairpin secondary structure, normally composed of residues situated between the fourth and sixth core half-cystines of the motif, the hairpin being stabilized by the structural crosslinking provided by the motif s three disulfide bonds. Note that additional cysteine/cystine or half-cystine amino acids may be present within the inhibitor cystine knot motif.

Cyclic cystine knot (CCK) or cyclotides are similar to ICKs, however, CCK peptides are cyclized. CCKs fall into two main structural subfamilies: Moebius cyclotides, the less common of the two, contain a cis-proline in loop 5 that induces a local 180° backbone twist; bracelet cyclotides, another subfamily, do not have this feature. The trypsin inhibitor cyclotides are classified in their own family based on sequence variation and natural activity. Trypsin inhibitor cyclotides are more homologous to a family of non-cyclic trypsin inhibitors from squash plants known as knottins or inhibitor cystine knots than they are to the other cyclotides. Here, “cyclic” or “cyclized” refers to a molecule comprising a sequence of amino acid residues or analogues thereof without free amino and carboxy termini. In some embodiments, a cyclized peptide comprises a linkage between all amino acids in the peptide via amide (peptide) bonds, but other chemical linkers are also possible.

The growth factor cystine knot (GFCK) likewise has a similar motif to ICK peptides, but its topology is such that the bond between the C^{I} and C^{IV} threads through the loop (formed between the C^{II} and C^{V} cysteine and the C^{III} and C^{VI} cysteine, respectively).

Arriving at the CK Architecture of Formula (II) by Removing a Bond

The present invention contemplates and teaches methods of engineering a recombinant CRP comprising, consisting essentially of, or consisting of, a cystine knot (CK) architecture according to Formula (II):

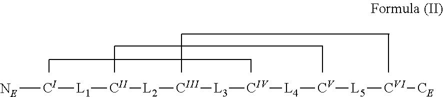

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; wherein said recombinant CRP is created by modifying a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, wherein the modifiable CRP is modified by removing one or more non-CK disulfide bonds from a modifiable CRP having one or more non-CK disulfide bonds; wherein removing the one or more disulfide bonds from the modifiable CRP having one or more non-CK disulfide bonds, results in the recombinant CRP having the CK architecture according to Formula (II); and wherein the recombinant CRP having the CK architecture according to Formula (II) has an increased level of expression relative to a level of expression of a modifiable CRP that does not have the CK architecture according to Formula (II).

In some embodiments, a CRIP comprising, consisting essentially of, or consisting of, the CK architecture according to Formula (II), is created according to the following process: removing one or more cysteine amino acid residues from a polypeptide having seven or more cysteine amino acid residues, wherein the polypeptide does not have a CK architecture according to Formula (II).

In some embodiments, removing the one or more cysteine amino acid residues from a modifiable CRP that does not have a CK architecture according to Formula (II), results in a removal of one or more disulfide bonds from the modifiable CRP.

In some embodiments, the removal of one or more disulfide bonds from a modifiable CRP that does not have a CK architecture according to Formula (II), results in a recombinant CRP having a CK architecture according to Formula (II); and results in the following effect: an increase in the expression of the recombinant CRP in, e.g., a recombinant protein expression system, relative to the polypeptide not having the CK architecture according to Formula (II).

There are a variety of methods for measuring peptide yield known to those having ordinary skill in the art. In some embodiments, the peptide yield can be a “normalized peptide yield,” which means the peptide yield in the conditioned medium divided by the corresponding cell density at the point the peptide yield is measured. The peptide yield can be represented by the mass of the produced peptide in a unit of volume, for example, mg per liter or mg/L, or by the UV absorbance peak area of the produced peptide in the HPLC chromatograph, for example, mAu·sec. The cell density can be represented by visible light absorbance of the culture at wavelength of 600 nm (OD600). “OD” refers to optical density. Typically, OD is measured using a spectrophotometer. When measuring growth over time of a cell population, OD600 is preferable to UV spectroscopy; this is because at a 600 nm wavelength, the cells will not be harmed as they would under too much UV light. “OD660 nm” or “OD₆₆₀ₙₘ” refers to optical densities at 660 nanometers (nm).

In some embodiments, a recombinant CRP of the present invention comprises, consists essentially of, or consists of, a protein having a CK architecture according to Formula (II). The CK architecture according to Formula (II) refers to a configuration of cysteines and disulfide bond topology, wherein proteins with the CK architecture according to Formula (II) possess a shared structural similarity. Here, the CK architecture according to Formula (II) comprises, consists essentially of, or consists of, six cysteine residues connected by three disulfide bonds, wherein the disulfide bonds are connected between cysteines C^{I} and C^{IV}; C^{II} and C^{V}; and C^{III} and C^{VI}.

In some embodiments, a recombinant CRP having the CK architecture according to Formula (II), has an increase of a level of expression that is equal to or greater than: 1%, 2%, 3%, 4%, %5%, 6%, 7%, 8%, 9%, 10, 11%, 12%, 13%, 14%, 1%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or greater than 100%, relative to a level of expression of a modifiable CRP that does not have the CK architecture according to Formula (II).

In some embodiments, the recombinant CRP of the present invention has a disulfide bond topology, wherein the disulfide bond topology forms one of the following cystine knot motifs: an inhibitor cystine knot (ICK) motif, a growth factor cystine knot (GFCK) motif, or a cyclic cystine knot (CCK) motif.

In some embodiments, the recombinant CRP of the present invention has a disulfide bond topology, wherein the disulfide bond topology forms an ICK motif.

In some embodiments, a modifiable CRP is a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif.

Accordingly, in some embodiments, the one or more non-CK disulfide bonds is any additional disulfide bond that is not the first disulfide bond, the second disulfide bond, and/or the third disulfide bond, as the first disulfide bond, the second disulfide bond, and the third disulfide bond are the only disulfide bonds that form the cystine knot motif. In other words, when there is an additional disulfide bond that is not the first disulfide bond, the second disulfide bond, and/or the third disulfide bond, and/or is not one of the two disulfide bonds that, in concert with their connecting backbone segments, form an internal ring in the structure, and/or the third disulfide bond that threads this ring to form an interlocking and cross braced structure, thus, forming a rotaxane substructure, then such an additional disulfide bond is a non-CK disulfide bond.

In some embodiments, a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, can be modified by removing one or more non-CK disulfide bonds from a modifiable CRP having one or more non-CK disulfide bonds.

In some embodiments, removing the one or more disulfide bonds from the modifiable CRP having one or more non-CK disulfide bonds, results in the recombinant CRP having the CK architecture according to Formula (II).

In some embodiments, removing the one or more disulfide bonds from the modifiable CRP having one or more non-CK disulfide bonds, results in the recombinant CRP having the CK architecture according to Formula (II), wherein the recombinant CRP having the CK architecture according to Formula (II) has an increased level of expression relative to a level of expression of a modifiable CRP that does not have the CK architecture according to Formula (II).

In some embodiments, the increase in the level of expression of the recombinant CRP having the CK architecture according to Formula (II), relative to a level of expression of a modifiable CRP that does not have the CK architecture according to Formula (II), can be an increase in expression in the recombinant CRP ranging from about at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 20.25%, at least about 2.5%, at least about 20.75%, at least about 3%, at least about 30.25%, at least about 3.5%, at least about 30.75%, at least about 4%, at least about 4 0.25%, at least about 4.5%, at least about 4 0.75%, at least about 5%, at least about 5.2 5%, at least about 5.5%, at least about 5.7 5%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8 0.75%, at least about 9%, at least about 9 0.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the level of expression of a modifiable CRP that does not have the CK architecture according to Formula (II).

In some embodiments, the increase in the level of expression of the recombinant CRP having the CK architecture according to Formula (II), relative to a level of expression of a modifiable CRP that does not have the CK architecture according to Formula (II), can be an increase ranging from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 2%, 13%, 14%, 1%, 16 5, 1⁷, 18%, %19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, to about 1000%, or greater than the level of expression of a modifiable CRP that does not have the CK architecture according to Formula (II).

In some embodiments, the modifiable CRP is modified by removing one or more non-CK disulfide bonds from the modifiable CRP having one or more non-CK disulfide bonds.

In some embodiments, the modifiable CRP is a wild-type μ-DGTX-Dc1a; a DVP; a Kappa-ACTX, an ApsIII, or a variant thereof.

In some embodiments, the modifiable CRP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 1-2, 193, 195, or 198.

In some embodiments, the modifiable CRP consists of an amino acid sequence set forth in any one of SEQ ID NOs: 1-2, 193, 195, or 198.

In some embodiments, the recombinant CRP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-14, 197, 199, or 201.

In some embodiments, the recombinant CRP consists of an amino acid sequence set forth in any one of SEQ ID NOs: 6-14, 197, 199, or 201.

Method of Making a Recombinant CRP Comprising a CK Architecture According to Formula (II)

In some embodiments, the present invention provides a method of making a recombinant cysteine-rich protein (CRP) comprising a cystine knot (CK) architecture according to Formula (II):

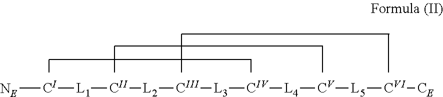

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; said method comprising: (a) providing a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, and (b) modifying the modifiable CRP by removing one or more non-CK disulfide bonds from a modifiable CRP having one or more non-CK disulfide bonds; wherein removing the one or more disulfide bonds from the modifiable CRP having one or more non-CK disulfide bonds, results in the recombinant CRP having the CK architecture according to Formula (II); and wherein the recombinant CRP having the CK architecture according to Formula (II) has an increased level of expression relative to a level of expression of a modifiable CRP that does not have the CK architecture according to Formula (II).

In some embodiments, the method provides a recombinant CRP that has a disulfide bond topology, wherein the disulfide bond topology forms one of the following cystine knot motifs: an inhibitor cystine knot (ICK) motif, a growth factor cystine knot (GFCK) motif, or a cyclic cystine knot (CCK) motif.

In some embodiments, the method provides recombinant CRP that has a disulfide bond topology, wherein the disulfide bond topology forms an ICK motif.

In some embodiments, the method provides a modifiable CRP that is a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif. Accordingly, in some embodiments, the one or more non-CK disulfide bonds is any additional disulfide bond that is not the first disulfide bond, the second disulfide bond, and/or the third disulfide bond, as the first disulfide bond, the second disulfide bond, and the third disulfide bond are the only disulfide bonds that form the cystine knot motif. In other words, when there is an additional disulfide bond that is not the first disulfide bond, the second disulfide bond, and/or the third disulfide bond, and/or is not one of the two disulfide bonds that, in concert with their connecting backbone segments, form an internal ring in the structure, and/or the third disulfide bond that threads this ring to form an interlocking and cross braced structure, thus, forming a rotaxane substructure, then such an additional disulfide bond is a non-CK disulfide bond.

In some embodiments, the method provides a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, can be modified by removing one or more non-CK disulfide bonds from a modifiable CRP having one or more non-CK disulfide bonds.

In some embodiments, removing the one or more disulfide bonds from the modifiable CRP having one or more non-CK disulfide bonds, results in the recombinant CRP having the CK architecture according to Formula (II), wherein the recombinant CRP having the CK architecture according to Formula (II) has an increased level of expression relative to a level of expression of a modifiable CRP that does not have the CK architecture according to Formula (II).

In some embodiments, the increase in the level of expression of the recombinant CRP having the CK architecture according to Formula (II), relative to a level of expression of a modifiable CRP that does not have the CK architecture according to Formula (II), can be an increase in expression in the recombinant CRP ranging from about at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 20.25%, at least about 2.5%, at least about 20.75%, at least about 3%, at least about 30.25%, at least about 3.5%, at least about 30.75%, at least about 4%, at least about 4 0.25%, at least about 4.5%, at least about 4 0.75%, at least about 5%, at least about 5.2 5%, at least about 5.5%, at least about 5.7 5%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8 0.75%, at least about 9%, at least about 9 0.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the level of expression of a modifiable CRP that does not have the CK architecture according to Formula (II).

In some embodiments, the increase in the level of expression of the recombinant CRP having the CK architecture according to Formula (II), relative to a level of expression of a modifiable CRP that does not have the CK architecture according to Formula (II), can be an increase ranging from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, to about 1000%, or greater than the level of expression of a modifiable CRP that does not have the CK architecture according to Formula (II).

In some embodiments, the modifiable CRP is modified by removing one or more non-CK disulfide bonds from the modifiable CRP having one or more non-CK disulfide bonds.

In some embodiments, the modifiable CRP is a wild-type μ-DGTX-Dc1a; a DVP; a Kappa-ACTX, an ApsIII, or a variant thereof.

In some embodiments, the method step of providing a modifiable CRP comprises providing a protein having an amino acid sequence as set forth in any one of SEQ ID NOs: 1-2, 193, 195, or 198.

In some embodiments, creating a recombinant CRP results in the creation of a recombinant CRP comprising an amino acid sequence as set forth in any one of SEQ ID NOs: 6-14, 197, 199, or 201.

In some embodiments, the method results in a recombinant CRP that has disulfide bond topology forming one of the following cystine knot motifs: an inhibitor cystine knot (ICK) motif, a growth factor cystine knot (GFCK) motif, or a cyclic cystine knot (CCK) motif.

In some embodiments, the method provides a recombinant CRP having a disulfide bond topology that forms an ICK motif.

In some embodiments, the method provides a modifiable CRP, wherein the modifiable CRP is a wild-type μ-DGTX-Dc1a; a DVP; a Kappa-ACTX, an ApsIII, or a variant thereof.

In some embodiments, the method provides a modifiable CRP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 1-2, 193, 195, or 198.

In some embodiments, the method provides a modifiable CRP consisting of an amino acid sequence set forth in any one of SEQ ID NOs: 1-2, 193, 195, or 198.

In some embodiments, the method creates a recombinant CRP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-14, 197, 199, or 201.

In some embodiments, the method creates a recombinant CRP consisting of an amino acid sequence set forth in any one of SEQ ID NOs: 6-14, 197, 199, or 201.

Method of Increasing Yield of a Recombinant CRP

In some embodiments, the present invention provides a method of increasing the yield of a recombinant cysteine-rich protein (CRP), said method comprising: (a) creating a recombinant CRP having a cystine knot (CK) architecture according to Formula (II):

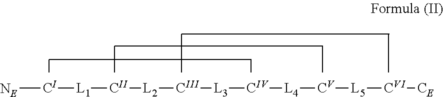

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; wherein said recombinant CRP is created according to the following process: (b) providing a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, and (c) modifying the modifiable CRP by removing one or more non-CK disulfide bonds from the modifiable CRP having one or more non-CK disulfide bonds; wherein removing the one or more disulfide bonds from the modifiable CRP having one or more non-CK disulfide bonds results in the recombinant CRP having the CK architecture according to Formula (II); wherein the recombinant CRP having the CK architecture according to Formula (II) has an increased level of expression relative to a level of expression of a modifiable CRP that does not have the CK architecture according to Formula (II).

In some embodiments, the method of increasing yield provides a recombinant CRP that has a disulfide bond topology, wherein the disulfide bond topology forms one of the following cystine knot motifs: an inhibitor cystine knot (ICK) motif, a growth factor cystine knot (GFCK) motif, or a cyclic cystine knot (CCK) motif.

In some embodiments, the method of increasing yield provides recombinant CRP that has a disulfide bond topology, wherein the disulfide bond topology forms an ICK motif.

In some embodiments, the method of increasing yield provides a modifiable CRP that is a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif. Accordingly, in some embodiments, the one or more non-CK disulfide bonds is any additional disulfide bond that is not the first disulfide bond, the second disulfide bond, and/or the third disulfide bond, as the first disulfide bond, the second disulfide bond, and the third disulfide bond are the only disulfide bonds that form the cystine knot motif. In other words, when there is an additional disulfide bond that is not the first disulfide bond, the second disulfide bond, and/or the third disulfide bond, and/or is not one of the two disulfide bonds that, in concert with their connecting backbone segments, form an internal ring in the structure, and/or the third disulfide bond that threads this ring to form an interlocking and cross braced structure, thus, forming a rotaxane substructure, then such an additional disulfide bond is a non-CK disulfide bond.

In some embodiments, the method of increasing yield provides a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, can be modified by removing one or more non-CK disulfide bonds from a modifiable CRP having one or more non-CK disulfide bonds.

In some embodiments, removing the one or more disulfide bonds from the modifiable CRP having one or more non-CK disulfide bonds, results in the recombinant CRP having the CK architecture according to Formula (II), wherein the recombinant CRP having the CK architecture according to Formula (II) has an increased level of expression of protein or yield of protein relative to a yield of protein or level of expression of protein of a modifiable CRP that does not have the CK architecture according to Formula (II).

In some embodiments, the increase in the level of expression of the recombinant CRP having the CK architecture according to Formula (II), relative to a level of expression of a modifiable CRP that does not have the CK architecture according to Formula (II), can be an increase in expression in the recombinant CRP ranging from about at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 20.25%, at least about 2.5%, at least about 20.75%, at least about 3%, at least about 30.25%, at least about 3.5%, at least about 30.75%, at least about 4%, at least about 4 0.25%, at least about 4.5%, at least about 4 0.75%, at least about 5%, at least about 5.2 5%, at least about 5.5%, at least about 5.7 5%, at least about 6%, at least about 6.25%, at least about 6.5%, at least about 6.75%, at least about 7%, at least about 7.25%, at least about 7.5%, at least about 7.75%, at least about 8%, at least about 8.25%, at least about 8.5%, at least about 8 0.75%, at least about 9%, at least about 9 0.25%, at least about 9.5%, at least about 9.75%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 80%, at least about 85%, at least about 90%, at least about 95%, at least about 100%, or a greater than a 100%, relative to the level of expression of a modifiable CRP that does not have the CK architecture according to Formula (II).

In some embodiments, the increase in the yield level of expression of the recombinant CRP having the CK architecture according to Formula (II), relative to a level of expression of a modifiable CRP that does not have the CK architecture according to Formula (II), can be an increase ranging from about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 2%, 13%, 14%, 15%, 16 , 17, 18%, %19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, to about 1000%, or greater than the level of expression of a modifiable CRP that does not have the CK architecture according to Formula (II).

In some embodiments, the method of increasing yield provides a modifiable CRP that is modified by removing one or more non-CK disulfide bonds from the modifiable CRP having one or more non-CK disulfide bonds.

In some embodiments, the method of increasing yield provides a modifiable CRP that is a wild-type μ-DGTX-Dc1a; a DVP; a Kappa-ACTX, an ApsIII, or a variant thereof.

In some embodiments, the method of increasing yield step of providing a modifiable CRP comprises providing a protein having an amino acid sequence as set forth in any one of SEQ ID NOs: 1-2, 193, 195, or 198.

In some embodiments, the method of increasing yield results in the creation of a recombinant CRP, wherein said recombinant CRP comprises an amino acid sequence as set forth in any one of SEQ ID NOs: 6-14, 197, 199, or 201.

In some embodiments, the method of increasing yield results in a recombinant CRP that has disulfide bond topology forming one of the following cystine knot motifs: an inhibitor cystine knot (ICK) motif, a growth factor cystine knot (GFCK) motif, or a cyclic cystine knot (CCK) motif.

In some embodiments, the method of increasing yield provides a recombinant CRP having a disulfide bond topology that forms an ICK motif.

In some embodiments, the method of increasing yield provides a modifiable CRP, wherein the modifiable CRP is a wild-type μ-DGTX-Dc1a; a DVP; a Kappa-ACTX, an ApsIII, or a variant thereof.

In some embodiments, the method of increasing yield provides a modifiable CRP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 1-2, 193, 195, or 198.

In some embodiments, the method of increasing yield provides a modifiable CRP consisting of an amino acid sequence set forth in any one of SEQ ID NOs: 1-2, 193, 195, or 198.

In some embodiments, the method of increasing yield creates a recombinant CRP comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-14, 197, 199, or 201.

In some embodiments, the method of increasing yield creates a recombinant CRP consisting of an amino acid sequence set forth in any one of SEQ ID NOs: 6-14, 197, 199, or 201.

In some embodiments, the present invention provides a recombinant CRP comprising, consisting essentially of, or consisting of, a cystine knot (CK) architecture according to Formula (II):

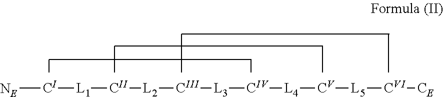

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; wherein said recombinant CRP is created by modifying a wild-type μ-DGTX-Dc1a; a DVP; a Kappa-ACTX, an ApsIII, or a variant thereof, according to the following process: removing one or more non-CK disulfide bonds from a modifiable CRP having one or more non-CK disulfide bonds; wherein removing the one or more disulfide bonds from the modifiable CRP having one or more non-CK disulfide bonds, results in the recombinant CRP having the CK architecture according to Formula (II); and wherein the recombinant CRP having the CK architecture according to Formula (II) has an increased level of expression relative to a level of expression of a wild-type μ-DGTX-Dc1a; a DVP; a Kappa-ACTX, an ApsIII, or a variant thereof that does not have the CK architecture according to Formula (II).

In some embodiments, the modifiable CRP is modified by removing one or more non-CK disulfide bonds from the modifiable CRP having one or more non-CK disulfide bonds.

In some embodiments, the modifiable CRP is a wild-type μ-DGTX-Dc1a; a DVP; a Kappa-ACTX, an ApsIII, or a variant thereof.

In some embodiments, the modifiable CRP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 1-2, 193, 195, or 198.

In some embodiments, the recombinant CRP comprises an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence as set forth in any one of SEQ ID NOs: 6-14, 197, 199, or 201.

Exemplary Cystine-Knot Architecture Embodiments

In some embodiments, a polypeptide can have cysteines and/or disulfide bonds, but not the CK architecture according to Formula (II) of the present invention. For example, in some embodiments, a polypeptide can have seven or more cysteine amino acid residues. In some embodiments, a polypeptide can have four or more disulfide bonds.

Here the inventors provide recombinant CRPs that are derived from modifiable CRPs in order to arrive at the CK architecture of Formula (II), and methods regarding the same. For example, in some embodiments, the present invention comprises, consists essentially of, or consists of a modifiable CRP with 7 cysteine residue that has been modified to include the removal of one cysteine residue, wherein the removal of the 1 cysteine residue results in the polypeptide having the CK architecture of Formula (II).

In some embodiments, the present invention comprises, consists essentially of, or consists of a modifiable CRP with 8 cysteine residues that has been modified to include the removal of 2 cysteine residues, wherein the removal of the 2 cysteine residues results in a recombinant CRP having the CK architecture of Formula (II).

In some embodiments, the present invention comprises, consists essentially of, or consists of a modifiable CRP with 9 cysteine residues that has been modified to include the removal of 3 cysteine residues, wherein the removal of the 3 cysteine residues results in a recombinant CRP having the CK architecture of Formula (II).

In some embodiments, the present invention comprises, consists essentially of, or consists of a modifiable CRP with 10 cysteine residues that has been modified to include the removal of 4 cysteine residues, wherein the removal of the 4 cysteine residues results in a recombinant CRP having the CK architecture of Formula (II).

In some embodiments, the present invention comprises, consists essentially of, or consists of a modifiable CRP with 4 or more disulfide bonds, wherein the modifiable CRP has been modified to have 3 disulfide bonds, by removing 1, 2, 3, 4, 5, or more disulfide bonds.

In some embodiments, a modifiable CRP of the present invention can be modified by removing one or more cysteine amino acid residues from a modifiable CRP having seven or more cysteine amino acid residues; wherein the modifiable CRP does not have a CK architecture according to Formula (II), and wherein removing the one or more cysteine amino acid residues from the polypeptide results in the removal of one or more non-CK disulfide bonds from the modifiable CRP.

In some embodiments, the present invention comprises, consists essentially of, or consists of, a polypeptide having with four disulfide bonds, wherein one disulfide bond is removed to create a CK architecture of Formula (II) wherein disulfide bonds are formed between cysteine residues: C^{I} and C^{IV}; C^{II} and C^{V}; and C^{III} and C^{VI}; e.g., a cystine knot with 1-4, 2-5, 3-6 disulfide bond connectivity.

In some embodiments, the present invention comprises, consists essentially of, or consists of, a polypeptide having with eight cysteines, wherein two cysteines are removed to create a CK architecture of Formula (II) wherein disulfide bonds are formed between cysteine residues: C^{I} and C^{IV}; C^{II} and C^{V}; and C^{III} and C^{VI}; e.g., a cystine knot with 1-4, 2-5, 3-6 disulfide bond connectivity.

In some embodiments, the present invention comprises, consists essentially of, or consists of, a method of increasing the expression of a polypeptide, wherein said method occurs by removing one or more cysteines, wherein the method comprises, consists essentially of, or consists of, one or more of the following steps: (a) obtaining and/or creating a 3-D structure of the modifiable CRP; (b) predicting one or more sites for the removal of one or more cysteines based on the 3-D structure of the modifiable CRP; and (c) modifying the modifiable CRP by removing one or more cysteines at one or more of the predicted sites; wherein the removal of said one or more cysteines permits the removal of at least one non-CK disulfide bond.

In some embodiments, the present invention comprises, consists essentially of, or consists of, a polypeptide which is the product of a single gene in nature, and which has been mutated by removing one or more cysteine residues, wherein the removal of said cysteine residues permits the removal of one or more non-CK disulfide bonds, which increases the expression of the recombinant CRP, relative to the modifiable CRP that does not contain said removed cysteine.

In some embodiments, the present invention comprises, consists essentially of, or consists of, recombinant cysteine rich protein (CRP), said CRP comprising a cystine knot architecture according to Formula (II):

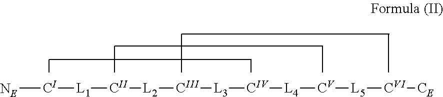

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; wherein said recombinant CRP is created by modifying a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, wherein the modifiable CRP is modified by removing one or more non-CK disulfide bonds from a modifiable CRP having one or more non-CK disulfide bonds; wherein removing the one or more disulfide bonds from the modifiable CRP having one or more non-CK disulfide bonds, results in the recombinant CRP having the CK architecture according to Formula (II); and wherein the recombinant CRP having the CK architecture according to Formula (II) has an increased level of expression relative to a level of expression of a modifiable CRP that does not have the CK architecture according to Formula (II); and wherein each amino acid sequence of the N_{E}, L₁, L₂, L₃, L₄, L₅, and C_{E} peptide subunits has at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following groups of amino acid sequences: N_{E} is AKDGDVEGPAG; L₁ is KKYDVE; L₂ is DSGE; L₃ is absent; L₄ is QKQYLWYKWRPLD; L₅ is RGLKSGFFSSKFV; and C_{E} is RDV.

In some embodiments, the present invention comprises, consists essentially of, or consists of, a recombinant cysteine rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence:

(SEQ ID NO: 5)
AKDGDVEGPAGCKKYDVECDSGECCQKQYLWYKWRPLDCRGLKSGFFSS
KFVCRDV.

In some embodiments, the present invention comprises, consists essentially of, or consists of, a recombinant cysteine rich protein (CRP), said CRP comprising an CK architecture according to Formula (II), and having an amino acid sequence that is:

(SEQ ID NO: 5)
AKDGDVEGPAGCKKYDVECDSGECCQKQYLWYKWRPLDCRGLKSGFFSS
KFVCRDV.

In some embodiments, the present invention comprises, consists essentially of, or consists of, a recombinant cysteine-rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence:

(SEQ ID NO: 199)
AICTGADRPCAAACPCCPGTSCKAESNGVSYCRKDEP.

In some embodiments, the present invention comprises, consists essentially of, or consists of, a recombinant cysteine rich protein (CRP), said CRP comprising an CK architecture according to Formula (II), and having an amino acid sequence that is:

(SEQ ID NO: 199)
AICTGADRPCAAACPCCPGTSCKAESNGVSYCRKDEP.

In some embodiments, the present invention comprises, consists essentially of, or consists of, a recombinant cysteine-rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence:

(SEQ ID NO: 201)
AICTGADRPCAAAAPCCPGTSCKAESNGVSYCRKDEP.

In some embodiments, the present invention comprises, consists essentially of, or consists of, a recombinant cysteine rich protein (CRP), said CRP comprising an CK architecture according to Formula (II), and having an amino acid sequence that is:

(SEQ ID NO: 201)
AICTGADRPCAAAAPCCPGTSCKAESNGVSYCRKDEP.

In some embodiments, the present invention comprises, consists essentially of, or consists of, a recombinant cysteine-rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence:

(SEQ ID NO: 197)
GSCNSKGTPCTNADECCGGKCAYNVWNAIGGGASKTCGY.

In some embodiments, the present invention comprises, consists essentially of, or consists of, a recombinant cysteine rich protein (CRP), said CRP comprising an CK architecture according to Formula (II), and having an amino acid sequence that is:

(SEQ ID NO: 197)
GSCNSKGTPCTNADECCGGKCAYNVWNAIGGGASKTCGY.

In some embodiments, the present invention comprises, consists essentially of, or consists of, a polynucleotide operable to encode a recombinant cysteine rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence: AKDGDVEGPAGCKKYDVECDSGECCQKQYLWYKWRPLDCRGLKSGFFSSKFVCRDV (SEQ ID NO:5), or a complementary nucleotide sequence thereof.

In some embodiments, the present invention comprises, consists essentially of, or consists of, a polynucleotide operable to encode a recombinant cysteine rich protein (CRP), said CRP comprising an CK architecture according to Formula (II), and having an amino acid sequence that is: AKDGDVEGPAGCKKYDVECDSGECCQKQYLWYKWRPLDCRGLKSGFFSSKFVCRDV (SEQ ID NO:5), or a complementary nucleotide sequence thereof.

In some embodiments, the present invention comprises, consists essentially of, or consists of, a polynucleotide operable to encode a recombinant cysteine-rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence:

AICTGADRPCAAACPCCPGTSCKAESNGVSYCRKDEP (SEQ ID NO: 199), or a complementary nucleotide sequence thereof.

In some embodiments, the present invention comprises, consists essentially of, or consists of, a polynucleotide operable to encode a recombinant cysteine rich protein (CRP), said CRP comprising an CK architecture according to Formula (II), and having an amino acid sequence that is: AICTGADRPCAAACPCCPGTSCKAESNGVSYCRKDEP (SEQ ID NO: 199), or a complementary nucleotide sequence thereof.

In some embodiments, the present invention comprises, consists essentially of, or consists of, a polynucleotide operable to encode a recombinant cysteine-rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence:

AICTGADRPCAAAAPCCPGTSCKAESNGVSYCRKDEP (SEQ ID NO: 201), or a complementary nucleotide sequence thereof.

In some embodiments, the present invention comprises, consists essentially of, or consists of, a polynucleotide operable to encode a recombinant cysteine rich protein (CRP), said CRP comprising an CK architecture according to Formula (II), and having an amino acid sequence that is: AICTGADRPCAAAAPCCPGTSCKAESNGVSYCRKDEP (SEQ ID NO: 201), or a complementary nucleotide sequence thereof.

In some embodiments, the present invention comprises, consists essentially of, or consists of, a polynucleotide operable to encode a recombinant cysteine-rich protein (CRP), said CRP comprising an CK architecture according to Formula (II), and having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the following amino acid sequence:

GSCNSKGTPCTNADECCGGKCAYNVWNAIGGGASKTCGY (SEQ ID NO: 197), or a complementary nucleotide sequence thereof.

In some embodiments, the present invention comprises, consists essentially of, or consists of, a polynucleotide operable to encode a recombinant cysteine rich protein (CRP), said CRP comprising an CK architecture according to Formula (II), and having an amino acid sequence that is: GSCNSKGTPCTNADECCGGKCAYNVWNAIGGGASKTCGY (SEQ ID NO: 197), or a complementary nucleotide sequence thereof.

### EXAMPLES

The Examples in this specification are not intended to, and should not be used to, limit the invention; they are provided only to illustrate the invention. The categories below for fold expression are: Reduced=<0.9; Similar=0.9-1.1; Slightly Increased=1.1-2.9; Increased=3.0-10.0; and Highly increased=>10. The categories below for fold activity are: Reduced=>1.5; and Similar=0.7-1.4.

### Example 1. Yeast Transformation

Yeast Transformation

Individual ORFs were constructed containing either a polynucleotide operable to encode a wild-type Dc1a, or a polynucleotide operable to encode a given DVP, and the sequence of the alpha mating factor secretion signal. These ORFs were then inserted into a pKlac1 vector (Catalog No. N3740; New England Biolabs®; 240 County Road, Ipswich, MA 01938-2723). The pKlac1 vector contains the Kluyveromyces lactis P_{LAC4-PBI} promoter (1), DNA encoding the K. lactis α-mating factor (α-MF) secretion domain (for secreted expression), a multiple cloning site (MCS), the Kluyveromyces lactis LAC4 transcription terminator (TT), and a fungal acetamidase selectable marker gene (amdS) expressed from the yeast ADH2 promoter (PADH₂). In addition, an E. coli replication origin (ORI) and ampicillin resistance gene (ApR) are present for propagation of pKLAC1 in E. coli.

The resulting vectors, i.e., pKlac1-WT-Dc1a, and the various pKlac1-DVP vectors, were then linearized, and transformed into electrocompetent Kluyveromyces lactis host cells, for stable integration of multiple copies of the linearized vectors into the Kluyveromyces lactis host genome at the LAC4 loci.

The transformed Kluyveromyces lactis were then plated on selection agar containing acetamide as the sole nitrogen source to identify strains containing multiple insertions of the expression cassette and its acetamidase selection.

### Example 2. Yield Analysis

Yield Analysis

WT Dc1a and DVP colonies were then cultured for 6 days at 23.5° C. in minimal media with 2% sorbitol and 0.2% corn steep liquor. Expression of folded WT Dc1a and DVP was assessed by HPLC separation on a Chromololith C18 column (EMD) and an elution gradient of 15-35% acetonitrile. Folded and misfolded WT Dc1a and DVP peaks were quantified and compared to controls (minimum of n=4). Because wild-type Dc1a did not have a visible folded peak on the chromatogram, it's total Dc1a produced was estimated by reducing SDS-PAGE Coomassie staining and the ratio of folded to unfolded Dc1a was estimated by quantifying the various Dc1a species after ion-exchange chromatography.

### Example 3. Ion-Exchange Chromatography

Ion-Exchange Chromatography

WT Dc1a and DVP were purified by cation-exchange using SP-Sephadex C-25 (GE Healthcare). Resin was equilibrated in 30 mM sodium acetate buffer, pH 4.0. Spent supernatant containing Dc1a was directly applied to the beads with a pH less than 3.0. Beads were washed and eluted stepwise with 2-3 column volumes (C^{V}) of (1) 30 mM sodium acetate, pH 4.0, (2) 30 mM 2-(N-morpholino)ethanesulfonic acid (MES), pH 6.0, (3) 30 mM MES, pH 6.0, 100 mM sodium chloride, and (4) 30 mM MES, pH 6.0, 200 mM sodium chloride.

For wild-type Dc1a and DVPs that did not contain a mutation that increased the net positive charge, the folded WT Dc1a and DVP eluted as a sharp peak in elution buffer (3) while misfolded versions of Dc1a eluted at the higher salt concentrations of buffer (4). Mutants that increased the overall charge of Dc1a eluted at higher salt concentrations, as expected. Fractions containing folded Dc1a were pooled and dialyzed repeatedly against water to remove salt and buffer. The purified material could be stored indefinitely at −80° C. with no loss in activity, or at 4° C. for longer than 6 months with no loss in activity.

### Example 4. HPLC Standard Curve

HPLC Standard Curve

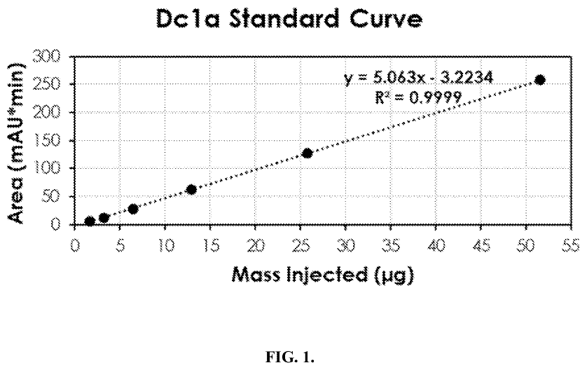

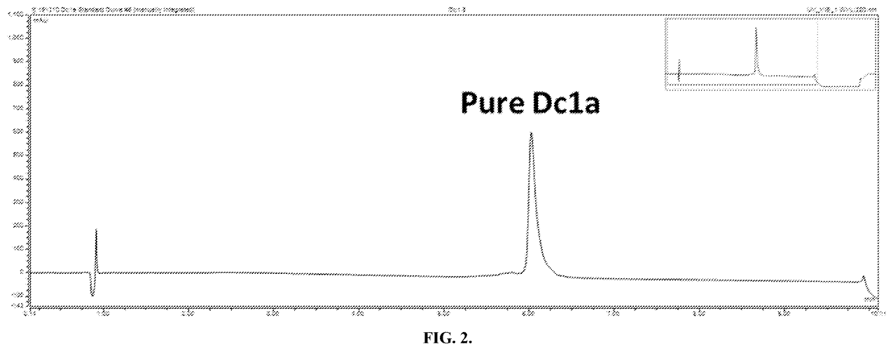

One to two milligrams of WT Dc1a was further purified to >99% purity using HPLC fractionation on a Chromolith C18 column (EMD). After lyophilization, Dc1a was quantified by A280 absorbance using an extinction coefficient (F) of 16180 M-1 cm-1. A HPLC standard curve was set up using a range of concentrations from 5-100 μg and slope was used for quantification of unknown samples. FIGS. 1 and 2.

Briefly, the HPLC standard curve was performed as follows: A serial dilution of purified Dc1a in water was injected onto a Chromolith C18 column (4.6×100 mm) and eluted at a flow rate of 2 mL min⁻¹ and a gradient of 18-3 6% acetonitrile over 8 min. Dc1a peak areas from six samples were plotted against concentration and the slope of the linear relationship was used to quantify the concentration of unknown samples. Samples that reached a height of 1 absorbance units were dropped from the calculation as they were assumed to be out of the linear range of the HPLC detector.

### Example 5. Removal of the Fourth Disulfide Bond at Residues Cys41 and Cys51

Removal of the fourth disulfide bond at residues Cys41 and Cys51

To test whether mutations at residues Cys41 and Cys51 of Dc1a (i.e., the residues where the fourth disulfide connects) could increase expression without affecting activity, a focused mutation scan was performed on each cysteine. Here it was hypothesized that replacing the wild-type amino acid sequence cysteine residues with similarly small amino acids would have a negligible effect on the peptides activity. The focused mutation scan proceeded by mutating the wild-type cysteine of Dc1a to alanine, threonine, serine, or valine. The results of the focused mutation scan revealed that all mutations resulted in improved overall expression and proper folding of Dc1a. Table 2.

The results of the focused mutation analysis of Dc1a revealed that the mutant “T/A” (or “C41T/C51A”) showed the best combination of expression and activity; accordingly, the C41T/C51A variant was used as a background for the alanine scan performed in subsequent experiments.

TABLE 2
Focused mutation of residues Cys41 and Cys51.

| | | | | | SEQ |
|---|---|---|---|---|---|
| | Position | Position | Expression | Insecticidal | ID |
| Name | 41 | 51 | Improvement | Activity | NO |
| WT | Cys | Cys | — | — | 2 |
| C41T/C51A | Thr | Ala | Increased | Similar | 6 |
| C41A/C51A | Ala | Ala | Increased | Similar | 7 |
| C41S/C51A | Ser | Ala | Increased | Similar | 8 |
| C41V/C51A | Val | Ala | Increased | Similar | 9 |
| C41N/C51A | Asn | Ala | No Expression | N/D | 182 |
| C41A/C51T | Ala | Thr | Increased | Similar | 10 |
| C41A/C51S | Ala | Ser | Increased | Similar | 11 |
| C41A/C51V | Ala | Val | Increased | N/D | 12 |
| C41T/C51S | Thr | Ser | Highly Increased | Reduced | 13 |
| C41S/C51S | Ser | Ser | Highly Increased | N/D | 14 |

The calculations used to derive the values displayed in Table 3 are based on the active, folded peak.

### Example 6. Alanine Scan of Dc1a

Alanine scan of Dc1a

To determine which residues might be responsible for either increased expression and/or activity, an alanine scan was performed on the C41T/C51A DVP.

An alanine scan of Dc1a was performed by designing single alanine point mutants at every position. Designed constructs were synthesized and cloned by Twist Biosciences (https://www.twistbioscience.com/; 681 Gateway Blvd South San Francisco, CA 94080). Next, 4-8 transformants were cultured for 6 days at 23.5° C. in minimal media with 2% sorbitol and 0.2% corn steep liquor and their expression was assessed by HPLC quantification. Expression was averaged and normalized to a control (C41T/C51A) and mutants with improved expression were assessed for bioactivity against houseflies.

The alanine scan demonstrated that the mutation of several residues resulted in an observable increase in expression. See Table 3, highlighted gray. These positions were further analyzed for expression, folding, and activity with a mutagenesis screen.

TABLE 3
Alanine scan of C41T/C51A.

| | | Expression | Insecticidal |
|---|---|---|---|
| Position | Residue | Improvement | Activity |
| NA | WT | — | — |
| 2 | Lys | No Change | N/D |
| 3 | Asp | No Change | N/D |
| 4 | Gly | Reduced | N/D |
| 5 | Asp | No Change | N/D |
| 6 | Val | Reduced | N/D |
| 7 | Glu | No Change | N/D |
| 9 | Pro | No Change | N/D |
| 13 | Lys | Reduced | N/D |
| 14 | Lys | No Change | N/D |
| 15 | Tyr | No Change | Reduced |
| 16 | Asp | No Change | N/D |
| 17 | Val | Increased | Similar |
| 18 | Glu | No Change | Reduced |
| 20 | Asp | Increased | Similar |
| 21 | Ser | Increased | Reduced |
| 23 | Glu | No Change | N/D |
| 26 | Gln | No Change | N/D |
| 27 | Lys | Reduced | N/D |
| 28 | Gln | No Change | Reduced |
| 29 | Tyr | Slightly Increased | Reduced |
| 30 | Leu | No Change | Reduced |
| 31 | Trp | Slightly Increased | Reduced |
| 32 | Tyr | Slightly Increased | Reduced |
| 33 | Lys | Reduced | Similar |
| 34 | Trp | No Change | Reduced |
| 35 | Arg | No Change | Reduced |
| 36 | Pro | Slightly Increased | Similar |
| 37 | Leu | Reduced | N/D |
| 38 | Asp | Increased | Similar |
| 40 | Arg | Reduced | N/D |
| 42 | Leu | Slightly Increased | Similar |
| 43 | Lys | Reduced | N/D |
| 44 | Ser | Reduced | N/D |
| 45 | Gly | Slightly Increased | Reduced |
| 46 | Phe | Reduced | N/D |
| 47 | Phe | Slightly Increased | Reduced |
| 48 | Ser | No Change | Similar |
| 49 | Ser | No Change | N/D |
| 50 | Lys | Reduced | N/D |
| 52 | Val | Increased | Reduced |
| 54 | Arg | Increased | Reduced |
| 55 | Asp | No Change | N/D |
| 56 | Val | Reduced | N/D |

A background mutant having a C41T/C51A mutation was further mutated with alanine residues at the indicated positions below.
N/D = not detected.

### Example 7. Mutagenesis Scan of Residues A10, W31, Y32, K33, and P36

Mutagenesis Scan of Residues A10, W31, Y32, K33, and P36

To further elucidate additional positions having an effect on expression and/or activity, a mutagenesis scan of residues A10, W31, Y32, K33, and P36 was performed.

Mutants were synthesized and cloned by Twist Biosciences (https://www.twistbioscience.com/; 681 Gateway Blvd South San Francisco, CA 94080). Here, 4-8 transformants were cultured for 6 days at 23.5° C. in minimal media with 2% sorbitol and 0.200 corn steep liquor and their expression was assessed by HHPLC quantification. Expression was averaged and normalized to a control (C41T/C51A) and mutants with improved expression were assessed for bioactivity against houseflies.

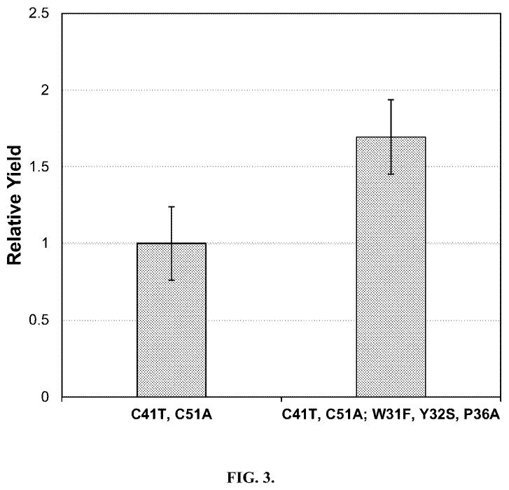

The results of the mutagenesis scan are shown below in Table 4. Position W31 had reduced activity when mutated to alanine, but replacement with phenylalanine resulted in a yield boost and no loss in activity. Position Y32 had a similar yield boost and activity reduction when mutated to alanine but displayed good activity and increased expression when mutated to serine. Mutation of P36 to alanine was superior to other mutations. Combining each mutation together (W31F, Y32S, P36A) resulted in a 6900 increase in expression over the unmodified version. FIG. 3.

TABLE 4
Mutagenesis Scan of residues A10, W31, Y32, K33, and P36.

| Mutation | Expression Improvement | Insecticidal Activity |
|---|---|---|
| A10P | No Change | Reduced |
| A10V | No Change | N/D |
| A10G | Reduced | N/D |
| A10Y | Reduced | N/D |
| A10S | No Change | N/D |
| A10T | No Change | N/D |
| A10K | No Change | N/D |
| A10E | No Change | N/D |
| W31A | Slightly Increased | Reduced |
| W31H | Similar | Reduced |
| W31Y | Similar | Similar |
| W31F | Slightly Increased | Similar |
| W31I | Similar | Reduced |
| W31L | Similar | Reduced |
| W31M | Similar | Reduced |
| W31K | Similar | Reduced |
| W31E | Similar | Reduced |
| W31Q | Similar | Reduced |
| Y32A | Slightly Increased | Reduced |
| Y32V | Reduced | N/D |
| Y32K | Similar | Similar |
| Y32S | Slightly Increased | Similar |
| Y32H | Similar | Similar |
| Y32F | Reduced | N/D |
| Y32L | Reduced | N/D |
| Y32I | Reduced | N/D |
| Y32Q | Similar | Similar |
| Y32E | Similar | Similar |
| K33A | Reduced | Similar |
| K33R | Reduced | N/D |
| K33L | Reduced | N/D |
| K33I | Reduced | N/D |
| K33Q | Reduced | N/D |
| K33N | Reduced | N/D |
| K33E | Reduced | N/D |
| P36A | Slightly Increased | Similar |
| P36Q | Reduced | N/D |
| P36E | Similar | N/D |
| P36K | Similar | N/D |
| P36S | Similar | N/D |
| P36V | Reduced | N/D |

N/D = not detected.

### Example 8. Mutagenesis Scan of Residues V17, D20, and S21

Mutagenesis Scan of Residues V17, D20, and S21

To further elucidate additional positions having an effect on expression and/or activity, a mutagenesis scan of residues V17, D20, and S21 was performed. Mutants were synthesized and cloned as described above.

The results of the mutagenesis scan are shown below in Table 5. Position D20 displayed good expression with no loss of activity; interestingly, only alanine performed better than the wild-type residue at that position. Combining D20A with other variations at positions V17 or L42 resulted in a decrease in expression. Position S21 showed an increase in expression when mutated to alanine, but with reduced activity. No other mutation of S21 could show the same increased expression, so it was not pursued further.

TABLE 5
Mutagenesis Scan of residues V17, D20, S21.

| Mutation | Expression Improvement | Insecticidal Activity |
|---|---|---|
| D20A | Increased | Similar |
| D20K | Similar | N/D |
| D20N | Similar | N/D |
| D20S | Similar | N/D |
| D20Y | Reduced | N/D |
| V17A | Increased | Similar |
| D20A, V17A | Similar | N/D |
| D20A, V17D | Similar | N/D |
| D20A, V17K | Similar | N/D |
| D20A, V17S | Similar | N/D |
| L42A | Slightly Increased | Similar |
| D20A, L42A | Similar | N/D |
| D20A, L42S | Reduced | N/D |
| D20A, L42N | Slightly Increased | N/D |
| D20A, L42V | Slightly Increased | N/D |
| D20A, L42F | Similar | N/D |
| S21A | Increased | Reduced |
| S21G | Reduced | N/D |
| S21P | Reduced | N/D |
| S21T | Reduced | N/D |
| S21V | Reduced | N/D |
| S21D | Reduced | N/D |
| S21N | Reduced | N/D |
| S21K | Similar | N/D |

The mutagenesis scan results shown here were performed on the C41T/C51A background; increases in expression and/or insecticidal activity are relative to that background.

### Example 9. Evaluation of Position D38

Evaluation of Position D38

To further elucidate additional positions having an effect on expression and/or activity, a mutagenesis scan of residue D38 was performed.

Because it gave a large expression boost when mutated to alanine, position D38 was screened by mutational scanning. Then, to identify an optimal combination of mutants for expression, D38A was assessed in combination with L42 or V52 mutants as well as with D20A with or without the previously identified optimized mutants consisting of W31F, Y32S, and P36A. Mutants were synthesized and cloned according to the methods described above. Expression was averaged and normalized to a control (C41T/C51A) and mutants with improved expression were assessed for bioactivity against houseflies.

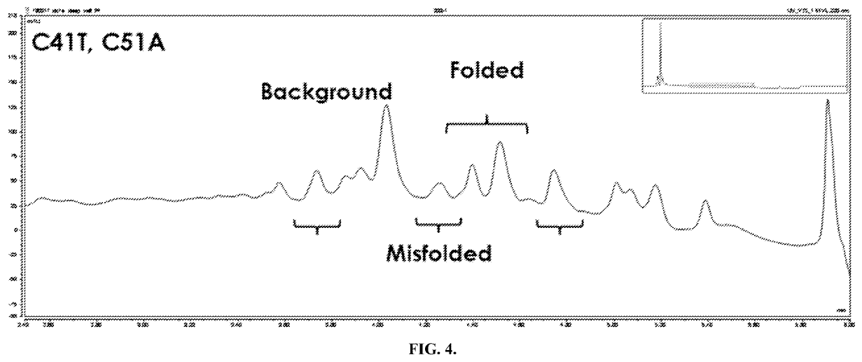

The results of the mutagenesis scan are shown below in Table 6. Position D38A showed a large increase in expression without loss in activity and the number of Dc1a peaks on the HPLC were reduced. See FIGS. 4 and 5. No other mutant showed a similar combination of characteristics. Next, the combination of L42 and V52 mutants were assessed with D38A. While V52 mutants reduced expression, several L42 mutants resulted in increased expression when combined with D38A, with L42V showing the strongest results.

TABLE 6
Evaluation of position D38.

| | Expression | Insecticidal |
|---|---|---|
| Mutant | Improvement | Activity |
| D38A | Increased | Similar |
| D38G | Similar | N/D |
| D38E | Similar | N/D |
| D38K | Increased | N/D |
| D38N | Similar | N/D |
| D38Q | Similar | N/D |
| D38L | Similar | N/D |
| D38S | Increased | N/D |
| D38T | Similar | N/D |
| D38A, V52L | no expression | N/D |
| D38A, V52I | no expression | N/D |
| D38A, V52S | no expression | N/D |
| D38A, V52T | Increased | N/D |
| D38A, V52A | Increased | N/D |
| D38A, V52N | no expression | N/D |
| D38A, V17E | Increased | Reduced |
| D38A, L42I | Similar | N/D |
| D38A, L42V | Increased | Similar |
| D38A, L42S | Increased | N/D |
| D38A, L42E | Increased | N/D |
| D38A, L42Q | Increased | Reduced |
| D38A, L42H | Increased | N/D |
| D20A | Increased | Similar |
| D20A, D38A | Increased | N/D |
| W31F, Y32S, P36A | Slightly Increased | Similar |
| D20A, Y32S | Increased | N/D |
| D38A, Y32S | Increased | N/D |
| D20A, D38A, Y32S | Increased | N/D |
| D20A, W31F, Y32S, P36A | Increased | N/D |
| D38A, W31F, Y32S, P36A | Reduced | N/D |
| D20A, D38A, W31F, | Reduced | N/D |
| Y32S, P36A | | |

The mutagenesis scan results shown here were performed on the C41T/C51A background; accordingly, the relative yields shown are in reference to that background.
N/D = not detected.
Insecticidal activity was assessed against houseflies.

### Example 10. Further Optimization of Cysteine Mutants

Further Optimization of Cysteine Mutants

Re-optimization of the cysteine residues at positions 41 and 51 (i.e., the residues providing connectivity for the fourth disulfide bond) was performed on the D38A DVP. Because two additional mutations were found to be optimal in the previous experiments (i.e., D38A and L42V) near the removed fourth disulfide, mutations of C41 and C51 were re-optimized to find the best possible combination of mutants at these positions when D38A is present. Mutants were synthesized and cloned according to the methods described above.

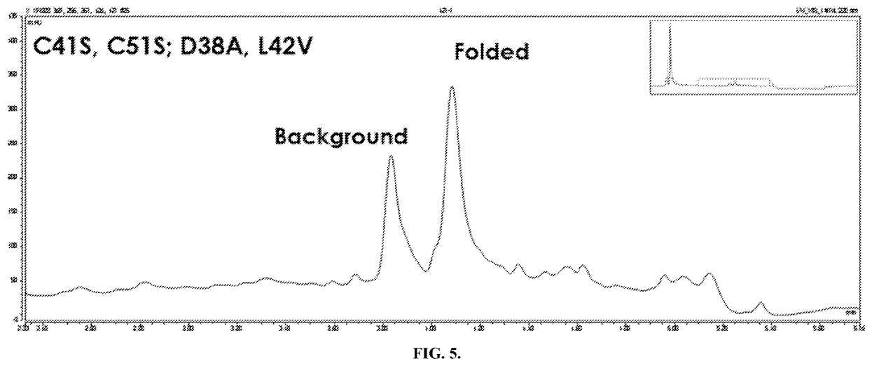

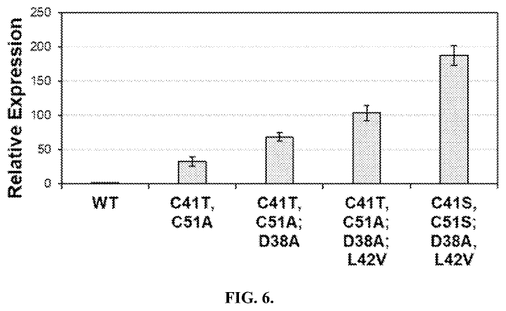

The results re-optimization are shown below in Table 7. Based on the results of the re-optimization scan of the D38A, C41S/C51S was chosen as the best set of disulfide mutants to combine with D38A and L42V. FIGS. 5 and 6.

TABLE 7
Re-optimization of the D38A variant.

| | Position | Position | Expression | Insecticidal |
|---|---|---|---|---|
| Name | 41 | 51 | Improvement | Activity |
| WT | Cys | Cys | — | — |
| C41T/C51A | Thr | Ala | Increased | Similar |
| C41T/C51A/D38A | Thr | Ala | Highly Increased | Similar |
| C41S/C51T/D38A | Ser | Thr | Highly Increased | Similar |
| C41T/C51T/D38A | Thr | Thr | Highly Increased | Similar |
| C41S/C51S/D38A | Ser | Ser | Highly Increased | Similar |
| C41T/C51S/D38A | Thr | Ser | Highly Increased | Similar |
| C41V/C51T/D38A | Val | Thr | Increased | N/D |
| C41T/C51V/D38A | Thr | Val | Increased | N/D |
| C41S/C51V/D38A | Ser | Val | Highly Increased | N/D |
| C41V/C51S/D38A | Val | Ser | No Expression | N/D |

N/D = not detected.

Consequently, individual and combined mutations were compared for expression and activity in a head-to-head assay. The DVP possessing a combination of the four mutations explored herein, i.e., C41S/C51S/D38A/L42V, having the amino acid sequence of “AKDGDVEGPAGCKKYDVECDSGECCQKQYLWYKWRPLACRSVKSGFFSSKSVCRDV” (SEQ ID NO:53), increased expression by nearly 200 fold over WT without loss in housefly bioactivity. FIG. 6.

### Example 11. Housefly Injections

Housefly Injections

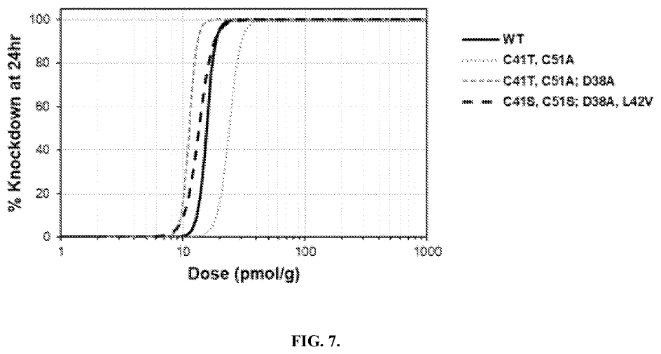

Adult houseflies (Musca domestica) weighing 14-20 mg were anesthetized using CO₂ and 0.5 μL was injected intrathoracically with WT Dc1a and the following DVPs: (1) C41T/C51A; (2) C41T/C51A/D38A; and (3) C41S/C51S/D38A/L42V. Results are shown in FIG. 7.

Dose-response curves were generated by assessing flies for percent knockdown (i.e., the inability to walk) at 24 hours (% Knockdown at 24 hr). Under CO₂ controls, the flies regain the ability to walk after several minutes, followed shortly thereafter by the ability to take flight. Flies dosed with intermediate levels of Dc1a regained the ability to stand and walk; however, these flies were unable to regain the ability to take flight. After 15-30 minutes post-injection, flies began to display flaccid paralysis interspersed by brief episodes of spastic paralysis that culminated in intensity after 1 hour, and resulted in the inability to stand. Flies that were still paralyzed but not dead at 24 hours displayed flaccid paralysis and never recovered even up to 72 hours post-injection, when mortality from dehydration occurred. FIG. 7.

As shown in FIG. 7, the DVPs C41T/C51A/D38A and C41S/C51S/D38A/L42V showed superior knockdown ability when compared to WT-Dc1a. To achieve 50% knockdown at 24-hours, C41T/C51A/D38A required a dose of 11.3 μmol/g, and C41S/C51S/D38A/L42V required a dose of 13.5 μmol/g; alternatively, WT-Dc1a required a dose of 15.6 μmol/g.

### Example 12. Corn Earworm (CEW) Injections

Corn Earworm (CEW) Injections

An assay evaluating DVPs injected into CEWs was performed as follows: Corn earworm (Helicoverpa zea) larvae were injected in their fourth instar. Eggs of H. zea were purchased (Benzon, Carlisle, PA) and reared to fourth instar on General Purpose Lepidoptera Diet (Frontier Agricultural Science, Newark, DE). Prior to injection larvae were weighed in order to calculate μmol/g doses. Injections volumes were 1 μL, and were performed with a 30 gauge needle and glass syringe in a hand microapplicator (Burkard, Rickmansworth, Herts, England). Following the injection, larvae were placed in a new enclosure with General Purpose Lepidoptera Diet and their condition (including mortality, sublethal effects, and behavior) was evaluated 24-hours post-injection.

Here, wild-type Dc1a, and C41T/C51A/D38A (SEQ ID NO:29) and C41S/C51S/D38A/L42V (SEQ ID NO:53) were injected into CEW, and percent knockdown was assessed at 24 hours.

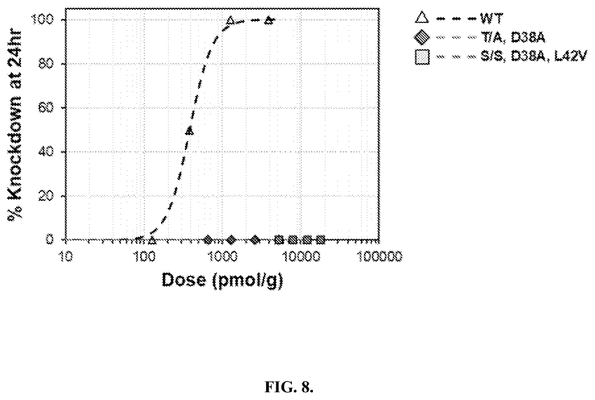

As shown in FIG. 8, injection of the cysteine removal mutants resulted in reduced CEW mortality by several orders of magnitude. The loss of activity appeared to be localized to the cystine bond positions (C41 and C51) as a C41A/C51A mutant had no activity at a high dose of 2500 μmol/g. A table comparing housefly and CEW knockdown is presented below.

TABLE 8
Comparison of housefly and CEW mortality.

| | Housefly LD₅₀ | CEW LD₅₀ |
|---|---|---|
| Name | (pmol/g) | (pmol/g) |
| WT | 38 | 385 |
| C41A/C51A | 45 | >2500 |
| C41T/C51A/D38A | 30 | >5288 |
| C41S/C51S/D38A/L42V | 50 | >18133 |

### Example 13. Mutations Improving CEW Insecticidal Activity

Mutations Improving CEW Insecticidal Activity

Mutations of Dc1a were made to screen for recovery of CEW activity. Here, mutants were synthesized and cloned according the methods described above. Briefly, 4 individual transformants were cultured for 6 days at 23.5° C. in minimal media with 200 sorbitol and 0.2% corn steep liquor. Supernatants were combined and concentrated 10× using centrifugal filtration cassettes (Pall) with a 3000 Da molecular weight cutoff. Concentrates were then injected into Corn earworm (Helicoverpa zea).

Corn earworm (CEW) larvae were injected in their fourth instar. Eggs of H zea were purchased (Benzon Research, 7 Kuhn Dr, Carlisle, PA, 17015) and reared to fourth instar on General Purpose Lepidoptera Diet (Frontier Agricultural Science, Newark, DE). Prior to injection larvae were weighed in order to calculate μmol/g doses. Injections volumes are 1 μL, and were performed with a 30 gauge needle and glass syringe in a hand microapplicator (Burkard, Rickmansworth, Herts, England). The injection site was near the base of one of the hindmost prolegs. Following the injection, larvae are placed in a new enclosure with General Purpose Lepidoptera Diet and their condition (including mortality, sublethal effects, and behavior) is evaluated 24-hours post-injection.

TABLE 9
Screen for mutants improving CEW activity.

| | | | SEQ |
|---|---|---|---|
| Mu-diguetoxin-Dc1a Variant | | | ID |
| Polypeptide Name | Activity | Amino Acid Sequence | NO |
| C41T/C51A/D38A/L42V | No | AKDGDVEGPAGCKKYDVECDSGECCQKQ | 35 |
| | | YLWYKWRPLACRTVKSGFFSSKAVCRDV | |
| C41N/C51A/D38A/L42V | No | AKDGDVEGPAGCKKYDVECDSGECCQKQ | 125 |
| | | YLWYKWRPLACRNVKSGFFSSKAVCRDV | |
| C41D/C51A/D38A/L42V | No | AKDGDVEGPAGCKKYDVECDSGECCQKQ | 126 |
| | | YLWYKWRPLACRDVKSGFFSSKAVCRDV | |
| C41S/C51A/D38A/L42V | No | AKDGDVEGPAGCKKYDVECDSGECCQKQ | 127 |
| | | YLWYKWRPLACRSVKSGFFSSKAVCRDV | |
| C41M/C51A/D38A/L42V | Yes | AKDGDVEGPAGCKKYDVECDSGECCQKQ | 128 |
| | | YLWYKWRPLACRMVKSGFFSSKAVCRDV | |
| C41T/C51G/D38A/L42V | No | AKDGDVEGPAGCKKYDVECDSGECCQKQ | 129 |
| | | YLWYKWRPLACRTVKSGFFSSKGVCRDV | |
| C41T/C51D/D38A/L42V | No | AKDGDVEGPAGCKKYDVECDSGECCQKQ | 130 |
| | | YLWYKWRPLACRTVKSGFFSSKDVCRDV | |
| C41T/C51N/D38A/L42V | No | AKDGDVEGPAGCKKYDVECDSGECCQKQ | 131 |
| | | YLWYKWRPLACRTVKSGFFSSKNVCRDV | |
| C41T/C51Q/D38A/L42V | No | AKDGDVEGPAGCKKYDVECDSGECCQKQ | 132 |
| | | YLWYKWRPLACRTVKSGFFSSKQVCRDV | |
| C41T/C51E/D38A/L42V | No | AKDGDVEGPAGCKKYDVECDSGECCQKQ | 133 |
| | | YLWYKWRPLACRTVKSGFFSSKEVCRDV | |
| C41T/C51V/D38A/L42V | No | AKDGDVEGPAGCKKYDVECDSGECCQKQ | 134 |
| | | YLWYKWRPLACRTVKSGFFSSKVVCRDV | |
| C41T/C51H/D38A/L42V | No | AKDGDVEGPAGCKKYDVECDSGECCQKQ | 135 |
| | | YLWYKWRPLACRTVKSGFFSSKHVCRDV | |
| C41T/C51M/D38A/L42V | Yes | AKDGDVEGPAGCKKYDVECDSGECCQKQ | 136 |
| | | YLWYKWRPLACRTVKSGFFSSKMVCRDV | |
| C41V/C51V/D38A/L42V | No | AKDGDVEGPAGCKKYDVECDSGECCQKQ | 137 |
| | | YLWYKWRPLACRVVKSGFFSSKVVCRDV | |
| C41M/C51M/D38A/L42V | No | AKDGDVEGPAGCKKYDVECDSGECCQKQ | 138 |
| | | YLWYKWRPLACRMVKSGFFSSKMVCRDV | |
| C41K/C51E/D38A/L42V | Yes | AKDGDVEGPAGCKKYDVECDSGECCQKQ | 139 |
| | | YLWYKWRPLACRKVKSGFFSSKEVCRDV | |
| C41E/C51K/D38A/L42V | Yes | AKDGDVEGPAGCKKYDVECDSGECCQKQ | 140 |
| | | YLWYKWRPLACREVKSGFFSSKKVCRDV | |
| C41T/C51A/D38A/L42V/D20V | No | AKDGDVEGPAGCKKYDVECVSGECCQKQ | 141 |
| | | YLWYKWRPLACRTVKSGFFSSKAVCRDV | |
| C41T/C51A/D38A/L42V/D20G | No | AKDGDVEGPAGCKKYDVECGSGECCQKQ | 142 |
| | | YLWYKWRPLACRTVKSGFFSSKAVCRDV | |
| C41T/C51A/D38A/L42V/D20K | No | AKDGDVEGPAGCKKYDVECKSGECCQKQ | 143 |
| | | YLWYKWRPLACRTVKSGFFSSKAVCRDV | |
| C41T/C51A/D38A/L42V/D20E | Yes | AKDGDVEGPAGCKKYDVECESGECCQKQ | 144 |
| | | YLWYKWRPLACRTVKSGFFSSKAVCRDV | |
| C41T/C51A/D38A/L42V/D20L | No | AKDGDVEGPAGCKKYDVECLSGECCQKQ | 145 |
| | | YLWYKWRPLACRTVKSGFFSSKAVCRDV | |
| C41T/C51A/D38A/L42V/D20N | Yes | AKDGDVEGPAGCKKYDVECNSGECCQKQ | 146 |
| | | YLWYKWRPLACRTVKSGFFSSKAVCRDV | |
| C41T/C51A/D38A/L42V/D20Y | Yes | AKDGDVEGPAGCKKYDVECYSGECCQKQ | 147 |
| | | YLWYKWRPLACRTVKSGFFSSKAVCRDV | |
| C41T/C51A/D38A/L42V/S21G | No | AKDGDVEGPAGCKKYDVECDGGECCQKQ | 148 |
| | | YLWYKWRPLACRTVKSGFFSSKAVCRDV | |
| C41T/C51A/D38A/L42V/E18P | No | AKDGDVEGPAGCKKYDVPCDSGECCQKQ | 149 |
| | | YLWYKWRPLACRTVKSGFFSSKAVCRDV | |
| C41T/C51A/D38A/L42V/E18K | No | AKDGDVEGPAGCKKYDVKCDSGECCQKQ | 150 |
| | | YLWYKWRPLACRTVKSGFFSSKAVCRDV | |
| C41T/C51A/D38A/L42V/E18S | No | AKDGDVEGPAGCKKYDVSCDSGECCQKQ | 151 |
| | | YLWYKWRPLACRTVKSGFFSSKAVCRDV | |
| C41T/C51A/D38A/L42V/E18D | No | AKDGDVEGPAGCKKYDVDCDSGECCQKQ | 152 |
| | | YLWYKWRPLACRTVKSGFFSSKAVCRDV | |

TABLE 10
CEW knockdown and expression analysis.

| | | | SEQ |
|---|---|---|---|
| Mu-diguetoxin-Dc1a | | | ID |
| Variant Polypeptide Name | CEW KD₅₀ | Expression | NO. |
| C41T/C51A/D38A/L42V | Reduced | Highly Increased | 35 |
| C41V/C51T/D38A/L42V | Similar | Reduced | 180 |
| C41V/C51V/D38A/L42V | Slightly | Reduced | 137 |
| | Reduced | | |
| C41M/C51A/D38A/L42V | Similar | Highly Increased | 128 |
| C41T/C51D/D38A/L42V | Reduced | Highly Increased | 130 |
| C41K/C51E/D38A/L42V | Reduced | Highly Increased | 139 |
| C41T/C51A/D38A/L42V/ | Similar | Highly Increased | 147 |
| D20Y | | | |

After characterization, a valine or methionine in position C41 was shown to result in higher activity similar to WT, though valine reduces yield. Mutation of D20 to tyrosine was also able to recover WT-like activity even when the mutant was C41T/C51A.

### Example 14. Expression of DVP-Insecticidal Proteins in Plants

Expression of DVP-Insecticidal Proteins in Plants

The expression of DVP-insecticidal proteins in a plant, plant tissue, plant cell, plant seed, or part thereof, was evaluated. Here, the cloning and expression of DVP-insecticidal proteins was performed using a tobacco transient expression system technology referred to as FECT (Liu Z & Kearney C M, BMC Biotechnology, 2010, 10:88, the disclosure of which is incorporated herein by reference in its entirety).

Briefly, the FECT vector contains a T-DNA region for agroinfection, which contains a CaMV 35S promoter that drives the expression of the foxtail mosaic virus RNA without the genes encoding the viral coating protein and the triple gene block. In the place of the coating protein and triple block are a pair of subcloning sites (Pac I and Avr II) that allow a DVP ORF to be subcloned N′ to C′ following the Pac I site for high levels of transient viral expression. This “disarmed” virus genome prevents plant to plant transmission. In addition to the FECT vector subcloned to express the DVPs, a second FECT vector is co-expressed that encodes P19, a RNA silencing suppressor protein from tomato bushy stunt virus, to prevent the post-transcriptional gene silencing (PTGS) of the introduced T-DNA. Agrobacterium containing the transient plant expression system were injected into the leaves of tobacco (Nicotiana benthamiana) as described below.

The DVP-insecticidal proteins examined here comprised the following components: an endoplasmic reticulum signal peptide (ERSP); a ubiquitin monomer; an intervening linker peptide; and a Histidine tag.

The ERSP motif used was the Barley Alpha-Amylase Signal peptide (BAAS), a 24 amino acid peptide with the following amino acid sequence (N′ to C′; one letter code):

(SEQ ID NO: 60)
MANKHLSLSLFLVLLGLSASLASG.

The Zea mays ubiquitin monomer used was a 75 amino acid peptide with the following amino acid sequence (N′ to C′, one letter code):

(SEQ ID NO: 183)
QIFVKTLTGKTITLEVESSDTIDNVKAKIQDKEGIPPDQQRLIFAGKQL
EDGRTLADYNIQKESTLHLVLRLRGG (NCBI Accession No.
XP_020404049.1)

The polynucleotide operable to encode a DVP ORF used in the DVP-insecticidal proteins are found in Table 11 below.

The intervening linking peptide used had the following amino acid sequence (N′to C′, one letter code): ALKFLV (SEQ ID NO:184) or IGER (SEQ ID NO:54).

The histidine tag used had the following amino acid sequence (N′ to C′, one letter code): HHHHHH (SEQ ID NO:185).

Thus, an exemplary DVP-insecticidal protein used in this example has a construct with the following elements and orientation:

ERSP-UBI-L-DVP-HIS

An example of a full amino acid sequence for DVP-insecticidal protein is as follows:

(SEQ ID NO: 186)
MANKHLSLSLFLVLLGLSASLASGQIFVKTLTGKTITLEVESSDTIDNV
KAKIQDKEGIPPDQQRLIFAGKQLEDGRTLADYNIQKESTLHLVLRLRG
GALKFLVAKDGDVEGPAGCKKYDVECDSGECCQKQYLWYKWRPLDCRCL
KSGFFSSKCVCRDVHHHHHH

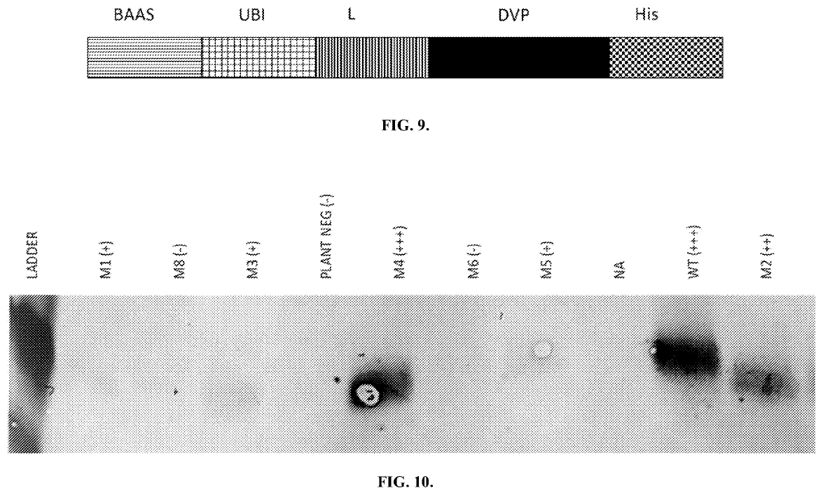

A general schematic of the DVP-insecticidal protein is shown in FIG. 9. Here, the foregoing construct has components that are defined as follows: “ERSP” refers to the endoplasmic reticulum signal peptide; “UBI” refers to the ubiquitin monomer; “DVP” refers to the Mu-diguetoxin-Dc1a toxin or DVP; “L” refers to intervening linker peptide; and “HIS” refers to the Histidine tag.

Next, a polynucleotide operable to encode the DVP-insecticidal protein, i.e., DNA with the following ORF: “BAAS:UBI:L:DVP:HIS” or “baas-ubi-1-dvp-his” (where BAAS is the ERSP; UBI is ubiquitin; and L is linking peptide), was cloned into the Pac I and Avr II restriction sites of the FECT expression vector to create the transient vectors. These transient vectors were then transformed into Agrobacterium tumefaciens strain, GV3101 cells using a freeze-thaw method as follows: the stored competent GV3101 cells were thawed on ice and then mixed with 1-5 μg pure transient vectors DNA. The cell-DNA mixture was then kept on ice for 5 minutes, and transferred to −80° C. for 5 minutes; the mixture was then incubated in a 37° C. water bath for 5 minutes. The freeze-thaw treated cells were then diluted into 1 mL LB medium, and shaken on a rocking table for 2-4 hours at room temperature. The cell-LB mixture was then spun down at 5,000 rcf for 2 minutes to pellet cells, and then 800 μL of LB supernatant was removed. The cells were then resuspended in the remaining liquid, and the entire volume (approximately 200 μL) of the transformed cell-LB mixture was spread onto LB agar plates with the appropriate antibiotics (i.e., 10 μg/mL rifampicin, 25 μg/mL gentamycin, and 50 μg/mL kanamycin), and incubated at 28° C. for two days. The resulting transformed colonies were then picked and cultured in 6 mL aliquots of LB medium with the appropriate antibiotics necessary for transformed DNA analysis and creating glycerol stocks of the transformed GV3101 cells.

The transformed GV3101 cells were then streaked onto an LB plate with the appropriate antibiotics (as described above) from the previously created glycerol stock, and incubated at 28° C. for two days. A colony of transformed GV3101 cells was used to inoculate 5 mL of LB-MESA medium (LB media supplemented with 10 mM MES, 20 μM acetosyringone), and the same antibiotics described above. The colony was then grown overnight at 28° C.; the cells were then collected by centrifugation at 5000 rpm for 10 minutes, and resuspended in the induction medium (10 mM MES, 10 mM MgCl2, 100 μM acetosyringone) at a final OD600 of 1.0. The cells were then incubated in the induction medium for 2 hours, to overnight, at room temperature. At this point, the cells were ready for transient transformation of tobacco leaves.

Because FECT uses a mixture of P19 expression and the gene of interest expression, cultures of cells for the pFECT-P19 transformed GV3101 cells and the gene of interest cultures were mixed together in equal amounts for infiltration of tobacco leaves before injection into the plant leaves. The treated cells were infiltrated into the underside of attached leaves of Nicotiana benthamiana plants by injection, using a 3 mL syringe without a needle attached. Protein expression in tobacco leaves was evaluated at 6-8 days post-infiltration.

Full length DVP-insecticidal protein was purified from the tobacco by using a manual extraction technique. Leaf tissue was obtained via 30 mm diameter punch, from the infiltrated area, rolled up and placed inside a 2 mL conical bottom tube with two, 5/32 inch diameter stainless steel grinding balls, and frozen in liquid nitrogen. The samples were then homogenized using a Troemner-Talboys High Throughput Homogenizer. Next, a 750 μL ice-cold total soluble protein (TSP) extraction solution (sodium phosphate solution 50 mM, EDTA 1 mM, pH 7.0) was added into the tube and vortexed. The microtube was then left to incubate at room temperature for 15 minutes, and then centrifuged at 16,000×g for 15 minutes at 4° C. Next, 100 μL of the resulting supernatant was taken and loaded into pre-Sephadex G-50-packed column in 0.45 μm Millipore MultiScreen filter microtiter plate with empty receiving Costar microtiter plate on bottom. The microtiter plates were then centrifuged at 800 g for 2 minutes at 4° C. The resulting filtrate solution (hereinafter “total soluble protein extract” or “TSP extract”) of the tobacco leaves, was ready for downstream analysis.

The samples were then analyzed using standard Western Blotting techniques. Samples were prepared for a protein gel by mixing 10 μL of protein sample with 9 μL Invitrogen 2X SDS loading buffer and 2 μL Novex 10X Reducing agent, and heating the sample at 85° C. for 5 minutes. The samples were then loaded and ran on a Novex Precast, 16% Tricine gel in 1× Invitrogen Tricine running buffer with 0.1% sodium thioglycolate in the top tank and Invitrogen SeeBlue Plus 2 MWM. The gel was run at 150V for 75 minutes. The gel was then transferred to a Novel PVDF membrane using a 7-minute transfer program on the iBLOT system. Once the transfer was complete, the blot membrane was then moved to a container and washed with Buffer A (1× TBS made from Quality Biological's 10× TBS (0.25M tris base, 1.37M NaCl, 0.03M KCL, pH 7.4)), for five minutes by rocking gently at room temperature. This was then followed with a blocking step using Buffer B (Buffer A with 1% BSA) for 1 hour. The blot was then rinsed three times with 5 minute washes of Buffer C (Buffer B with 0.05% Tween 20). This was followed with a 1:10000 dilution of Maine Biotech Anti-His antibody in Buffer C for 1 hour. The blot was then rinsed three times with Buffer C for 5 minutes each. This was followed with a 1:3000 dilution of BioRad goat anti-mouse AP conjugated antibody (secondary antibody) in Buffer C for 1 hour. The blot was then rinsed with two times with Buffer C for 5 minutes each and once with Buffer A for 5 minutes. The blot is then developed with BioRad AP developer and stopped by rinsing with water.

FIG. 10 depicts a His-Tag western blot of plant expressed dc1a and mutants. Each well represents crude plant extracts run under denaturing protein gel conditions and visualized with standard western blot techniques. The short name for the samples tested in the western blot are listed above the image along with a rating system for expression. The symbol (−) indicates that there is no protein detected on the blot and if protein is detected, the symbol (+) to (+++) indicate the relative amount detected by visual inspection. The lane indicated “LADDER” shows the molecular weight marker. Lanes “PLANT NEG” show the negative control (i.e., GFP expressing tobacco protein extract). Lanes labeled with “M #” indicate the short name for the DVP-insecticidal protein evaluated, which can be found in the table below. Lane “WT” shows the DVP-insecticidal protein with the WT Mu-diguetoxin-Dc1a protein.

TABLE 11
Summary of DVP-insecticidal proteins tested and results for transient
plant expression and insect activity (insect activity assessed in housefly assay
in Example 15, below). Here, the “DVP sequence” refers to the DVP in the DVP-
insecticidal construct: “ERSP-UBI-L-DVP-HIS”; all other peptide elements in the
construct remain the same as described above.

| Western Blot | | | SEQ | | |
|---|---|---|---|---|---|
| Lane | Mutations | DVP sequence | ID NO | Expression | Activity |
| WT | NA | AKDGDVEGPAGCKKYDVECDS | 2 | Y | Y |
| | | GECCQKQYLWYKWRPLDCRCL | | | |
| | | KSGFFSSKCVCRDV | | | |
| M1 | Y32S, P36A | AKDGDVEGPAGCKKYDVECDS | 187 | Y | Y |
| | | GECCQKQYLWSKWRALDCRCL | | | |
| | | KSGFFSSKCVCRDV | | | |
| M2 | Y32K, P36A | AKDGDVEGPAGCKKYDVECDS | 188 | Y | Y |
| | | GECCQKQYLWKKWRALDCRCL | | | |
| | | KSGFFSSKCVCRDV | | | |
| M3 | Y32H, P36A | AKDGDVEGPAGCKKYDVECDS | 189 | Y | Y |
| | | GECCQKQYLWHKWRALDCRCL | | | |
| | | KSGFFSSKCVCRDV | | | |
| M4 | W31F, Y32S | AKDGDVEGPAGCKKYDVECDS | 190 | Y | Y |
| | | GECCQKQYLFSKWRPLDCRCL | | | |
| | | KSGFFSSKCVCRDV | | | |
| M5 | W31F, Y32S, | AKDGDVEGPAGCKKYDVECDS | 191 | Y | Y |
| | P36A | GECCQKQYLFSKWRALDCRCL | | | |
| | | KSGFFSSKCVCRDV | | | |
| M6* | C41A, C51A | AKDGDVEGPAGCKKYDVECDS | 7 | ND | NA |
| | | GECCQKQYLWYKWRPLDCRAL | | | |
| | | KSGFFSSKAVCRDV | | | |
| M8* | Y32H, P36A, | AKDGDVEGPAGCKKYDVECDS | 192 | ND | NA |
| | C41A, C51A | GECCQKQYLWHKWRALDCRAL | | | |
| | | KSGFFSSKAVCRDV | | | |

*“ND” means not detected. “NA” means not applicable. Expression of the M6 and M8 DVP-insecticidal proteins comprising the construct: ERSP-UBI-L-DVP-HIS, wherein the DVP is the M6 or M8 corresponding DVP in the table above, were not detected in this experiment using Nicotiana benthamiana; accordingly, activity of the M6 and M8 mutants could not be assessed, and are therefore not applicable.

### Example 15. Housefly Injection Assay with Plant-Expressed Proteins

Housefly Injection Assay with Plant-Expressed Proteins

Houseflies were injected with the TSP extract obtained from the plant extraction process described above. Prior to injection, adult houseflies (Musca domestica) were immobilized with CO₂, and selected for injection based on weight (12-20 mg). A microapplicator, loaded with a 1 cc syringe and 30-gauge needle, was used to inject 0.5.L of a given treatment (negative control or non-naturally occurring Mu-diguetoxin-Dc1a insecticidal proteins) per fly into houseflies through the body wall of the dorsal thorax. The injected houseflies were placed into closed containers with moist filter paper and breathing holes on the lids, and were evaluated based on impacted scoring 2 hours post-injection. Impacted scores include knock-down and dead.

The results of the housefly injection assay are presented below.

TABLE 12
Fly injection results for percent impacted 2-hours post-injection
of DVP-insecticidal proteins expressed in plants. Here,
the short name M# are the same as described above.

| Sample | Mutation | % Impacted |
|---|---|---|
| Plant Extract (Neg) | NA | 0 |
| CO₂ Control | NA | 0 |
| WT | NA | 100 |
| M1 | Y32S, P36A | 60 |
| M2 | Y32K, P36A | 60 |
| M3 | Y32H, P36A | 80 |
| M4 | W31F, Y32S | 100 |
| M5 | W31F, Y32S, P36A | 60 |
| M6 | C41A, C51A | 0 |
| M8 | Y32H, P36A, C41A, C51A | 0 |

### Example 16. High Yield DVPs

The DVP having amino acid substitutions of C41S, C5S, and D38A, i.e., “AKDGDVEGPAGCKKYDVECDSGECCQKQYLWYKWRPLACRSLKSGFFSSKSVCRDV” (C41S/C51S/D38A; SEQ TD NO: 47) was further evaluated to determine if point mutations to SEQ TD NO: 47 could result in improved expression. To this C41S/C51S/D38A DVP background, the following additional mutations were made: L42I; K2L; Y32S; K2L+Y32S; D38T; D38S; and D38M.

Polynucleotide constructs were synthesized, cloned, and expressed as described above, and yield was normalized to the average yield of the C41S/C51S/D38A DVP (SEQ TD NO: 47). Constructs were created that were operable to encode the DVPs shown in the table below.

TABLE 13
High yield DVPs. The C41S/C51S/D38A DVP (SEQ ID NO: 47) was
further mutated to include the following mutations: L42I; K2L; Y32S; K2L +
Y32S; D38T; D38S; and D38M.

| Name | Sequence | SEQ ID NO. |
|---|---|---|
| C41S/C51S/D38A | AKDGDVEGPAGCKKYDVECDSGECCQKQYLWYKWRPLACRSLKS | 47 |
| | GFFSSKSVCRDV | |
| D38A/L42I/C41S/ | AKDGDVEGPAGCKKYDVECDSGECCQKQYLWYKWRPLACRSIKS | 210 |
| C51S | GFFSSKSVCRDV | |
| K2L/D38A/C41S/ | ALDGDVEGPAGCKKYDVECDSGECCQKQYLWYKWRPLACRSLKS | 211 |
| C51S | GFFSSKSVCRDV | |
| Y32S/D38A/C41S/ | AKDGDVEGPAGCKKYDVECDSGECCQKQYLWSKWRPLACRSLKS | 212 |
| C51S | GFFSSKSVCRDV | |
| K2L/Y32S/D38A/ | ALDGDVEGPAGCKKYDVECDSGECCQKQYLWSKWRPLACRSLKS | 213 |
| C41S/C51S | GFFSSKSVCRDV | |
| D38T/C41S/C51S | AKDGDVEGPAGCKKYDVECDSGECCQKQYLWYKWRPLTCRSLKS | 214 |
| | GFFSSKSVCRDV | |
| D38S/C41S/C51S | AKDGDVEGPAGCKKYDVECDSGECCQKQYLWYKWRPLSCRSLKS | 215 |
| | GFFSSKSVCRDV | |
| D38M/C41S/C51S | AKDGDVEGPAGCKKYDVECDSGECCQKQYLWYKWRPLMCRSLKS | 216 |
| | GFFSSKSVCRDV | |

The polynucleotide constructs operable to encode the DV-Ps in Table 13 were inserted into a pKlac1 vector (Catalog No. N3740; New England Biolabs®; 240 County Road, Ipswich, MA 01938-2723) as described above (see Example 1). The resulting vectors were then linearized, and transformed into electrocompetent Kluyveromyces lactis host cells, for stable integration of multiple copies of the linearized vectors into the Kluyveromyces lactis host genome at the LAC4 loci. The transformed Kluyveromyces lactis were then plated on selection agar containing acetamide as the sole nitrogen source to identify strains containing multiple insertions of the expression cassette and its acetamidase selection.

Colonies were then cultured for 6 days at 23.5° C. in minimal media with 2% sorbitol and 0.2% corn steep liquor. Expression of the DVPs was assessed by HPLC separation on a Chromololith C18 column (EMD) and an elution gradient of 15-35% acetonitrile. One to two milligrams of WT ApsIII was further purified to >99% purity using HPLC fractionation on a Chromolith C18 column (EMD). After lyophilization, WT ApsIII was quantified by A280 absorbance using an extinction coefficient (F) of 16180 M-1 cm-1. A HPLC standard curve was set up using a range of concentrations from 5-100 μg and slope was used for quantification of unknown samples.

The yield of DVPs of SEQ ID NOs: 210-219 was compared the yield of the DVP of SEQ ID NO: 47. Yield was determined based on rpHPLC peak area and then normalized to total integrated gene copies. To determine gene copy measurement, gDNA was extracted using a Yeast gDNA Extraction kit (ThermoFisherScientific) and copy number was determined by qPCR analysis using the delta delta Ct (ΔΔCt) method. Peak areas were normalized to the C41S/C51S/D38A DVP background (SEQ ID NO: 47).

As shown in FIG. 11, the additional mutations to the C41S/C51S/D38A DVP background, i.e., L42I; K2L; Y32S; K2L+Y32S; D38T; and D38S; all possessed improved yield relative to the C41S/C51S/D38A DVP background (SEQ ID NO: 47) control.

### Example 17. Mutations Improving High Yield DVPs

DVPs identified in Example 16 were further evaluated. Here, the following DVPs were compared to wild-type Dc1a (SEQ ID NO:2): (1) a K2L/Y32S/L42I DVP having the amino acid sequence:

“ALDGDVEGPAGCKKYDVECDSGECCQKQYLWSKWRPLDCRCIKSGFFSSKCVCRDV” (SEQ ID NO: 217); and (2) a K2L/Y32S/D38A/L42I/C41S/C51S DVP having the amino acid sequence:

(SEQ ID NO: 218)
“ALDGDVEGPAGCKKYDVECDSGECCQKQYLWSKWRPLACRSIKSGFFS
SKSVCRDV”.

The foregoing DVPs were synthesized and cloned according to the methods described in Example 16. Expression was determined by rpHPLC as described in Example 16. Peak areas were normalized to wildtype Dc1a (SEQ ID NO: 2). The table below provides a summary of the DVPs, and their fold improvement in expression.

TABLE 14
Mutations improving the yield of DVPs. The yield of the DVPs:
K2L/Y32S/L42I DVP (SEQ ID NO: 217); and K2L/Y32S/D38A/L42I/C41S/C51S
DVP (SEQ ID NO: 218), were compared to WT Dc1a (SEQ ID NO: 2).

| | | Fold | SEQ ID |
|---|---|---|---|
| Name | Sequence | improvement | NO. |
| K2L/Y32S/L42I | ALDGDVEGPAGCKKYDVECDSGECCQK | 19 | 217 |
| | QYLWSKWRPLDCRCIKSGFFSSKCVCR | | |
| | DV | | |
| K2L/Y32S/D38A/ | ALDGDVEGPAGCKKYDVECDSGECCQK | 63 | 218 |
| L42I/C41S/C51S | QYLWSKWRPLACRSIKSGFFSSKSVCR | | |
| | DV | | |

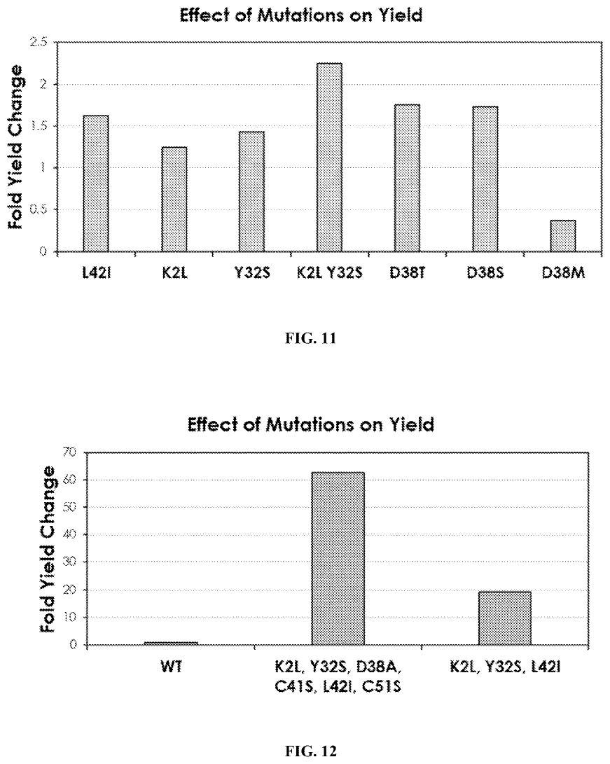

As shown in FIG. 12, and Table 14, combining the mutations K2L, Y32S, and L42I (SEQ ID NO: 217) dramatically improved the yield compared to wild-type Dc1a (19-fold improvement). Furthermore, combining the K2L, Y32S, and L42I mutations in the C41S/C51S/D38A DVP background mutant, which includes a disulfide bond deletion (i.e., C41S/C41S), resulted in an even greater increase in expression (63-fold improvement). FIG. 12.

### Example 18. Cystine-Knot Architecture: Overview

Cystine-Knot Architecture

The present invention contemplates and teaches methods of engineering a recombinant CRP comprising, consisting essentially of, or consisting of, a cystine knot (CK) architecture according to Formula (II):

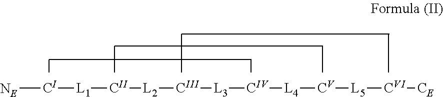

wherein C^{I} to C^{VI} are cysteine residues; wherein cysteine residues C^{I} and C^{IV} are connected by a first disulfide bond; C^{II} and C^{V} are connected by a second disulfide bond; and C^{III} and C^{VI} are connected by a third disulfide bond; wherein the first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif, wherein N_{E}, L₁, L₂, L₃, L₄, L₅, and C_{E} are peptide subunits comprising an amino acid sequence having a length of 1 to 13 amino acid residues; wherein N_{E}, L₃, C_{E}, or any combination thereof, are optionally absent; wherein said recombinant CRP is created by modifying a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif, wherein the modifiable CRP is modified by removing one or more non-CK disulfide bonds from a modifiable CRP having one or more non-CK disulfide bonds; wherein removing the one or more disulfide bonds from the modifiable CRP having one or more non-CK disulfide bonds, results in the recombinant CRP having the CK architecture according to Formula (II); and wherein the recombinant CRP having the CK architecture according to Formula (II) has an increased level of expression relative to a level of expression of a modifiable CRP that does not have the CK architecture according to Formula (II).

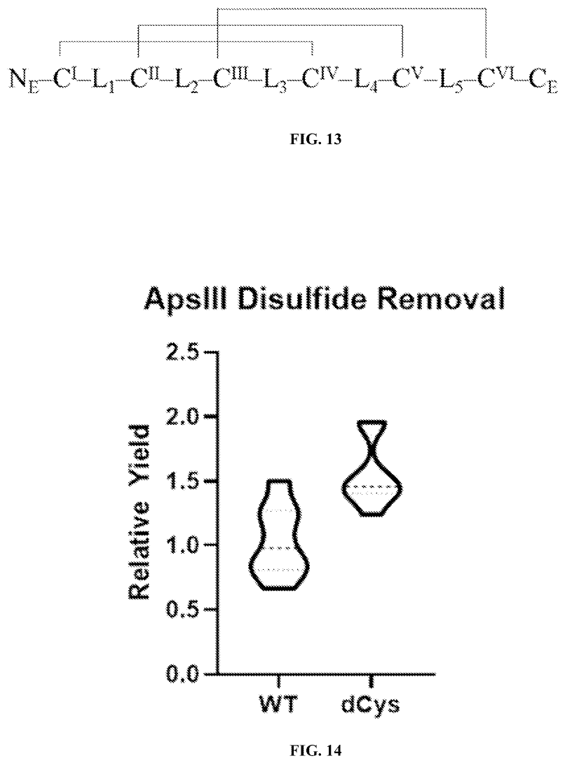

FIG. 13 depicts a schematic showing Formula (II), which describes a recombinant cysteine rich protein (CRP) having a cystine knot (CK) architecture. Here, C^{I} to C^{VI} are cysteine residues; cysteine residues C^{I} and C^{IV} are connected by a first disulfide bond; C^{II} and C^{V} are connected by a second disulfide bond; and C^{III} and C^{VI} are connected by a third disulfide bond; (disulfide bonds are indicated by lines connecting cysteine residues). The first disulfide bond, the second disulfide bond, and the third disulfide bond have a disulfide bond topology that forms a cystine knot motif, wherein the first disulfide bond, second disulfide bond, and third disulfide bond are the only disulfide bonds that form the cystine knot motif. N_{E}, L₁, L₂, L₃, L₄, L₅, and C_{E} are peptide subunits each comprising an amino acid sequence having a length of 1 to 13 amino acid residues. In some embodiments, N_{E}, L₃, C_{E}, or any combination thereof, are optionally absent.

### Example 19. Arriving at the CK Architecture of Formula (II): ApsIII

The protein Mu-cyrtautoxin-As1a (also known as “ApsIII” or “Aps-3”) is a modifiable CRP that was modified to have a CK architecture according to Formula (II). ApsIII is an insecticidal protein found in the trap-door spider, Apomastus schlingeri. An exemplary wild-type ApsIII protein is provided herein, having the amino acid sequence of a

SEQ ID NO: 193
“CNSKGTPCTNADECCGGKCAYNVWNCIGGGCSKTCGY” (NCBI
Accession No. P49268.1).

The wild-type ApsIII protein has four disulfide bonds at positions 1 to 15; 8 to 19; 14 to 35; and 26 to 31. The disulfide bonds at positions 1 to 15; 8 to 19; 14 to 35 have a disulfide bond topology that forms a cystine knot motif, and, the disulfide bond spanning positions 26 to 31 represents a non-CK disulfide bond, i.e., a disulfide bond that does not take part in creating the cystine knot motif. Accordingly, the non-CK disulfide bond spanning positions 26 to 31 was removed to create a recombinant ApsIII having a CK architecture according to Formula (II).

Polynucleotide constructs were synthesized, cloned, and expressed as described above and yield was normalized to the average of wildtype ApsIII. Results show a near 50% improvement in yield when the extra, non-core ICK disulfide was removed.

Briefly, the following constructs were created: a polynucleotide (SEQ ID NO: 194) operable to encode a recombinant wild-type ApsIII (SEQ ID NO: 195) and a polynucleotide (SEQ ID NO: 196) operable to encode an ApsIII dCys mutant (SEQ ID NO: 197). Here, the ApsIII dCys mutant has a C26A and a C31A mutation relative to the WT ApsIII sequence set forth in SEQ ID NO: 193. The C26A and C31A mutations remove the fourth disulfide bond.

These polynucleotide constructs were inserted into a pKlac1 vector (Catalog No. N3740; New England Biolabs®; 240 County Road, Ipswich, MA 01938-2723) as described above (see Example 1). The resulting vectors were then linearized, and transformed into electrocompetent Kluyveromyces lactis host cells, for stable integration of multiple copies of the linearized vectors into the Kluyveromyces lactis host genome at the LAC4 loci. The transformed Kluyveromyces lactis were then plated on selection agar containing acetamide as the sole nitrogen source to identify strains containing multiple insertions of the expression cassette and its acetamidase selection.

Colonies were then cultured for 6 days at 23.5° C. in minimal media with 2% sorbitol and 0.2% corn steep liquor. Expression of WT ApsIII and ApsIII dCys was assessed by HPLC separation on a Chromololith C18 column (EMD) and an elution gradient of 15-35% acetonitrile. One to two milligrams of WT ApsIII was further purified to >99% purity using HPLC fractionation on a Chromolith C18 column (EMD). After lyophilization, WT ApsIII was quantified by A280 absorbance using an extinction coefficient (F) of 16180 M-1 cm-1. A HPLC standard curve was set up using a range of concentrations from 5-100 μg and slope was used for quantification of unknown samples.

The yield of WT ApsIII and ApsIII dCys (n=8 for each) was determined based on rpHPLC peak area and then normalized to total integrated gene copies. To determine gene copy measurement, gDNA was extracted using a Yeast gDNA Extraction kit (ThermoFisherScientific) and copy number was determined by qPCR analysis using the delta delta Ct (ΔΔCt) method. Violin plots showing relative yield were generated using Graphpad Prism ver. 9.2.0. FIG. 14. Results show a 50.2% improvement in yield when the extra, non-CK disulfide was removed.

### Example 20. Arriving at the CK Architecture of Formula (I): K-ACTX Peptide

The modifiable CRP, Kappa-ACTX peptide (also known “Kappa-ACTX” or “x-ACTX”), was modified to have a CK architecture according to Formula (II).

Kappa-ACTX is a member of a family of insecticidal inhibitor cystine knot (ICK) peptides that have been isolated from an Australian funnel-web spiders belonging to the Atracinae subfamily. One such spider is known as the Australian Blue Mountains Funnel-web Spider, which has the scientific name Haydronyche versuta. An exemplary wild-type Kappa-ACTX peptide is provided herein, having the amino acid sequence:

(SEQ ID NO: 198)
“AICTGADRPCAACCPCCPGTSCKAESNGVSYCRKDEP”
(UniProtKB/Swiss-Prot No. P82228.1).

The wild-type Kappa-ACTX protein has four disulfide bonds at positions 3-17; 10-22; 13-14; and 16-32. The disulfide bonds at positions 3-17, 10-22, and 16-32 are disulfide bonds that form a cystine knot motif, and the disulfide bond topology forms an ICK. The disulfide bond spanning positions 13-14 represents a non-CK disulfide bond, i.e., a disulfide bond that does not take part in creating the cystine knot motif (i.e., the ICK). Accordingly, the non-CK disulfide bond spanning positions 13-14 was removed to create a recombinant Kappa-ACTX having a CK architecture according to Formula (II).

Polynucleotide constructs encoding Kappa-ACTX ORFs were synthesized and cloned by Twist Biosciences (https://www.twistbioscience.com; 681 Gateway Blvd South San Francisco, CA 94080). The Kappa-ACTX ORFs encoded the following proteins: WT Kappa-ACTX (SEQ ID NO: 198); a Kappa-ACTX mutant having a C13A mutation (SEQ ID NO: 199); a Kappa-ACTX mutant having a C14A mutation (SEQ ID NO: 200); and a Kappa-ACTX mutant having a C13A and C14A mutation (SEQ ID NO: 201).

The constructs were codon optimized and synthesized as a fusion with Kluyveromyces lactis alpha mating factor pre/pro sequence (αMF) and ligated into the NotI and HindIII restriction sites of pKlac1 (Catalog No. N3740; New England Biolabs®; 240 County Road, Ipswich, MA 01938-2723). The pKlac1 vector contains the Kluyveromyces lactis P_{LAC4-PBI} promoter (1), DNA encoding the K. lactis α-mating factor (α-MF) secretion domain (for secreted expression), a multiple cloning site (MCS), the Kluyveromyces lactis LAC4 transcription terminator (TT), and a fungal acetamidase selectable marker gene (amdS) expressed from the yeast ADH2 promoter (P_{ADH2}). In addition, an E. coli replication origin (ORI) and ampicillin resistance gene (Ap^{R}) are present for propagation of pKLAC1 in E. coli.

The vector was digested with SacII to linearize and remove the bacterial Ori and selection marker, then electroporated into electrocompetent Kluyveromyces lactis cells. Colonies were then cultured for 6 days at 23.5° C. in minimal media with 2% sorbitol and 0.2% corn steep liquor. Multiple gene copy transformants were selected on selection plates containing acetamide as the sole nitrogen source.

Yield comparisons were based on peak area (mAU) as determined in the HPLC procedure described above. Briefly, clones expressing protein were assessed by HPLC on a Chromolith C18 column (4.6×100 mm) and eluted at a flow rate of 2 mL min-1 and gradient of 5-30% acetonitrile over 5 min.

The Table below shows the results of removing the vicinal disulfide bond from Kappa-ACTX in order to arrive at the CK architecture according to Formula (II).

TABLE 15
Increase in level of expression in Kappa-ACTX mutants having the CK
architecture according to Formula (II).

| | Expression | | SEQ ID |
|---|---|---|---|
| Mutant | Improvement | Sequence | NO. |
| WT | 1 | AICTGADRPCAACCPCCPGTSCKAESNGVSYCRKDEP | 198 |
| C13A | 8.5x higher | AICTGADRPCAAACPCCPGTSCKAESNGVSYCRKDEP | 199 |
| C14A | 0 | AICTGADRPCAACAPCCPGTSCKAESNGVSYCRKDEP | 200 |
| C13A, C14A | 6.3x higher | AICTGADRPCAAAAPCCPGTSCKAESNGVSYCRKDEP | 201 |

As shown in Table 14 above, modifying Kappa-ACTX to have a CK architecture according to Formula (II) resulted in an increased yield.

1.-100. (canceled)

101. A diguetoxin variant polypeptide (DVP) having insecticidal activity against one or more insect species, said DVP comprising an amino acid sequence that is at least 80%, 85%, 90%, 95%, or at least 100% identical to the amino acid sequence according to Formula (I): A-Xₗ-D-G-D-V-E-G-P-A-G-C-K-K-Y-D-X₂-E-C-X₃-X₄-G-E-C-C-Q-K-Q-Y-L-X₅-X₆-K-W-R- X₇-L-X8-C-R-X₉-X₁₀K-S-G-F-F-S-S-K-X₁₁X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ iS C, G, T, A, S, M, or V; X₁₀ is L, A, N, V, S, E, I, or Q; X₁₁ is C, F, A, T, S, M, or V; and X₁₂ is V, A, or T, or a pharmaceutically acceptable salt thereof.

102. The DVP of claim 101, wherein if X₉ is G, T, A, S, M or V, or X₁₁ is F, A, T, S, M or V, then a disulfide bond is removed.

103. The DVP of claim 101, wherein the DVP comprises an amino sequence as set forth in any one of SEQ ID NOs: 6-_{43, 45}-51, 53, 128, 130, 136, 139-140, 144, 146-147, 187-191, 202-215, or 217-219, or a pharmaceutically acceptable salt thereof.

104. A composition comprising a DVP of claim 101, and an excipient.

105. A polynucleotide operable to encode a DVP, said DVP comprising an amino acid sequence that is at least 80%, 85%, 90%, 95%, or at least 100% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W-R-X₇-L-X₈-C-R-X₉-X₁₀-K-S-G-F-F-S-S-K-X₁₁ wherein the polypeptide comprises at least one amino acid substitution relative to the wild-type sequence of the diguetoxin as set forth in SEQ ID NO:2, wherein X₁ is K or L; X₂ is V, A, or E; X₃ is D, Y, or A; X₄ is S or A; X₅ is W, A, F; X₆ is Y, A, S, H, or K; X₇is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T, or a complementary nucleotide sequence thereof.

106. The polynucleotide of claim 105, wherein if the polynucleotide encodes a DVP wherein if X₉ is G, T, A, S, M or V, or X₁₁ is F, A, T, S, M or V, then a disulfide bond is removed.

107. The polynucleotide of claim 105, wherein the polynucleotide encodes a DVP having an amino sequence as set forth in any one of SEQ ID NOs: 6-43, 45-51, 53, 128, 130, 136, 139-140, 144, 146-147, 187-191, 202-215, or 217-219.

108. A method of producing a DVP, the method comprising:
(a) preparing a vector comprising a first expression cassette comprising a polynucleotide operable to encode a DVP, or complementary nucleotide sequence thereof, said DVP comprising an amino acid sequence that is at least 80%, 85%, 90%, or at least 95% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K-W R-X₇-L-X₈-C-R-X₉-X₁₀-K-S-G-F-F-S-S-K-X₁₁ X₁₂-C-R-D-V, wherein the polypeptide comprises at least one amino acid substitution relative to the wild-type sequence of the diguetoxin as set forth in SEQ ID NO:2, and wherein X₁ is K or L; X₂ is V, A, or E; X₃ is D, Y, or A; X₄ is S or A; X₅ is W, A, F; X₆ is Y, A, S, H, or K; X₇is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T;
(b) introducing the vector into a yeast cell; and
(c) growing the yeast cell in a growth medium under conditions operable to enable expression of the DVP and secretion into the growth medium.

109. The method of claim 108, wherein the DVP comprises an amino sequence as set forth in any one of SEQ ID NOs: 6-43, 45-51, 53, 128, 130, 136, 139-140, 144, 146-147, 187-191, 202-215, or 217-219, or a pharmaceutically acceptable salt thereof.

110. The method of claim 108, wherein the yeast cell is selected from any species of the genera Saccharomyces, Pichia, Kluyveromyces, Hansenula, Yarrowia or Schizosaccharomyces.

111. The method of claim 110, wherein the yeast cell is selected from the group consisting of Kluyveromyces lactis, Kluyveromyces marxianus, Saccharomyces cerevisiae, and Pichia pastoris.

112. The method of claim 111, wherein the yeast cell is Kluyveromyces lactis.

113. The method of claim 108, wherein the vector comprises two or three expression cassettes, each expression cassette operable to encode the DVP of the first expression cassette, or a DVP of a different expression cassette, and wherein each of the expression cassette encodes a DVP having an amino acid sequence as set forth in any one of SEQ ID NOs: 6-43, 45-51, 53, 128, 130, 136, 139-140, 144, 146-147, 187-191, 202-215, or 217-219.

114. A method of combating, controlling, or inhibiting a pest, comprising applying a pesticidally effective amount of the composition of claim 104, to the locus of the pest, or to a plant or animal susceptible to an attack by a pest, wherein the pest may be selected from the group consisting of: Achema Sphinx Moth (Hornworm) (Eumorpha achemon); Alfalfa Caterpillar (Colias eurytheme); Almond Moth (Caudra cautella); Amorbia Moth (Amorbia humerosana); Armyworm (Spodoptera spp., e.g. exigua, frugiperda, littoralis, Pseudaletia unipuncta); Artichoke Plume Moth (Platyptilia carduidactyla); Azalea Caterpillar (Datana major); Bagworm (Thyridopteryx); ephemeraeformis); Banana Moth (Hypercompe scribonia); Banana Skipper (Erionota thrax); Blackheaded Budworm (Acleris gloverana); California Oakworm (Phryganidia californica); Spring Cankerworm (Paleacrita merriccata); Cherry Fruitworm (Grapholita packardi); China Mark Moth (Nymphula stagnata); Citrus Cutworm (Xylomyges curialis); Codling Moth (Cydia pomonella); Cranberry Fruitworm (Acrobasis vaccinii); Cross-striped Cabbageworm (Evergestis rimosalis); Cutworm (Noctuid species, Agrotis ipsilon); Douglas Fir Tussock Moth (Orgyia pseudotsugata); Ello Moth (Hornworm) (Erinnyis ello); Elm Spanworm (Ennomos subsignaria); European Grapevine Moth (Lobesia botrana); European Skipper (Thymelicus lineola); Essex Skipper; Fall Webworm (Melissopus latiferreanus)); Filbert Leafroller (Archips rosanus)); Fruittree Leafroller (Archips argyrospilia)); Grape Berry Moth (Paralobesia viteana)); Grape Leafroller (Platynota stultana)); Grapeleaf Skeletonizer (Harrisina americana); Green Cloverworm (Plathypena scabra)); Greenstriped Mapleworm (Dryocampa rubicunda)); Gummosos-Batrachedra comosae (Hodges); Gypsy Moth (Lymantria dispar); Hemlock Looper (Lambdina fiscellaria); Hornworm (Manduca spp.); Imported Cabbageworm (Pieris rapae); Io Moth (Automeris io); Jack Pine Budworm (Choristoneura pinus); Light Brown Apple Moth (Epiphyas postvittana); Melonworm (Diaphania hyalinata); Mimosa Webworm (Homadaula anisocentra); Obliquebanded Leafroller (Choristoneura rosaceana); Oleander Moth (Syntomeida epilais); Omnivorous Leafroller (Playnota stultana); Omnivorous Looper (Sabulodes aegrotata); Orangedog (Papilio cresphontes); Orange Tortrix (Argyrotaenia citrana); Oriental Fruit Moth (Grapholita molesta); Peach Twig Borer (Anarsia lineatella); Pine Butterfly (Neophasia menapia); Podworm; Redbanded Leafroller (Argyrotaenia velutinana); Redhumped Caterpillar (Schizura concinna); Rindworm Complex (Various Leps.); Saddleback Caterpillar (Sibine stimulea); Saddle Prominent Caterpillar Heterocampa guttivitta); Saltmarsh Caterpillar (Estigmene acrea); Sod Webworm (Crambus spp.); Spanworm (Ennomos subsignaria); Fall Cankerworm (Alsophila pometaria); Spruce Budworm (Choristoneura fumiferana); Tent Caterpillar (Various Lasiocampidae); Thecla-Thecla Basilides (Geyr) (Thecla basilides); Tobacco Hornworm (Manduca sexta); Tobacco Moth (Ephestia elutella); Tufted Apple Budmoth (Platynota idaeusalis); Twig Borer (Anarsia lineatella); Variegated Cutworm (Peridroma saucia); Variegated Leafroller (Platynota flavedana); Velvetbean Caterpillar (Anticarsia gemmatalis); Walnut Caterpillar (Datana integerrima); Webworm (Hyphantria cunea); Western Tussock Moth (Orgyia vetusta); Southern Cornstalk Borer (Diatraea crambidoides); Corn Earworm; Sweet potato weevil; Pepper weevil; Citrus root weevil; Strawberry root weevil; Pecan weevil); Filbert weevil; Ricewater weevil; Alfalfa weevil; Clover weevil; Tea shot-hole borer; Root weevil; Sugarcane beetle; Coffee berry borer; Annual blue grass weevil (Listronotus maculicollis); Asiatic garden beetle (Maladera castanea); European chafer (Rhizotroqus majalis); Green June beetle (Cotinis nitida); Japanese beetle (Popillia japonica); May or June beetle (Phyllophaga sp.);
Northern masked chafer (Cyclocephala borealis); Oriental beetle (Anomala orientalis); Southern masked chafer (Cyclocephala lurida); Billbug (Curculionoidea); Aedes aegypti; Busseola fusca; Chilo suppressalis; Culex pipiens; Culex quinquefasciatus; Diabrotica virgifera; Diatraea saccharalis; Helicoverpa armigera; Helicoverpa zea; Heliothis virescens; Leptinotarsa decemlineata; Ostrinia furnacalis; Ostrinia nubilalis; Pectinophora gossypiella; Plodia interpunctella; Plutella xylostella; Pseudoplusia includens; Spodoptera exigua; Spodoptera frugiperda; Spodoptera littoralis; Trichoplusia ni; and Xanthogaleruca luteola.

115. A vector comprising a polynucleotide operable to encode a DVP having an amino acid sequence that is at least 80%, 85%, 90%, 95%, or at least 100% identical to an amino acid sequence as set forth in any one of SEQ ID NOs: 6-43, 45-51, 53, 128, 130, 136, 139-140, 144, 146-147, 187-191, 202-215, or 217-219.

116. A yeast cell comprising:
a first expression cassette comprising a polynucleotide operable to encode a DVP, said DVP comprising an amino acid sequence that is at least 80%, 85%, 90%, 95%, or at least 100% identical to the amino acid sequence according to Formula (I): A-X₁-D-G-D-V-E-G-P-A-G-C-K-K-Y-D-X₂-E-C-X₃-X₄-G-E-C-C-Q-K-Q-Y-L-X₅-X₆-K W-R-X₇-L-X₈-C-R-X₉-X₁₀- K-S-G-F-F-S-S-K-X₁₁-X₁₂-C-R-D-V, wherein the polypeptide comprises at least one amino acid substitution relative to the wild-type sequence of the diguetoxin as set forth in SEQ ID NO:2, wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T; or complementary nucleotide sequence thereof.

117. The yeast cell of claim 116, wherein if X₉ is G, T, A, S, M or V, or X₁₁ is F, A, T, S, M or V, then a disulfide bond is removed.

118. The yeast cell of claim 116, wherein the DVP comprises an amino sequence as set forth in any one of SEQ ID NOs: 6-43, 45-51, 53, 128, 130, 136, 139-140, 144, 146-147, 187-191, 202-215, or 217-219.

119. The yeast cell of claim 116, wherein the yeast cell is selected from any species of the genera Saccharomyces, Pichia, Kluyveromyces, Hansenula, Yarrowia or Schizosaccharomyces.

120. The yeast cell of claim 119, wherein the yeast cell is selected from the group consisting of Kluyveromyces lactis, Kluyveromyces marxianus, Saccharomyces cerevisiae, and Pichia pastoris.

121. A recombinant cysteine-rich protein (CRP), said recombinant CRP comprising a cystine knot (CK) architecture according to Formula (II): 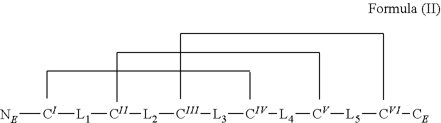
wherein C^{I} to C^{VI} are cysteine residues;
wherein cysteine residues C^{I} and C^{IV} are connected by a first disulfide bond; C^{II} and C^{V} are connected by a second disulfide bond; and C^{III} and C^{VI} re connected by a third disulfide bond;
wherein the first disulfide bond, the second disulfide bond, and the third disulfide bond have a disulfide bond topology that forms a cystine knot motif;
wherein the first disulfide bond, second disulfide bond, and third disulfide bond are the only disulfide bonds that form the cystine knot motif;
wherein N_{E}, L₁, L₂, L₃, L₄, L₅, and C_{E} are peptide subunits comprising an amino acid sequence having a length of 1 to 13 amino acid residues;
wherein N_{E}, L₃, C_{E}, or a combination thereof, are optionally absent;
wherein said recombinant CRP is created by modifying a modifiable CRP having: one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif;
wherein the modifiable CRP is modified by removing one or more non-CK disulfide bonds from a modifiable CRP having one or more non-CK disulfide bonds;
wherein removing the one or more disulfide bonds from the modifiable CRP having one or more non-CK disulfide bonds, results in the recombinant CRP having the CK architecture according to Formula (II); and
wherein the recombinant CRP having the CK architecture according to Formula (II) has an increased level of expression relative to a level of expression of a modifiable CRP that does not have the CK architecture according to Formula (II).

122. The recombinant CRP of claim 121, wherein the disulfide bond topology forms one of the following cystine knot motifs: an inhibitor cystine knot (ICK) motif; a growth factor cystine knot (GFCK) motif; or a cyclic cystine knot (CCK) motif.

123. The recombinant CRP of claim 122, wherein the disulfide bond topology forms an ICK motif.

124. The recombinant CRP of claim 121, wherein the modifiable CRP is a wild-type μ-DGTX-Dcla; a DVP; a Kappa-ACTX, an ApsIII, or a variant thereof.

125. The recombinant CRP of claim 121, wherein the modifiable or recombinant CRP comprises an amino acid sequence as set forth in any one of SEQ ID NOs: 1-2, 6-14, 193, 195, 197, 198, 199, or 201.

126. A method of making a recombinant cysteine-rich protein (CRP) comprising a cystine knot (CK) architecture according to Formula (II): 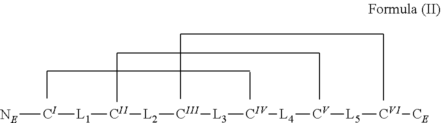
wherein C^{I} to C^{VI} are cysteine residues;
wherein cysteine residues C^{I} and C^{IV} are connected by a first disulfide bond; C^{II} and C^{V} are connected by a second disulfide bond; and C^{III} and C^{VI} are connected by a third disulfide bond;
wherein the first disulfide bond, the second disulfide bond, and the third disulfide bond have a disulfide bond topology that forms a cystine knot motif;
wherein the first disulfide bond, second disulfide bond, and third disulfide bond are the only disulfide bonds that form the cystine knot motif;
wherein N_{E}, L_{,}L₂, L₃, L₄, L₅, and C_{E} are peptide subunits comprising an amino acid sequence having a length of 1 to 13 amino acid residues;
wherein N_{E}, L₃, C_{E}, or a combination thereof, are optionally absent;
said method comprising:
(a) providing a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif; and
(b) modifying the modifiable CRP by removing one or more non-CK disulfide bonds from a modifiable CRP having one or more non-CK disulfide bonds;
wherein removing the one or more disulfide bonds from the modifiable CRP having one or more non-CK disulfide bonds, results in the recombinant CRP having the CK architecture according to Formula (II); and
wherein the recombinant CRP having the CK architecture according to Formula (II) has an increased level of expression relative to a level of expression of a modifiable CRP that does not have the CK architecture according to Formula (II).

127. The method of claim 126, wherein the disulfide bond topology forms one of the following cystine knot motifs: an inhibitor cystine knot (ICK) motif; a growth factor cystine knot (GFCK) motif; or a cyclic cystine knot (CCK) motif.

128. The method of claim 126, wherein the modifiable CRP is a wild-type μ-DGTX-Dcla; a DVP; a Kappa-ACTX, an ApsIII, or a variant thereof.

129. The method of claim 126, wherein the modifiable or recombinant CRP comprises an amino acid sequence as set forth in any one of SEQ ID NOs: 1-2, 6-14, 193, 195, 197, 198, 199, or 201.

130. A method of increasing the yield of a recombinant cysteine-rich protein (CRP), said method comprising:
(a) creating a recombinant CRP having a cystine knot (CK) architecture according to Formula (II): 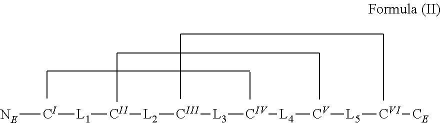
wherein C^{I} to C^{VI} are cysteine residues;
wherein cysteine residues C^{I} and C^{IV} are connected by a first disulfide bond; C^{II} and C^{V} are connected by a second disulfide bond; and C^{III} and C^{VI} are connected by a third disulfide bond;
wherein the first disulfide bond, the second disulfide bond, and the third disulfide bond have a disulfide bond topology that forms a cystine knot motif;
wherein the first disulfide bond, second disulfide bond, and third disulfide bond are the only disulfide bonds that form the cystine knot motif;
wherein N_{E}, L₁, L₂, L₃, L₄, L₅, and C_{E} are peptide subunits comprising an amino acid sequence having a length of 1 to 13 amino acid residues;
wherein N_{E}, L₃, C_{E}, or any combination thereof, are optionally absent;
wherein said recombinant CRP is created according to the following process:
(b) providing a modifiable CRP having one or more non-CK disulfide bonds, wherein the one or more non-CK disulfide bonds are not the first disulfide bond, the second disulfide bond, or the third disulfide bond, and wherein the one or more non-CK disulfide bonds do not form the CK motif;
(c) modifying the modifiable CRP by removing one or more non-CK disulfide bonds from the modifiable CRP having one or more non-CK disulfide bonds;
wherein removing the one or more disulfide bonds from the modifiable CRP having one or more non-CK disulfide bonds results in the recombinant CRP having the CK architecture according to Formula (II); and
wherein the recombinant CRP having the CK architecture according to Formula (II) has an increased level of expression relative to a level of expression of a modifiable CRP that does not have the CK architecture according to Formula (II).

131. The method of claim 130, wherein the disulfide bond topology forms one of the following cystine knot motifs: an inhibitor cystine knot (ICK) motif; a growth factor cystine knot (GFCK) motif; or a cyclic cystine knot (CCK) motif.

132. The method of claim 131, wherein the disulfide bond topology forms an ICK motif.

133. The method of claim 130, wherein the modifiable CRP is a wild-type μ-DGTX-Dcla; a DVP; a Kappa-ACTX, an ApsIII, or a variant thereof.

134. The method of claim 130, wherein the modifiable or recombinant CRP comprises an amino acid sequence as set forth in any one of SEQ ID NOs: 1-2, 6-14, 193, 195, 197, 198, 199, or 201.

135. A diguetoxin variant polypeptide (DVP) having insecticidal activity against one or more insect species, said DVP consisting of an amino acid sequence that is at least 80%, 85%, 90%, 95%, or at least 100% identical to the amino acid sequence set forth in any one of SEQ ID NOs: 6-43, 45-51, 53, 128, 130, 136, 139-140, 144, 146-147, 187-191, 202-215, or 217-219, or a pharmaceutically acceptable salt thereof.

136. The diguetoxin variant polypeptide (DVP) of claim 135, wherein the DVP consists of an amino acid sequence that is at least 80%, 85%, 90%, 95%, or at least 100% identical to the amino acid sequence set forth in any one of SEQ ID NOs: 47, 53, 136, 139-140, 144, 146-147, 187-191, 210-215, or 217-219, or a pharmaceutically acceptable salt thereof.

137. A fusion protein comprising one or more DVPs operably linked to an alpha mating factor (alpha-MF) peptide; wherein said one or more DVPs have an amino acid sequence that is at least 80%, 85%, 90%, 95%, or at least 100% identical to the amino acid sequence according to Formula (I): A-X₁-D-G-D-V-E-G-P-A-G-C-K-K-Y-D-X₂-E-C-X₃-X₄-G-E-C-Q-K-Q-Y-L-X₅-X₆-K-W-R-X₇-L-X₈-C-R-X₉-X₁₀-K-S-G-F-F-S-S-K-X₁₁-X₁₂-C- R-D-V, wherein the DVP comprises at least one amino acid substitution relative to the wild-type sequence of the diguetoxin as set forth in SEQ ID NO:2, wherein X₁ is K or L; X₂ is V, A, or E; X₃ is D, Y, or A; X₄ is S or A; X₅ is W, A, F; X₆ is Y, A, S, H, or K; X₇ is P or A; X₈ is D, A, K, S, T or M; X₉ is C, G, T, A, S, M, or V; X₁₀ is L, A, N, V, S, E, I, or Q; X₁₁ is C, F, A, T, S, M, or V; and X₁₂ is V, A, or T, or a pharmaceutically acceptable salt thereof.

138. The fusion protein of claim 137, wherein if X₉ is G, T, A, S, M or V, or X₁₁ is F, A, T, S, M or V, then a disulfide bond is removed.

139. The fusion protein of claim 137, wherein the one or more DVPs comprise an amino sequence as set forth in any one of SEQ ID NOs: 6-43, 45-51, 53, 128, 130, 136, 139-140, 144, 146-147, 187-191, 202-215, or 217-219.

140. The fusion protein of claim 137, wherein the one or more DVPs, the alpha-MF, or a combination thereof, are separated by a cleavable linker or non-cleavable linker.

141. The fusion protein of claim 140, wherein the alpha-MF peptide is an alpha-MF peptide derived from a yeast species, such as a species selected from any species of the genera Saccharomyces, Pichia, Kluyveromyces, Hansenula, Yarrowia or Schizosaccharomyces.


SEQUENCE LISTING (249 sequences)

SEQ ID NO: 1 - protein, 94 aa, Diguetia canities
MKVFVVLLCLSLAAVYALEERLDKDADIMLDSPADMERAKDGDVEGPAGCKKYDVECDSGECCQKQYLWYKWRPLDCRCLKSGFFSSKCVCRDV

SEQ ID NO: 2 - protein, 56 aa, Diguetia canities
AKDGDVEGPAGCKKYDVECDSGECCQKQYLWYKWRPLDCRCLKSGFFSSKCVCRDV

SEQ ID NO: 3 - protein, 56 aa, Diguetia canities; identical to SEQ ID NO: 2

SEQ ID NO: 4 - protein, 56 aa, Artificial sequence
  Disulfide deletion
AKDGDVEGPAGCKKYDVECDSGECCQKQYLWYKWRPLDCRGLKSGFFSSKFVCRDV

SEQ ID NO: 5 - protein, 56 aa, Artificial sequence; identical to SEQ ID NO: 4
  Disulfide deletion

SEQ ID NO: 6 - protein, 56 aa, Artificial sequence
  C41T/ C51A DVP
AKDGDVEGPAGCKKYDVECDSGECCQKQYLWYKWRPLDCRTLKSGFFSSKAVCRDV

SEQ ID NO: 7 - protein, 56 aa, Artificial sequence
  C41A/ C51A (A/A) DVP
AKDGDVEGPAGCKKYDVECDSGECCQKQYLWYKWRPLDCRALKSGFFSSKAVCRDV

SEQ ID NO: 8 - protein, 56 aa, Artificial sequence
  C41S/ C51A (S/A) DVP
AKDGDVEGPAGCKKYDVECDSGECCQKQYLWYKWRPLDCRSLKSGFFSSKAVCRDV

SEQ ID NO: 9 - protein, 56 aa, Artificial sequence
  C41V/ C51A (V/A) DVP
AKDGDVEGPAGCKKYDVECDSGECCQKQYLWYKWRPLDCRVLKSGFFSSKAVCRDV

SEQ ID NO: 10 - protein, 56 aa, Artificial sequence
  C41A/ C51T (A/T) DVP
AKDGDVEGPAGCKKYDVECDSGECCQKQYLWYKWRPLDCRALKSGFFSSKTVCRDV

SEQ ID NO: 11 - protein, 56 aa, Artificial sequence
  C41A/ C51S (A/S) DVP
AKDGDVEGPAGCKKYDVECDSGECCQKQYLWYKWRPLDCRALKSGFFSSKSVCRDV

SEQ ID NO: 12 - protein, 56 aa, Artificial sequence
  C41A/ C51V (A/V) DVP
AKDGDVEGPAGCKKYDVECDSGECCQKQYLWYKWRPLDCRALKSGFFSSKVVCRDV

SEQ ID NO: 13 - protein, 56 aa, Artificial sequence
  C41T/ C51S DVP
AKDGDVEGPAGCKKYDVECDSGECCQKQYLWYKWRPLDCRTLKSGFFSSKSVCRDV

SEQ ID NO: 14 - protein, 56 aa, Artificial sequence
  C41S/ C51S DVP
AKDGDVEGPAGCKKYDVECDSGECCQKQYLWYKWRPLDCRSLKSGFFSSKSVCRDV

SEQ ID NO: 15 - protein, 56 aa, Artificial sequence
  C41T/ C51A/ V17A DVP
AKDGDVEGPAGCKKYDAECDSGECCQKQYLWYKWRPLDCRTLKSGFFSSKAVCRDV

SEQ ID NO: 16 - protein, 56 aa, Artificial sequence
  C41T/ C51A/ D20A DVP
AKDGDVEGPAGCKKYDVECASGECCQKQYLWYKWRPLDCRTLKSGFFSSKAVCRDV

SEQ ID NO: 17 - protein, 56 aa, Artificial sequence
  C41T/ C51A/ S21A DVP
AKDGDVEGPAGCKKYDVECDAGECCQKQYLWYKWRPLDCRTLKSGFFSSKAVCRDV

SEQ ID NO: 18 - protein, 56 aa, Artificial sequence
  C41T/ C51A/ W31A DVP
AKDGDVEGPAGCKKYDVECDSGECCQKQYLAYKWRPLDCRTLKSGFFSSKAVCRDV

SEQ ID NO: 19 - protein, 56 aa, Artificial sequence
  C41T/ C51A/ Y32A DVP
AKDGDVEGPAGCKKYDVECDSGECCQKQYLWAKWRPLDCRTLKSGFFSSKAVCRDV

SEQ ID NO: 20 - protein, 56 aa, Artificial sequence
  C41T/ C51A/ P36A DVP
AKDGDVEGPAGCKKYDVECDSGECCQKQYLWYKWRALDCRTLKSGFFSSKAVCRDV

SEQ ID NO: 21 - protein, 56 aa, Artificial sequence
  C41T/ C51A/ D38A DVP
AKDGDVEGPAGCKKYDVECDSGECCQKQYLWYKWRPLACRTLKSGFFSSKAVCRDV

SEQ ID NO: 22 - protein, 56 aa, Artificial sequence
  C41T/ C51A/ L42A DVP
AKDGDVEGPAGCKKYDVECDSGECCQKQYLWYKWRPLDCRTAKSGFFSSKAVCRDV

SEQ ID NO: 23 - protein, 56 aa, Artificial sequence
  C41T/ C51A/ V52A DVP
AKDGDVEGPAGCKKYDVECDSGECCQKQYLWYKWRPLDCRTLKSGFFSSKAACRDV

SEQ ID NO: 24 - protein, 56 aa, Artificial sequence
  C41T/ C51A/ W31F DVP
AKDGDVEGPAGCKKYDVECDSGECCQKQYLFYKWRPLDCRTLKSGFFSSKAVCRDV

SEQ ID NO: 25 - protein, 56 aa, Artificial sequence
  C41T/ C51A/ Y32S DVP
AKDGDVEGPAGCKKYDVECDSGECCQKQYLWSKWRPLDCRTLKSGFFSSKAVCRDV

SEQ ID NO: 26 - protein, 56 aa, Artificial sequence
  C41T/ C51A/ W31F/ Y32S/ P36A DVP
AKDGDVEGPAGCKKYDVECDSGECCQKQYLFSKWRALDCRTLKSGFFSSKAVCRDV

SEQ ID NO: 27 - protein, 56 aa, Artificial sequence
  C41T/ C51A/ D20A/ L42N DVP
AKDGDVEGPAGCKKYDVECASGECCQKQYLWYKWRPLDCRTNKSGFFSSKAVCRDV

SEQ ID NO: 28 - protein, 56 aa, Artificial sequence
  C41T/ C51A/ D20A/ L42V DVP
AKDGDVEGPAGCKKYDVECASGECCQKQYLWYKWRPLDCRTVKSGFFSSKAVCRDV

SEQ ID NO: 29 - protein, 56 aa, Artificial sequence; identical to SEQ ID NO: 21
  C41T/ C51A/ D38A DVP

SEQ ID NO: 30 - protein, 56 aa, Artificial sequence
  C41T/ C51A/ D38K DVP
AKDGDVEGPAGCKKYDVECDSGECCQKQYLWYKWRPLKCRTLKSGFFSSKAVCRDV

SEQ ID NO: 31 - protein, 56 aa, Artificial sequence
  C41T/ C51A/ D38S DVP
AKDGDVEGPAGCKKYDVECDSGECCQKQYLWYKWRPLSCRTLKSGFFSSKAVCRDV

SEQ ID NO: 32 - protein, 56 aa, Artificial sequence
  C41T/ C51A/ D38A/ V52T DVP
AKDGDVEGPAGCKKYDVECDSGECCQKQYLWYKWRPLACRTLKSGFFSSKATCRDV

SEQ ID NO: 33 - protein, 56 aa, Artificial sequence
  C41T/ C51A/ D38A/ V52A DVP
AKDGDVEGPAGCKKYDVECDSGECCQKQYLWYKWRPLACRTLKSGFFSSKAACRDV

SEQ ID NO: 34 - protein, 56 aa, Artificial sequence
  C41T/ C51A/ D38A/ V17E DVP
AKDGDVEGPAGCKKYDEECDSGECCQKQYLWYKWRPLACRTLKSGFFSSKAVCRDV

SEQ ID NO: 35 - protein, 56 aa, Artificial sequence
  C41T/ C51A/ D38A/ L42V DVP
AKDGDVEGPAGCKKYDVECDSGECCQKQYLWYKWRPLACRTVKSGFFSSKAVCRDV

SEQ ID NO: 36 - protein, 56 aa, Artificial sequence
  C41T/ C51A/ D38A/ L42S DVP
AKDGDVEGPAGCKKYDVECDSGECCQKQYLWYKWRPLACRTSKSGFFSSKAVCRDV

SEQ ID NO: 37 - protein, 56 aa, Artificial sequence
  C41T/ C51A/ D38A/ L42E DVP
AKDGDVEGPAGCKKYDVECDSGECCQKQYLWYKWRPLACRTEKSGFFSSKAVCRDV

SEQ ID NO: 38 - protein, 56 aa, Artificial sequence
  C41T/ C51A/ D38A/ L42Q DVP
AKDGDVEGPAGCKKYDVECDSGECCQKQYLWYKWRPLACRTQKSGFFSSKAVCRDV

SEQ ID NO: 39 - protein, 56 aa, Artificial sequence
  C41T/ C51A/ D38A/ D20A DVP
AKDGDVEGPAGCKKYDVECASGECCQKQYLWYKWRPLACRTLKSGFFSSKAVCRDV

SEQ ID NO: 40 - protein, 56 aa, Artificial sequence
  C41T/ C51A/ D20A/ Y32S DVP
AKDGDVEGPAGCKKYDVECASGECCQKQYLWSKWRPLDCRTLKSGFFSSKAVCRDV

SEQ ID NO: 41 - protein, 56 aa, Artificial sequence
  C41T/ C51A/ D38A/ Y32S DVP
AKDGDVEGPAGCKKYDVECDSGECCQKQYLWSKWRPLACRTLKSGFFSSKAVCRDV

SEQ ID NO: 42 - protein, 56 aa, Artificial sequence
  C41T/ C51A/ D20A/ D38A/ Y32S DVP
AKDGDVEGPAGCKKYDVECASGECCQKQYLWSKWRPLACRTLKSGFFSSKAVCRDV

SEQ ID NO: 43 - protein, 56 aa, Artificial sequence
  C41T/ C51A/ D20A/ W31F/ Y32S/ P36A DVP
AKDGDVEGPAGCKKYDVECASGECCQKQYLFSKWRALDCRTLKSGFFSSKAVCRDV

SEQ ID NO: 44 - protein, 56 aa, Artificial sequence
  D38A DVP
AKDGDVEGPAGCKKYDVECDSGECCQKQYLWYKWRPLACRCLKSGFFSSKCVCRDV

SEQ ID NO: 45 - protein, 56 aa, Artificial sequence
  C41S/ C51T/ D38A (S/T + D38A) DVP
AKDGDVEGPAGCKKYDVECDSGECCQKQYLWYKWRPLACRSLKSGFFSSKTVCRDV

SEQ ID NO: 46 - protein, 56 aa, Artificial sequence
  C41T/ C51T/ D38A (T/T + D38A) DVP
AKDGDVEGPAGCKKYDVECDSGECCQKQYLWYKWRPLACRTLKSGFFSSKTVCRDV

SEQ ID NO: 47 - protein, 56 aa, Artificial sequence
  C41S/ C51S/ D38A (S/S + D38A) DVP
AKDGDVEGPAGCKKYDVECDSGECCQKQYLWYKWRPLACRSLKSGFFSSKSVCRDV

SEQ ID NO: 48 - protein, 56 aa, Artificial sequence
  C41T/ C51S/ D38A (T/S + D38A) DVP
AKDGDVEGPAGCKKYDVECDSGECCQKQYLWYKWRPLACRTLKSGFFSSKSVCRDV

SEQ ID NO: 49 - protein, 56 aa, Artificial sequence
  C41V/ C51T/ D38A (V/T + D38A) DVP
AKDGDVEGPAGCKKYDVECDSGECCQKQYLWYKWRPLACRVLKSGFFSSKTVCRDV

SEQ ID NO: 50 - protein, 56 aa, Artificial sequence
  C41T/ C51V/ D38A (T/V + D38A) DVP
AKDGDVEGPAGCKKYDVECDSGECCQKQYLWYKWRPLACRTLKSGFFSSKVVCRDV

SEQ ID NO: 51 - protein, 56 aa, Artificial sequence
  C41S/ C51V/ D38A (S/V + D38A) DVP
AKDGDVEGPAGCKKYDVECDSGECCQKQYLWYKWRPLACRSLKSGFFSSKVVCRDV

SEQ ID NO: 52 - protein, 56 aa, Artificial sequence
  C41V/ C51S/ D38A (V/S + D38A) DVP
AKDGDVEGPAGCKKYDVECDSGECCQKQYLWYKWRPLACRVLKSGFFSSKSVCRDV

SEQ ID NO: 53 - protein, 56 aa, Artificial sequence
  C41S/ C51S/ D38A/L42V  (S/S + D38A/L42V) DVP
AKDGDVEGPAGCKKYDVECDSGECCQKQYLWYKWRPLACRSVKSGFFSSKSVCRDV

SEQ ID NO: 54 - protein, 4 aa, Artificial sequence
  IGER
IGER

SEQ ID NO: 55 - protein, 5 aa, Artificial sequence
  EEKKN
EEKKN

SEQ ID NO: 56 - protein, 8 aa, Artificial sequence
  ETMFKHGL
ETMFKHGL

SEQ ID NO: 57 - protein, 238 aa, Aequorea victoria
ASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 58 - protein, 157 aa, Galanthus nivalis
MAKASLLILATIFLGVITPSCLSENILYSGETLPTGGFLSSGSFVFIMQEDCNLVLYNVDKPIWATNTGGLSSDCSLSMQNDGNLVVFTPSNKPIWASNTDGQNGNYVCILQKDRNVVIYGTNRWATGTYTGAVGIPESPPSEKYPSAGKIKLVTAK

SEQ ID NO: 59 - protein, 225 aa, Juniperus ashei
MARVSELAFLLAATLAISLHMQEAGVVKFDIKNQCGYTVWAAGLPGGGKRLDQGQTWTVNLAAGTASARFWGRTGCTFDASGKGSCQTGDCGGQLSCTVSGAVPATLAEYTQSDQDYYDVSLVDGFNIPLAINPTNAQCTAPACKADINAVCPSELKVDGGCNSACNVFKTDQYCCRNAYVDNCPATNYSKIFKNQCPQAYSYAKDDTATFACASGTDYSIVFCP

SEQ ID NO: 60 - protein, 24 aa, Hordeum vulgare
MANKHLSLSLFLVLLGLSASLASG

SEQ ID NO: 61 - protein, 27 aa, Nicotiana plumbaginifolia
EMGKMASLFASLLVVLVSLSLASESSA

SEQ ID NO: 62 - protein, 26 aa, Nicotiana plumbaginifolia
MGKMASLFATFLVVLVSLSLASESSA

SEQ ID NO: 63 - protein, 78 aa, Nicotiana plumbaginifolia
ATGGGTAAGATGGCTTCTCTGTTTGCTTCTCTGCTGGTTGTTCTGGTTTCTCTGTCTCTGGCTTCTGAATCTTCTGCT

SEQ ID NO: 64 - protein, 78 aa, Nicotiana plumbaginifolia
ATGGGTAAGATGGCTTCTCTGTTTGCTACTTTTCTGGTTGTTCTGGTTTCTCTGTCTCTGGCTTCTGAATCTTCTGCT

SEQ ID NO: 65 - protein, 224 aa, Juniperus ashei
KFDIKNQCGYTVWAAGLPGGGKRLDQGQTWTVNLAAGTASARFWGRTGCTFDASGKGSCQTGDCGGQLSCTVSGAVPATLAEYTQSDQDYYDVSLVDGFNIPLAINPTNAQCTAPACKADINAVCPSELKVDGGCNSACNVFKTDQYCCRNAYVDNCPATNYSKIFKNQCPQAYSYAKDDTATFACASGTDYSIVFCMARVSELAFLLAATLAISLHMQEAGVV

SEQ ID NO: 66 - protein, 105 aa, Galanthus nivalis
DNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATG

SEQ ID NO: 67 - protein, 27 aa, Anemonia viridis
RSCCPCYWGGCPWGQNCYPEGCSGPKV

SEQ ID NO: 68 - protein, 47 aa, Anemonia viridis
GVPCLCDSDGPSVRGNTLSGIIWLAGCPSGWHNCKKHGPTIGWCCKQ

SEQ ID NO: 69 - protein, 27 aa, Artificial sequence
  Av3a
KSCCPCYWGGCPWGQNCYPEGCSGPKV

SEQ ID NO: 70 - protein, 26 aa, Artificial sequence
  Av3a-C1
RSCCPCYWGGCPWGQNCYPEGCSGPK

SEQ ID NO: 71 - protein, 26 aa, Artificial sequence
  Av3b
KSCCPCYWGGCPWGQNCYPEGCSGPK

SEQ ID NO: 72 - DNA, 282 bp, Diguetia canities
atgaaggtttttgttgtactgttgtgcttgtctctggcagcagtttacgccttggaggaaagactagacaaagacgccgacatcatgcttgattcaccagccgacatggaaagagcgaaggacggtgacgtggaagggcctgcgggctgcaagaaatacgacgtagagtgcgacagtggagagtgctgccagaagcagtacctgtggtacaagtggcgacccctggattgccgatgcctaaagagcggtttcttcagcagcaagtgcgtttgcagagacgtg

SEQ ID NO: 73 - DNA, 168 bp, Diguetia canities
gctaaagatggtgatgttgaaggtccagctggttgtaaaaaatatgatgttgaatgtgattctggtgaatgttgtcaaaaacaatatttgtggtataaatggagaccattggattgtagatgtttgaaatctggtttcttctcttctaaatgtgtttgtagagatgtt

SEQ ID NO: 74 - DNA, 168 bp, Diguetia canities; identical to SEQ ID NO: 73

SEQ ID NO: 75 - DNA, 168 bp, Artificial sequence
  Disulfide deletion
gctaaagatggtgatgttgaaggtccagctggttgtaaaaaatatgatgttgaatgtgattctggtgaatgttgtcaaaaacaatatttgtggtataaatggagaccattggattgtagaggcttgaaatctggtttcttctcttctaaatttgtttgtagagatgtt

SEQ ID NO: 76 - DNA, 168 bp, Artificial sequence; identical to SEQ ID NO: 75
  Disulfide deletion

SEQ ID NO: 77 - DNA, 168 bp, Artificial sequence
  C41T/ C51A DVP
gctaaagatggtgatgttgaaggtccagctggttgtaaaaaatatgatgttgaatgtgattctggtgaatgttgtcaaaaacaatatttgtggtataaatggagaccattggattgtagaactttgaaatctggtttcttctcttctaaagctgtttgtagagatgtt

SEQ ID NO: 78 - DNA, 168 bp, Artificial sequence
  C41A/ C51A (A/A) DVP
gctaaagatggtgatgttgaaggtccagctggttgtaaaaaatatgatgttgaatgtgattctggtgaatgttgtcaaaaacaatatttgtggtataaatggagaccattggattgtagagcattgaaatctggtttcttctcttctaaagctgtttgtagagatgtt

SEQ ID NO: 79 - DNA, 168 bp, Artificial sequence
  C41S/ C51A (S/A) DVP
gctaaagatggtgatgttgaaggtccagctggttgtaaaaaatatgatgttgaatgtgattctggtgaatgttgtcaaaaacaatatttgtggtataaatggagaccattggattgtagatctttgaaatctggtttcttctcttctaaagctgtttgtagagatgtt

SEQ ID NO: 80 - DNA, 168 bp, Artificial sequence
  C41V/ C51A (V/A) DVP
gctaaagatggtgatgttgaaggtccagctggttgtaaaaaatatgatgttgaatgtgattctggtgaatgttgtcaaaaacaatatttgtggtataaatggagaccattggattgtagagttttgaaatctggtttcttctcttctaaagctgtttgtagagatgtt

SEQ ID NO: 81 - DNA, 168 bp, Artificial sequence
  C41A/ C51T (A/T) DVP
gctaaagatggtgatgttgaaggtccagctggttgtaaaaaatatgatgttgaatgtgattctggtgaatgttgtcaaaaacaatatttgtggtataaatggagaccattggattgtagagcattgaaatctggtttcttctcttctaaaactgtttgtagagatgtt

SEQ ID NO: 82 - DNA, 168 bp, Artificial sequence
  C41A/ C51S (A/S) DVP
gctaaagatggtgatgttgaaggtccagctggttgtaaaaaatatgatgttgaatgtgattctggtgaatgttgtcaaaaacaatatttgtggtataaatggagaccattggattgtagagcattgaaatctggtttcttctcttctaaatctgtttgtagagatgtt

SEQ ID NO: 83 - DNA, 168 bp, Artificial sequence
  C41A/ C51V (A/V) DVP
gctaaagatggtgatgttgaaggtccagctggttgtaaaaaatatgatgttgaatgtgattctggtgaatgttgtcaaaaacaatatttgtggtataaatggagaccattggattgtagagcattgaaatctggtttcttctcttctaaagttgtttgtagagatgtt

SEQ ID NO: 84 - DNA, 168 bp, Artificial sequence
  C41T/ C51S DVP
gctaaagatggtgatgttgaaggtccagctggttgtaaaaaatatgatgttgaatgtgattctggtgaatgttgtcaaaaacaatatttgtggtataaatggagaccattggattgtagaactttgaaatctggtttcttctcttctaaatctgtttgtagagatgtt

SEQ ID NO: 85 - DNA, 169 bp, Artificial sequence
  C41S/ C51S DVP
gctaaagatggtgatgttgaaggtccagctggttgtaaaaaatatgatgttgaatgtgattctggtgaatgttgtcaaaaacaatatttgtggtataaatggagaccattggattgtagaagtttgaaatctggtttcttctcttctaaatcagtttgtagagatgttt

SEQ ID NO: 86 - DNA, 168 bp, Artificial sequence
  C41T/ C51A/ V17A DVP
gctaaagatggtgatgttgaaggtccagctggttgtaaaaaatatgatgctgaatgtgattctggtgaatgttgtcaaaaacaatatttgtggtataaatggagaccattggattgtagaactttgaaatctggtttcttctcttctaaagctgtttgtagagatgtt

SEQ ID NO: 87 - DNA, 168 bp, Artificial sequence
  C41T/ C51A/ D20A DVP
gctaaagatggtgatgttgaaggtccagctggttgtaaaaaatatgatgttgaatgtgcttctggtgaatgttgtcaaaaacaatatttgtggtataaatggagaccattggattgtagaactttgaaatctggtttcttctcttctaaagctgtttgtagagatgtt

SEQ ID NO: 88 - DNA, 168 bp, Artificial sequence
  C41T/ C51A/ S21A DVP
gctaaagatggtgatgttgaaggtccagctggttgtaaaaaatatgatgttgaatgtgatgctggtgaatgttgtcaaaaacaatatttgtggtataaatggagaccattggattgtagaactttgaaatctggtttcttctcttctaaagctgtttgtagagatgtt

SEQ ID NO: 89 - DNA, 168 bp, Artificial sequence
  C41T/ C51A/ W31A DVP
gctaaagatggtgatgttgaaggtccagctggttgtaaaaaatatgatgttgaatgtgattctggtgaatgttgtcaaaaacaatatttggcttataaatggagaccattggattgtagaactttgaaatctggtttcttctcttctaaagctgtttgtagagatgtt

SEQ ID NO: 90 - DNA, 168 bp, Artificial sequence
  C41T/ C51A/ Y32A DVP
gctaaagatggtgatgttgaaggtccagctggttgtaaaaaatatgatgttgaatgtgattctggtgaatgttgtcaaaaacaatatttgtgggctaaatggagaccattggattgtagaactttgaaatctggtttcttctcttctaaagctgtttgtagagatgtt

SEQ ID NO: 91 - DNA, 168 bp, Artificial sequence
  C41T/ C51A/ P36A DVP
gctaaagatggtgatgttgaaggtccagctggttgtaaaaaatatgatgttgaatgtgattctggtgaatgttgtcaaaaacaatatttgtggtataaatggagagctttggattgtagaactttgaaatctggtttcttctcttctaaagctgtttgtagagatgtt

SEQ ID NO: 92 - DNA, 168 bp, Artificial sequence
  C41T/ C51A/ D38A DVP
gctaaagatggtgatgttgaaggtccagctggttgtaaaaaatatgatgttgaatgtgattctggtgaatgttgtcaaaaacaatatttgtggtataaatggagaccattggcttgtagaactttgaaatctggtttcttctcttctaaagctgtttgtagagatgtt

SEQ ID NO: 93 - DNA, 168 bp, Artificial sequence
  C41T/ C51A/ L42A DVP
gctaaagatggtgatgttgaaggtccagctggttgtaaaaaatatgatgttgaatgtgattctggtgaatgttgtcaaaaacaatatttgtggtataaatggagaccattggattgtagaactgctaaatctggtttcttctcttctaaagctgtttgtagagatgtt

SEQ ID NO: 94 - DNA, 168 bp, Artificial sequence
  C41T/ C51A/ V52A DVP
gctaaagatggtgatgttgaaggtccagctggttgtaaaaaatatgatgttgaatgtgattctggtgaatgttgtcaaaaacaatatttgtggtataaatggagaccattggattgtagaactttgaaatctggtttcttctcttctaaagctgcttgtagagatgtt

SEQ ID NO: 95 - DNA, 168 bp, Artificial sequence
  C41T/ C51A/ W31F DVP
gctaaagatggtgatgttgaaggtccagctggttgtaaaaaatatgatgttgaatgtgattctggtgaatgttgtcaaaaacaatatttgttttataaatggagaccattggattgtagaactttgaaatctggtttcttctcttctaaagctgtttgtagagatgtt

SEQ ID NO: 96 - DNA, 168 bp, Artificial sequence
  C41T/ C51A/ Y32S DVP
gctaaagatggtgatgttgaaggtccagctggttgtaaaaaatatgatgttgaatgtgattctggtgaatgttgtcaaaaacaatatttgtggtctaaatggagaccattggattgtagaactttgaaatctggtttcttctcttctaaagctgtttgtagagatgtt

SEQ ID NO: 97 - DNA, 168 bp, Artificial sequence
  C41T/ C51A/ W31F/ Y32S/ P36A DVP
gctaaagatggtgatgttgaaggtccagctggttgtaaaaaatatgatgttgaatgtgattctggtgaatgttgtcaaaaacaatatttgttttctaaatggagagctttggattgtagaactttgaaatctggtttcttctcttctaaagctgtttgtagagatgtt

SEQ ID NO: 98 - DNA, 168 bp, Artificial sequence
  C41T/ C51A/ D20A/ L42N DVP
gctaaagatggtgatgttgaaggtccagctggttgtaaaaaatatgatgttgaatgtgcttctggtgaatgttgtcaaaaacaatatttgtggtataaatggagaccattggattgtagaactaataaatctggtttcttctcttctaaagctgtttgtagagatgtt

SEQ ID NO: 99 - DNA, 168 bp, Artificial sequence
  C41T/ C51A/ D20A/ L42V DVP
gctaaagatggtgatgttgaaggtccagctggttgtaaaaaatatgatgttgaatgtgcttctggtgaatgttgtcaaaaacaatatttgtggtataaatggagaccattggattgtagaactgttaaatctggtttcttctcttctaaagctgtttgtagagatgtt

SEQ ID NO: 100 - DNA, 168 bp, Artificial sequence; identical to SEQ ID NO: 92
  C41T/ C51A/ D38A DVP

SEQ ID NO: 101 - DNA, 168 bp, Artificial sequence
  C41T/ C51A/ D38K DVP
gctaaagatggtgatgttgaaggtccagctggttgtaaaaaatatgatgttgaatgtgattctggtgaatgttgtcaaaaacaatatttgtggtataaatggagaccattgagatgtagaactttgaaatctggtttcttctcttctaaagctgtttgtagagatgtt

SEQ ID NO: 102 - DNA, 168 bp, Artificial sequence
  C41T/ C51A/ D38S DVP
gctaaagatggtgatgttgaaggtccagctggttgtaaaaaatatgatgttgaatgtgattctggtgaatgttgtcaaaaacaatatttgtggtataaatggagaccattgtcttgtagaactttgaaatctggtttcttctcttctaaagctgtttgtagagatgtt

SEQ ID NO: 103 - DNA, 168 bp, Artificial sequence
  C41T/ C51A/ D38A/ V52T DVP
gctaaagatggtgatgttgaaggtccagctggttgtaaaaaatatgatgttgaatgtgattctggtgaatgttgtcaaaaacaatatttgtggtataaatggagaccattggcttgtagaactttgaaatctggtttcttctcttctaaagctacttgtagagatgtt

SEQ ID NO: 104 - DNA, 168 bp, Artificial sequence
  C41T/ C51A/ D38A/ V52A DVP
gctaaagatggtgatgttgaaggtccagctggttgtaaaaaatatgatgttgaatgtgattctggtgaatgttgtcaaaaacaatatttgtggtataaatggagaccattggcttgtagaactttgaaatctggtttcttctcttctaaagctgcttgtagagatgtt

SEQ ID NO: 105 - DNA, 168 bp, Artificial sequence
  C41T/ C51A/ D38A/ V17E DVP
gctaaagatggtgatgttgaaggtccagctggttgtaaaaaatatgatgaagaatgtgattctggtgaatgttgtcaaaaacaatatttgtggtataaatggagaccattggcttgtagaactttgaaatctggtttcttctcttctaaagctgtttgtagagatgtt

SEQ ID NO: 106 - DNA, 168 bp, Artificial sequence
  C41T/ C51A/ D38A/ L42V DVP
gctaaagatggtgatgttgaaggtccagctggttgtaaaaaatatgatgttgaatgtgattctggtgaatgttgtcaaaaacaatatttgtggtataaatggagaccattggcttgtagaactgttaaatctggtttcttctcttctaaagctgtttgtagagatgtt

SEQ ID NO: 107 - DNA, 168 bp, Artificial sequence
  C41T/ C51A/ D38A/ L42S DVP
gctaaagatggtgatgttgaaggtccagctggttgtaaaaaatatgatgttgaatgtgattctggtgaatgttgtcaaaaacaatatttgtggtataaatggagaccattggcttgtagaacttctaaatctggtttcttctcttctaaagctgtttgtagagatgtt

SEQ ID NO: 108 - DNA, 168 bp, Artificial sequence
  C41T/ C51A/ D38A/ L42E DVP
gctaaagatggtgatgttgaaggtccagctggttgtaaaaaatatgatgttgaatgtgattctggtgaatgttgtcaaaaacaatatttgtggtataaatggagaccattggcttgtagaactgaaaaatctggtttcttctcttctaaagctgtttgtagagatgtt

SEQ ID NO: 109 - DNA, 168 bp, Artificial sequence
  C41T/ C51A/ D38A/ L42Q DVP
gctaaagatggtgatgttgaaggtccagctggttgtaaaaaatatgatgttgaatgtgattctggtgaatgttgtcaaaaacaatatttgtggtataaatggagaccattggcttgtagaactcaaaaatctggtttcttctcttctaaagctgtttgtagagatgtt

SEQ ID NO: 110 - DNA, 168 bp, Artificial sequence
  C41T/ C51A/ D38A/ D20A DVP
gctaaagatggtgatgttgaaggtccagctggttgtaaaaaatatgatgttgaatgtgcttctggtgaatgttgtcaaaaacaatatttgtggtataaatggagaccattggcttgtagaactttgaaatctggtttcttctcttctaaagctgtttgtagagatgtt

SEQ ID NO: 111 - DNA, 168 bp, Artificial sequence
  C41T/ C51A/ D20A/ Y32S DVP
gctaaagatggtgatgttgaaggtccagctggttgtaaaaaatatgatgttgaatgtgcttctggtgaatgttgtcaaaaacaatatttgtggtctaaatggagaccattggattgtagaactttgaaatctggtttcttctcttctaaagctgtttgtagagatgtt

SEQ ID NO: 112 - DNA, 168 bp, Artificial sequence
  C41T/ C51A/ D38A/ Y32S DVP
gctaaagatggtgatgttgaaggtccagctggttgtaaaaaatatgatgttgaatgtgattctggtgaatgttgtcaaaaacaatatttgtggtctaaatggagaccattggcttgtagaactttgaaatctggtttcttctcttctaaagctgtttgtagagatgtt

SEQ ID NO: 113 - DNA, 168 bp, Artificial sequence
  C41T/ C51A/ D20A/ D38A/ Y32S DVP
gctaaagatggtgatgttgaaggtccagctggttgtaaaaaatatgatgttgaatgtgcttctggtgaatgttgtcaaaaacaatatttgtggtctaaatggagaccattggcttgtagaactttgaaatctggtttcttctcttctaaagctgtttgtagagatgtt

SEQ ID NO: 114 - DNA, 168 bp, Artificial sequence
  C41T/ C51A/ D20A/ W31F/ Y32S/ P36A DVP
gctaaagatggtgatgttgaaggtccagctggttgtaaaaaatatgatgttgaatgtgcttctggtgaatgttgtcaaaaacaatatttgttttctaaatggagagctttggattgtagaactttgaaatctggtttcttctcttctaaagctgtttgtagagatgtt

SEQ ID NO: 115 - DNA, 168 bp, Artificial sequence
  D38A DVP
gctaaagatggtgatgttgaaggtccagctggttgtaaaaaatatgatgttgaatgtgattctggtgaatgttgtcaaaaacaatatttgtggtataaatggagaccattggcttgtagatgtttgaaatctggtttcttctcttctaaatgtgtttgtagagatgtt

SEQ ID NO: 116 - DNA, 168 bp, Artificial sequence
  C41S/ C51T/ D38A (S/T + D38A) DVP
gctaaagatggtgatgttgaaggtccagctggttgtaaaaaatatgatgttgaatgtgattctggtgaatgttgtcaaaaacaatatttgtggtataaatggagaccattggcttgtagatctttgaaatctggtttcttctcttctaaaagtgtttgtagagatgtt

SEQ ID NO: 117 - DNA, 168 bp, Artificial sequence
  C41T/ C51T/ D38A (T/T + D38A) DVP
gctaaagatggtgatgttgaaggtccagctggttgtaaaaaatatgatgttgaatgtgattctggtgaatgttgtcaaaaacaatatttgtggtataaatggagaccattggcttgtagaactttgaaatctggtttcttctcttctaaaagtgtttgtagagatgtt

SEQ ID NO: 118 - DNA, 168 bp, Artificial sequence
  C41S/ C51S/ D38A (S/S + D38A) DVP
gctaaagatggtgatgttgaaggtccagctggttgtaaaaaatatgatgttgaatgtgattctggtgaatgttgtcaaaaacaatatttgtggtataaatggagaccattggcttgtagatctttgaaatctggtttcttctcttctaaatctgtttgtagagatgtt

SEQ ID NO: 119 - DNA, 168 bp, Artificial sequence
  C41T/ C51S/ D38A (T/S + D38A) DVP
gctaaagatggtgatgttgaaggtccagctggttgtaaaaaatatgatgttgaatgtgattctggtgaatgttgtcaaaaacaatatttgtggtataaatggagaccattggcttgtagaactttgaaatctggtttcttctcttctaaatctgtttgtagagatgtt

SEQ ID NO: 120 - DNA, 168 bp, Artificial sequence
  C41V/ C51T/ D38A (V/T + D38A) DVP
gctaaagatggtgatgttgaaggtccagctggttgtaaaaaatatgatgttgaatgtgattctggtgaatgttgtcaaaaacaatatttgtggtataaatggagaccattggcttgtagagttttgaaatctggtttcttctcttctaaaactgtttgtagagatgtt

SEQ ID NO: 121 - DNA, 168 bp, Artificial sequence
  C41T/ C51V/ D38A (T/V + D38A) DVP
gctaaagatggtgatgttgaaggtccagctggttgtaaaaaatatgatgttgaatgtgattctggtgaatgttgtcaaaaacaatatttgtggtataaatggagaccattggcttgtagaactttgaaatctggtttcttctcttctaaagttgtttgtagagatgtt

SEQ ID NO: 122 - DNA, 168 bp, Artificial sequence
  C41S/ C51V/ D38A (S/V + D38A) DVP
gctaaagatggtgatgttgaaggtccagctggttgtaaaaaatatgatgttgaatgtgattctggtgaatgttgtcaaaaacaatatttgtggtataaatggagaccattggcttgtagatctttgaaatctggtttcttctcttctaaagttgtttgtagagatgtt

SEQ ID NO: 123 - DNA, 168 bp, Artificial sequence
  C41V/ C51S/ D38A (V/S + D38A) DVP
gctaaagatggtgatgttgaaggtccagctggttgtaaaaaatatgatgttgaatgtgattctggtgaatgttgtcaaaaacaatatttgtggtataaatggagaccattggcttgtagagttttgaaatctggtttcttctcttctaaatctgtttgtagagatgtt

SEQ ID NO: 124 - DNA, 168 bp, Artificial sequence
  C41S/ C51S/ D38A/L42V  (S/S + D38A/L42V) DVP
gccaaagacggagacgtagaaggcccagcaggttgtaagaaatatgacgtagaatgtgactcgggggagtgttgtcagaagcaatacctttggtacaaatggagaccattggcttgtcgtagtgtgaagagtggattcttttcttcaaaatcggtttgcagggatgtt

SEQ ID NO: 125 - protein, 56 aa, Artificial sequence
  C41N/ C51A/ D38A/ L42V DVP
AKDGDVEGPAGCKKYDVECDSGECCQKQYLWYKWRPLACRNVKSGFFSSKAVCRDV

SEQ ID NO: 126 - protein, 56 aa, Artificial sequence
  C41D/ C51A/ D38A/ L42V DVP
AKDGDVEGPAGCKKYDVECDSGECCQKQYLWYKWRPLACRDVKSGFFSSKAVCRDV

SEQ ID NO: 127 - protein, 56 aa, Artificial sequence
  C41S/ C51A/ D38A/ L42V DVP
AKDGDVEGPAGCKKYDVECDSGECCQKQYLWYKWRPLACRSVKSGFFSSKAVCRDV

SEQ ID NO: 128 - protein, 56 aa, Artificial sequence
  C41M/ C51A/ D38A/ L42V DVP
AKDGDVEGPAGCKKYDVECDSGECCQKQYLWYKWRPLACRMVKSGFFSSKAVCRDV

SEQ ID NO: 129 - protein, 56 aa, Artificial sequence
  C41T/ C51G/ D38A/ L42V DVP
AKDGDVEGPAGCKKYDVECDSGECCQKQYLWYKWRPLACRTVKSGFFSSKGVCRDV

SEQ ID NO: 130 - protein, 56 aa, Artificial sequence
  C41T/ C51D/ D38A/ L42V DVP
AKDGDVEGPAGCKKYDVECDSGECCQKQYLWYKWRPLACRTVKSGFFSSKDVCRDV

SEQ ID NO: 131 - protein, 56 aa, Artificial sequence
  C41T/ C51N/ D38A/ L42V DVP
AKDGDVEGPAGCKKYDVECDSGECCQKQYLWYKWRPLACRTVKSGFFSSKNVCRDV

SEQ ID NO: 132 - protein, 56 aa, Artificial sequence
  C41T/ C51Q/ D38A/ L42V DVP
AKDGDVEGPAGCKKYDVECDSGECCQKQYLWYKWRPLACRTVKSGFFSSKQVCRDV

SEQ ID NO: 133 - protein, 56 aa, Artificial sequence
  C41T/ C51E/ D38A/ L42V DVP
AKDGDVEGPAGCKKYDVECDSGECCQKQYLWYKWRPLACRTVKSGFFSSKEVCRDV

SEQ ID NO: 134 - protein, 56 aa, Artificial sequence
  C41T/ C51V/ D38A/ L42V DVP
AKDGDVEGPAGCKKYDVECDSGECCQKQYLWYKWRPLACRTVKSGFFSSKVVCRDV

SEQ ID NO: 135 - protein, 56 aa, Artificial sequence
  C41T/ C51H/ D38A/ L42V DVP
AKDGDVEGPAGCKKYDVECDSGECCQKQYLWYKWRPLACRTVKSGFFSSKHVCRDV

SEQ ID NO: 136 - protein, 56 aa, Artificial sequence
  C41T/ C51M/ D38A/ L42V DVP
AKDGDVEGPAGCKKYDVECDSGECCQKQYLWYKWRPLACRTVKSGFFSSKMVCRDV

SEQ ID NO: 137 - protein, 56 aa, Artificial sequence
  C41V/ C51V/ D38A/ L42V DVP
AKDGDVEGPAGCKKYDVECDSGECCQKQYLWYKWRPLACRVVKSGFFSSKVVCRDV

SEQ ID NO: 138 - protein, 56 aa, Artificial sequence
  C41M/ C51M/ D38A/ L42V DVP
AKDGDVEGPAGCKKYDVECDSGECCQKQYLWYKWRPLACRMVKSGFFSSKMVCRDV

SEQ ID NO: 139 - protein, 56 aa, Artificial sequence
  C41K/ C51E/ D38A/ L42V DVP
AKDGDVEGPAGCKKYDVECDSGECCQKQYLWYKWRPLACRKVKSGFFSSKEVCRDV

SEQ ID NO: 140 - protein, 56 aa, Artificial sequence
  C41E/ C51K/ D38A/ L42V DVP
AKDGDVEGPAGCKKYDVECDSGECCQKQYLWYKWRPLACREVKSGFFSSKKVCRDV

SEQ ID NO: 141 - protein, 56 aa, Artificial sequence
  C41T/ C51A/ D20V/ D38A/ L42V DVP
AKDGDVEGPAGCKKYDVECVSGECCQKQYLWYKWRPLACRTVKSGFFSSKAVCRDV

SEQ ID NO: 142 - protein, 56 aa, Artificial sequence
  C41T/ C51A/ D20G/ D38A/ L42V DVP
AKDGDVEGPAGCKKYDVECGSGECCQKQYLWYKWRPLACRTVKSGFFSSKAVCRDV

SEQ ID NO: 143 - protein, 56 aa, Artificial sequence
  C41T/ C51A/ D20K/ D38A/ L42V DVP
AKDGDVEGPAGCKKYDVECKSGECCQKQYLWYKWRPLACRTVKSGFFSSKAVCRDV

SEQ ID NO: 144 - protein, 56 aa, Artificial sequence
  C41T/ C51A/ D20E/ D38A/ L42V DVP
AKDGDVEGPAGCKKYDVECESGECCQKQYLWYKWRPLACRTVKSGFFSSKAVCRDV

SEQ ID NO: 145 - protein, 56 aa, Artificial sequence
  C41T/ C51A/ D20L/ D38A/ L42V DVP
AKDGDVEGPAGCKKYDVECLSGECCQKQYLWYKWRPLACRTVKSGFFSSKAVCRDV

SEQ ID NO: 146 - protein, 56 aa, Artificial sequence
  C41T/ C51A/ D20N/ D38A/ L42V DVP
AKDGDVEGPAGCKKYDVECNSGECCQKQYLWYKWRPLACRTVKSGFFSSKAVCRDV

SEQ ID NO: 147 - protein, 56 aa, Artificial sequence
  C41T/ C51A/ D20Y/ D38A/ L42V DVP
AKDGDVEGPAGCKKYDVECYSGECCQKQYLWYKWRPLACRTVKSGFFSSKAVCRDV

SEQ ID NO: 148 - protein, 56 aa, Artificial sequence
  C41T/ C51A/ S21G/ D38A/ L42V DVP
AKDGDVEGPAGCKKYDVECDGGECCQKQYLWYKWRPLACRTVKSGFFSSKAVCRDV

SEQ ID NO: 149 - protein, 56 aa, Artificial sequence
  C41T/ C51A/ E18P/ D38A/ L42V DVP
AKDGDVEGPAGCKKYDVPCDSGECCQKQYLWYKWRPLACRTVKSGFFSSKAVCRDV

SEQ ID NO: 150 - protein, 56 aa, Artificial sequence
  C41T/ C51A/ E18K/ D38A/ L42V DVP
AKDGDVEGPAGCKKYDVKCDSGECCQKQYLWYKWRPLACRTVKSGFFSSKAVCRDV

SEQ ID NO: 151 - protein, 56 aa, Artificial sequence
  C41T/ C51A/ E18S/ D38A/ L42V DVP
AKDGDVEGPAGCKKYDVSCDSGECCQKQYLWYKWRPLACRTVKSGFFSSKAVCRDV

SEQ ID NO: 152 - protein, 56 aa, Artificial sequence
  C41T/ C51A/ E18D/ D38A/ L42V DVP
AKDGDVEGPAGCKKYDVDCDSGECCQKQYLWYKWRPLACRTVKSGFFSSKAVCRDV

SEQ ID NO: 153 - DNA, 168 bp, Artificial sequence
  C41N/ C51A/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtaatgtgaagagtggattcttttcttcaaaagcagtttgcagggatgtt

SEQ ID NO: 154 - DNA, 168 bp, Artificial sequence
  C41D/ C51A/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtgatgtgaagagtggattcttttcttcaaaagcagtttgcagggatgtt

SEQ ID NO: 155 - DNA, 168 bp, Artificial sequence
  C41S/ C51A/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgttctgtgaagagtggattcttttcttcaaaagcagtttgcagggatgtt

SEQ ID NO: 156 - DNA, 168 bp, Artificial sequence
  C41M/ C51A/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtatggtgaagagtggattcttttcttcaaaagcagtttgcagggatgtt

SEQ ID NO: 157 - DNA, 168 bp, Artificial sequence
  C41T/ C51G/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaaggtgtttgcagggatgtt

SEQ ID NO: 158 - DNA, 168 bp, Artificial sequence
  C41T/ C51D/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaagatgtttgcagggatgtt

SEQ ID NO: 159 - DNA, 168 bp, Artificial sequence
  C41T/ C51N/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaaaatgtttgcagggatgtt

SEQ ID NO: 160 - DNA, 168 bp, Artificial sequence
  C41T/ C51Q/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaacaagtttgcagggatgtt

SEQ ID NO: 161 - DNA, 168 bp, Artificial sequence
  C41T/ C51E/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaagaagtttgcagggatgtt

SEQ ID NO: 162 - DNA, 168 bp, Artificial sequence
  C41T/ C51V/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaagtcgtttgcagggatgtt

SEQ ID NO: 163 - DNA, 168 bp, Artificial sequence
  C41T/ C51H/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaacatgtttgcagggatgtt

SEQ ID NO: 164 - DNA, 168 bp, Artificial sequence
  C41T/ C51M/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtactgtgaagagtggattcttttcttcaaaaatggtttgcagggatgtt

SEQ ID NO: 165 - DNA, 168 bp, Artificial sequence
  C41V/ C51V/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtgtcgtgaagagtggattcttttcttcaaaagtagtttgcagggatgtt

SEQ ID NO: 166 - DNA, 168 bp, Artificial sequence
  C41M/ C51M/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtatggtgaagagtggattcttttcttcaaaaatggtttgcagggatgtt

SEQ ID NO: 167 - DNA, 168 bp, Artificial sequence
  C41K/ C51E/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtaaagtgaagagtggattcttttcttcaaaagaagtttgcagggatgtt

SEQ ID NO: 168 - DNA, 168 bp, Artificial sequence
  C41E/ C51K/ D38A/ L42V DVP
gccaaagacggagacgtagaaggcccagcaggttgtaagaaatatgacgtagaatgtgactcgggggagtgttgtcagaagcaatacctttggtacaaatggagaccattggcttgtcgtgaagtgaagagtggattcttttcttcaaaaaaagtttgcagggatgtt

SEQ ID NO: 169 - DNA, 168 bp, Artificial sequence
  C41T/ C51A/ D20V/ D38A/ L42V DVP
gccaaagacggagacgtagaaggcccagcaggttgtaagaaatatgacgtagaatgtgtctcgggggagtgttgtcagaagcaatacctttggtacaaatggagaccattggcttgtcgtactgtgaagagtggattcttttcttcaaaagctgtttgcagggatgtt

SEQ ID NO: 170 - DNA, 168 bp, Artificial sequence
  C41T/ C51A/ D20G/ D38A/ L42V DVP
gccaaagacggagacgtagaaggcccagcaggttgtaagaaatatgacgtagaatgtggctcgggggagtgttgtcagaagcaatacctttggtacaaatggagaccattggcttgtcgtactgtgaagagtggattcttttcttcaaaagctgtttgcagggatgtt

SEQ ID NO: 171 - DNA, 168 bp, Artificial sequence
  C41T/ C51A/ D20K/ D38A/ L42V DVP
gccaaagacggagacgtagaaggcccagcaggttgtaagaaatatgacgtagaatgtaaatcgggggagtgttgtcagaagcaatacctttggtacaaatggagaccattggcttgtcgtactgtgaagagtggattcttttcttcaaaagctgtttgcagggatgtt

SEQ ID NO: 172 - DNA, 168 bp, Artificial sequence
  C41T/ C51A/ D20E/ D38A/ L42V DVP
gccaaagacggagacgtagaaggcccagcaggttgtaagaaatatgacgtagaatgtgaatcgggggagtgttgtcagaagcaatacctttggtacaaatggagaccattggcttgtcgtactgtgaagagtggattcttttcttcaaaagctgtttgcagggatgtt

SEQ ID NO: 173 - DNA, 168 bp, Artificial sequence
  C41T/ C51A/ D20L/ D38A/ L42V DVP
gccaaagacggagacgtagaaggcccagcaggttgtaagaaatatgacgtagaatgtttatcgggggagtgttgtcagaagcaatacctttggtacaaatggagaccattggcttgtcgtactgtgaagagtggattcttttcttcaaaagctgtttgcagggatgtt

SEQ ID NO: 174 - DNA, 168 bp, Artificial sequence
  C41T/ C51A/ D20N/ D38A/ L42V DVP
gccaaagacggagacgtagaaggcccagcaggttgtaagaaatatgacgtagaatgtaattcgggggagtgttgtcagaagcaatacctttggtacaaatggagaccattggcttgtcgtactgtgaagagtggattcttttcttcaaaagctgtttgcagggatgtt

SEQ ID NO: 175 - DNA, 168 bp, Artificial sequence
  C41T/ C51A/ D20Y/ D38A/ L42V DVP
gccaaagacggagacgtagaaggcccagcaggttgtaagaaatatgacgtagaatgttattcgggggagtgttgtcagaagcaatacctttggtacaaatggagaccattggcttgtcgtactgtgaagagtggattcttttcttcaaaagctgtttgcagggatgtt

SEQ ID NO: 176 - DNA, 168 bp, Artificial sequence
  C41T/ C51A/ S21G/ D38A/ L42V DVP
gccaaagacggagacgtagaaggcccagcaggttgtaagaaatatgacgtagaatgtgacggaggggagtgttgtcagaagcaatacctttggtacaaatggagaccattggcttgtcgtactgtgaagagtggattcttttcttcaaaagctgtttgcagggatgtt

SEQ ID NO: 177 - DNA, 168 bp, Artificial sequence
  C41T/ C51A/ E18P/ D38A/ L42V DVP
gccaaagacggagacgtagaaggcccagcaggttgtaagaaatatgacgtaccttgtgactcgggggagtgttgtcagaagcaatacctttggtacaaatggagaccattggcttgtcgtactgtgaagagtggattcttttcttcaaaagctgtttgcagggatgtt

SEQ ID NO: 178 - DNA, 168 bp, Artificial sequence
  C41T/ C51A/ E18K/ D38A/ L42V DVP
gccaaagacggagacgtagaaggcccagcaggttgtaagaaatatgacgtaaaatgtgactcgggggagtgttgtcagaagcaatacctttggtacaaatggagaccattggcttgtcgtactgtgaagagtggattcttttcttcaaaagctgtttgcagggatgtt

SEQ ID NO: 179 - DNA, 168 bp, Artificial sequence
  C41T/ C51A/ E18S/ D38A/ L42V DVP
gccaaagacggagacgtagaaggcccagcaggttgtaagaaatatgacgtatcttgtgactcgggggagtgttgtcagaagcaatacctttggtacaaatggagaccattggcttgtcgtactgtgaagagtggattcttttcttcaaaagctgtttgcagggatgtt

SEQ ID NO: 180 - DNA, 168 bp, Artificial sequence
  C41T/ C51A/ E18D/ D38A/ L42V DVP
gccaaagacggagacgtagaaggcccagcaggttgtaagaaatatgacgtagactgtgactcgggggagtgttgtcagaagcaatacctttggtacaaatggagaccattggcttgtcgtactgtgaagagtggattcttttcttcaaaagctgtttgcagggatgtt

SEQ ID NO: 181 - protein, 56 aa, Artificial sequence; identical to SEQ ID NO: 49
  C41V/ C51T/ D38A/ L42V DVP

SEQ ID NO: 182 - protein, 56 aa, Artificial sequence
  C41N/ C51A DVP
AKDGDVEGPAGCKKYDVECDSGECCQKQYLWYKWRPLDCRNLKSGFFSSKAVCRDV

SEQ ID NO: 183 - protein, 75 aa, Zea mays
QIFVKTLTGKTITLEVESSDTIDNVKAKIQDKEGIPPDQQRLIFAGKQLEDGRTLADYNIQKESTLHLVLRLRGG

SEQ ID NO: 184 - protein, 6 aa, Artificial sequence
  Linker
ALKFLV

SEQ ID NO: 185 - protein, 6 aa, Artificial sequence
  Histidine tag
HHHHHH

SEQ ID NO: 186 - protein, 167 aa, Artificial sequence
  DVP-insecticidal protein
MANKHLSLSLFLVLLGLSASLASGQIFVKTLTGKTITLEVESSDTIDNVKAKIQDKEGIPPDQQRLIFAGKQLEDGRTLADYNIQKESTLHLVLRLRGGALKFLVAKDGDVEGPAGCKKYDVECDSGECCQKQYLWYKWRPLDCRCLKSGFFSSKCVCRDVHHHHHH

SEQ ID NO: 187 - protein, 56 aa, Artificial sequence
  Y32S/ P36A DVP
AKDGDVEGPAGCKKYDVECDSGECCQKQYLWSKWRALDCRCLKSGFFSSKCVCRDV

SEQ ID NO: 188 - protein, 56 aa, Artificial sequence
  Y32K/ P36A DVP
AKDGDVEGPAGCKKYDVECDSGECCQKQYLWKKWRALDCRCLKSGFFSSKCVCRDV

SEQ ID NO: 189 - protein, 56 aa, Artificial sequence
  Y32H/ P36A DVP
AKDGDVEGPAGCKKYDVECDSGECCQKQYLWHKWRALDCRCLKSGFFSSKCVCRDV

SEQ ID NO: 190 - protein, 56 aa, Artificial sequence
  W31F/ Y32S DVP
AKDGDVEGPAGCKKYDVECDSGECCQKQYLFSKWRPLDCRCLKSGFFSSKCVCRDV

SEQ ID NO: 191 - protein, 56 aa, Artificial sequence
  W31F/ Y32S/ P36A DVP
AKDGDVEGPAGCKKYDVECDSGECCQKQYLFSKWRALDCRCLKSGFFSSKCVCRDV

SEQ ID NO: 192 - protein, 56 aa, Artificial sequence
  Y32H/ P36A/ C41A/ C51A DVP
AKDGDVEGPAGCKKYDVECDSGECCQKQYLWHKWRALDCRALKSGFFSSKAVCRDV

SEQ ID NO: 193 - protein, 37 aa, Apomastus schlingeri
CNSKGTPCTNADECCGGKCAYNVWNCIGGGCSKTCGY

SEQ ID NO: 194 - protein, 120 aa, Artificial sequence
  rWT AspII DNA
GGCAGTTGCAATTCAAAAGGTACTCCATGTACCAATGCTGATGAATGTTGTGGTGGTAAATGTGCATACAACGTTTGGAACTGTATTGGTGGAGGATGCTCTAAGACATGCGGGTATTAA

SEQ ID NO: 195 - protein, 39 aa, Artificial sequence
  rWT ApsIII
GSCNSKGTPCTNADECCGGKCAYNVWNCIGGGCSKTCGY

SEQ ID NO: 196 - protein, 120 aa, Artificial sequence
  rApsIII dCys DNA
GGCAGTTGCAATTCAAAAGGTACTCCATGTACCAATGCTGATGAATGTTGTGGTGGTAAATGTGCATACAACGTTTGGAACGCTATTGGTGGAGGAGCTTCTAAGACATGCGGGTATTAA

SEQ ID NO: 197 - protein, 39 aa, Artificial sequence
  rApsIII dCys
GSCNSKGTPCTNADECCGGKCAYNVWNAIGGGASKTCGY

SEQ ID NO: 198 - protein, 37 aa, Hadronyche versuta
AICTGADRPCAACCPCCPGTSCKAESNGVSYCRKDEP

SEQ ID NO: 199 - protein, 37 aa, Artificial sequence
  Kappa-ACTX C13A
AICTGADRPCAAACPCCPGTSCKAESNGVSYCRKDEP

SEQ ID NO: 200 - protein, 37 aa, Artificial sequence
  Kappa-ACTX C14A
AICTGADRPCAACAPCCPGTSCKAESNGVSYCRKDEP

SEQ ID NO: 201 - protein, 37 aa, Artificial sequence
  Kappa-ACTX C13A/ C14A
AICTGADRPCAAAAPCCPGTSCKAESNGVSYCRKDEP

SEQ ID NO: 202 - protein, 56 aa, Artificial sequence
  C41T/ C51A/ Y29A DVP
AKDGDVEGPAGCKKYDVECDSGECCQKQALWYKWRPLDCRTLKSGFFSSKAVCRDV

SEQ ID NO: 203 - protein, 56 aa, Artificial sequence
  C41T/ C51A/ G45A DVP
AKDGDVEGPAGCKKYDVECDSGECCQKQYLWYKWRPLDCRTLKSAFFSSKAVCRDV

SEQ ID NO: 204 - protein, 56 aa, Artificial sequence
  C41T/ C51A/ F47A DVP
AKDGDVEGPAGCKKYDVECDSGECCQKQYLWYKWRPLDCRTLKSGFASSKAVCRDV

SEQ ID NO: 205 - protein, 56 aa, Artificial sequence
  C41T/ C51A/ R54A DVP
AKDGDVEGPAGCKKYDVECDSGECCQKQYLWYKWRPLDCRTLKSGFFSSKAVCADV

SEQ ID NO: 206 - protein, 56 aa, Artificial sequence; identical to SEQ ID NO: 19
  C41T/ C51A/ Y32A DVP

SEQ ID NO: 207 - protein, 56 aa, Artificial sequence; identical to SEQ ID NO: 20
  C41T/ C51A/ P36A DVP

SEQ ID NO: 208 - protein, 56 aa, Artificial sequence
  C41T/ C51A/ D38A/ L42H DVP
AKDGDVEGPAGCKKYDVECASGECCQKQYLWYKWRPLDCRTHKSGFFSSKAVCRDV

SEQ ID NO: 209 - protein, 56 aa, Artificial sequence
  Y32S/ D38A/ C41S/ L42I/ C51S DVP
AKDGDVEGPAGCKKYDVECDSGECCQKQYLWSKWRPLACRSIKSGFFSSKSVCRDV

SEQ ID NO: 210 - protein, 56 aa, Artificial sequence
  D38A/ L42I/ C41S/ C51S DVP
AKDGDVEGPAGCKKYDVECDSGECCQKQYLWYKWRPLACRSIKSGFFSSKSVCRDV

SEQ ID NO: 211 - protein, 56 aa, Artificial sequence
  K2L/ D38A/ C41S/ C51S DVP
ALDGDVEGPAGCKKYDVECDSGECCQKQYLWYKWRPLACRSLKSGFFSSKSVCRDV

SEQ ID NO: 212 - protein, 56 aa, Artificial sequence
  Y32S/ D38A/ C41S/ C51S DVP
AKDGDVEGPAGCKKYDVECDSGECCQKQYLWSKWRPLACRSLKSGFFSSKSVCRDV

SEQ ID NO: 213 - protein, 56 aa, Artificial sequence
  K2L/ Y32S/ D38A/ C41S/ C51S DVP
ALDGDVEGPAGCKKYDVECDSGECCQKQYLWSKWRPLACRSLKSGFFSSKSVCRDV

SEQ ID NO: 214 - protein, 56 aa, Artificial sequence
  D38T/ C41S/ C51S DVP
AKDGDVEGPAGCKKYDVECDSGECCQKQYLWYKWRPLTCRSLKSGFFSSKSVCRDV

SEQ ID NO: 215 - protein, 56 aa, Artificial sequence
  D38S/ C41S/ C51S DVP
AKDGDVEGPAGCKKYDVECDSGECCQKQYLWYKWRPLSCRSLKSGFFSSKSVCRDV

SEQ ID NO: 216 - protein, 56 aa, Artificial sequence
  D38M/ C41S/ C51S DVP
AKDGDVEGPAGCKKYDVECDSGECCQKQYLWYKWRPLMCRSLKSGFFSSKSVCRDV

SEQ ID NO: 217 - protein, 56 aa, Artificial sequence
  K2L/ Y32S/ L42I DVP
ALDGDVEGPAGCKKYDVECDSGECCQKQYLWSKWRPLDCRCIKSGFFSSKCVCRDV

SEQ ID NO: 218 - protein, 56 aa, Artificial sequence
  K2L/ Y32S/ D38A/ L42I/ C41S/ C51S DVP
ALDGDVEGPAGCKKYDVECDSGECCQKQYLWSKWRPLACRSIKSGFFSSKSVCRDV

SEQ ID NO: 219 - protein, 56 aa, Artificial sequence
  K2L/ D38A/ L42I/ C41S/ C51S DVP
ALDGDVEGPAGCKKYDVECDSGECCQKQYLWYKWRPLACRSIKSGFFSSKSVCRDV

SEQ ID NO: 220 - DNA, 168 bp, Artificial sequence
  D38A/ L42I/ C41S/ C51S DVP
gctaaagatggtgatgttgaaggtccagctggttgtaaaaaatatgatgttgaatgtgattctggtgaatgttgtcaaaaacaatatttgtggtataaatggagaccattggcttgtagatctattaaatctggtttcttctcttctaaatctgtttgtagagatgtt

SEQ ID NO: 221 - DNA, 168 bp, Artificial sequence
  K2L/ D38A/ C41S/ C51S DVP
gctttggatggtgatgttgaaggtccagctggttgtaaaaaatatgatgttgaatgtgattctggtgaatgttgtcaaaaacaatatttgtggtataaatggagaccattggcttgtagatctttgaaatctggtttcttctcttctaaatctgtttgtagagatgtt

SEQ ID NO: 222 - DNA, 168 bp, Artificial sequence
  Y32S/ D38A/ C41S/ C51S DVP
gctaaagatggtgatgttgaaggtccagctggttgtaaaaaatatgatgttgaatgtgattctggtgaatgttgtcaaaaacaatatttgtggtctaaatggagaccattggcttgtagatctttgaaatctggtttcttctcttctaaatctgtttgtagagatgtt

SEQ ID NO: 223 - DNA, 168 bp, Artificial sequence
  K2L/ Y32S/ D38A/ C41S/ C51S DVP
gctttggatggtgatgttgaaggtccagctggttgtaaaaaatatgatgttgaatgtgattctggtgaatgttgtcaaaaacaatatttgtggtctaaatggagaccattggcttgtagatctttgaaatctggtttcttctcttctaaatctgtttgtagagatgtt

SEQ ID NO: 224 - DNA, 168 bp, Artificial sequence
  D38T/ C41S/ C51S DVP
gctaaagatggtgatgttgaaggtccagctggttgtaaaaaatatgatgttgaatgtgattctggtgaatgttgtcaaaaacaatatttgtggtataaatggagaccattgacttgtagatctttgaaatctggtttcttctcttctaaatctgtttgtagagatgtt

SEQ ID NO: 225 - DNA, 168 bp, Artificial sequence
  D38S/ C41S/ C51S DVP
gctaaagatggtgatgttgaaggtccagctggttgtaaaaaatatgatgttgaatgtgattctggtgaatgttgtcaaaaacaatatttgtggtataaatggagaccattgtcttgtagatctttgaaatctggtttcttctcttctaaatctgtttgtagagatgtt

SEQ ID NO: 226 - DNA, 168 bp, Artificial sequence
  D38M/ C41S/ C51S DVP
gctaaagatggtgatgttgaaggtccagctggttgtaaaaaatatgatgttgaatgtgattctggtgaatgttgtcaaaaacaatatttgtggtataaatggagaccattgatgtgtagatctttgaaatctggtttcttctcttctaaatctgtttgtagagatgtt

SEQ ID NO: 227 - DNA, 168 bp, Artificial sequence
  K2L/ Y32S/ L42I DVP
gctttggatggtgatgttgaaggtccagctggttgtaaaaaatatgatgttgaatgtgattctggtgaatgttgtcaaaaacaatatttgtggtctaaatggagaccattggattgtagatgtattaaatctggtttcttctcttctaaatgtgtttgtagagatgtt

SEQ ID NO: 228 - DNA, 168 bp, Artificial sequence
  K2L/ Y32S/ D38A/ L42I/ C41S/ C51S DVP
gctttggatggtgatgttgaaggtccagctggttgtaaaaaatatgatgttgaatgtgattctggtgaatgttgtcaaaaacaatatttgtggtctaaatggagaccattggcttgtagatctattaaatctggtttcttctcttctaaatctgtttgtagagatgtt

SEQ ID NO: 229 - DNA, 168 bp, Artificial sequence
  K2L/ D38A/ L42I/ C41S/ C51S DVP
gctttggatggtgatgttgaaggtccagctggttgtaaaaaatatgatgttgaatgtgattctggtgaatgttgtcaaaaacaatatttgtggtataaatggagaccattggcttgtagatctattaaatctggtttcttctcttctaaatctgtttgtagagatgtt

SEQ ID NO: 230 - DNA, 168 bp, Artificial sequence; identical to SEQ ID NO: 120
  C41V/ C51T/ D38A/ L42V DVP

SEQ ID NO: 231 - DNA, 168 bp, Artificial sequence
  C41N/ C51A DVP
gctaaagatggtgatgttgaaggtccagctggttgtaaaaaatatgatgttgaatgtgattctggtgaatgttgtcaaaaacaatatttgtggtataaatggagaccattggattgtagaaatttgaaatctggtttcttctcttctaaagctgtttgtagagatgtt

SEQ ID NO: 232 - DNA, 168 bp, Artificial sequence
  Y32S/ P36A DVP
gctaaagatggtgatgttgaaggtccagctggttgtaaaaaatatgatgttgaatgtgattctggtgaatgttgtcaaaaacaatatttgtggtctaaatggagagctttggattgtagatgtttgaaatctggtttcttctcttctaaatgtgtttgtagagatgtt

SEQ ID NO: 233 - DNA, 168 bp, Artificial sequence
  Y32K/ P36A DVP
gctaaagatggtgatgttgaaggtccagctggttgtaaaaaatatgatgttgaatgtgattctggtgaatgttgtcaaaaacaatatttgtggaaaaaatggagagctttggattgtagatgtttgaaatctggtttcttctcttctaaatgtgtttgtagagatgtt

SEQ ID NO: 234 - DNA, 168 bp, Artificial sequence
  Y32H/ P36A DVP
gctaaagatggtgatgttgaaggtccagctggttgtaaaaaatatgatgttgaatgtgattctggtgaatgttgtcaaaaacaatatttgtggcataaatggagagctttggattgtagatgtttgaaatctggtttcttctcttctaaatgtgtttgtagagatgtt

SEQ ID NO: 235 - DNA, 168 bp, Artificial sequence
  W31F/ Y32S DVP
gctaaagatggtgatgttgaaggtccagctggttgtaaaaaatatgatgttgaatgtgattctggtgaatgttgtcaaaaacaatatttgttctctaaatggagaccattggattgtagatgtttgaaatctggtttcttctcttctaaatgtgtttgtagagatgtt

SEQ ID NO: 236 - DNA, 168 bp, Artificial sequence
  W31F/ Y32S/ P36A DVP
gctaaagatggtgatgttgaaggtccagctggttgtaaaaaatatgatgttgaatgtgattctggtgaatgttgtcaaaaacaatatttgttctctaaatggagagctttggattgtagatgtttgaaatctggtttcttctcttctaaatgtgtttgtagagatgtt

SEQ ID NO: 237 - DNA, 168 bp, Artificial sequence
  Y32H/ P36A/ C41A/ C51A DVP
gctaaagatggtgatgttgaaggtccagctggttgtaaaaaatatgatgttgaatgtgattctggtgaatgttgtcaaaaacaatatttgtggcataaatggagagctttggattgtagagctttgaaatctggtttcttctcttctaaagctgtttgtagagatgtt

SEQ ID NO: 238 - DNA, 168 bp, Artificial sequence
  C41T/ C51A/ Y29A DVP
gctaaagatggtgatgttgaaggtccagctggttgtaaaaaatatgatgttgaatgtgattctggtgaatgttgtcaaaaacaagctttgtggtataaatggagaccattggattgtagaactttgaaatctggtttcttctcttctaaagctgtttgtagagatgtt

SEQ ID NO: 239 - DNA, 168 bp, Artificial sequence
  C41T/ C51A/ G45A DVP
gctaaagatggtgatgttgaaggtccagctggttgtaaaaaatatgatgttgaatgtgattctggtgaatgttgtcaaaaacaatatttgtggtataaatggagaccattggattgtagaactttgaaatctgctttcttctcttctaaagctgtttgtagagatgtt

SEQ ID NO: 240 - DNA, 168 bp, Artificial sequence
  C41T/ C51A/ F47A DVP
gctaaagatggtgatgttgaaggtccagctggttgtaaaaaatatgatgttgaatgtgattctggtgaatgttgtcaaaaacaatatttgtggtataaatggagaccattggattgtagaactttgaaatctggtttcgcttcttctaaagctgtttgtagagatgtt

SEQ ID NO: 241 - DNA, 168 bp, Artificial sequence
  C41T/ C51A/ R54A DVP
gctaaagatggtgatgttgaaggtccagctggttgtaaaaaatatgatgttgaatgtgattctggtgaatgttgtcaaaaacaatatttgtggtataaatggagaccattggattgtagaactttgaaatctggtttcttctcttctaaagctgtttgtgctgatgtt

SEQ ID NO: 242 - DNA, 168 bp, Artificial sequence; identical to SEQ ID NO: 90
  C41T/ C51A/ Y32A DVP

SEQ ID NO: 243 - DNA, 168 bp, Artificial sequence; identical to SEQ ID NO: 91
  C41T/ C51A/ P36A DVP

SEQ ID NO: 244 - DNA, 168 bp, Artificial sequence
  C41T/ C51A/ D38A/ L42H DVP
gctaaagatggtgatgttgaaggtccagctggttgtaaaaaatatgatgttgaatgtgcttctggtgaatgttgtcaaaaacaatatttgtggtataaatggagaccattggattgtagaactcataaatctggtttcttctcttctaaagctgtttgtagagatgtt

SEQ ID NO: 245 - DNA, 168 bp, Artificial sequence
  Y32S/ D38A/ C41S/ L42I/ C51S DVP
gctaaagatggtgatgttgaaggtccagctggttgtaaaaaatatgatgttgaatgtgattctggtgaatgttgtcaaaaacaatatttgtggtctaaatggagaccattggcttgtagatctattaaatctggtttcttctcttctaaatctgtttgtagagatgtt

SEQ ID NO: 246 - protein, 106 aa, Artificial sequence
  Kluyveromyces lactis alpha mating factor pre-pro secretion leader
MKFSTILAASTALISVVMAAPVSTETDIDDLPISVPEEALIGFIDLTGDEVSLLPVNNGTHTGILFLNTTIAEAAFADKDDLEKREAEARRARSPRGTVDGAPAAA

SEQ ID NO: 247 - protein, 187 aa, Artificial sequence
  Alpha-MF peptide
MKFSTILAASTALISVVMAAPVSTETDIDDLPISVPEEALIGFIDLTGDEVSLLPVNNGTHTGILFLNTTIAEAAFADKDDLKKREADASPWSWITLRPGQPIFKREANADANAEASPWSWITLRPGQPIFKREANADANADASPWSWITLRPGQPIFKREANPEAEADAKPSAWSWITLRPGQPIF

SEQ ID NO: 248 - protein, 160 aa, Artificial sequence
  Mf(alpha)1/Mf(alpha)2
MKFSTIFAASTALISVVMAAPVSTETDIDDLPISVPEEALIGFIDLTGDEVSLLPVNNGTHTGILFLNTTIAEAAFADKDDLTKREADASPWSWITLRPGQPIFKREANADANADASPWSWITLRPGQPIFKREASAEAEADAKPSAWSWITLRPGQPIF

SEQ ID NO: 249 - protein, 145 aa, Artificial sequence
  Mating factor alpha precursor N-terminus
MRLSAVFVSAIALLSTVIAAPITEKESDDSSIKVPSEAILGFLDLTADDDVGLVKINNGTHSGILFLNTTIASIAYANETILSKREASAEADPWKEASPEAEAEADPWKWLSFRVGQPIYKREASPEAEADPWKWLSFRIGQPIY